SEQUENCE LISTING (368 sequences)

SEQ ID NO: 1 - protein, 29 aa, Human
PTSSSTKKTQLQLEHLLLDLQMILNGINN

SEQ ID NO: 2 - protein, 46 aa, Human
YKNPKLTRMLTFKFYMPKKATELKHLQCLEEELKPLEEVLNLAQSK

SEQ ID NO: 3 - protein, 57 aa, Artificial Sequence
  SEQ C with C49S
NFHLRPRDLISNINVIVLELKGSETTFMCEYADETATIVEFLNRWITFSQSIISTLT

SEQ ID NO: 4 - protein, 57 aa, Human
NFHLRPRDLISNINVIVLELKGSETTFMCEYADETATIVEFLNRWITFCQSIISTLT

SEQ ID NO: 5 - protein, 153 aa, Human
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 6 - protein, 133 aa, Human [sequence omitted]

SEQ ID NO: 7 - DNA, 486 bp, Artificial Sequence
  DNA sequence for protein 1a
atggatatgcgggtccctgctcagctgctgggcctgctgctgctgtggtttcctgggtcaaggtgcgcccccacatctagttctacaaagaagacacaattacaattagagcacttattattagacttacaaatgatattaaatggcataaataattataagaatcccaagttaactttcggcaacagcaccaagttttatatgcctaagaaggctacagagttaaagcacttacaatgtttagaggaggagttaaagccattagaggaggtattaaatttagcccaatcaaagaattttcacttacggccccgggacttaatttccaatattaatgttattgtgttagagttaaagggctctgagacaacttttatgtgtgagtatgccgacgagacagccacaattgttgagttcttaaatcggtggattactttttcacaatccattatctccactttaactcatcatcaccatcaccactaa

SEQ ID NO: 8 - DNA, 486 bp, Artificial Sequence
  DNA sequence for protein 1b
atggatatgcgggtccctgctcagctgctgggcctgctgctgctgtggtttcctgggtcaaggtgcgcccccacatctagttctacaaagaagacacaattacaattagagcacttattattagacttacaaatgatattaaatggcataaataattataagaatcccaagttaactagaatgttattcggcaacagcaccatgcctaagaaggctacagagttaaagcacttacaatgtttagaggaggagttaaagccattagaggaggtattaaatttagcccaatcaaagaattttcacttacggccccgggacttaatttccaatattaatgttattgtgttagagttaaagggctctgagacaacttttatgtgtgagtatgccgacgagacagccacaattgttgagttcttaaatcggtggattactttttcacaatccattatctccactttaactcatcatcaccatcaccactaa

SEQ ID NO: 9 - protein, 132 aa, Artificial Sequence
  IL-2 variant with glycosylation motif
PTSSSTKKTQLQLEHLLLDLQMILNGINNYKNPKLTFGNSTKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 10 - protein, 133 aa, Artificial Sequence
  IL-2 variant with glycosylation motif
APTSSSTKKTQLQLEHLLLDLQMILNGINNYKNPKLTFGNSTKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 11 - protein, 46 aa, Artificial Sequence
  SEQ B with glycosylation motif
YKNPKLTFGNSTKFYMPKKATELKHLQCLEEELKPLEEVLNLAQSK

SEQ ID NO: 12 - protein, 132 aa, Artificial Sequence
  IL-2 variant with glycosylation motif
PTSSSTKKTQLQLEHLLLDLQMILNGINNYKNPKLTFANSTKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 13 - protein, 133 aa, Artificial Sequence
  IL-2 variant with glycosylation motif
APTSSSTKKTQLQLEHLLLDLQMILNGINNYKNPKLTFANSTKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 14 - protein, 46 aa, Artificial Sequence
  SEQ B with glycosylation motif
YKNPKLTFANSTKFYMPKKATELKHLQCLEEELKPLEEVLNLAQSK

SEQ ID NO: 15 - protein, 132 aa, Artificial Sequence
  IL-2 variant with glycosylation motif
PTSSSTKKTQLQLEHLLLDLQMILNGINNYKNPKLTRMLFGNSTMPKKATELKHLQCLEEELKPLEEVLNLAQSKNFHLRPRDLISNINVIVLELKGSETTFMCEYADETATIVEFLNRWITFSQSIISTLT

SEQ ID NO: 16 - protein, 133 aa, Artificial Sequence
  IL-2 variant with glycosylation motif
APTSSSTKKTQLQLEHLLLDLQMILNGINNYKNPKLTRMLFGNSTMPKKATELKHLQCLEEELKPLEEVLNLAQSKNFHLRPRDLISNINVIVLELKGSETTFMCEYADETATIVEFLNRWITFSQSIISTLT

SEQ ID NO: 17 - protein, 46 aa, Artificial Sequence
  SEQ B with glycosylation motif
YKNPKLTRMLFGNSTMPKKATELKHLQCLEEELKPLEEVLNLAQSK

SEQ ID NO: 18 - protein, 132 aa, Artificial Sequence
  IL-2 variant with glycosylation motif
PTSSSTKKTQLQLEHLLLDLQMILNGINNYKNPKLTRMLFANSTMPKKATELKHLQCLEEELKPLEEVLNLAQSKNFHLRPRDLISNINVIVLELKGSETTFMCEYADETATIVEFLNRWITFSQSIISTLT

SEQ ID NO: 19 - protein, 133 aa, Artificial Sequence
  IL-2 variant with glycosylation motif
APTSSSTKKTQLQLEHLLLDLQMILNGINNYKNPKLTRMLFANSTMPKKATELKHLQCLEEELKPLEEVLNLAQSKNFHLRPRDLISNINVIVLELKGSETTFMCEYADETATIVEFLNRWITFSQSIISTLT

SEQ ID NO: 20 - protein, 46 aa, Artificial Sequence
  SEQ B with glycosylation motif
YKNPKLTRMLFANSTMPKKATELKHLQCLEEELKPLEEVLNLAQSK

SEQ ID NO: 21 - protein, 132 aa, Artificial Sequence
  IL-2 variant with glycosylation motif
PTSSSTKKTQLQLEHLLLDLQMILNGINNYKNPKLTRMLTFKFYMPKKATELKHLQCLEFGNSTLEEVLNLAQSKNFHLRPRDLISNINVIVLELKGSETTFMCEYADETATIVEFLNRWITFSQSIISTLT

SEQ ID NO: 22 - protein, 133 aa, Artificial Sequence
  IL-2 variant with glycosylation motif
APTSSSTKKTQLQLEHLLLDLQMILNGINNYKNPKLTRMLTFKFYMPKKATELKHLQCLEFGNSTLEEVLNLAQSKNFHLRPRDLISNINVIVLELKGSETTFMCEYADETATIVEFLNRWITFSQSIISTLT

SEQ ID NO: 23 - protein, 46 aa, Artificial Sequence
  SEQ B with glycosylation motif
YKNPKLTRMLTFKFYMPKKATELKHLQCLEFGNSTLEEVLNLAQSK

SEQ ID NO: 24 - protein, 132 aa, Artificial Sequence
  IL-2 variant with glycosylation motif
PTSSSTKKTQLQLEHLLLDLQMILNGINNYKNPKLTRMLTFKFYMPKKATELKHLQCLEFANSTLEEVLNLAQSKNFHLRPRDLISNINVIVLELKGSETTFMCEYADETATIVEFLNRWITFSQSIISTLT

SEQ ID NO: 25 - protein, 133 aa, Artificial Sequence
  IL-2 variant with glycosylation motif
APTSSSTKKTQLQLEHLLLDLQMILNGINNYKNPKLTRMLTFKFYMPKKATELKHLQCLEFANSTLEEVLNLAQSKNFHLRPRDLISNINVIVLELKGSETTFMCEYADETATIVEFLNRWITFSQSIISTLT

SEQ ID NO: 26 - protein, 46 aa, Artificial Sequence
  SEQ B with glycosylation motif
YKNPKLTRMLTFKFYMPKKATELKHLQCLEFANSTLEEVLNLAQSK

SEQ ID NO: 27 - protein, 133 aa, Artificial Sequence
  IL-2 variant with glycosylation motif
APTSSSTKKTQLQLEHLLLDLQMILNGINNYKNPELTFGNSTKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 28 - protein, 132 aa, Artificial Sequence
  IL-2 variant with glycosylation motif
PASSSTKKTQLQLEHLLLDLQMILNGINNYKNPELTFGNSTKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 29 - protein, 133 aa, Artificial Sequence
  IL-2 variant with glycosylation motif
APASSSTKKTQLQLEHLLLDLQMILNGINNYKNPELTFGNSTKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 30 - protein, 132 aa, Artificial Sequence
  IL-2 variant with glycosylation motif
PTSSSTKKTQLQLEHLLLDLQMILNGINNYKNPKLTRMLTFKFYMPKKATELKHLQCLEEELKPLEEFANSTQSKNFHLRPRDLISNINVIVLELKGSETTFMCEYADETATIVEFLNRWITFSQSIISTLT

SEQ ID NO: 31 - protein, 133 aa, Artificial Sequence
  IL-2 variant with glycosylation motif
APTSSSTKKTQLQLEHLLLDLQMILNGINNYKNPKLTRMLTFKFYMPKKATELKHLQCLEEELKPLEEFANSTQSKNFHLRPRDLISNINVIVLELKGSETTFMCEYADETATIVEFLNRWITFSQSIISTLT

SEQ ID NO: 32 - protein, 46 aa, Artificial Sequence
  SEQ B with glycosylation motif
YKNPKLTRMLTFKFYMPKKATELKHLQCLEEELKPLEEFANSTQSK

SEQ ID NO: 33 - protein, 132 aa, Artificial Sequence
  IL-2 variant with glycosylation motif
PTSSSTKKTQLQLEHLLLDLQMILNGINNYKNPKLTRMLTFKFYMPKKATELKHLQCLEEELKPLEEFGNSTQSKNFHLRPRDLISNINVIVLELKGSETTFMCEYADETATIVEFLNRWITFSQSIISTLT

SEQ ID NO: 34 - protein, 133 aa, Artificial Sequence
  IL-2 variant with glycosylation motif
APTSSSTKKTQLQLEHLLLDLQMILNGINNYKNPKLTRMLTFKFYMPKKATELKHLQCLEEELKPLEEFGNSTQSKNFHLRPRDLISNINVIVLELKGSETTFMCEYADETATIVEFLNRWITFSQSIISTLT

SEQ ID NO: 35 - protein, 46 aa, Artificial Sequence
  SEQ B with glycosylation motif
YKNPKLTRMLTFKFYMPKKATELKHLQCLEEELKPLEEFGNSTQSK

SEQ ID NO: 36 - protein, 29 aa, Artificial Sequence
  SEQ B with glycosylation motif
PASSSTKKTQLQLEHLLLDLQMILNGINN

SEQ ID NO: 37 - protein, 132 aa, Artificial Sequence
  IL-2 variant with glycosylation motif
PASSSTKKTQLQLEHLLLDLQMILNGINNYKNPKLTFGNSTKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 38 - protein, 133 aa, Artificial Sequence
  IL-2 variant with glycosylation motif
APASSSTKKTQLQLEHLLLDLQMILNGINNYKNPKLTFGNSTKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 39 - protein, 132 aa, Artificial Sequence
  IL-2 variant with glycosylation motif
PTSSSTKKTQLQLEHLLLDLQMILNGINNYKNPKLTFGNSTKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 40 - protein, 133 aa, Artificial Sequence
  IL-2 variant with glycosylation motif
APTSSSTKKTQLQLEHLLLDLQMILNGINNYKNPKLTFGNSTKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 41 - protein, 132 aa, Artificial Sequence
  IL-2 variant with glycosylation motif
PASSSTKKTQLQLEHLLLDLQMILNGINNYKNPKLTFGNSTKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 42 - protein, 133 aa, Artificial Sequence
  IL-2 variant with glycosylation motif
APASSSTKKTQLQLEHLLLDLQMILNGINNYKNPKLTFGNSTKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 43 - protein, 132 aa, Artificial Sequence
  IL-2 variant with glycosylation motif
PASSSTKKTQLQLEHLLLDLQMILNGINNYKNPKLTFANSTKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 44 - protein, 133 aa, Artificial Sequence
  IL-2 variant with glycosylation motif
APASSSTKKTQLQLEHLLLDLQMILNGINNYKNPKLTFANSTKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 45 - protein, 132 aa, Artificial Sequence
  IL-2 variant with glycosylation motif
PTSSSTKKTQLQLEHLLLDLQMILNGINNYKNPKLTFANSTKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 46 - protein, 133 aa, Artificial Sequence
  IL-2 variant with glycosylation motif
APTSSSTKKTQLQLEHLLLDLQMILNGINNYKNPKLTFANSTKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 47 - protein, 132 aa, Artificial Sequence
  IL-2 variant with glycosylation motif
PASSSTKKTQLQLEHLLLDLQMILNGINNYKNPKLTFANSTKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 48 - protein, 133 aa, Artificial Sequence
  IL-2 variant with glycosylation motif
APASSSTKKTQLQLEHLLLDLQMILNGINNYKNPKLTFANSTKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 49 - protein, 132 aa, Artificial Sequence
  IL-2 variant with glycosylation motif
PASSSTKKTQLQLEHLLLDLQMILNGINNYKNPKLTRMLFGNSTMPKKATELKHLQCLEEELKPLEEVLNLAQSKNFHLRPRDLISNINVIVLELKGSETTFMCEYADETATIVEFLNRWITFSQSIISTLT

SEQ ID NO: 50 - protein, 133 aa, Artificial Sequence
  IL-2 variant with glycosylation motif
APASSSTKKTQLQLEHLLLDLQMILNGINNYKNPKLTRMLFGNSTMPKKATELKHLQCLEEELKPLEEVLNLAQSKNFHLRPRDLISNINVIVLELKGSETTFMCEYADETATIVEFLNRWITFSQSIISTLT

SEQ ID NO: 51 - protein, 132 aa, Artificial Sequence
  IL-2 variant with glycosylation motif
PTSSSTKKTQLQLEHLLLDLQMILNGINNYKNPKLTRMLFGNSTMPKKATELKHLQCLEEELKPLEEVLNLAQSKNFHLRPRDLISNINVIVLELKGSETTFMCEYADETATIVEFLNRWITFCQSIISTLT

SEQ ID NO: 52 - protein, 133 aa, Artificial Sequence
  IL-2 variant with glycosylation motif
APTSSSTKKTQLQLEHLLLDLQMILNGINNYKNPKLTRMLFGNSTMPKKATELKHLQCLEEELKPLEEVLNLAQSKNFHLRPRDLISNINVIVLELKGSETTFMCEYADETATIVEFLNRWITFCQSIISTLT

SEQ ID NO: 53 - protein, 132 aa, Artificial Sequence
  IL-2 variant with glycosylation motif
PASSSTKKTQLQLEHLLLDLQMILNGINNYKNPKLTRMLFGNSTMPKKATELKHLQCLEEELKPLEEVLNLAQSKNFHLRPRDLISNINVIVLELKGSETTFMCEYADETATIVEFLNRWITFCQSIISTLT

SEQ ID NO: 54 - protein, 133 aa, Artificial Sequence
  IL-2 variant with glycosylation motif
APASSSTKKTQLQLEHLLLDLQMILNGINNYKNPKLTRMLFGNSTMPKKATELKHLQCLEEELKPLEEVLNLAQSKNFHLRPRDLISNINVIVLELKGSETTFMCEYADETATIVEFLNRWITFCQSIISTLT

SEQ ID NO: 55 - protein, 132 aa, Artificial Sequence
  IL-2 variant with glycosylation motif
PASSSTKKTQLQLEHLLLDLQMILNGINNYKNPKLTRMLFANSTMPKKATELKHLQCLEEELKPLEEVLNLAQSKNFHLRPRDLISNINVIVLELKGSETTFMCEYADETATIVEFLNRWITFSQSIISTLT

SEQ ID NO: 56 - protein, 133 aa, Artificial Sequence
  IL-2 variant with glycosylation motif
APASSSTKKTQLQLEHLLLDLQMILNGINNYKNPKLTRMLFANSTMPKKATELKHLQCLEEELKPLEEVLNLAQSKNFHLRPRDLISNINVIVLELKGSETTFMCEYADETATIVEFLNRWITFSQSIISTLT

SEQ ID NO: 57 - protein, 132 aa, Artificial Sequence
  IL-2 variant with glycosylation motif
PTSSSTKKTQLQLEHLLLDLQMILNGINNYKNPKLTRMLFANSTMPKKATELKHLQCLEEELKPLEEVLNLAQSKNFHLRPRDLISNINVIVLELKGSETTFMCEYADETATIVEFLNRWITFCQSIISTLT

SEQ ID NO: 58 - protein, 133 aa, Artificial Sequence
  IL-2 variant with glycosylation motif
APTSSSTKKTQLQLEHLLLDLQMILNGINNYKNPKLTRMLFANSTMPKKATELKHLQCLEEELKPLEEVLNLAQSKNFHLRPRDLISNINVIVLELKGSETTFMCEYADETATIVEFLNRWITFCQSIISTLT

SEQ ID NO: 59 - protein, 132 aa, Artificial Sequence
  IL-2 variant with glycosylation motif
PASSSTKKTQLQLEHLLLDLQMILNGINNYKNPKLTRMLFANSTMPKKATELKHLQCLEEELKPLEEVLNLAQSKNFHLRPRDLISNINVIVLELKGSETTFMCEYADETATIVEFLNRWITFCQSIISTLT

SEQ ID NO: 60 - protein, 133 aa, Artificial Sequence
  IL-2 variant with glycosylation motif
APASSSTKKTQLQLEHLLLDLQMILNGINNYKNPKLTRMLFANSTMPKKATELKHLQCLEEELKPLEEVLNLAQSKNFHLRPRDLISNINVIVLELKGSETTFMCEYADETATIVEFLNRWITFCQSIISTLT

SEQ ID NO: 61 - protein, 132 aa, Artificial Sequence
  IL-2 variant with glycosylation motif
PASSSTKKTQLQLEHLLLDLQMILNGINNYKNPKLTRMLTFKFYMPKKATELKHLQCLEFGNSTLEEVLNLAQSKNFHLRPRDLISNINVIVLELKGSETTFMCEYADETATIVEFLNRWITFSQSIISTLT

SEQ ID NO: 62 - protein, 133 aa, Artificial Sequence
  IL-2 variant with glycosylation motif
APASSSTKKTQLQLEHLLLDLQMILNGINNYKNPKLTRMLTFKFYMPKKATELKHLQCLEFGNSTLEEVLNLAQSKNFHLRPRDLISNINVIVLELKGSETTFMCEYADETATIVEFLNRWITFSQSIISTLT

SEQ ID NO: 63 - protein, 132 aa, Artificial Sequence
  IL-2 variant with glycosylation motif
PTSSSTKKTQLQLEHLLLDLQMILNGINNYKNPKLTRMLTFKFYMPKKATELKHLQCLEFGNSTLEEVLNLAQSKNFHLRPRDLISNINVIVLELKGSETTFMCEYADETATIVEFLNRWITFCQSIISTLT

SEQ ID NO: 64 - protein, 133 aa, Artificial Sequence
  IL-2 variant with glycosylation motif
APTSSSTKKTQLQLEHLLLDLQMILNGINNYKNPKLTRMLTFKFYMPKKATELKHLQCLEFGNSTLEEVLNLAQSKNFHLRPRDLISNINVIVLELKGSETTFMCEYADETATIVEFLNRWITFCQSIISTLT

SEQ ID NO: 65 - protein, 132 aa, Artificial Sequence
  IL-2 variant with glycosylation motif
PASSSTKKTQLQLEHLLLDLQMILNGINNYKNPKLTRMLTFKFYMPKKATELKHLQCLEFGNSTLEEVLNLAQSKNFHLRPRDLISNINVIVLELKGSETTFMCEYADETATIVEFLNRWITFCQSIISTLT

SEQ ID NO: 66 - protein, 133 aa, Artificial Sequence
  IL-2 variant with glycosylation motif
APASSSTKKTQLQLEHLLLDLQMILNGINNYKNPKLTRMLTFKFYMPKKATELKHLQCLEFGNSTLEEVLNLAQSKNFHLRPRDLISNINVIVLELKGSETTFMCEYADETATIVEFLNRWITFCQSIISTLT

SEQ ID NO: 67 - protein, 132 aa, Artificial Sequence
  IL-2 variant with glycosylation motif
PASSSTKKTQLQLEHLLLDLQMILNGINNYKNPKLTRMLTFKFYMPKKATELKHLQCLEFANSTLEEVLNLAQSKNFHLRPRDLISNINVIVLELKGSETTFMCEYADETATIVEFLNRWITFSQSIISTLT

SEQ ID NO: 68 - protein, 133 aa, Artificial Sequence
  IL-2 variant with glycosylation motif
APASSSTKKTQLQLEHLLLDLQMILNGINNYKNPKLTRMLTFKFYMPKKATELKHLQCLEFANSTLEEVLNLAQSKNFHLRPRDLISNINVIVLELKGSETTFMCEYADETATIVEFLNRWITFSQSIISTLT

SEQ ID NO: 69 - protein, 132 aa, Artificial Sequence
  IL-2 variant with glycosylation motif
PTSSSTKKTQLQLEHLLLDLQMILNGINNYKNPKLTRMLTFKFYMPKKATELKHLQCLEFANSTLEEVLNLAQSKNFHLRPRDLISNINVIVLELKGSETTFMCEYADETATIVEFLNRWITFCQSIISTLT

SEQ ID NO: 70 - protein, 133 aa, Artificial Sequence
  IL-2 variant with glycosylation motif
APTSSSTKKTQLQLEHLLLDLQMILNGINNYKNPKLTRMLTFKFYMPKKATELKHLQCLEFANSTLEEVLNLAQSKNFHLRPRDLISNINVIVLELKGSETTFMCEYADETATIVEFLNRWITFCQSIISTLT

SEQ ID NO: 71 - protein, 132 aa, Artificial Sequence
  IL-2 variant with glycosylation motif
PASSSTKKTQLQLEHLLLDLQMILNGINNYKNPKLTRMLTFKFYMPKKATELKHLQCLEFANSTLEEVLNLAQSKNFHLRPRDLISNINVIVLELKGSETTFMCEYADETATIVEFLNRWITFCQSIISTLT

SEQ ID NO: 72 - protein, 133 aa, Artificial Sequence
  IL-2 variant with glycosylation motif
APASSSTKKTQLQLEHLLLDLQMILNGINNYKNPKLTRMLTFKFYMPKKATELKHLQCLEFANSTLEEVLNLAQSKNFHLRPRDLISNINVIVLELKGSETTFMCEYADETATIVEFLNRWITFCQSIISTLT

SEQ ID NO: 73 - protein, 132 aa, Artificial Sequence
  IL-2 variant with glycosylation motif
PASSSTKKTQLQLEHLLLDLQMILNGINNYKNPKLTRMLTFKFYMPKKATELKHLQCLEEELKPLEEFANSTQSKNFHLRPRDLISNINVIVLELKGSETTFMCEYADETATIVEFLNRWITFSQSIISTLT

SEQ ID NO: 74 - protein, 133 aa, Artificial Sequence
  IL-2 variant with glycosylation motif
APASSSTKKTQLQLEHLLLDLQMILNGINNYKNPKLTRMLTFKFYMPKKATELKHLQCLEEELKPLEEFANSTQSKNFHLRPRDLISNINVIVLELKGSETTFMCEYADETATIVEFLNRWITFSQSIISTLT

SEQ ID NO: 75 - protein, 132 aa, Artificial Sequence
  IL-2 variant with glycosylation motif
PTSSSTKKTQLQLEHLLLDLQMILNGINNYKNPKLTRMLTFKFYMPKKATELKHLQCLEEELKPLEEFANSTQSKNFHLRPRDLISNINVIVLELKGSETTFMCEYADETATIVEFLNRWITFCQSIISTLT

SEQ ID NO: 76 - protein, 133 aa, Artificial Sequence
  IL-2 variant with glycosylation motif
APTSSSTKKTQLQLEHLLLDLQMILNGINNYKNPKLTRMLTFKFYMPKKATELKHLQCLEEELKPLEEFANSTQSKNFHLRPRDLISNINVIVLELKGSETTFMCEYADETATIVEFLNRWITFCQSIISTLT

SEQ ID NO: 77 - protein, 132 aa, Artificial Sequence
  IL-2 variant with glycosylation motif
PASSSTKKTQLQLEHLLLDLQMILNGINNYKNPKLTRMLTFKFYMPKKATELKHLQCLEEELKPLEEFANSTQSKNFHLRPRDLISNINVIVLELKGSETTFMCEYADETATIVEFLNRWITFCQSIISTLT

SEQ ID NO: 78 - protein, 133 aa, Artificial Sequence
  IL-2 variant with glycosylation motif
APASSSTKKTQLQLEHLLLDLQMILNGINNYKNPKLTRMLTFKFYMPKKATELKHLQCLEEELKPLEEFANSTQSKNFHLRPRDLISNINVIVLELKGSETTFMCEYADETATIVEFLNRWITFCQSIISTLT

SEQ ID NO: 79 - protein, 132 aa, Artificial Sequence
  IL-2 variant with glycosylation motif
PASSSTKKTQLQLEHLLLDLQMILNGINNYKNPKLTRMLTFKFYMPKKATELKHLQCLEEELKPLEEFGNSTQSKNFHLRPRDLISNINVIVLELKGSETTFMCEYADETATIVEFLNRWITFSQSIISTLT

SEQ ID NO: 80 - protein, 133 aa, Artificial Sequence
  IL-2 variant with glycosylation motif
APASSSTKKTQLQLEHLLLDLQMILNGINNYKNPKLTRMLTFKFYMPKKATELKHLQCLEEELKPLEEFGNSTQSKNFHLRPRDLISNINVIVLELKGSETTFMCEYADETATIVEFLNRWITFSQSIISTLT

SEQ ID NO: 81 - protein, 132 aa, Artificial Sequence
  IL-2 variant with glycosylation motif
PTSSSTKKTQLQLEHLLLDLQMILNGINNYKNPKLTRMLTFKFYMPKKATELKHLQCLEEELKPLEEFGNSTQSKNFHLRPRDLISNINVIVLELKGSETTFMCEYADETATIVEFLNRWITFCQSIISTLT

SEQ ID NO: 82 - protein, 133 aa, Artificial Sequence
  IL-2 variant with glycosylation motif
APTSSSTKKTQLQLEHLLLDLQMILNGINNYKNPKLTRMLTFKFYMPKKATELKHLQCLEEELKPLEEFGNSTQSKNFHLRPRDLISNINVIVLELKGSETTFMCEYADETATIVEFLNRWITFCQSIISTLT

SEQ ID NO: 83 - protein, 132 aa, Artificial Sequence
  IL-2 variant with glycosylation motif
PASSSTKKTQLQLEHLLLDLQMILNGINNYKNPKLTRMLTFKFYMPKKATELKHLQCLEEELKPLEEFGNSTQSKNFHLRPRDLISNINVIVLELKGSETTFMCEYADETATIVEFLNRWITFCQSIISTLT

SEQ ID NO: 84 - protein, 133 aa, Artificial Sequence
  IL-2 variant with glycosylation motif
APASSSTKKTQLQLEHLLLDLQMILNGINNYKNPKLTRMLTFKFYMPKKATELKHLQCLEEELKPLEEFGNSTQSKNFHLRPRDLISNINVIVLELKGSETTFMCEYADETATIVEFLNRWITFCQSIISTLT

SEQ ID NO: 85 - protein, 46 aa, Artificial Sequence
  SEQ B with glycosylation motif
  MISC_FEATURE (2)..(2): any proteinogenic or non-proteinogenic amino acid except proline
  MISC_FEATURE (3)..(3): T, S or C
NXXPKLTRMLTFKFYMPKKATELKHLQCLEEELKPLEEVLNLAQSK

SEQ ID NO: 86 - protein, 46 aa, Artificial Sequence
  SEQ B with glycosylation motif
  MISC_FEATURE (3)..(3): any proteinogenic or non-proteinogenic amino acid except proline
  MISC_FEATURE (4)..(4): T, S or C
YNXXKLTRMLTFKFYMPKKATELKHLQCLEEELKPLEEVLNLAQSK

SEQ ID NO: 87 - protein, 46 aa, Artificial Sequence
  SEQ B with glycosylation motif
  MISC_FEATURE (4)..(4): any proteinogenic or non-proteinogenic amino acid except proline
  MISC_FEATURE (5)..(5): T, S or C
YKNXXLTRMLTFKFYMPKKATELKHLQCLEEELKPLEEVLNLAQSK

SEQ ID NO: 88 - protein, 46 aa, Artificial Sequence
  SEQ B with glycosylation motif
  misc_feature (5)..(5): any proteinogenic or non-proteinogenic amino acid except proline
  misc_feature (6)..(6): T, S or C
YKNNXXTRMLTFKFYMPKKATELKHLQCLEEELKPLEEVLNLAQSK

SEQ ID NO: 89 - protein, 46 aa, Artificial Sequence
  SEQ B with glycosylation motif
  MISC_FEATURE (6)..(6): any proteinogenic or non-proteinogenic amino acid except proline
  MISC_FEATURE (7)..(7): T, S or C
YKNPNXXRMLTFKFYMPKKATELKHLQCLEEELKPLEEVLNLAQSK

SEQ ID NO: 90 - protein, 46 aa, Artificial Sequence
  SEQ B with glycosylation motif
  MISC_FEATURE (7)..(7): any proteinogenic or non-proteinogenic amino acid except proline
  MISC_FEATURE (8)..(8): T, S or C
YKNPKNXXMLTFKFYMPKKATELKHLQCLEEELKPLEEVLNLAQSK

SEQ ID NO: 91 - protein, 46 aa, Artificial Sequence
  SEQ B with glycosylation motif
  MISC_FEATURE (8)..(8): any proteinogenic or non-proteinogenic amino acid except proline
  MISC_FEATURE (9)..(9): T, S or C
YKNPKLNXXLTFKFYMPKKATELKHLQCLEEELKPLEEVLNLAQSK

SEQ ID NO: 92 - protein, 46 aa, Artificial Sequence
  SEQ B with glycosylation motif
  MISC_FEATURE (9)..(9): any proteinogenic or non-proteinogenic amino acid except proline
  MISC_FEATURE (10)..(10): T, S or C
YKNPKLTNXXTFKFYMPKKATELKHLQCLEEELKPLEEVLNLAQSK

SEQ ID NO: 93 - protein, 46 aa, Artificial Sequence
  SEQ B with glycosylation motif
  MISC_FEATURE (10)..(10): any proteinogenic or non-proteinogenic amino acid except proline
  MISC_FEATURE (11)..(11): T, S or C
YKNPKLTRNXXFKFYMPKKATELKHLQCLEEELKPLEEVLNLAQSK

SEQ ID NO: 94 - protein, 46 aa, Artificial Sequence
  SEQ B with glycosylation motif
  misc_feature (11)..(11): any proteinogenic or non-proteinogenic amino acid except proline
  misc_feature (12)..(12): T, S or C
YKNPKLTRMNXXKFYMPKKATELKHLQCLEEELKPLEEVLNLAQSK

SEQ ID NO: 95 - protein, 46 aa, Artificial Sequence
  SEQ B with glycosylation motif
  MISC_FEATURE (12)..(12): any proteinogenic or non-proteinogenic amino acid except proline
  MISC_FEATURE (13)..(13): T, S or C
YKNPKLTRMLNXXFYMPKKATELKHLQCLEEELKPLEEVLNLAQSK

SEQ ID NO: 96 - protein, 46 aa, Artificial Sequence
  SEQ B with glycosylation motif
  MISC_FEATURE (13)..(13): any proteinogenic or non-proteinogenic amino acid except proline
  MISC_FEATURE (14)..(14): T, S or C
YKNPKLTRMLTNXXYMPKKATELKHLQCLEEELKPLEEVLNLAQSK

SEQ ID NO: 97 - protein, 46 aa, Artificial Sequence
  SEQ B with glycosylation motif
  MISC_FEATURE (14)..(14): any proteinogenic or non-proteinogenic amino acid except proline
  MISC_FEATURE (15)..(15): T, S or C
YKNPKLTRMLTFNXXMPKKATELKHLQCLEEELKPLEEVLNLAQSK

SEQ ID NO: 98 - protein, 46 aa, Artificial Sequence
  SEQ B with glycosylation motif
  MISC_FEATURE (15)..(15): any proteinogenic or non-proteinogenic amino acid except proline
  MISC_FEATURE (16)..(16): T, S or C
YKNPKLTRMLTFKNXXPKKATELKHLQCLEEELKPLEEVLNLAQSK

SEQ ID NO: 99 - protein, 46 aa, Artificial Sequence
  SEQ B with glycosylation motif
  MISC_FEATURE (16)..(16): any proteinogenic or non-proteinogenic amino acid except proline
  MISC_FEATURE (17)..(17): T, S or C
YKNPKLTRMLTFKFNXXKKATELKHLQCLEEELKPLEEVLNLAQSK

SEQ ID NO: 100 - protein, 46 aa, Artificial Sequence
  SEQ B with glycosylation motif
  MISC_FEATURE (17)..(17): any proteinogenic or non-proteinogenic amino acid except proline
  MISC_FEATURE (18)..(18): T, S or C
YKNPKLTRMLTFKFYNXXKATELKHLQCLEEELKPLEEVLNLAQSK

SEQ ID NO: 101 - protein, 46 aa, Artificial Sequence
  SEQ B with glycosylation motif
  MISC_FEATURE (18)..(18): any proteinogenic or non-proteinogenic amino acid except proline
  MISC_FEATURE (19)..(19): T, S or C
YKNPKLTRMLTFKFYMNXXATELKHLQCLEEELKPLEEVLNLAQSK

SEQ ID NO: 102 - protein, 46 aa, Artificial Sequence
  SEQ B with glycosylation motif
  MISC_FEATURE (19)..(19): any proteinogenic or non-proteinogenic amino acid except proline
  MISC_FEATURE (20)..(20): T, S or C
YKNPKLTRMLTFKFYMPNXXTELKHLQCLEEELKPLEEVLNLAQSK

SEQ ID NO: 103 - protein, 46 aa, Artificial Sequence
  SEQ B with glycosylation motif
  MISC_FEATURE (20)..(20): any proteinogenic or non-proteinogenic amino acid except proline
  MISC_FEATURE (21)..(21): T, S or C
YKNPKLTRMLTFKFYMPKNXXELKHLQCLEEELKPLEEVLNLAQSK

SEQ ID NO: 104 - protein, 46 aa, Artificial Sequence
  SEQ B with glycosylation motif
  MISC_FEATURE (21)..(21): any proteinogenic or non-proteinogenic amino acid except proline
  MISC_FEATURE (22)..(22): T, S or C
YKNPKLTRMLTFKFYMPKKNXXLKHLQCLEEELKPLEEVLNLAQSK

SEQ ID NO: 105 - protein, 46 aa, Artificial Sequence
  SEQ B with glycosylation motif
  MISC_FEATURE (22)..(22): any proteinogenic or non-proteinogenic amino acid except proline
  MISC_FEATURE (23)..(23): T, S or C
YKNPKLTRMLTFKFYMPKKANXXKHLQCLEEELKPLEEVLNLAQSK

SEQ ID NO: 106 - protein, 46 aa, Artificial Sequence
  SEQ B with glycosylation motif
  MISC_FEATURE (23)..(23): any proteinogenic or non-proteinogenic amino acid except proline
  MISC_FEATURE (24)..(24): T, S or C
YKNPKLTRMLTFKFYMPKKATNXXHLQCLEEELKPLEEVLNLAQSK

SEQ ID NO: 107 - protein, 46 aa, Artificial Sequence
  SEQ B with glycosylation motif
  MISC_FEATURE (24)..(24): any proteinogenic or non-proteinogenic amino acid except proline
  MISC_FEATURE (25)..(25): T, S or C
YKNPKLTRMLTFKFYMPKKATENXXLQCLEEELKPLEEVLNLAQSK

SEQ ID NO: 108 - protein, 46 aa, Artificial Sequence
  SEQ B with glycosylation motif
  MISC_FEATURE (25)..(25): any proteinogenic or non-proteinogenic amino acid except proline
  MISC_FEATURE (26)..(26): T, S or C
YKNPKLTRMLTFKFYMPKKATELNXXQCLEEELKPLEEVLNLAQSK

SEQ ID NO: 109 - protein, 46 aa, Artificial Sequence
  SEQ B with glycosylation motif
  MISC_FEATURE (26)..(26): any proteinogenic or non-proteinogenic amino acid except proline
  MISC_FEATURE (27)..(27): T, S or C
YKNPKLTRMLTFKFYMPKKATELKNXXCLEEELKPLEEVLNLAQSK

SEQ ID NO: 110 - protein, 46 aa, Artificial Sequence
  SEQ B with glycosylation motif
  MISC_FEATURE (27)..(27): any proteinogenic or non-proteinogenic amino acid except proline
  MISC_FEATURE (28)..(28): T, S or C
YKNPKLTRMLTFKFYMPKKATELKHNXXLEEELKPLEEVLNLAQSK

SEQ ID NO: 111 - protein, 46 aa, Artificial Sequence
  SEQ B with glycosylation motif
  MISC_FEATURE (28)..(28): any proteinogenic or non-proteinogenic amino acid except proline
  MISC_FEATURE (29)..(29): T, S or C
YKNPKLTRMLTFKFYMPKKATELKHLNXXEEELKPLEEVLNLAQSK

SEQ ID NO: 112 - protein, 46 aa, Artificial Sequence
  SEQ B with glycosylation motif
YKNPKLTRMLTFKFYMPKKATELKHLQCLENSTKPLEEVLNLAQSK

SEQ ID NO: 113 - protein, 46 aa, Artificial Sequence
  SEQ B with glycosylation motif
  MISC_FEATURE (29)..(29): any proteinogenic or non-proteinogenic amino acid except proline
  MISC_FEATURE (30)..(30): T, S or C
YKNPKLTRMLTFKFYMPKKATELKHLQNXXEELKPLEEVLNLAQSK

SEQ ID NO: 114 - protein, 46 aa, Artificial Sequence
  SEQ B with glycosylation motif
  MISC_FEATURE (30)..(30): any proteinogenic or non-proteinogenic amino acid except proline
  MISC_FEATURE (31)..(31): T, S or C
YKNPKLTRMLTFKFYMPKKATELKHLQCNXXELKPLEEVLNLAQSK

SEQ ID NO: 115 - protein, 46 aa, Artificial Sequence
  SEQ B with glycosylation motif
  MISC_FEATURE (31)..(31): any proteinogenic or non-proteinogenic amino acid except proline
  MISC_FEATURE (32)..(32): T, S or C
YKNPKLTRMLTFKFYMPKKATELKHLQCLNXXLKPLEEVLNLAQSK

SEQ ID NO: 116 - protein, 46 aa, Artificial Sequence
  SEQ B with glycosylation motif
  MISC_FEATURE (32)..(32): any proteinogenic or non-proteinogenic amino acid except proline
  MISC_FEATURE (33)..(33): T, S or C
YKNPKLTRMLTFKFYMPKKATELKHLQCLENXXKPLEEVLNLAQSK

SEQ ID NO: 117 - protein, 46 aa, Artificial Sequence
  SEQ B with glycosylation motif
  MISC_FEATURE (33)..(33): any proteinogenic or non-proteinogenic amino acid except proline
  MISC_FEATURE (34)..(34): T, S or C
YKNPKLTRMLTFKFYMPKKATELKHLQCLEENXXPLEEVLNLAQSK

SEQ ID NO: 118 - protein, 46 aa, Artificial Sequence
  SEQ B with glycosylation motif
  MISC_FEATURE (34)..(34): any proteinogenic or non-proteinogenic amino acid except proline
  MISC_FEATURE (35)..(35): T, S or C
YKNPKLTRMLTFKFYMPKKATELKHLQCLEEENXXLEEVLNLAQSK

SEQ ID NO: 119 - protein, 46 aa, Artificial Sequence
  SEQ B with glycosylation motif
  MISC_FEATURE (35)..(35): any proteinogenic or non-proteinogenic amino acid except proline
  MISC_FEATURE (36)..(36): T, S or C
YKNPKLTRMLTFKFYMPKKATELKHLQCLEEELNXXEEVLNLAQSK

SEQ ID NO: 120 - protein, 46 aa, Artificial Sequence
  SEQ B with glycosylation motif
  MISC_FEATURE (36)..(36): any proteinogenic or non-proteinogenic amino acid except proline
  MISC_FEATURE (37)..(37): T, S or C
YKNPKLTRMLTFKFYMPKKATELKHLQCLEEELKNXXEVLNLAQSK

SEQ ID NO: 121 - protein, 46 aa, Artificial Sequence
  SEQ B with glycosylation motif
  MISC_FEATURE (37)..(37): any proteinogenic or non-proteinogenic amino acid except proline
  MISC_FEATURE (38)..(38): T, S or C
YKNPKLTRMLTFKFYMPKKATELKHLQCLEEELKPNXXVLNLAQSK

SEQ ID NO: 122 - protein, 46 aa, Artificial Sequence
  SEQ B with glycosylation motif
  MISC_FEATURE (38)..(38): any proteinogenic or non-proteinogenic amino acid except proline
  MISC_FEATURE (39)..(39): T, S or C
YKNPKLTRMLTFKFYMPKKATELKHLQCLEEELKPLNXXLNLAQSK

SEQ ID NO: 123 - protein, 46 aa, Artificial Sequence
  SEQ B with glycosylation motif
  MISC_FEATURE (39)..(39): any proteinogenic or non-proteinogenic amino acid except proline
  MISC_FEATURE (40)..(40): T, S or C
YKNPKLTRMLTFKFYMPKKATELKHLQCLEEELKPLENXXNLAQSK

SEQ ID NO: 124 - protein, 46 aa, Artificial Sequence
  SEQ B with glycosylation motif
  MISC_FEATURE (40)..(40): any proteinogenic or non-proteinogenic amino acid except proline
  MISC_FEATURE (41)..(41): T, S or C
YKNPKLTRMLTFKFYMPKKATELKHLQCLEEELKPLEENXXLAQSK

SEQ ID NO: 125 - protein, 46 aa, Artificial Sequence
  SEQ B with glycosylation motif
  MISC_FEATURE (41)..(41): any proteinogenic or non-proteinogenic amino acid except proline
  MISC_FEATURE (42)..(42): T, S or C
YKNPKLTRMLTFKFYMPKKATELKHLQCLEEELKPLEEVNXXAQSK

SEQ ID NO: 126 - protein, 46 aa, Artificial Sequence
  SEQ B with glycosylation motif
  MISC_FEATURE (42)..(42): any proteinogenic or non-proteinogenic amino acid except proline
  MISC_FEATURE (43)..(43): T, S or C
YKNPKLTRMLTFKFYMPKKATELKHLQCLEEELKPLEEVLNXXQSK

SEQ ID NO: 127 - protein, 46 aa, Artificial Sequence
  SEQ B with glycosylation motif
  MISC_FEATURE (43)..(43): any proteinogenic or non-proteinogenic amino acid except proline
  MISC_FEATURE (44)..(44): T, S or C
YKNPKLTRMLTFKFYMPKKATELKHLQCLEEELKPLEEVLNNXXSK

SEQ ID NO: 128 - protein, 46 aa, Artificial Sequence
  SEQ B with glycosylation motif
  MISC_FEATURE (44)..(44): any proteinogenic or non-proteinogenic amino acid except proline
  MISC_FEATURE (45)..(45): T, S or C
YKNPKLTRMLTFKFYMPKKATELKHLQCLEEELKPLEEVLNLNXXK

SEQ ID NO: 129 - protein, 46 aa, Artificial Sequence
  SEQ B with glycosylation motif
  MISC_FEATURE (45)..(45): any proteinogenic or non-proteinogenic amino acid except proline
  MISC_FEATURE (46)..(46): T, S or C
YKNPKLTRMLTFKFYMPKKATELKHLQCLEEELKPLEEVLNLANXX

SEQ ID NO: 130 - protein, 46 aa, Artificial Sequence
  SEQ B with glycosylation motif
  MISC_FEATURE (1)..(1): any proteinogenic or non-proteinogenic amino acid
  MISC_FEATURE (2)..(2): any proteinogenic or non-proteinogenic amino acid
  MISC_FEATURE (4)..(4): any proteinogenic or non-proteinogenic amino acid except proline
  MISC_FEATURE (5)..(5): T, S or C
XXNXXLTRMLTFKFYMPKKATELKHLQCLEEELKPLEEVLNLAQSK

SEQ ID NO: 131 - protein, 46 aa, Artificial Sequence
  SEQ B with glycosylation motif
  MISC_FEATURE (2)..(2): any proteinogenic or non-proteinogenic amino acid
  MISC_FEATURE (3)..(3): any proteinogenic or non-proteinogenic amino acid
  MISC_FEATURE (5)..(5): any proteinogenic or non-proteinogenic amino acid except proline
  MISC_FEATURE (6)..(6): T, S or C
YXXNXXTRMLTFKFYMPKKATELKHLQCLEEELKPLEEVLNLAQSK

SEQ ID NO: 132 - protein, 46 aa, Artificial Sequence
  SEQ B with glycosylation motif
  MISC_FEATURE (3)..(3): any proteinogenic or non-proteinogenic amino acid
  MISC_FEATURE (4)..(4): any proteinogenic or non-proteinogenic amino acid
  MISC_FEATURE (6)..(6): any proteinogenic or non-proteinogenic amino acid except proline
  MISC_FEATURE (7)..(7): T, S or C
YKXXNXXRMLTFKFYMPKKATELKHLQCLEEELKPLEEVLNLAQSK

SEQ ID NO: 133 - protein, 46 aa, Artificial Sequence
  SEQ B with glycosylation motif
  MISC_FEATURE (4)..(4): any proteinogenic or non-proteinogenic amino acid
  MISC_FEATURE (5)..(5): any proteinogenic or non-proteinogenic amino acid
  MISC_FEATURE (7)..(7): any proteinogenic or non-proteinogenic amino acid except proline
  MISC_FEATURE (8)..(8): T, S or C
YKNXXNXXMLTFKFYMPKKATELKHLQCLEEELKPLEEVLNLAQSK

SEQ ID NO: 134 - protein, 46 aa, Artificial Sequence
  SEQ B with glycosylation motif
  MISC_FEATURE (5)..(5): any proteinogenic or non-proteinogenic amino acid
  MISC_FEATURE (6)..(6): any proteinogenic or non-proteinogenic amino acid
  MISC_FEATURE (8)..(8): any proteinogenic or non-proteinogenic amino acid except proline
  MISC_FEATURE (9)..(9): T, S or C
YKNPXXNXXLTFKFYMPKKATELKHLQCLEEELKPLEEVLNLAQSK

SEQ ID NO: 135 - protein, 46 aa, Artificial Sequence
  SEQ B with glycosylation motif
  MISC_FEATURE (6)..(6): any proteinogenic or non-proteinogenic amino acid
  MISC_FEATURE (7)..(7): any proteinogenic or non-proteinogenic amino acid
  MISC_FEATURE (9)..(9): any proteinogenic or non-proteinogenic amino acid except proline
  MISC_FEATURE (10)..(10): T, S or C
YKNPKXXNXXTFKFYMPKKATELKHLQCLEEELKPLEEVLNLAQSK

SEQ ID NO: 136 - protein, 46 aa, Artificial Sequence
  SEQ B with glycosylation motif
  MISC_FEATURE (7)..(7): any proteinogenic or non-proteinogenic amino acid
  MISC_FEATURE (8)..(8): any proteinogenic or non-proteinogenic amino acid
  MISC_FEATURE (10)..(10): any proteinogenic or non-proteinogenic amino acid except proline
  MISC_FEATURE (11)..(11): any proteinogenic or non-proteinogenic amino acid except proline
YKNPKLXXNXXFKFYMPKKATELKHLQCLEEELKPLEEVLNLAQSK

SEQ ID NO: 137 - protein, 46 aa, Artificial Sequence
  SEQ B with glycosylation motif
  MISC_FEATURE (8)..(8): any proteinogenic or non-proteinogenic amino acid
  MISC_FEATURE (9)..(9): any proteinogenic or non-proteinogenic amino acid
  MISC_FEATURE (11)..(11): any proteinogenic or non-proteinogenic amino acid except proline
  MISC_FEATURE (12)..(12): T, S or C
YKNPKLTXXNXXKFYMPKKATELKHLQCLEEELKPLEEVLNLAQSK

SEQ ID NO: 138 - protein, 46 aa, Artificial Sequence
  SEQ B with glycosylation motif
  MISC_FEATURE (9)..(9): any proteinogenic or non-proteinogenic amino acid
  MISC_FEATURE (10)..(10): any proteinogenic or non-proteinogenic amino acid
  MISC_FEATURE (12)..(12): any proteinogenic or non-proteinogenic amino acid except proline
  MISC_FEATURE (13)..(13): T, S or C
YKNPKLTRXXNXXFYMPKKATELKHLQCLEEELKPLEEVLNLAQSK

SEQ ID NO: 139 - protein, 46 aa, Artificial Sequence
  SEQ B with glycosylation motif
  MISC_FEATURE (10)..(10): any proteinogenic or non-proteinogenic amino acid
  MISC_FEATURE (11)..(11): any proteinogenic or non-proteinogenic amino acid
  MISC_FEATURE (13)..(13): any proteinogenic or non-proteinogenic amino acid except proline
  MISC_FEATURE (14)..(14): T, S or C
YKNPKLTRMXXNXXYMPKKATELKHLQCLEEELKPLEEVLNLAQSK

SEQ ID NO: 140 - protein, 46 aa, Artificial Sequence
  SEQ B with glycosylation motif
  MISC_FEATURE (11)..(11): any proteinogenic or non-proteinogenic amino acid
  MISC_FEATURE (12)..(12): any proteinogenic or non-proteinogenic amino acid
  MISC_FEATURE (14)..(14): any proteinogenic or non-proteinogenic amino acid except proline
  T, S or C (15)..(15): any proteinogenic or non-proteinogenic amino acid except proline
YKNPKLTRMLXXNXXMPKKATELKHLQCLEEELKPLEEVLNLAQSK

SEQ ID NO: 141 - protein, 46 aa, Artificial Sequence
  SEQ B with glycosylation motif
  MISC_FEATURE (12)..(12): any proteinogenic or non-proteinogenic amino acid
  MISC_FEATURE (13)..(13): any proteinogenic or non-proteinogenic amino acid
  MISC_FEATURE (15)..(15): any proteinogenic or non-proteinogenic amino acid except proline
  MISC_FEATURE (16)..(16): T, S or C
YKNPKLTRMLTXXNXXPKKATELKHLQCLEEELKPLEEVLNLAQSK

SEQ ID NO: 142 - protein, 46 aa, Artificial Sequence
  SEQ B with glycosylation motif
  MISC_FEATURE (13)..(13): any proteinogenic or non-proteinogenic amino acid
  MISC_FEATURE (14)..(14): any proteinogenic or non-proteinogenic amino acid
  MISC_FEATURE (16)..(16): any proteinogenic or non-proteinogenic amino acid except proline
  MISC_FEATURE (17)..(17): T, S or C
YKNPKLTRMLTFXXNXXKKATELKHLQCLEEELKPLEEVLNLAQSK

SEQ ID NO: 143 - protein, 46 aa, Artificial Sequence
  SEQ B with glycosylation motif
  MISC_FEATURE (14)..(14): any proteinogenic or non-proteinogenic amino acid
  MISC_FEATURE (15)..(15): any proteinogenic or non-proteinogenic amino acid
  MISC_FEATURE (17)..(17): any proteinogenic or non-proteinogenic amino acid except proline
  MISC_FEATURE (18)..(18): T, S or C
YKNPKLTRMLTFKXXNXXKATELKHLQCLEEELKPLEEVLNLAQSK

SEQ ID NO: 144 - protein, 46 aa, Artificial Sequence
  SEQ B with glycosylation motif
  MISC_FEATURE (15)..(15): any proteinogenic or non-proteinogenic amino acid
  MISC_FEATURE (16)..(16): any proteinogenic or non-proteinogenic amino acid
  MISC_FEATURE (18)..(18): any proteinogenic or non-proteinogenic amino acid except proline
  MISC_FEATURE (19)..(19): T, S or C
YKNPKLTRMLTFKFXXNXXATELKHLQCLEEELKPLEEVLNLAQSK

SEQ ID NO: 145 - protein, 46 aa, Artificial Sequence
  SEQ B with glycosylation motif
  MISC_FEATURE (16)..(16): any proteinogenic or non-proteinogenic amino acid
  MISC_FEATURE (17)..(17): any proteinogenic or non-proteinogenic amino acid
  MISC_FEATURE (19)..(19): any proteinogenic or non-proteinogenic amino acid except proline
  MISC_FEATURE (20)..(20): T, S or C
YKNPKLTRMLTFKFYXXNXXTELKHLQCLEEELKPLEEVLNLAQSK

SEQ ID NO: 146 - protein, 46 aa, Artificial Sequence
  SEQ B with glycosylation motif
  MISC_FEATURE (17)..(17): any proteinogenic or non-proteinogenic amino acid
  MISC_FEATURE (18)..(18): any proteinogenic or non-proteinogenic amino acid
  MISC_FEATURE (20)..(20): any proteinogenic or non-proteinogenic amino acid except proline
  MISC_FEATURE (21)..(21): T, S or C
YKNPKLTRMLTFKFYMXXNXXELKHLQCLEEELKPLEEVLNLAQSK

SEQ ID NO: 147 - protein, 46 aa, Artificial Sequence
  SEQ B with glycosylation motif
  MISC_FEATURE (18)..(18): any proteinogenic or non-proteinogenic amino acid
  MISC_FEATURE (19)..(19): any proteinogenic or non-proteinogenic amino acid
  MISC_FEATURE (21)..(21): any proteinogenic or non-proteinogenic amino acid except proline
  MISC_FEATURE (22)..(22): T, S or C
YKNPKLTRMLTFKFYMPXXNXXLKHLQCLEEELKPLEEVLNLAQSK

SEQ ID NO: 148 - protein, 46 aa, Artificial Sequence
  SEQ B with glycosylation motif
  MISC_FEATURE (19)..(19): any proteinogenic or non-proteinogenic amino acid
  MISC_FEATURE (20)..(20): any proteinogenic or non-proteinogenic amino acid
  MISC_FEATURE (22)..(22): any proteinogenic or non-proteinogenic amino acid except proline
  MISC_FEATURE (23)..(23): T, S or C
YKNPKLTRMLTFKFYMPKXXNXXKHLQCLEEELKPLEEVLNLAQSK

SEQ ID NO: 149 - protein, 46 aa, Artificial Sequence
  SEQ B with glycosylation motif
  MISC_FEATURE (20)..(20): any proteinogenic or non-proteinogenic amino acid
  MISC_FEATURE (21)..(21): any proteinogenic or non-proteinogenic amino acid
  MISC_FEATURE (23)..(23): any proteinogenic or non-proteinogenic amino acid except proline
  MISC_FEATURE (24)..(24): T, S or C
YKNPKLTRMLTFKFYMPKKXXNXXHLQCLEEELKPLEEVLNLAQSK

SEQ ID NO: 150 - protein, 46 aa, Artificial Sequence
  SEQ B with glycosylation motif
  MISC_FEATURE (21)..(21): any proteinogenic or non-proteinogenic amino acid
  MISC_FEATURE (22)..(22): any proteinogenic or non-proteinogenic amino acid
  MISC_FEATURE (24)..(24): any proteinogenic or non-proteinogenic amino acid except proline
  MISC_FEATURE (25)..(25): T, S or C
YKNPKLTRMLTFKFYMPKKAXXNXXLQCLEEELKPLEEVLNLAQSK

SEQ ID NO: 151 - protein, 46 aa, Artificial Sequence
  SEQ B with glycosylation motif
  MISC_FEATURE (22)..(22): any proteinogenic or non-proteinogenic amino acid
  MISC_FEATURE (23)..(23): any proteinogenic or non-proteinogenic amino acid
  MISC_FEATURE (25)..(25): any proteinogenic or non-proteinogenic amino acid except proline
  MISC_FEATURE (26)..(26): T, S or C
YKNPKLTRMLTFKFYMPKKATXXNXXQCLEEELKPLEEVLNLAQSK

SEQ ID NO: 152 - protein, 46 aa, Artificial Sequence
  SEQ B with glycosylation motif
  MISC_FEATURE (23)..(23): any proteinogenic or non-proteinogenic amino acid
  MISC_FEATURE (24)..(24): any proteinogenic or non-proteinogenic amino acid
  MISC_FEATURE (26)..(26): any proteinogenic or non-proteinogenic amino acid except proline
  MISC_FEATURE (27)..(27): T, S or C
YKNPKLTRMLTFKFYMPKKATEXXNXXCLEEELKPLEEVLNLAQSK

SEQ ID NO: 153 - protein, 46 aa, Artificial Sequence
  SEQ B with glycosylation motif
  MISC_FEATURE (24)..(24): any proteinogenic or non-proteinogenic amino acid
  MISC_FEATURE (25)..(25): any proteinogenic or non-proteinogenic amino acid
  MISC_FEATURE (27)..(27): any proteinogenic or non-proteinogenic amino acid except proline
  MISC_FEATURE (28)..(28): T, S or C
YKNPKLTRMLTFKFYMPKKATELXXNXXLEEELKPLEEVLNLAQSK

SEQ ID NO: 154 - protein, 46 aa, Artificial Sequence
  SEQ B with glycosylation motif
  MISC_FEATURE (25)..(25): any proteinogenic or non-proteinogenic amino acid
  MISC_FEATURE (26)..(26): any proteinogenic or non-proteinogenic amino acid
  MISC_FEATURE (28)..(28): any proteinogenic or non-proteinogenic amino acid except proline
  MISC_FEATURE (29)..(29): T, S or C
YKNPKLTRMLTFKFYMPKKATELKXXNXXEEELKPLEEVLNLAQSK

SEQ ID NO: 155 - protein, 46 aa, Artificial Sequence
  SEQ B with glycosylation motif
  MISC_FEATURE (26)..(26): any proteinogenic or non-proteinogenic amino acid
  MISC_FEATURE (27)..(27): any proteinogenic or non-proteinogenic amino acid
  MISC_FEATURE (29)..(29): any proteinogenic or non-proteinogenic amino acid except proline
  MISC_FEATURE (30)..(30): T, S or C
YKNPKLTRMLTFKFYMPKKATELKHXXNXXEELKPLEEVLNLAQSK

SEQ ID NO: 156 - protein, 46 aa, Artificial Sequence
  SEQ B with glycosylation motif
  MISC_FEATURE (27)..(27): any proteinogenic or non-proteinogenic amino acid
  MISC_FEATURE (28)..(28): any proteinogenic or non-proteinogenic amino acid
  MISC_FEATURE (30)..(30): any proteinogenic or non-proteinogenic amino acid except proline
  MISC_FEATURE (31)..(31): T, S or C
YKNPKLTRMLTFKFYMPKKATELKHLXXNXXELKPLEEVLNLAQSK

SEQ ID NO: 157 - protein, 46 aa, Artificial Sequence
  SEQ B with glycosylation motif
  MISC_FEATURE (28)..(28): any proteinogenic or non-proteinogenic amino acid
  MISC_FEATURE (29)..(29): any proteinogenic or non-proteinogenic amino acid
  MISC_FEATURE (31)..(31): any proteinogenic or non-proteinogenic amino acid except proline
  MISC_FEATURE (32)..(32): T, S or C
YKNPKLTRMLTFKFYMPKKATELKHLQXXNXXLKPLEEVLNLAQSK

SEQ ID NO: 158 - protein, 46 aa, Artificial Sequence
  SEQ B with glycosylation motif
  MISC_FEATURE (29)..(29): any proteinogenic or non-proteinogenic amino acid
  MISC_FEATURE (30)..(30): any proteinogenic or non-proteinogenic amino acid
  MISC_FEATURE (32)..(32): any proteinogenic or non-proteinogenic amino acid except proline
  MISC_FEATURE (33)..(33): T, S or C
YKNPKLTRMLTFKFYMPKKATELKHLQCXXNXXKPLEEVLNLAQSK

SEQ ID NO: 159 - protein, 46 aa, Artificial Sequence
  SEQ B with glycosylation motif
  MISC_FEATURE (30)..(30): any proteinogenic or non-proteinogenic amino acid
  MISC_FEATURE (31)..(31): any proteinogenic or non-proteinogenic amino acid
  MISC_FEATURE (33)..(33): any proteinogenic or non-proteinogenic amino acid except proline
  MISC_FEATURE (34)..(34): T, S or C
YKNPKLTRMLTFKFYMPKKATELKHLQCLXXNXXPLEEVLNLAQSK

SEQ ID NO: 160 - protein, 46 aa, Artificial Sequence
  SEQ B with glycosylation motif
  MISC_FEATURE (31)..(31): any proteinogenic or non-proteinogenic amino acid
  MISC_FEATURE (32)..(32): any proteinogenic or non-proteinogenic amino acid
  MISC_FEATURE (34)..(34): any proteinogenic or non-proteinogenic amino acid except proline
  MISC_FEATURE (35)..(35): T, S or C
YKNPKLTRMLTFKFYMPKKATELKHLQCLEXXNXXLEEVLNLAQSK

SEQ ID NO: 161 - protein, 46 aa, Artificial Sequence
  SEQ B with glycosylation motif
  MISC_FEATURE (32)..(32): any proteinogenic or non-proteinogenic amino acid
  MISC_FEATURE (33)..(33): any proteinogenic or non-proteinogenic amino acid
  MISC_FEATURE (35)..(35): any proteinogenic or non-proteinogenic amino acid except proline
  MISC_FEATURE (36)..(36): T, S or C
YKNPKLTRMLTFKFYMPKKATELKHLQCLEEXXNXXEEVLNLAQSK

SEQ ID NO: 162 - protein, 46 aa, Artificial Sequence
  SEQ B with glycosylation motif
  MISC_FEATURE (33)..(33): any proteinogenic or non-proteinogenic amino acid
  MISC_FEATURE (34)..(34): any proteinogenic or non-proteinogenic amino acid
  MISC_FEATURE (36)..(36): any proteinogenic or non-proteinogenic amino acid except proline
  MISC_FEATURE (37)..(37): T, S or C
YKNPKLTRMLTFKFYMPKKATELKHLQCLEEEXXNXXEVLNLAQSK

SEQ ID NO: 163 - protein, 46 aa, Artificial Sequence
  SEQ B with glycosylation motif
  MISC_FEATURE (34)..(34): any proteinogenic or non-proteinogenic amino acid
  MISC_FEATURE (35)..(35): any proteinogenic or non-proteinogenic amino acid
  MISC_FEATURE (37)..(37): any proteinogenic or non-proteinogenic amino acid except proline
  MISC_FEATURE (38)..(38): T, S or C
YKNPKLTRMLTFKFYMPKKATELKHLQCLEEELXXNXXVLNLAQSK

SEQ ID NO: 164 - protein, 46 aa, Artificial Sequence
  SEQ B with glycosylation motif
  MISC_FEATURE (35)..(35): any proteinogenic or non-proteinogenic amino acid
  MISC_FEATURE (36)..(36): any proteinogenic or non-proteinogenic amino acid
  MISC_FEATURE (38)..(38): any proteinogenic or non-proteinogenic amino acid except proline
  MISC_FEATURE (39)..(39): T, S or C
YKNPKLTRMLTFKFYMPKKATELKHLQCLEEELKXXNXXLNLAQSK

SEQ ID NO: 165 - protein, 46 aa, Artificial Sequence
  SEQ B with glycosylation motif
  MISC_FEATURE (36)..(36): any proteinogenic or non-proteinogenic amino acid
  MISC_FEATURE (37)..(37): any proteinogenic or non-proteinogenic amino acid
  MISC_FEATURE (39)..(39): any proteinogenic or non-proteinogenic amino acid except proline
  MISC_FEATURE (40)..(40): T, S or C
YKNPKLTRMLTFKFYMPKKATELKHLQCLEEELKPXXNXXNLAQSK

SEQ ID NO: 166 - protein, 46 aa, Artificial Sequence
  SEQ B with glycosylation motif
  MISC_FEATURE (37)..(37): any proteinogenic or non-proteinogenic amino acid
  MISC_FEATURE (38)..(38): any proteinogenic or non-proteinogenic amino acid
  MISC_FEATURE (40)..(40): any proteinogenic or non-proteinogenic amino acid except proline
  MISC_FEATURE (41)..(41): T, S or C
YKNPKLTRMLTFKFYMPKKATELKHLQCLEEELKPLXXNXXLAQSK

SEQ ID NO: 167 - protein, 46 aa, Artificial Sequence
  SEQ B with glycosylation motif
  MISC_FEATURE (38)..(38): any proteinogenic or non-proteinogenic amino acid
  MISC_FEATURE (39)..(39): any proteinogenic or non-proteinogenic amino acid
  MISC_FEATURE (41)..(41): any proteinogenic or non-proteinogenic amino acid except proline
  MISC_FEATURE (42)..(42): T, S or C
YKNPKLTRMLTFKFYMPKKATELKHLQCLEEELKPLEXXNXXAQSK

SEQ ID NO: 168 - protein, 46 aa, Artificial Sequence
  SEQ B with glycosylation motif
  MISC_FEATURE (39)..(39): any proteinogenic or non-proteinogenic amino acid
  MISC_FEATURE (40)..(40): any proteinogenic or non-proteinogenic amino acid
  MISC_FEATURE (42)..(42): any proteinogenic or non-proteinogenic amino acid except proline
  MISC_FEATURE (43)..(43): T, S or C
YKNPKLTRMLTFKFYMPKKATELKHLQCLEEELKPLEEXXNXXQSK

SEQ ID NO: 169 - protein, 46 aa, Artificial Sequence
  SEQ B with glycosylation motif
  MISC_FEATURE (40)..(40): any proteinogenic or non-proteinogenic amino acid
  MISC_FEATURE (41)..(41): any proteinogenic or non-proteinogenic amino acid
  MISC_FEATURE (43)..(43): any proteinogenic or non-proteinogenic amino acid except proline
  MISC_FEATURE (44)..(44): T, S or C
YKNPKLTRMLTFKFYMPKKATELKHLQCLEEELKPLEEVXXNXXSK

SEQ ID NO: 170 - protein, 46 aa, Artificial Sequence
  SEQ B with glycosylation motif
  MISC_FEATURE (41)..(41): any proteinogenic or non-proteinogenic amino acid
  MISC_FEATURE (42)..(42): any proteinogenic or non-proteinogenic amino acid
  MISC_FEATURE (44)..(44): any proteinogenic or non-proteinogenic amino acid except proline
  MISC_FEATURE (45)..(45): T, S or C
YKNPKLTRMLTFKFYMPKKATELKHLQCLEEELKPLEEVLXXNXXK

SEQ ID NO: 171 - protein, 46 aa, Artificial Sequence
  SEQ B with glycosylation motif
  MISC_FEATURE (42)..(42): any proteinogenic or non-proteinogenic amino acid
  MISC_FEATURE (43)..(43): any proteinogenic or non-proteinogenic amino acid
  MISC_FEATURE (45)..(45): any proteinogenic or non-proteinogenic amino acid except proline
  MISC_FEATURE (46)..(46): T, S or C
YKNPKLTRMLTFKFYMPKKATELKHLQCLEEELKPLEEVLNXXNXX

SEQ ID NO: 172 - protein, 46 aa, Artificial Sequence
  SEQ B with glycosylation motif
YKNPNSTRMLTFKFYMPKKATELKHLQCLEEELKPLEEVLNLAQSK

SEQ ID NO: 173 - protein, 46 aa, Artificial Sequence
  SEQ B with glycosylation motif
YKNPKLTNSTTFKFYMPKKATELKHLQCLEEELKPLEEVLNLAQSK

SEQ ID NO: 174 - protein, 46 aa, Artificial Sequence
  SEQ B with glycosylation motif
YKNPKLTRNSTFKFYMPKKATELKHLQCLEEELKPLEEVLNLAQSK

SEQ ID NO: 175 - protein, 46 aa, Artificial Sequence
  SEQ B with glycosylation motif
YKNPKLTRMLNSTFYMPKKATELKHLQCLEEELKPLEEVLNLAQSK

SEQ ID NO: 176 - protein, 46 aa, Artificial Sequence
  SEQ B with glycosylation motif
YKNPKLTRMLTNSTYMPKKATELKHLQCLEEELKPLEEVLNLAQSK

SEQ ID NO: 177 - protein, 46 aa, Artificial Sequence
  SEQ B with glycosylation motif
YKNPKLTRMLTFNSTMPKKATELKHLQCLEEELKPLEEVLNLAQSK

SEQ ID NO: 178 - protein, 46 aa, Artificial Sequence
  SEQ B with glycosylation motif
YKNPKLTRMLTFKNSTPKKATELKHLQCLEEELKPLEEVLNLAQSK

SEQ ID NO: 179 - protein, 46 aa, Artificial Sequence
  SEQ B with glycosylation motif
YKNPKLTRMLTFKFNSTKKATELKHLQCLEEELKPLEEVLNLAQSK

SEQ ID NO: 180 - protein, 46 aa, Artificial Sequence
  SEQ B with glycosylation motif
YKNPKLTRMLTFKFYMPKKATELKHLQCLEENSTPLEEVLNLAQSK

SEQ ID NO: 181 - protein, 46 aa, Artificial Sequence
  SEQ B with glycosylation motif
YKNPKLTRMLTFKFYMPKKATELKHLQCLEEELKPLEEVLNNSTSK

SEQ ID NO: 182 - protein, 46 aa, Artificial Sequence
  SEQ B with glycosylation motif
YKNPFANSTLTFKFYMPKKATELKHLQCLEEELKPLEEVLNLAQSK

SEQ ID NO: 183 - protein, 46 aa, Artificial Sequence
  SEQ B with glycosylation motif
YKNPKLTRFANSTFYMPKKATELKHLQCLEEELKPLEEVLNLAQSK

SEQ ID NO: 184 - protein, 46 aa, Artificial Sequence
  SEQ B with glycosylation motif
YKNPKLTRMLTFANSTPKKATELKHLQCLEEELKPLEEVLNLAQSK

SEQ ID NO: 185 - protein, 46 aa, Artificial Sequence
  SEQ B with glycosylation motif
YKNPKLTRMLTFFANSTKKATELKHLQCLEEELKPLEEVLNLAQSK

SEQ ID NO: 186 - protein, 46 aa, Artificial Sequence
  SEQ B with glycosylation motif
YKNPKLTRMLTFKFANSTKATELKHLQCLEEELKPLEEVLNLAQSK

SEQ ID NO: 187 - protein, 46 aa, Artificial Sequence
  SEQ B with glycosylation motif
YKNPKLTRMLTFKFFANSTATELKHLQCLEEELKPLEEVLNLAQSK

SEQ ID NO: 188 - protein, 46 aa, Artificial Sequence
  SEQ B with glycosylation motif
YKNPKLTRMLTFKFYMPKKATELKHLQCLEEFANSTEEVLNLAQSK

SEQ ID NO: 189 - protein, 46 aa, Artificial Sequence
  SEQ B with glycosylation motif
YKNPKLTRMLTFKFYMPKKATELKHLQCLEEELKPLEEVLNFANST

SEQ ID NO: 190 - protein, 46 aa, Artificial Sequence
  SEQ B with glycosylation motif
YKNPFGNSTLTFKFYMPKKATELKHLQCLEEELKPLEEVLNLAQSK

SEQ ID NO: 191 - protein, 46 aa, Artificial Sequence
  SEQ B with glycosylation motif
YKNPKLTRFGNSTFYMPKKATELKHLQCLEEELKPLEEVLNLAQSK

SEQ ID NO: 192 - protein, 46 aa, Artificial Sequence
  SEQ B with glycosylation motif
YKNPKLTRMLTFGNSTPKKATELKHLQCLEEELKPLEEVLNLAQSK

SEQ ID NO: 193 - protein, 46 aa, Artificial Sequence
  SEQ B with glycosylation motif
YKNPKLTRMLTFFGNSTKKATELKHLQCLEEELKPLEEVLNLAQSK

SEQ ID NO: 194 - protein, 46 aa, Artificial Sequence
  SEQ B with glycosylation motif
YKNPKLTRMLTFKFGNSTKATELKHLQCLEEELKPLEEVLNLAQSK

SEQ ID NO: 195 - protein, 46 aa, Artificial Sequence
  SEQ B with glycosylation motif
YKNPKLTRMLTFKFFGNSTATELKHLQCLEEELKPLEEVLNLAQSK

SEQ ID NO: 196 - protein, 46 aa, Artificial Sequence
  SEQ B with glycosylation motif
YKNPKLTRMLTFKFYMPKKATELKHLQCLEEFGNSTEEVLNLAQSK

SEQ ID NO: 197 - protein, 46 aa, Artificial Sequence
  SEQ B with glycosylation motif
YKNPKLTRMLTFKFYMPKKATELKHLQCLEEELKPLEEVLNFGNST

SEQ ID NO: 198 - protein, 46 aa, Artificial Sequence
  SEQ B with glycosylation motif
YKNSTLTRMLTFKFYMPKKATELKHLQCLEEELKPLEEVLNLAQSK

SEQ ID NO: 199 - protein, 46 aa, Artificial Sequence
  SEQ B with glycosylation motif
FANSTLTRMLTFKFYMPKKATELKHLQCLEEELKPLEEVLNLAQSK

SEQ ID NO: 200 - protein, 46 aa, Artificial Sequence
  SEQ B with glycosylation motif
FGNSTLTRMLTFKFYMPKKATELKHLQCLEEELKPLEEVLNLAQSK

SEQ ID NO: 201 - protein, 46 aa, Artificial Sequence
  SEQ B with glycosylation motif
YKNPKLTRMLTFKFYMPKKATELKHLQCLEEELKPLEEVLNSTQSK

SEQ ID NO: 202 - DNA, 486 bp, Artificial Sequence
  DNA sequences for protein 1c
atggatatgcgggtccctgctcagctgctgggcctgctgctgctgtggtttcctgggtcaaggtgcgcccccacatctagttctacaaagaagacacaattacaattagagcacttattattagacttacaaatgatattaaatggcataaataattataagaatcccaagttaactagaatgttaacattcaagttttatatgcctaagaaggctacagagttaaagcacttacaatgtttagagttcggcaacagcaccttagaggaggtattaaatttagcccaatcaaagaattttcacttacggccccgggacttaatttccaatattaatgttattgtgttagagttaaagggctctgagacaacttttatgtgtgagtatgccgacgagacagccacaattgttgagttcttaaatcggtggattactttttcacaatccattatctccactttaactcatcatcaccatcaccactaa

SEQ ID NO: 203 - DNA, 486 bp, Artificial Sequence
  DNA sequence for protein 1d
atggatatgcgggtccctgctcagctgctgggcctgctgctgctgtggtttcctgggtcaaggtgcgcccccacatctagttctacaaagaagacacaattacaattagagcacttattattagacttacaaatgatattaaatggcataaataattataagaatcccaagttaactagaatgttaacattcaagttttatatgcctaagaaggctacagagttaaagcacttacaatgtttagaggaggagttaaagccattagaggaggtattaaatttagcccaatcaaagaattttcacttacggccccgggacttaatttccaatattaatgttattgtgttagagttaaagggctctgagacaacttttatgtgtgagtatgccgacgagacagccacaattgttgagttcttaaatcggtggattactttttcacaatccattatctccactttaactcatcatcaccatcaccactaa

SEQ ID NO: 204 - protein, 138 aa, Artificial Sequence
  IL-2 variant with glycosylation motif and His-tag
PTSSSTKKTQLQLEHLLLDLQMILNGINNYKNPKLTFGNSTKFYMPKKATELKHLQCLEEELKPLEEVLNLAQSKNFHLRPRDLISNINVIVLELKGSETTFMCEYADETATIVEFLNRWITFSQSIISTLTHHHHHH

SEQ ID NO: 205 - protein, 139 aa, Artificial Sequence
  IL-2 variant with glycosylation motif and His-tag
APTSSSTKKTQLQLEHLLLDLQMILNGINNYKNPKLTFGNSTKFYMPKKATELKHLQCLEEELKPLEEVLNLAQSKNFHLRPRDLISNINVIVLELKGSETTFMCEYADETATIVEFLNRWITFSQSIISTLTHHHHHH

SEQ ID NO: 206 - protein, 138 aa, Artificial Sequence
  IL-2 variant with glycosylation motif and His-tag
PASSSTKKTQLQLEHLLLDLQMILNGINNYKNPKLTFGNSTKFYMPKKATELKHLQCLEEELKPLEEVLNLAQSKNFHLRPRDLISNINVIVLELKGSETTFMCEYADETATIVEFLNRWITFSQSIISTLTHHHHHH

SEQ ID NO: 207 - protein, 139 aa, Artificial Sequence
  IL-2 variant with glycosylation motif and His-tag
APASSSTKKTQLQLEHLLLDLQMILNGINNYKNPKLTFGNSTKFYMPKKATELKHLQCLEEELKPLEEVLNLAQSKNFHLRPRDLISNINVIVLELKGSETTFMCEYADETATIVEFLNRWITFSQSIISTLTHHHHHH

SEQ ID NO: 208 - protein, 138 aa, Artificial Sequence
  IL-2 variant with glycosylation motif and His-tag
PTSSSTKKTQLQLEHLLLDLQMILNGINNYKNPKLTFGNSTKFYMPKKATELKHLQCLEEELKPLEEVLNLAQSKNFHLRPRDLISNINVIVLELKGSETTFMCEYADETATIVEFLNRWITFCQSIISTLTHHHHHH

SEQ ID NO: 209 - protein, 139 aa, Artificial Sequence
  IL-2 variant with glycosylation motif and His-tag
APTSSSTKKTQLQLEHLLLDLQMILNGINNYKNPKLTFGNSTKFYMPKKATELKHLQCLEEELKPLEEVLNLAQSKNFHLRPRDLISNINVIVLELKGSETTFMCEYADETATIVEFLNRWITFCQSIISTLTHHHHHH

SEQ ID NO: 210 - protein, 138 aa, Artificial Sequence
  IL-2 variant with glycosylation motif and His-tag
PASSSTKKTQLQLEHLLLDLQMILNGINNYKNPKLTFGNSTKFYMPKKATELKHLQCLEEELKPLEEVLNLAQSKNFHLRPRDLISNINVIVLELKGSETTFMCEYADETATIVEFLNRWITFCQSIISTLTHHHHHH

SEQ ID NO: 211 - protein, 139 aa, Artificial Sequence
  IL-2 variant with glycosylation motif and His-tag
APASSSTKKTQLQLEHLLLDLQMILNGINNYKNPKLTFGNSTKFYMPKKATELKHLQCLEEELKPLEEVLNLAQSKNFHLRPRDLISNINVIVLELKGSETTFMCEYADETATIVEFLNRWITFCQSIISTLTHHHHHH

SEQ ID NO: 212 - protein, 57 aa, Artificial Sequence
  SEQ C with C49A
NFHLRPRDLISNINVIVLELKGSETTFMCEYADETATIVEFLNRWITFAQSIISTLT

SEQ ID NO: 213 - protein, 134 aa, Artificial Sequence
  IL-2 variant
MAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 214 - protein, 46 aa, Artificial Sequence
  SEQ B with glycosylation motif
YKNPDLTFGNSTKFYMPKKATELKHLQCLEEELKPLEEVLNLAQSK

SEQ ID NO: 215 - protein, 46 aa, Artificial Sequence
  SEQ B with glycosylation motif
YKNPELTFGNSTKFYMPKKATELKHLQCLEEELKPLEEVLNLAQSK

SEQ ID NO: 216 - protein, 132 aa, Artificial Sequence
  IL-2 variant with glycosylation motif
PTSSSTKKTQLQLEHLLLDLQMILNGINNYKNPDLTFGNSTKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 217 - protein, 133 aa, Artificial Sequence
  IL-2 variant with glycosylation motif
APTSSSTKKTQLQLEHLLLDLQMILNGINNYKNPDLTFGNSTKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 218 - protein, 132 aa, Artificial Sequence
  IL-2 variant with glycosylation motif
PASSSTKKTQLQLEHLLLDLQMILNGINNYKNPDLTFGNSTKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 219 - protein, 133 aa, Artificial Sequence
  IL-2 variant with glycosylation motif
APASSSTKKTQLQLEHLLLDLQMILNGINNYKNPDLTFGNSTKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 220 - protein, 132 aa, Artificial Sequence
  IL-2 variant with glycosylation motif
PTSSSTKKTQLQLEHLLLDLQMILNGINNYKNPDLTFGNSTKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 221 - protein, 133 aa, Artificial Sequence
  IL-2 variant with glycosylation motif
APTSSSTKKTQLQLEHLLLDLQMILNGINNYKNPDLTFGNSTKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 222 - protein, 132 aa, Artificial Sequence
  IL-2 variant with glycosylation motif
PASSSTKKTQLQLEHLLLDLQMILNGINNYKNPDLTFGNSTKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 223 - protein, 133 aa, Artificial Sequence
  IL-2 variant with glycosylation motif
APASSSTKKTQLQLEHLLLDLQMILNGINNYKNPDLTFGNSTKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 224 - protein, 132 aa, Artificial Sequence
  IL-2 variant with glycosylation motif
PTSSSTKKTQLQLEHLLLDLQMILNGINNYKNPELTFGNSTKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 225 - protein, 133 aa, Artificial Sequence
  IL-2 variant with glycosylation motif
APTSSSTKKTQLQLEHLLLDLQMILNGINNYKNPELTFGNSTKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 226 - protein, 132 aa, Artificial Sequence
  IL-2 variant with glycosylation motif
PASSSTKKTQLQLEHLLLDLQMILNGINNYKNPELTFGNSTKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 227 - protein, 133 aa, Artificial Sequence
  IL-2 variant with glycosylation motif
APASSSTKKTQLQLEHLLLDLQMILNGINNYKNPELTFGNSTKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 228 - protein, 132 aa, Artificial Sequence
  IL-2 variant with glycosylation motif
PTSSSTKKTQLQLEHLLLDLQMILNGINNYKNPELTFGNSTKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 229 - protein, 139 aa, Artificial Sequence
  IL-2 variant with glycosylation motif and His-tag
APTSSSTKKTQLQLEHLLLDLQMILNGINNYKNPKLTRMLFGNSTMPKKATELKHLQCLEEELKPLEEVLNLAQSKNFHLRPRDLISNINVIVLELKGSETTFMCEYADETATIVEFLNRWITFSQSIISTLTHHHHHH

SEQ ID NO: 230 - protein, 139 aa, Artificial Sequence
  IL-2 variant with glycosylation motif and His-tag
APTSSSTKKTQLQLEHLLLDLQMILNGINNYKNPKLTRMLTFKFYMPKKATELKHLQCLEFGNSTLEEVLNLAQSKNFHLRPRDLISNINVIVLELKGSETTFMCEYADETATIVEFLNRWITFSQSIISTLTHHHHHH

SEQ ID NO: 231 - protein, 147 aa, Artificial Sequence
  IL-2 variant with glycosylation motif and cleavable His-tag
AHHHHHHGSDDDDKAPTSSSTKKTQLQLEHLLLDLQMILNGINNYKNPKLTFGNSTKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 232 - protein, 139 aa, Artificial Sequence
  IL-2 variant with His-tag
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTHHHHHH

SEQ ID NO: 233 - DNA, 510 bp, Artificial Sequence
  DNA sequence encoding protein 1e
atggatatgcgggtccctgctcagctgctgggcctgctgctgctgtggtttcctgggtcaaggtgcgcccatcatcaccatcaccacggcagcgacgacgacgacaaggcccccacatctagttctacaaagaagacacaattacaattagagcacttattattagacttacaaatgatattaaatggcataaataattataagaatcccaagttaactttcggcaacagcaccaagttttatatgcctaagaaggctacagagttaaagcacttacaatgtttagaggaggagttaaagccattagaggaggtattaaatttagcccaatcaaagaattttcacttacggccccgggacttaatttccaatattaatgttattgtgttagagttaaagggctctgagacaacttttatgtgtgagtatgccgacgagacagccacaattgttgagttcttaaatcggtggattactttttcacaatccattatctccactttaacttaa

SEQ ID NO: 234 - protein, 14 aa, Artificial Sequence
  His tag
AHHHHHHGSDDDDK

SEQ ID NO: 235 - protein, 46 aa, Artificial Sequence
  SEQ B with glycosylation motif FANST
YKNPDLTFANSTKFYMPKKATELKHLQCLEEELKPLEEVLNLAQSK

SEQ ID NO: 236 - protein, 46 aa, Artificial Sequence
  SEQ B with glycosylation motif FANST
YKNPELTFANSTKFYMPKKATELKHLQCLEEELKPLEEVLNLAQSK

SEQ ID NO: 237 - protein, 133 aa, Artificial Sequence
  protein 2a
APTSSSTKKTQLQLEHLLLDLQMILNGINNYKNPKLTCMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 238 - protein, 24 aa, Artificial Sequence
  Signal peptide 3a
MAWKTLPIYLLLLLSVFVIQQVSS

SEQ ID NO: 239 - protein, 19 aa, Artificial Sequence
  Signal peptide 3b
MEFGLSWVFLVAIIKGVQC

SEQ ID NO: 240 - protein, 20 aa, Artificial Sequence
  Signal peptide 3c
MYRMQLLSCIALSLALVTNS

SEQ ID NO: 241 - DNA, 471 bp, Artificial Sequence
  DNA sequence encoding protein 2b
atggcctggaagactctccccatctacctcctgctgctcctgtcggtcttcgtcatccagcaggtgtcctccgcccccacgtcgtcctccaccaagaagacccagctgcagctcgagcacctcctgctggacctgcagatgatcctgaacgggatcaacaactacaagaacccgaagctgacttgcatgctgaccttcaagttctacatgccgaagaaggcgaccgagctgaagcacctgcagtgcctggaggaggagctcaagccgctcgaggaggtgctgaatctggcccagtccaagaacttccacctgcgcccgcgggacctcatcagcaacatcaacgtcatcgtgctggagctgaagggcagcgagacgacgtttatgtgcgagtacgcagacgagacggccaccatcgtcgagttcttgaaccggtggatcaccttcagccagtccatcatctccaccctgacc

SEQ ID NO: 242 - DNA, 456 bp, Artificial Sequence
  DNA sequence encoding protein 2d
atggagttcggtctgtcctgggtgttcctggtcgccatcatcaagggcgtgcagtgcgcccccacgtcgtcctccaccaagaagacccagctgcagctcgagcacctcctgctggacctgcagatgatcctgaacgggatcaacaactacaagaacccgaagctgacttgcatgctgaccttcaagttctacatgccgaagaaggcgaccgagctgaagcacctgcagtgcctggaggaggagctcaagccgctcgaggaggtgctgaatctggcccagtccaagaacttccacctgcgcccgcgggacctcatcagcaacatcaacgtcatcgtgctggagctgaagggcagcgagacgacgtttatgtgcgagtacgcagacgagacggccaccatcgtcgagttcttgaaccggtggatcaccttcagccagtccatcatctccaccctgacc

SEQ ID NO: 243 - DNA, 459 bp, Artificial Sequence
  DNA sequence encoding protein 2c
atgtaccgtatgcagctgctctcgtgcatcgcactgagcctcgccctggtgaccaactcagcccccacgtcgtcctccaccaagaagacccagctgcagctcgagcacctcctgctggacctgcagatgatcctgaacgggatcaacaactacaagaacccgaagctgacttgcatgctgaccttcaagttctacatgccgaagaaggcgaccgagctgaagcacctgcagtgcctggaggaggagctcaagccgctcgaggaggtgctgaatctggcccagtccaagaacttccacctgcgcccgcgggacctcatcagcaacatcaacgtcatcgtgctggagctgaagggcagcgagacgacgtttatgtgcgagtacgcagacgagacggccaccatcgtcgagttcttgaaccggtggatcaccttcagccagtccatcatctccaccctgacc

SEQ ID NO: 244 - DNA, 471 bp, Artificial Sequence
  DNA sequence encoding protein 2e
atggcctggaagactctccccatctacctcctgctgctcctgtcggtcttcgtcatccagcaggtgtcctccgcgcccacttcctcgtccaccaagaagacccagctgcagctcgaacacctgctcctcgacctccagatgatcctcaacggaatcaacaactacaagaaccccaagctgacgttcggcaactccaccaagttctacatgcccaagaaggccaccgagctgaagcatctgcagtgcctggaagaggagctcaagccgctcgaggaagtgctgaacctggcccagagcaagaacttccacctccgcccgagggacctgatctcgaacatcaacgtcatcgtgctggagctgaagggatcggagaccacgttcatgtgcgaatacgccgacgagacggccaccatcgtggagttcctcaaccgctggatcaccttctcccagagcataatctccaccctgacc

SEQ ID NO: 245 - DNA, 456 bp, Artificial Sequence
  DNA sequence encoding protein 2g
atggagttcggtctgtcctgggtgttcctggtcgccatcatcaagggcgtgcagtgcgcgcccacttcctcgtccaccaagaagacccagctgcagctcgaacacctgctcctcgacctccagatgatcctcaacggaatcaacaactacaagaaccccaagctgacgttcggcaactccaccaagttctacatgcccaagaaggccaccgagctgaagcatctgcagtgcctggaagaggagctcaagccgctcgaggaagtgctgaacctggcccagagcaagaacttccacctccgcccgagggacctgatctcgaacatcaacgtcatcgtgctggagctgaagggatcggagaccacgttcatgtgcgaatacgccgacgagacggccaccatcgtggagttcctcaaccgctggatcaccttctcccagagcataatctccaccctgacc

SEQ ID NO: 246 - DNA, 459 bp, Artificial Sequence
  DNA sequence encoding protein 2f
atgtaccgcatgcagctgctgtcgtgcatcgcgctcagcctggcgctggtcacgaactcggcgcccacttcctcgtccaccaagaagacccagctgcagctcgaacacctgctcctcgacctccagatgatcctcaacggaatcaacaactacaagaaccccaagctgacgttcggcaactccaccaagttctacatgcccaagaaggccaccgagctgaagcatctgcagtgcctggaagaggagctcaagccgctcgaggaagtgctgaacctggcccagagcaagaacttccacctccgcccgagggacctgatctcgaacatcaacgtcatcgtgctggagctgaagggatcggagaccacgttcatgtgcgaatacgccgacgagacggccaccatcgtggagttcctcaaccgctggatcaccttctcccagagcataatctccaccctgacc

SEQ ID NO: 247 - protein, 157 aa, Artificial Sequence
  Protein 2b
MAWKTLPIYLLLLLSVFVIQQVSSAPTSSSTKKTQLQLEHLLLDLQMILNGINNYKNPKLTCMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 248 - protein, 157 aa, Artificial Sequence
  Protein 2e
MAWKTLPIYLLLLLSVFVIQQVSSAPTSSSTKKTQLQLEHLLLDLQMILNGINNYKNPKLTFGNSTKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 249 - protein, 152 aa, Artificial Sequence
  Protein 2d
MEFGLSWVFLVAIIKGVQCAPTSSSTKKTQLQLEHLLLDLQMILNGINNYKNPKLTCMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 250 - protein, 152 aa, Artificial Sequence
  Protein 2g
MEFGLSWVFLVAIIKGVQCAPTSSSTKKTQLQLEHLLLDLQMILNGINNYKNPKLTFGNSTKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 251 - protein, 153 aa, Artificial Sequence
  Protein 2c
MYRMQLLSCIALSLALVTNSAPTSSSTKKTQLQLEHLLLDLQMILNGINNYKNPKLTCMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 252 - protein, 153 aa, Artificial Sequence
  Protein 2f
MYRMQLLSCIALSLALVTNSAPTSSSTKKTQLQLEHLLLDLQMILNGINNYKNPKLTFGNSTKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 253 - protein, 5 aa, Artificial Sequence
  example peptide sequence
ALMGR

SEQ ID NO: 254 - protein, 5 aa, Artificial Sequence
  glycosylation motif X1X2NX3X4
  MISC_FEATURE (1)..(1): any proteinogenic or non-proteinogenic amino acid or absent
  MISC_FEATURE (2)..(2): any proteinogenic or non-proteinogenic amino acid or absent
  MISC_FEATURE (4)..(4): any proteinogenic or non-proteinogenic amino acid except proline
  MISC_FEATURE (5)..(5): threonine, serine or cysteine
XXNXX

SEQ ID NO: 255 - protein, 4 aa, Artificial Sequence
  glycosylation motif X2NX3X4
  MISC_FEATURE (1)..(1): any proteinogenic or non-proteinogenic amino acid or absent
  MISC_FEATURE (3)..(3): any proteinogenic or non-proteinogenic amino acid except proline
  MISC_FEATURE (4)..(4): threonine, serine or cysteine
XNXX

SEQ ID NO: 256 - protein, 4 aa, Artificial Sequence
  X2NX3T
  MISC_FEATURE (1)..(1): any proteinogenic or non-proteinogenic amino acid or absent
  MISC_FEATURE (1)..(1): any proteinogenic or non-proteinogenic amino acid except proline
  misc_feature (3)..(3): Xaa can be any naturally occurring amino acid
XNXT

SEQ ID NO: 257 - protein, 4 aa, Artificial Sequence
  glycosylation motif X2NX3S
  MISC_FEATURE (1)..(1): any proteinogenic or non-proteinogenic amino acid or absent
  MISC_FEATURE (3)..(3): any proteinogenic or non-proteinogenic amino acid except proline
XNXS

SEQ ID NO: 258 - protein, 4 aa, Artificial Sequence
  glycosylation motif X2NX3C
  MISC_FEATURE (1)..(1): any proteinogenic or non-proteinogenic amino acid or absent
  MISC_FEATURE (3)..(3): any proteinogenic or non-proteinogenic amino acid except proline
XNXC

SEQ ID NO: 259 - protein, 4 aa, Artificial Sequence
  glycosylation motif X2NSX4
  MISC_FEATURE (1)..(1): any proteinogenic or non-proteinogenic amino acid or absent
  MISC_FEATURE (4)..(4): threonine, serine or cysteine
XNSX

SEQ ID NO: 260 - protein, 4 aa, Artificial Sequence
  glycosylation motif X2NST
  MISC_FEATURE (1)..(1): any proteinogenic or non-proteinogenic amino acid or absent
XNST

SEQ ID NO: 261 - protein, 4 aa, Artificial Sequence
  glycosylation motif X2NSS
  MISC_FEATURE (1)..(1): any proteinogenic or non-proteinogenic amino acid or absent
XNSS

SEQ ID NO: 262 - protein, 4 aa, Artificial Sequence
  glycosylation motif X2NSC
  MISC_FEATURE (1)..(1): any proteinogenic or non-proteinogenic amino acid or absent
XNSC

SEQ ID NO: 263 - protein, 4 aa, Artificial Sequence
  glycosylation motif GNX3X4
  MISC_FEATURE (3)..(3): any proteinogenic or non-proteinogenic amino acid except proline
  MISC_FEATURE (4)..(4): threonine, serine and cysteine
GNXX

SEQ ID NO: 264 - protein, 4 aa, Artificial Sequence
  glycosylation motif GNX3T
  MISC_FEATURE (3)..(3): any proteinogenic or non-proteinogenic amino acid except proline
GNXT

SEQ ID NO: 265 - protein, 4 aa, Artificial Sequence
  glycosylation motif GNX3S
  MISC_FEATURE (3)..(3): any proteinogenic or non-proteinogenic amino acid except proline
GNXS

SEQ ID NO: 266 - protein, 4 aa, Artificial Sequence
  glycosylation motif GNX3C
  MISC_FEATURE (3)..(3): any proteinogenic or non-proteinogenic amino acid except proline
GNXC

SEQ ID NO: 267 - protein, 4 aa, Artificial Sequence
  glycosylation motif GNSX4
  MISC_FEATURE (4)..(4): threonine, serine or cysteine
GNSX

SEQ ID NO: 268 - protein, 4 aa, Artificial Sequence
  glycosylation motif GNST
GNST

SEQ ID NO: 269 - protein, 4 aa, Artificial Sequence
  glycosylation motif GNSS
GNSS

SEQ ID NO: 270 - protein, 4 aa, Artificial Sequence
  glycosylation motif GNSC
GNSC

SEQ ID NO: 271 - protein, 4 aa, Artificial Sequence
  glycosylation motif ANX3X4
  MISC_FEATURE (3)..(3): any proteinogenic or non-proteinogenic amino acid except proline
  MISC_FEATURE (4)..(4): threonine, serine or cysteine
ANXX

SEQ ID NO: 272 - protein, 4 aa, Artificial Sequence
  glycosylation motif ANX3T
  MISC_FEATURE (3)..(3): any proteinogenic or non-proteinogenic amino acid except proline
ANXT

SEQ ID NO: 273 - protein, 4 aa, Artificial Sequence
  glycosylation motif ANX3S
  MISC_FEATURE (3)..(3): any proteinogenic or non-proteinogenic amino acid except proline
ANXS

SEQ ID NO: 274 - protein, 4 aa, Artificial Sequence
  glycosylation motif ANX3C
  MISC_FEATURE (3)..(3): any proteinogenic or non-proteinogenic amino acid except proline
ANXC

SEQ ID NO: 275 - protein, 4 aa, Artificial Sequence
  glycosylation motif ANSX4
  MISC_FEATURE (4)..(4): threonine, serine or cysteine
ANSX

SEQ ID NO: 276 - protein, 4 aa, Artificial Sequence
  glycosylation motif ANSC
ANSC

SEQ ID NO: 277 - protein, 4 aa, Artificial Sequence
  glycosylation motif ANST
ANST

SEQ ID NO: 278 - protein, 4 aa, Artificial Sequence
  glycosylation motif ANSS
ANSS

SEQ ID NO: 279 - protein, 5 aa, Artificial Sequence
  glycosylation motif X1X2NX3T
  MISC_FEATURE (1)..(1): any proteinogenic or non-proteinogenic amino acid or absent
  MISC_FEATURE (2)..(2): any proteinogenic or non-proteinogenic amino acid or absent
  MISC_FEATURE (4)..(4): any proteinogenic or non-proteinogenic amino acid except proline
XXNXT

SEQ ID NO: 280 - protein, 5 aa, Artificial Sequence
  glycosylation motif X1X2NX3S
  MISC_FEATURE (1)..(1): any proteinogenic or non-proteinogenic amino acid or absent
  MISC_FEATURE (2)..(2): any proteinogenic or non-proteinogenic amino acid or absent
  MISC_FEATURE (4)..(4): any proteinogenic or non-proteinogenic amino acid except proline
XXNXS

SEQ ID NO: 281 - protein, 5 aa, Artificial Sequence
  glycosylation motif X1X2NX3C
  MISC_FEATURE (1)..(1): any proteinogenic or non-proteinogenic amino acid or absent
  MISC_FEATURE (2)..(2): any proteinogenic or non-proteinogenic amino acid or absent
  MISC_FEATURE (4)..(4): any proteinogenic or non-proteinogenic amino acid except proline
XXNXC

SEQ ID NO: 282 - protein, 5 aa, Artificial Sequence
  glycosylation motif X1X2NSX4
  MISC_FEATURE (1)..(1): any proteinogenic or non-proteinogenic amino acid or absent
  MISC_FEATURE (2)..(2): any proteinogenic or non-proteinogenic amino acid or absent
  MISC_FEATURE (5)..(5): threonine, serine or cysteine
XXNSX

SEQ ID NO: 283 - protein, 5 aa, Artificial Sequence
  glycosylation motif X1X2NST
  MISC_FEATURE (1)..(1): any proteinogenic or non-proteinogenic amino acid or absent
  MISC_FEATURE (2)..(2): any proteinogenic or non-proteinogenic amino acid or absent
XXNST

SEQ ID NO: 284 - protein, 5 aa, Artificial Sequence
  glycosylation motif X1X2NSS
  MISC_FEATURE (1)..(1): any proteinogenic or non-proteinogenic amino acid or absent
  MISC_FEATURE (2)..(2): any proteinogenic or non-proteinogenic amino acid or absent
XXNSS

SEQ ID NO: 285 - protein, 5 aa, Artificial Sequence
  glycosylation motif X1X2NSC
  MISC_FEATURE (1)..(1): any proteinogenic or non-proteinogenic amino acid or absent
  MISC_FEATURE (2)..(2): any proteinogenic or non-proteinogenic amino acid or absent
XXNSC

SEQ ID NO: 286 - protein, 5 aa, Artificial Sequence
  glycosylation motif X1GNX3X4
  MISC_FEATURE (1)..(1): any proteinogenic or non-proteinogenic amino acid or absent
  MISC_FEATURE (4)..(4): any proteinogenic or non-proteinogenic amino acid except proline
  MISC_FEATURE (5)..(5): threonine, serine or cysteine
XGNXX

SEQ ID NO: 287 - protein, 5 aa, Artificial Sequence
  glycosylation motif X1GNX3T
  MISC_FEATURE (1)..(1): any proteinogenic or non-proteinogenic amino acid or absent
  MISC_FEATURE (4)..(4): any proteinogenic or non-proteinogenic amino acid except proline
XGNXT

SEQ ID NO: 288 - protein, 5 aa, Artificial Sequence
  glycosylation motif X1GNX3S
  MISC_FEATURE (1)..(1): any proteinogenic or non-proteinogenic amino acid or absent
  MISC_FEATURE (4)..(4): any proteinogenic or non-proteinogenic amino acid except proline
XGNXS

SEQ ID NO: 289 - protein, 5 aa, Artificial Sequence
  glycosylation motif X1GNX3C
  MISC_FEATURE (1)..(1): any proteinogenic or non-proteinogenic amino acid or absent
  MISC_FEATURE (4)..(4): any proteinogenic or non-proteinogenic amino acid except proline
XGNXC

SEQ ID NO: 290 - protein, 5 aa, Artificial Sequence
  glycosylation motif X1GNSX4
  MISC_FEATURE (1)..(1): any proteinogenic or non-proteinogenic amino acid or absent
  MISC_FEATURE (5)..(5): threonine, serine or cysteine
XGNSX

SEQ ID NO: 291 - protein, 5 aa, Artificial Sequence
  glycosylation motif X1GNST
  MISC_FEATURE (1)..(1): any proteinogenic or non-proteinogenic amino acid or absent
XGNST

SEQ ID NO: 292 - protein, 5 aa, Artificial Sequence
  glycosylation motif X1GNSS
  MISC_FEATURE (1)..(1): any proteinogenic or non-proteinogenic amino acid or absent
XGNSS

SEQ ID NO: 293 - protein, 5 aa, Artificial Sequence
  glycosylation motif X1GNSC
  MISC_FEATURE (1)..(1): any proteinogenic or non-proteinogenic amino acid or absent
XGNSC

SEQ ID NO: 294 - protein, 5 aa, Artificial Sequence
  glycosylation motif FX2NX3X4
  MISC_FEATURE (2)..(2): any proteinogenic or non-proteinogenic amino acid or absent
  MISC_FEATURE (4)..(4): any proteinogenic or non-proteinogenic amino acid except proline
  MISC_FEATURE (5)..(5): threonine, serine or cysteine
FXNXX

SEQ ID NO: 295 - protein, 5 aa, Artificial Sequence
  glycosylation motif FX2NX3T
  MISC_FEATURE (2)..(2): any proteinogenic or non-proteinogenic amino acid or absent
  MISC_FEATURE (4)..(4): any proteinogenic or non-proteinogenic amino acid except proline
FXNXT

SEQ ID NO: 296 - protein, 5 aa, Artificial Sequence
  glycosylation motif FX2NX3S
  MISC_FEATURE (2)..(2): any proteinogenic or non-proteinogenic amino acid or absent
  MISC_FEATURE (4)..(4): any proteinogenic or non-proteinogenic amino acid except proline
FXNXS

SEQ ID NO: 297 - protein, 5 aa, Artificial Sequence
  glycosylation motif FX2NX3C
  MISC_FEATURE (2)..(2): any proteinogenic or non-proteinogenic amino acid or absent
  MISC_FEATURE (4)..(4): any proteinogenic or non-proteinogenic amino acid except proline
FXNXC

SEQ ID NO: 298 - protein, 5 aa, Artificial Sequence
  glycosylation motif FX2NSX4
  MISC_FEATURE (2)..(2): any proteinogenic or non-proteinogenic amino acid or absent
  MISC_FEATURE (5)..(5): threonine, serine or cysteine
FXNSX

SEQ ID NO: 299 - protein, 5 aa, Artificial Sequence
  glycosylation motif FX2NST
  MISC_FEATURE (2)..(2): any proteinogenic or non-proteinogenic amino acid or absent
FXNST

SEQ ID NO: 300 - protein, 5 aa, Artificial Sequence
  glycosylation motif FX2NSS
  MISC_FEATURE (2)..(2): any proteinogenic or non-proteinogenic amino acid or absent
FXNSS

SEQ ID NO: 301 - protein, 5 aa, Artificial Sequence
  glycosylation motif FX2NSC
  MISC_FEATURE (2)..(2): any proteinogenic or non-proteinogenic amino acid or absent
FXNSC

SEQ ID NO: 302 - protein, 5 aa, Artificial Sequence
  glycosylation motif FGNX3X4
  MISC_FEATURE (4)..(4): any proteinogenic or non-proteinogenic amino acid except proline
  MISC_FEATURE (5)..(5): threonine, serine or cysteine
FGNXX

SEQ ID NO: 303 - protein, 5 aa, Artificial Sequence
  glycosylation motif FGNX3T
  MISC_FEATURE (4)..(4): any proteinogenic or non-proteinogenic amino acid except proline
FGNXT

SEQ ID NO: 304 - protein, 5 aa, Artificial Sequence
  glycosylation motif FGNX3S
  MISC_FEATURE (4)..(4): any proteinogenic or non-proteinogenic amino acid except proline
FGNXS

SEQ ID NO: 305 - protein, 5 aa, Artificial Sequence
  glycosylation motif FGNX3C
  MISC_FEATURE (4)..(4): any proteinogenic or non-proteinogenic amino acid except proline
FGNXC

SEQ ID NO: 306 - protein, 5 aa, Artificial Sequence
  glycosylation motif FGNSX4
  MISC_FEATURE (5)..(5): threonine, serine or cysteine
FGNSX

SEQ ID NO: 307 - protein, 5 aa, Artificial Sequence
  glycosylation motif FGNST
FGNST

SEQ ID NO: 308 - protein, 5 aa, Artificial Sequence
  glycosylation motif FGNSS
FGNSS

SEQ ID NO: 309 - protein, 5 aa, Artificial Sequence
  glycosylation motif FGNSC
FGNSC

SEQ ID NO: 310 - protein, 5 aa, Artificial Sequence
  glycosylation motif X1ANX3X4
  MISC_FEATURE (1)..(1): any proteinogenic or non-proteinogenic amino acid or absent
  MISC_FEATURE (4)..(4): any proteinogenic or non-proteinogenic amino acid except proline
  MISC_FEATURE (5)..(5): threonine, serine or cysteine
XANXX

SEQ ID NO: 311 - protein, 5 aa, Artificial Sequence
  glycosylation motif X1ANX3T
  MISC_FEATURE (1)..(1): any proteinogenic or non-proteinogenic amino acid or absent
  MISC_FEATURE (4)..(4): any proteinogenic or non-proteinogenic amino acid except proline
XANXT

SEQ ID NO: 312 - protein, 5 aa, Artificial Sequence
  glycosylation motif X1ANX3S
  MISC_FEATURE (1)..(1): any proteinogenic or non-proteinogenic amino acid or absent
  MISC_FEATURE (4)..(4): any proteinogenic or non-proteinogenic amino acid except proline
XANXS

SEQ ID NO: 313 - protein, 5 aa, Artificial Sequence
  glycosylation motif X1ANX3C
  MISC_FEATURE (1)..(1): any proteinogenic or non-proteinogenic amino acid or is absent
  MISC_FEATURE (4)..(4): any proteinogenic or non-proteinogenic amino acid except proline
XANXC

SEQ ID NO: 314 - protein, 5 aa, Artificial Sequence
  glycosylation motif X1ANSX4
  MISC_FEATURE (1)..(1): any proteinogenic or non-proteinogenic amino acid or absent
  MISC_FEATURE (4)..(4): threonine, serine or cysteine
  misc_feature (5)..(5): Xaa can be any naturally occurring amino acid
XANSX

SEQ ID NO: 315 - protein, 5 aa, Artificial Sequence
  glycosylation motif X1ANST
  MISC_FEATURE (1)..(1): any proteinogenic or non-proteinogenic amino acid or absent
XANST

SEQ ID NO: 316 - protein, 5 aa, Artificial Sequence
  glycosylation motif X1ANSS
  MISC_FEATURE (1)..(1): any proteinogenic or non-proteinogenic amino acid or is absent
XANSS

SEQ ID NO: 317 - protein, 5 aa, Artificial Sequence
  glycosylation motif X1ANSC
  MISC_FEATURE (1)..(1): any proteinogenic or non-proteinogenic amino acid or absent
XANSC

SEQ ID NO: 318 - protein, 5 aa, Artificial Sequence
  glycosylation motif FX2NX3X4
  MISC_FEATURE (2)..(2): any proteinogenic or non-proteinogenic amino acid or absent
  MISC_FEATURE (4)..(4): any proteinogenic or non-proteinogenic amino acid except proline
  MISC_FEATURE (5)..(5): threonine, serine or cysteine
FXNXX

SEQ ID NO: 319 - protein, 5 aa, Artificial Sequence
  glycosylation motif FX2NX3T
  MISC_FEATURE (2)..(2): any proteinogenic or non-proteinogenic amino acid or absent
  MISC_FEATURE (4)..(4): any proteinogenic or non-proteinogenic amino acid except proline
FXNXT

SEQ ID NO: 320 - protein, 5 aa, Artificial Sequence
  glycosylation motif FX2NX3S
  MISC_FEATURE (2)..(2): any proteinogenic or non-proteinogenic amino acid or absent
  MISC_FEATURE (4)..(4): any proteinogenic or non-proteinogenic amino acid except proline
FXNXS

SEQ ID NO: 321 - protein, 5 aa, Artificial Sequence
  glycosylation motif FX2NX3C
  MISC_FEATURE (2)..(2): any proteinogenic or non-proteinogenic amino acid or absent
  MISC_FEATURE (4)..(4): any proteinogenic or non-proteinogenic amino acid except proline
FXNXC

SEQ ID NO: 322 - protein, 5 aa, Artificial Sequence
  glycosylation motif FX2NSX4
  MISC_FEATURE (2)..(2): any proteinogenic or non-proteinogenic amino acid or absent
  MISC_FEATURE (5)..(5): threonine, serine or cysteine
FXNSX

SEQ ID NO: 323 - protein, 5 aa, Artificial Sequence
  glycosylation motif FX2NST
  MISC_FEATURE: any proteinogenic or non-proteinogenic amino acid or absent
  misc_feature (2)..(2): Xaa can be any naturally occurring amino acid
FXNST

SEQ ID NO: 324 - protein, 5 aa, Artificial Sequence
  glycosylation motif FX2NSS
  MISC_FEATURE (2)..(2): any proteinogenic or non-proteinogenic amino acid or absent
FXNSS

SEQ ID NO: 325 - protein, 5 aa, Artificial Sequence
  glycosylation motif FX2NSC
  MISC_FEATURE (2)..(2): any proteinogenic or non-proteinogenic amino acid or is absent
FXNSC

SEQ ID NO: 326 - protein, 5 aa, Artificial Sequence
  glycosylation motif FANX3X4
  MISC_FEATURE (4)..(4): any proteinogenic or non-proteinogenic amino acid except proline
  MISC_FEATURE (5)..(5): threonine, serine or cysteine
FANXX

SEQ ID NO: 327 - protein, 5 aa, Artificial Sequence
  glycosylation motif FANX3T
  MISC_FEATURE (4)..(4): any proteinogenic or non-proteinogenic amino acid except proline
FANXT

SEQ ID NO: 328 - protein, 5 aa, Artificial Sequence
  glycosylation motif FANX3S
  MISC_FEATURE (4)..(4): any proteinogenic or non-proteinogenic amino acid except proline
FANXS

SEQ ID NO: 329 - protein, 5 aa, Artificial Sequence
  glycosylation motif FANX3C
  MISC_FEATURE (4)..(4): any proteinogenic or non-proteinogenic amino acid except proline
FANXC

SEQ ID NO: 330 - protein, 5 aa, Artificial Sequence
  glycosylation motif FANSX4
  MISC_FEATURE (5)..(5): threonine, serine or cysteine
FANSX

SEQ ID NO: 331 - protein, 5 aa, Artificial Sequence
  glycosylation motif FANST
FANST

SEQ ID NO: 332 - protein, 5 aa, Artificial Sequence
  glycosylation motif FANSS
FANSS

SEQ ID NO: 333 - protein, 5 aa, Artificial Sequence
  glycosylation motif FANSC
FANSC

SEQ ID NO: 334 - protein, 132 aa, Artificial Sequence
  IL-2 protein of formula (I-ii)
PTSSSTKKTQLQLEHLLLDLQMILNGINNYKNPKLTFGDSTKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 335 - protein, 132 aa, Artificial Sequence
  IL-2 protein of formula (I-ii)
PTSSSTKKTQLQLEHLLLDLQMILNGINNYKNPKLTFADSTKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 336 - protein, 132 aa, Artificial Sequence
  IL-2 protein of formula (I-ii)
PTSSSTKKTQLQLEHLLLDLQMILNGINNYKNPKLTRMLFGDSTMPKKATELKHLQCLEEELKPLEEVLNLAQSKNFHLRPRDLISNINVIVLELKGSETTFMCEYADETATIVEFLNRWITFSQSIISTLT

SEQ ID NO: 337 - protein, 132 aa, Artificial Sequence
  IL-2 protein of formula (I-ii)
PTSSSTKKTQLQLEHLLLDLQMILNGINNYKNPKLTRMLFADSTMPKKATELKHLQCLEEELKPLEEVLNLAQSKNFHLRPRDLISNINVIVLELKGSETTFMCEYADETATIVEFLNRWITFSQSIISTLT

SEQ ID NO: 338 - protein, 132 aa, Artificial Sequence
  IL-2 protein of formula (I-ii)
PTSSSTKKTQLQLEHLLLDLQMILNGINNYKNPKLTRMLTFKFYMPKKATELKHLQCLEFGDSTLEEVLNLAQSKNFHLRPRDLISNINVIVLELKGSETTFMCEYADETATIVEFLNRWITFSQSIISTLT

SEQ ID NO: 339 - protein, 132 aa, Artificial Sequence
  IL-2 protein of formula (I-ii)
PTSSSTKKTQLQLEHLLLDLQMILNGINNYKNPKLTRMLTFKFYMPKKATELKHLQCLEFADSTLEEVLNLAQSKNFHLRPRDLISNINVIVLELKGSETTFMCEYADETATIVEFLNRWITFSQSIISTLT

SEQ ID NO: 340 - protein, 133 aa, Artificial Sequence
  IL-2 protein of formula (I-ii)
APTSSSTKKTQLQLEHLLLDLQMILNGINNYKNPKLTFGDSTKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 341 - protein, 133 aa, Artificial Sequence
  IL-2 protein of formula (I-ii)
APTSSSTKKTQLQLEHLLLDLQMILNGINNYKNPKLTFADSTKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 342 - protein, 133 aa, Artificial Sequence
  IL-2 protein of formula (I-ii)
APTSSSTKKTQLQLEHLLLDLQMILNGINNYKNPKLTRMLFGDSTMPKKATELKHLQCLEEELKPLEEVLNLAQSKNFHLRPRDLISNINVIVLELKGSETTFMCEYADETATIVEFLNRWITFSQSIISTLT

SEQ ID NO: 343 - protein, 133 aa, Artificial Sequence
  IL-2 protein of formula (I-ii)
APTSSSTKKTQLQLEHLLLDLQMILNGINNYKNPKLTRMLFADSTMPKKATELKHLQCLEEELKPLEEVLNLAQSKNFHLRPRDLISNINVIVLELKGSETTFMCEYADETATIVEFLNRWITFSQSIISTLT

SEQ ID NO: 344 - protein, 133 aa, Artificial Sequence
  IL-2 protein of formula (I-ii)
APTSSSTKKTQLQLEHLLLDLQMILNGINNYKNPKLTRMLTFKFYMPKKATELKHLQCLEFGDSTLEEVLNLAQSKNFHLRPRDLISNINVIVLELKGSETTFMCEYADETATIVEFLNRWITFSQSIISTLT

SEQ ID NO: 345 - protein, 133 aa, Artificial Sequence
  IL-2 protein of formula (I-ii)
APTSSSTKKTQLQLEHLLLDLQMILNGINNYKNPKLTRMLTFKFYMPKKATELKHLQCLEFADSTLEEVLNLAQSKNFHLRPRDLISNINVIVLELKGSETTFMCEYADETATIVEFLNRWITFSQSIISTLT

SEQ ID NO: 346 - protein, 46 aa, Artificial Sequence
  SEQ D
YKNPFADSTLTFKFYMPKKATELKHLQCLEEELKPLEEVLNLAQSK

SEQ ID NO: 347 - protein, 46 aa, Artificial Sequence
  SEQ D
YKNPKLTFADSTKFYMPKKATELKHLQCLEEELKPLEEVLNLAQSK

SEQ ID NO: 348 - protein, 46 aa, Artificial Sequence
  SEQ D
YKNPKLTRFADSTFYMPKKATELKHLQCLEEELKPLEEVLNLAQSK

SEQ ID NO: 349 - protein, 46 aa, Artificial Sequence
  SEQ D
YKNPKLTRMLFADSTMPKKATELKHLQCLEEELKPLEEVLNLAQSK

SEQ ID NO: 350 - protein, 46 aa, Artificial Sequence
  SEQ D
YKNPKLTRMLTFADSTPKKATELKHLQCLEEELKPLEEVLNLAQSK

SEQ ID NO: 351 - protein, 46 aa, Artificial Sequence
  SEQ D
YKNPKLTRMLTFFADSTKKATELKHLQCLEEELKPLEEVLNLAQSK

SEQ ID NO: 352 - protein, 46 aa, Artificial Sequence
  SEQ D
YKNPKLTRMLTFKFADSTKATELKHLQCLEEELKPLEEVLNLAQSK

SEQ ID NO: 353 - protein, 46 aa, Artificial Sequence
  SEQ D
YKNPKLTRMLTFKFFADSTATELKHLQCLEEELKPLEEVLNLAQSK

SEQ ID NO: 354 - protein, 46 aa, Artificial Sequence
  SEQ D
YKNPKLTRMLTFKFYMPKKATELKHLQCLEFADSTLEEVLNLAQSK

SEQ ID NO: 355 - protein, 46 aa, Artificial Sequence
  SEQ D
YKNPKLTRMLTFKFYMPKKATELKHLQCLEEFADSTEEVLNLAQSK

SEQ ID NO: 356 - protein, 46 aa, Artificial Sequence
  SEQ D
YKNPKLTRMLTFKFYMPKKATELKHLQCLEEELKPLEEVLNFADST

SEQ ID NO: 357 - protein, 46 aa, Artificial Sequence
  SEQ D
YKNPFGDSTLTFKFYMPKKATELKHLQCLEEELKPLEEVLNLAQSK

SEQ ID NO: 358 - protein, 46 aa, Artificial Sequence
  SEQ D
YKNPKLTFGDSTKFYMPKKATELKHLQCLEEELKPLEEVLNLAQSK

SEQ ID NO: 359 - protein, 46 aa, Artificial Sequence
  SEQ D
YKNPKLTRFGDSTFYMPKKATELKHLQCLEEELKPLEEVLNLAQSK

SEQ ID NO: 360 - protein, 46 aa, Artificial Sequence
  SEQ D
YKNPKLTRMLFGDSTMPKKATELKHLQCLEEELKPLEEVLNLAQSK

SEQ ID NO: 361 - protein, 46 aa, Artificial Sequence
  SEQ D
YKNPKLTRMLTFGDSTPKKATELKHLQCLEEELKPLEEVLNLAQSK

SEQ ID NO: 362 - protein, 46 aa, Artificial Sequence
  SEQ D
YKNPKLTRMLTFFGDSTKKATELKHLQCLEEELKPLEEVLNLAQSK

SEQ ID NO: 363 - protein, 46 aa, Artificial Sequence
  SEQ D
YKNPKLTRMLTFKFGDSTKATELKHLQCLEEELKPLEEVLNLAQSK

SEQ ID NO: 364 - protein, 46 aa, Artificial Sequence
  SEQ D
YKNPKLTRMLTFKFFGDSTATELKHLQCLEEELKPLEEVLNLAQSK

SEQ ID NO: 365 - protein, 46 aa, Artificial Sequence
  SEQ D
YKNPKLTRMLTFKFYMPKKATELKHLQCLEFGDSTLEEVLNLAQSK

SEQ ID NO: 366 - protein, 46 aa, Artificial Sequence
  SEQ D
YKNPKLTRMLTFKFYMPKKATELKHLQCLEEFGDSTEEVLNLAQSK

SEQ ID NO: 367 - protein, 46 aa, Artificial Sequence
  SEQ D
YKNPKLTRMLTFKFYMPKKATELKHLQCLEEELKPLEEVLNFGDST

SEQ ID NO: 368 - protein, 5 aa, Artificial Sequence
  example sequence
FGDST

(19) United States
(12) Patent Application Publication
(10) Pub. No.: US 2023/0340055 A1
(43) Pub. Date: Oct. 26, 2023

(54) GLYCOSYLATED IL-2 PROTEINS AND USES THEREOF

(71) Applicant: ASCENDIS PHARMA ONCOLOGY DIVISION A/S, Hellerup (DK)

(72) Inventors: Sigurd OKKELS, Hellerup (DK); David B. ROSEN, Palo Alto, CA (US); Burkhardt LAUFER, Heidelberg (DE); Thomas KNAPPE, Heidelberg (DE)

(21) Appl. No.: 18/043,319

(22) PCT Filed: Aug. 27, 2021
(86) PCT No.: PCT/EP2021/073735

§ 371 Date: Feb. 27, 2023

(30) Foreign Application Priority Data
Aug. 28, 2020 (US) 63/071627
Oct. 16, 2020 (EP) 20202306.5
Dec. 21, 2020 (EP) 20216067.7
Mar. 3, 2021 (EP) 21160466.5
Mar. 11, 2021 (EP) 21162023.2

(51) Int. Cl. A61P 35/00; C07K 14/55

(52) U.S. Cl. CPC C07K 14/55; A61P 35/00; C07K 2319/91

(57) ABSTRACT

The present invention relates to an IL-2 protein sequence of the formula (Tag¹)_{y} - (Ala)ₓ - SEQA - SEQ B - SEQ C -(Tag²)_{z} (I), wherein SEQ A has at least 89% sequence identity with SEQ ID NO:1; SEQ B has at least 76% sequence identity to SEQ ID NO:2 and comprises at least one glycosylation motif; SEQ C has at least 91% sequence identity with SEQ ID NO:4; Tag 1 and Tag² are independently a tag moiety; Ala is an alanine residue; x is 0 or 1; y is 0 or 1; and z is 0 or 1; to conjugates thereof and their uses in the treatment of cell-proliferation disorders.

The present invention relates to an IL-2 protein sequence of the formula (Tag¹)y - (Ala)ₓ - SEQ A - SEQ B - SEQ C - (Tag²)_{z} (I), wherein SEQ A has at least 89% sequence identity with SEQ ID NO:1; SEQ B has at least 76% sequence identity to SEQ ID NO:2 and comprises at least one glycosylation motif; SEQ C has at least 91% sequence identity with SEQ ID NO:4; Tag¹ and Tag² are independently a tag moiety; Ala is an alanine residue; x is 0 or 1; y is 0 or 1; and z is 0 or 1; to conjugates thereof and their uses in the treatment of cell-proliferation disorders.

In healthy humans, the immune system can often discriminate between healthy cells and cancerous cells. Upon identifying a given cell as cancerous, the immune system typically eliminates it. However, when the immune system is compromised from e.g. acute or chronic defects or is overwhelmed, cancers can develop resulting from a compromised immune system’s inability to differentiate, and then eliminate, cancer cells. In a patient suffering from cancer, administration of an immunomodulatory protein to the patient may help activate that patient’s immune system so that the immune system’s ability to eliminate cancer cells is enhanced. In a patient suffering from a viral infection, administration of an immunomodulatory protein to the patient may help activate that patient’s immune system so that the immune system’s ability to eliminate the viral infection is enhanced. Similarly, even in a healthy patient the immune response to a vaccine can be enhanced by the addition of such immunomodulatory proteins.

One such immunomodulatory protein used in the treatment of patients suffering from certain cancers is interleukin-2 (IL-2). Human IL-2 is synthesized as a 153-amino-acid precursor polypeptide (full-length) and is then processed to mature IL-2. Mature human IL-2 is a 15.5 kDa four-alpha-helix bundle glycoprotein that consists of 133 amino acids. IL-2 plays a central role in the generation, differentiation, survival and homeostasis of immune effector cells. IL-2 is synthesized by activated CD4+ helper T cells, and through differential receptor interaction IL-2 can modulate the immune response towards immunity or tolerance.

IL-2 acts by binding to IL-2 receptors (IL-2R). Association of the α- (CD25), β- (CD122) and common γ- (γc, CD132) subunits results in the trimeric high-affinity IL-2Rαβγ. The dimeric intermediate affinity IL-2Rβγ consists of the β- and γ-subunits and binds IL-2 with 50-fold lower affinity. IL-2Rα is not required for IL-2 signaling but confers the high affinity binding of the trimeric receptor, whereas the β- and γ-subunits mediate signal transduction. IL-2Rβγ is expressed on NK cells, monocytes, macrophages, γδ T cells, in particular Vγ9Vδ2 T cells, and resting CD4+ and CD8+ T cells, while IL-2Rαβγ is transiently induced on activated T and NK cells, and is constitutively expressed on T regulatory cells as well as type 2 innate lymphocyte cells (ILC2s), eosinophils and endothelial cells. The ability of IL-2 to expand and activate innate and adaptive effector cells is the basis of its antitumor activity.

In patients, IL-2 can stimulate antitumor efficacy, characterized by increases in cytotoxic lymphocytes, including effector T and NK cells, when given at high-doses (i.e., 600000-720000 IU/kg body weight three times daily for up to 14 doses per cycle in humans). Presumably during this therapy all T cells are stimulated by IL-2 after high-doses are administered. However, when the therapy cycle ends as well as at later timepoints after any individual dose IL-2 levels will drop. As a result, IL-2 will become limiting and T regulatory (Treg) cells expressing IL-2Rαβγ will outcompete effector T cells expressing IL-2Rβγ for the remaining wild type IL-2.

However, IL-2′s antitumor immunity is dose limited by severe cardiovascular, pulmonary, hepatic, gastrointestinal, neurologic and hematological side effects, such that it is only given to patients at specialized centers. Many of these adverse events are characterized by a vascular leak syndrome (VLS), also known as capillary leak syndrome. There are several proposed mechanisms for causing VLS, many of which involve interaction between wild type IL-2 and IL-2Rαβγ expressing cells such as ILC2s, eosinophils, and endothelial cells.

Effector CD4+ T cells, CD8+ T-cells, γδ T cells, in particular Vγ9Vδ2 T cells, and NK cells, which significantly enhance anti-tumor immune responses, preferentially express the IL-2Rβγ form of the IL-2R. Thus, administration of compounds that bind to and are agonists for IL-2Rβγ can be expected to enhance the immune response against tumors (by, e.g., increasing the proliferation and activity of effect of CD4+ T cells, CD8+ T-cells, γδ T cells, in particular Vγ9Vδ2 T cells, and NK cells).

Thus, administration of IL-2Rβγ-selective agonists (having reduced or no binding to IL-2Rα or enhanced binding to IL-2Rβγ) would be beneficial to patients suffering from certain cancers as doing so is expected to reduce systemic vascular leak side effects such as pulmonary edema, hypotension, and eosinophilia, providing an improved therapeutic window.

One way of synthesizing such biased IL-2, i.e. an IL-2 protein that preferentially binds to IL-2Rβγ, is introducing steric hindrance so that binding of IL-2 to the IL-2Rα subunit is blocked. This can be achieved by conjugating certain moieties to endogenous or mutated amino acids in the IL-2 sequence to mediate the non-IL-2Rα binding bias, such as shown in WO2019/185705A1. However, manufacturing such mutated or biased IL-2 proteins in sufficient yield and quality may be challenging and conjugating said moieties to IL-2 requires additional synthesis steps, which increase costs and may have a negative impact on quality and overall yield.

It is therefore an object of the present invention to at least partially overcome the above-mentioned disadvantage.

This object is achieved with an IL-2 protein sequence of formula (I)

$${\left({\text{Tag}}^{\text{1}}\right)}_{\text{y}} - {\left(\text{Ala}\right)}_{\text{x}} - \text{SEQ} \text{A} - \text{SEQ} \text{B} - \text{SEQ} \text{C} - {\left({\text{Tag}}^{\text{2}}\right)}_{\text{z}} \qquad \text{(I)}$$

wherein
SEQ A has at least 89% sequence identity with SEQ ID NO:1;
SEQ B has at least 76% sequence identity to SEQ ID NO:2 and comprises at least one glycosylation motif;
SEQ C has at least 91% sequence identity to SEQ ID NO:4;
Tag¹ and Tag² are independently a tag moiety;
Ala is an alanine residue;
x is 0 or 1;
y is 0 or 1; and
z is 0 or 1.

It was surprisingly found that without being bound by theory introduction of glycosylation motifs at specific locations of the amino acid sequence of IL-2 variants that are mediating IL-2Rα binding resulted in efficient site-specific glycosylation and provided efficient IL-2Rβγ binding bias as demonstrated by reduced Treg activation and sustained CD8+ T-cells activity, while avoiding any additional conjugation steps.

Surprisingly, insertion of a glycosylation motif into the IL-2Rα binding region resulted in certain embodiments in a particularly high glycosylation rate at the intended sites and the expression of glycosylated IL-2 variants was surprisingly more efficient than the expression of a non-glycosylated IL-2 variant. Furthermore, without being bound by theory, this glycosylation effectively blocked activation of IL-2Rα⁺ cells without the need for the conjugation of any additional moiety, thus making manufacturing of a biased IL-2 more efficient.

Within the present invention the terms are used having the meaning as follows.

In general, the term “interleukin-2” or “IL-2” refers to all IL-2 proteins, preferably from mammalian species, more preferably from primate species and most preferably from human, as well as their variants, analogs, orthologs, homologs, and derivatives and fragments and fusion proteins thereof, that are characterized by playing a central role in lymphocyte generation, survival and homeostasis, and also encompasses naturally occurring variants of IL-2, e.g. splice variants or allelic variants. In the context of this invention the terms “interleukin-2” and “IL-2” in particular refer to the protein having the sequence of formula (I).

Human full-length IL-2 has the following sequence:

MYRMQLLSCIALSLALVTNSAPTSSSTKKTQLQLEHLLLDLQMILNGINN
YKNPKLTRMLTFKFYMPKKATELKHLQCLEEELKPLEEVLNLAQSKNFHL
RPRDLISNINVIVLELKGSETTFMCEYADETATIVEFLNRWITFCQSIIS
TLT (SEQ ID NO:5)

The mature form of human IL-2 has the following sequence:

APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRWITFCQSIISTLT (SEQ ID NO:6)

As used herein the term “biased IL-2” refers to modified IL-2, in which the ratio of the K_{D} of said biased IL-2 to IL-2Rα to the K_{D} of said biased IL-2 to IL-2Rβ is larger than the ratio of the K_{D} of IL-2 of SEQ ID NO:213 to IL-2Rα to the K_{D} of IL-2 of SEQ ID NO:213 to IL-2Rβ. This is described by the following formula:

$$\frac{{\text{Ratio}}_{\text{biased} \text{IL-2}}}{{\text{Ratio}}_{\text{IL-2} \text{of} \text{SEQ} \text{ID} \text{NO:213}}} > 1$$

wherein

$${\text{Ratio}}_{\text{biased} \text{IL-2}} \text{=} \frac{{\text{K}}_{\text{D}} \text{biased} \text{IL-2} \text{to} \text{IL-2R} \text{α}}{{\text{K}}_{\text{D}} \text{biased} \text{IL-2} \text{to} \text{IL-2R} \text{β}}$$

$${\text{Ratio}}_{\text{IL-2} \text{of} \text{SEQ} \text{ID} \text{NO:213}} \frac{{\text{K}}_{\text{D}} \text{IL-2} \text{of} \text{SEQ} \text{ID} \text{NO:213} \text{to} \text{IL-2R} \text{α}}{{\text{K}}_{\text{D}} \text{IL-2} \text{of} \text{SEQ} \text{ID} \text{NO:213} \text{to} \text{IL-2R} \text{β}}$$

with
“K_{D} biased IL-2 to IL-2Rα” being the K_{D} of biased IL-2 to IL-2Rα,
“K_{D} biased IL-2 to IL-2Rβ” being the K_{D} of biased IL-2 to IL-2Rβ,
“K_{D} IL-2 of SEQ ID NO:213 to IL-2Rα” being the K_{D} of IL-2 of SEQ ID NO:213 to IL-2Rα, and
“K_{D} IL-2 of SEQ ID NO:213 to IL-2Rβ” being the K_{D} of IL-2 of SEQ ID NO:213 to IL-2Rβ.

IL-2 of SEQ ID NO:213 has the following sequence:

MAPTSSSTKKTQLQLEHLLLDLQMILNGINNYKNPKLTRMLTFKFYMPKK
ATELKHLQCLEEELKPLEEVLNLAQSKNFHLRPRDLISNINVIVLELKGS
ETTFMCEYADETATIVEFLNRWITFAQSIISTLT (SEQ ID NO:213)

Binding affinity/kinetics needed to determine the K_{D} of biased IL-2 to IL-2Rα, the K_{D} of biased IL-2 to IL-2Rβ, the K_{D} of IL-2 of SEQ ID NO:213 to IL-2Rα and the K_{D} of IL-2 of SEQ ID NO:213 to IL-2Rβ may be assessed using surface plasmon resonance (SPR), measured on a Biacore instrument (GE Healthcare) as follows: A human Fc capture surface on a CM5 (or alternatively Cl or CM4) chip is prepared by covalent coating with anti-human Fc antibody or alternatively a protein A chip is used. Next, IL-2Rβ-Fc or IL2-Rα-Fc is immobilized on the chip. To measure the affinity/kinetic constants, serial dilutions of the analytes are made starting at for example between 1 nM and 2 µM or at 30 nM and 500 nM for IL-2 compounds. Analytes are each exposed to the receptor-modified chip for a suitable amount of time, such as for 1 to 30 minutes, which may for example be 2 minutes or may be 3 minutes and are then washed away for a suitable amount of time, such as 2 to 60 minutes, which may for example be 10 minutes. The resulting binding curves from the dilution series are fit to a 1:1 kinetic model to correlate observed response units (R) to the association and dissociation rate constants, kₐ and k_{d}:

$$\text{R =} \text{} \frac{{\text{k}}_{\text{a}} {\text{CR}}_{\text{max}}}{{\text{k}}_{\text{a}} {\text{C + k}}_{\text{d}}} \times \left(1 - {e}^{- \left({\text{k}}_{\text{a}} {\text{C+k}}_{\text{d}}\right) \text{t}}\right)$$

wherein
t is time;
C is the concentration of the analyte; and
Rₘₐₓ is the maximum binding capacity of the surface.

If determined via a kinetic 1:1 model the ratio of the dissociation and association rates provides the equilibrium dissociation constant K_{D}.

Alternatively, the resulting binding curves from the dilution series are fit to a 1:1 steady state interaction model which calculates K_{D} for a 1:1 interaction from a plot of steady-state binding levels (R_{eq}) against analyte concentration (C):

$${\text{R}}_{\text{eq}} \text{=} \frac{\text{C} \times {\text{R}}_{\text{max}}}{{\text{K}}_{\text{D}} \text{+} \text{C}}$$

wherein
R_{eq} is the steady-state binding level;
C is the concentration of the analyte; and
Rₘₐₓ is the maximum binding capacity of the surface.

It is understood that not every calculation method may be possible for every biased IL-2 molecule. If, for example, the reactions are too fast, it may not be possible to use a 1:1 kinetic model and a 1:1 steady state interaction model may be used. If, for example, no equilibrium is obtained, it may not be possible to use a 1:1 interaction model and a 1:1 kinetic model may be used.

As used herein the term “proteinogenic amino acids” relates to amino acids selected from the group consisting of alanine (Ala, A), arginine (Arg, R), asparagine (Asn, N), aspartic acid (Asp, D), cysteine (Cys, C), glutamic acid (Glu, E), glutamine (Gln, Q), glycine (Gly, G), histidine (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), and valine (Val, V). The respective three- and one-letter codes are provided in brackets.

As used herein the term “non-proteinogenic amino acids” relates to amino acids selected form the group consisting of D-stereoisomers of each of the proteinogenic amino acids, pyrrolysine (Pyl, O), selenocysteine (Sec, U), 2-aminoadipic acid (ad), 3-aminoadipic acid (bAad), beta-alanine (bAla), 2-aminobutyric acid (Abu), 4-aminobutyric acid (4Abu), 6-aminocaproic acid (Acp), 2-aminoheptanoic acid (Ahe), 2-aminoisobutyric acid (Aib), 3-aminoisobutyric acid (bAib), 2-aminopimelic acid (Apm), 2,4-diaminobutyric acid (Dbu), desmosine (Des), 2,2′-diaminopimelic acid (Dpm), 2,3-diaminoproprionic acid (Dpr), N-ethylglycine (EtGly), N-ethylasparagine (EtAsn), hydroxylysine (Hyl), allo-hydroxylysine (aHyl), 3-hydroxyproline (3Hyp), 4-hydroxyproline (4Hyp), isodesmosine (Ide), allo-isoleucine (alle), N-methylglycine (MeGly), N-methylisoleucine (Melle), 6-N-methyllysine (MeLys), N-methylvaline (MeVal), norvaline (Nva), norleucine (Nle), and ornithine (Orn). The respective abbreviations are provided in brackets.

It is understood that in the context of amino acid sequences single letters refer to the one-letter code of amino acids and not chemical atoms.

It is understood that the deletion or insertion of one or more amino acids in a sequence, such as SEQ A, SEQ B, SEQ C or SEQ D, may change the number, i.e. position, of a particular amino acid within such sequence and that in such case the corresponding, i.e. homologous, amino acid positions are included. This may be indicated by the phrase “or at the corresponding positions of homologs or variants thereof”, but such corresponding positions are included even in the absence of this phrase.

As used herein the terms “identical” and percent “identity”, in the context of two or more polynucleotide or polypeptide/protein sequences, refer to two or more sequences or subsequences that are the same or have a specified percentage of nucleotides or amino acid residues that are the same, when compared and aligned for maximum correspondence, as measured using a sequence comparison algorithms. An example of such an algorithm that is suitable for determining percent sequence identity is the BLAST algorithm (Altschul et al., J. Mol. Biol., 215: 403-410 (1990); Henikoff & Henikoff, Proc. Natl. Acad. Sci. USA, 89: 10915 (1989); Karlin & Altschul, Proc. Natl. Acad. Sci. USA, 90: 5873 5787 (1993)). Software for performing BLAST analyses is publicly available through the National Center for Biotechnology Information.

As used herein the term “glycosylation” refers to posttranslational modifications of a protein in which a carbohydrate, in particular a glycan, is attached to a functional group of an amino acid. Glycosylation comprises the attachment of a carbohydrate to a nitrogen of an asparagine or arginine side-chain (N-glycosylation), to the hydroxyl oxygen of a serine, threonine, tyrosine, hydroxylysine, or hydroxyproline side-chain (O-glycosylation); to the phosphate of a phosphoserine (phosphoglycosylation); or to the carbon on a tryptophan side-chain (C-glycosylation). The O-linked glycan may for example be N1 (NeuAc(a2-3)Gal(b1-3)GalNAc-ol) or N2 (NeuAc(a2-3)Gal(b1-3)(NeuAc(a2-6))GalNAc-ol), wherein NeuAc is N-acetylneuraminic acid (sialic acid), Gal is galactose and GalNAc-ol is acetylgalactosaminitol. The N-linked glycan may be a paucimannose glycan (Man₃GlcNAc₂) with the two innermost sugar residues being N-acetylglucosamine (GlcNAc), further extended by a trimannosyl core (Man₃). It may for example be a biantennary G0 glycan (GlcNAc₂ Man₃ GlcNAc₂), where paucimannose is bound to two GlcNAc residues, one at each of the two outer mannoses of the trimannosyl core, resulting in a bi-antennary structure. Such a biantennary glycoform may be further decorated with a range of additional sugars, including fucose (Fuc) residues attached at the innermost GlcNAc residue, and branches comprising GlcNAc, Gal, and NeuAc residues. For example, the N-linked glycan may be a G1 glycan (NeuAcGalGlcNAc₂Man₃ GlcNAc₂) carrying one additional galactose and one additional sialic acid residue compared to the G0 structure, or a G2 glycan (NeuAc₂Gal₂GlcNAc₂Man₃GlcNAc₂) comprising two additional galactose and two additional sialic acid residues compared to the G0 structure. Furthermore, the G0, G1 and G2 glycans may be fucosylated at the innermost GlcNAc residue resulting in G0F (GlcNAc₂Man₃GlcNAc₂Fuc), G1F (NeuAcGalGlcNAc₂Man₃GlcNAc₂Fuc) and G2F (NeuAc₂Gal₂GlcNAc₂Man₃GlcNAc₂Fuc) glycans. The above described glycans may also be decorated with an additional GlcNAc residue attached to the trimannosyl core, resulting in G0B (GlcNAc₃Man₃GlcNAc₂), G1B (NeuAcGalGlcNAc₃Man₃GlcNAc₂) and G2B (NeuAc₂Gal₂GlcNAc₃Man₃GlcNAc₂) glycans. Fucosylation and additional GlcNAc attachment at the trimannosyl core may also be combined, resulting in G0BF(GlcNAc₃Man₃GlcNAc2Fuc), G1BF (NeuAcGalGlcNAc₃Man₃GlcNAc₂Fuc) and G2BF (NeuAc₂Gal₂GlcNAc₃Man₃GlcNAc₂Fuc) glycans. The N-linked glycan may also be a tetra-antennary glycan formed by transfer of additional GlcNAc residues to the biantennary G0 glycans through the action of GlcNAc glycosyltransferases GNTIV and GNTV. The N-linked glycan may also be a bi-or tetrantennary paucimannose glycan modified with GlcNAc-Gal extensions (LacNAc). The N-linked glycan may also be a bi- or tetrantennary paucimannose glycan modified with GlcNAc-Gal extensions (LacNAc) and additionally coupled to a Gal residue and one or several NeuAc residues. Additional examples of N-linked glycans can be found in “Antibody glycoengineering strategies in mammalian cells”, Wang et al., Biotechnology and Bioengineering, 2018 Jun;115(6):1378-1393 and “SnapShot: N-Glycosylation Processing Pathways across Kingdoms”, Chung et al., Cell, 2017 Sep 21;171(1):258-258.el, which are hereby incorporated by reference. N-glycosylated proteins often comprise a mixture of N-glycans, rather than being homogenously glycosylated with one specific N-glycan. This is often referred to as microheterogeneity when concerning glycan structural variations at a specific site, and macroheterogeneity when concerning variability of glycosylation sites and site occupancy. The degree of heterogeneity of N-glycosylation may vary depending on the protein and the expression host. For example, the N-glycosylation site in the Fc domain of IgG1 antibodies is mainly occupied by G0F, G1F and G2F glycans in humans as well as in mammalian expression systems, but while recombinant therapeutic antibodies produced in CHO cells may typically comprise G0F, G1F and G2F glycans in the ratio GOF>G1F>>G2F, recombinant antibodies produced in mouse myeloma SP2/0 cells may comprise glycans in the ratio G1F>G0F>>G2F. Removal of N-linked or O-linked glycans can be achieved by enzymatic or chemical treatment. Removal of N-linked glycans by Peptide-N-glycosidase F (PNgaseF) treatment causes an amino acid change of the arginine (N) to aspartic acid (D), such as for example a glycosylation motif of sequence FGNST (SEQ ID NO:307) is thereby transformed to FGDST (SEQ ID NO:368).

As used herein the term “site occupancy” refers to the percentage of glycosylation found at a particular glycosylation motif. It is understood that the phrase “at least X% site occupancy” refers to a multitude of IL-2 molecules or moieties, such as are present in a preparation or pharmaceutical composition, of which X% of all IL-2 molecules or moieties are glycosylated at the particular site, and does not refer to individual molecules or moieties. N-glycan structure and site occupancy of a glycoprotein may affect important properties such as receptor binding affinity, stability, solubility, antigenicity, clearance rate and half-life. Thus, for recombinant glycoprotein manufacturing processes in general it is important to obtain a reproducibly and homogenously glycosylated product. It is therefore advantageous if the product is produced in a fully N-glycosylated form (full site occupancy) by the production host cells. To be “fully glycosylated” at least 90%, such as at least 91%, at least 92%, at least 93%, at least 94%, at least 95%, of all proteins are glycosylated at a particular site. It may be possible to separate N-glycosylated forms from forms lacking N-glycosylation during the downstream purification process, however, a more complicated process and lower recovery yields of purified compound can be expected if the starting material is heterogenous, with large effects on the overall process costs. In addition, it may be difficult or even impossible to obtain a high purity of the N-glycosylated forms if the starting material contains a large fraction of the variant lacking N-glycosylation. This is particularly relevant in cases where the non-glycosylated form has unwanted properties. For example, for biased N-glycosylated IL-2 proteins, the presence of forms not carrying an N-glycan is highly undesired since these IL-2 forms would have higher residual potency than the N-glycosylated form to activate the IL-2Rα.

As used herein the expression “an amino acid in -X position” or “an amino acid in +X position”, wherein X is an integer, refers to the position of an amino acid relative to a specified amino acid in a protein or peptide sequence, whereby an amino acid in -X position is the amino acid located N-terminally in a distance of X to the specified amino acid and an amino acid in +X position is the amino acid located C-terminally in a distance of X to the specified amino acid. For example, in a peptide sequence “ALMGR” (SEQ ID NO:253), A is the amino acid in -2 position of M, L is the amino acid in -1 position of M, G is the amino acid in +1 position of M, and R is the amino acid in +2 position of M.

As used herein the term “glycosylation motif” refers to an amino acid sequence which directs the site-specific glycosylation of a protein. The glycosylation motif may be an N-glycosylation motif, O-glycosylation motif, phosphoglycosylation motif, or C-glycosylation motif. An N-glycosylation motif may comprise two, three, four, five or six amino acids. An O-glycosylation motif may comprise one or two amino acids.

As used herein the term “N-glycosylation site” refers to the position in an amino acid sequence at which the asparagine (N) of the N-glycosylation motif is located.

As used herein the term “affinity” refers to the strength of the sum of non-covalent interactions between a single binding site of a molecule (such as a receptor) and its binding partner (such as a ligand). Unless indicated otherwise, as used herein, “affinity” refers to intrinsic binding affinity which reflects a 1:1 interaction between members of a binding pair (such as between a receptor and a ligand). The affinity of a molecule X for its partner Y can generally be represented by the equilibrium dissociation constant (K_{D}), which is the ratio of dissociation and association rate constants (k_{d} and kₐ, respectively) measured in a state of equilibrium. Thus, equivalent affinities may comprise different rate constants, as long as the ratio of the rate constants remains the same. Affinity can be measured by well-established methods known in the art, including those described herein.

As used herein the terms “α-subunit of the IL-2 receptor” and “IL-2Rα” refer to human CD25.

As used herein the terms “β-subunit of the IL-2 receptor” and “IL-2Rβ” refer to human CD122.

As used herein the terms “γ-subunit of the IL-2 receptor” and “IL-2Rγ” refer to human CD132.

As used herein the term “pattern recognition receptor agonist” (“PRRA”) refers to a molecule that binds to and activates one or more immune cell-associated receptor that recognizes pathogen-associated molecular patterns (PAMPs) or damage-associated molecular patterns (DAMPs), leading to immune cell activation and/or pathogen- or damage-induced inflammatory responses. Pattern recognition receptors are typically expressed by cells of the innate immune system such as monocytes, macrophages, dendritic cells (DCs), neutrophils, and epithelial cells, as well as cells of the adaptive immune system.

As used herein the terms “cytotoxic agent” and “chemotherapeutic agent” are used synonymously and refer to compounds that are toxic to cells, and which prevent cellular replication or growth, leading to cellular destruction/death. Examples of cytotoxic agents include chemotherapeutic agents and toxins, such as small molecule toxins or enzymatically active toxins of bacterial, fungal, plant or animal origin, including synthetic analogues and derivatives thereof.

As used herein the terms “immune checkpoint inhibitor” and “immune checkpoint antagonist” are used synonymously and refer to compounds that interfere with the function of, or inhibit binding of ligands that induce signaling through, cell-membrane expressed receptors that inhibit inflammatory immune cell function upon receptor activation. Such compounds may for example be biologics, such as antibodies, antibody fragments, affibodies, affilins, affimers, affitins, alphamabs, alphabodies, anticalins, avimers, DARPins, Fynomers®, Kunitz domain peptides, monobodies, nanoCLAMPs, cyclic peptides, peptides, Heavy Chain only antibodies, VHH antibodies or Nanobodies®, single chain variable Fragments (scFvs), natural or modified ligands or binding partners for these receptors or small molecule inhibitors.

As used herein the term “immune activating agonist” refers to compounds that directly or indirectly activate cell-membrane expressed checkpoint receptors.

As used herein the term “immune activating receptor agonist” refers to compounds that stimulate immune cell function upon activating or costimulatory receptor activation. Examples of such stimulatory receptors include CD3 subunits CD3γ, CD3δ, CD3ε and CD3ζ (CD247), T cell receptor (TCR) subunits TCRα, TCRβ, TCRγ, and TCRδ, B cell receptor (BCR) chains or signaling units CD79a or CD79b, CD2, CD4, CD8, CD16, CD32a, CD64, CD27, CD28, CD134 (OX40), CD137 (41BB), CD244 (2B4), CD278 (ICOS), CD357 (GITR), CRACC(CS1), LFA-1, NKG2D, NKG2C, NKp30, NKp46, NKp44, NKp80, NTB-A, activating short form KIR (KIR2DS1, KIR2DS2, KIR2DS3, KIR2DS4, KIR2DS5, KIR3DS1), CD40, SIRP-β, Dectin-1, Dectin-2, TREM1, TREM2, ILT1, ILT6, ILT7, ILT8, LIR-6, MDL1, and other immune receptors which utilize an immunotyrosine receptor based activation motif (ITAM) or induce signaling through the PI3K, JAK/STAT, MyD88, IRF, NFKB or JNK/AP1 pathways. Many multi-specific drugs are types of immune activating receptor agonists.

As used herein the terms “multi-specific” and “multi-specific drugs” refer to compounds that simultaneously bind to two or more different antigens and can mediate antagonistic, agonistic, or specific antigen binding activity in a target-dependent manner.

As used herein the term “antibody-drug conjugate” (ADC) refers to compounds typically consisting of an antibody linked to a biologically active cytotoxic payload, radiotherapy, or other drug designed to deliver cytotoxic agents to the tumor environment. ADCs are particularly effective for reducing tumor burden without significant systemic toxicity and may act to improve the effectiveness of the immune response induced by checkpoint inhibitor antibodies.

As used herein the term “antibody-adjuvant conjugate” (AAC) refers to compounds consisting of an antibody linked to a biologically active adjuvant, either directly or through a linker.

As used herein, the term “adjuvant” refers to a substance which enhances the body’s immune response to an antigen.

As used herein the term “boltbody” refers to an antibody-adjuvant conjugate comprising (a) an antibody moiety comprising (i) an antigen binding domain and (ii) an Fc domain, (b) an adjuvant moiety, and (c) a linker comprising an ethylene glycol group or a glycine residue, wherein each adjuvant moiety is covalently bonded to the antibody moiety via the linker, which linker can be cleavable or non-cleavable.

As used herein the term “radionuclides” refers to radioactive isotopes that emit ionizing radiation leading to cellular destruction/death. Radionuclides conjugated to tumor targeting carriers are referred to as “targeted radionuclide therapeutics”.

As used herein the term “DNA damage repair inhibitor” refers to a drug that targets DNA damage repair elements, such as for example CHK1, CHK2, ATM, ATR and PARP. Certain cancers are more susceptive to targeting these pathways due to existing mutations or pathway alterations, such as BRCA1 mutated patients or homologous recombination pathway deficient patients to PARP inhibitors due to the concept of synthetic lethality.

As used herein the term “tumor metabolism inhibitor” refers to a compound that interferes with the function of one or more enzymes expressed in the tumor environment that produce metabolic intermediates that may inhibit immune cell function.

As used herein the term “protein kinase inhibitor” refers to compounds that inhibit the activity of one or more protein kinases. Protein kinases are enzymes that phosphorylate proteins, which in turn can modulate protein function. It is understood that a protein kinase inhibitor may target more than one kinase and any classification for protein kinase inhibitors used herein refers to the main or most characterized target.

As used herein the term “chemokine receptor and chemoattractant receptor agonist” refers to compounds that activate chemokine or chemoattractant receptors, a subset of G-protein coupled receptors or G-protein coupled-like receptors that are expressed on a wide variety of cells and are primarily involved in controlling cell motility (chemotaxis or chemokinesis). These receptors may also participate in non-cell migratory processes, such as angiogenesis, cell maturation or inflammation.

As used herein the term “cytokine receptor agonist” refers to soluble proteins which control immune cell activation and proliferation. Cytokines include for example interferons, interleukins, lymphokines, and tumor necrosis factor.

As used herein the term “death receptor agonist” refers to a molecule which is capable of inducing pro-apoptotic signaling through one or more of the death receptors, such as DR4 (TRAIL-R1) or DR5 (TRAIL-R2). The death receptor agonist may be selected from the group consisting of antibodies, death ligands, cytokines, death receptor agonist expressing vectors, peptides, small molecule agonists, cells (such as for example stem cells) expressing the death receptor agonist, and drugs inducing the expression of death ligands.

As used herein the term “antigen-presenting cell” or “APC” refers to a cell, such as a macrophage, a B cell, or a dendritic cell, that presents processed antigenic peptides via MHC class II molecules to the T cell receptor on CD4 T cells. APCs can be identified by a person skilled in the art by using phenotypic techniques such as flow cytometry. Phenotypic markers used to identify APCs vary by species and by tissue but may include myeloid or dendritic cell surface markers (e.g. CD11b, CD11c, CD14, CD16, CD33, CD34, CD68, CD206, MHC-II, CD163, Ly6C, Ly6G, GR-1, F4/80, TREM1, TREM2) or B cell surface markers (e.g. CD19, CD20, B220).

As used herein the term “MHCII” refers to a class of major histocompatibility complex (MHC) molecules normally found only on antigen-presenting cells such as myeloid cells, dendritic cells, and B cells. MHCII presents processed antigenic peptides to the T cell receptor on CD4 T cells. MHCII expression can be measured by a person skilled in the art using protein expression profiling techniques such as flow cytometry. Changes in MHCII expression can be determined by analyzing changes in the median fluorescence intensity signal of MHCII, or the percentage of cells positive for MHCII, in a specific cell subset of interest.

As used herein the term “T cells” refers to a type of immune cell that plays a central role in the adaptive immune response. T cells are distinguished from other immune cells by the presence of either an αβ or γδ T cell receptor (TCR) on their cell surface. T cells also express CD3 - a protein complex critical for TCR signaling. αβ T cells can be divided into either CD4, CD8, or CD4/CD8 double negative subsets. Due to the high surface density of CD4 and CD8 on CD4⁺ and CD8⁺ T cells, CD4 and CD8 alone can often be used to identify CD4⁺ and CD8⁺ T cells respectively. γδ T cells are equipped with a TCR consisting of a γ chain and δ chain, which, like the αβ TCR, is central for recognition of antigens and cellular activation. This TCR is also used to distinguish between the different subsets of γδ T cells, being Vδ1 and Vδ2. Vδ1 T cells are the minority (<5%) and a heterogeneous population of γδ T cells with both anti- and pro-inflammatory functions. Vδ2 T cells are a single relatively homogenous T cell population of Vγ9Vδ2 (Vδ2) T cells that make up ~95% of γδ T cells in circulation. Due to the unique properties of their TCR and additional innate immune receptors, Vδ2 T cells are endowed with potent anti-tumor properties that can be harnessed for immunotherapy. Following activation via TCR recognition of cognate antigen presented by MHC molecules, T cells can mature and divide to generate effector or memory T cells. Memory T cells are a subset of T cells that have previously encountered and responded to their cognate antigen. Such T cells can recognize pathogenic antigens, such as antigens derived from bacteria or viruses, as well as cancer-associated antigens. T cells can be identified by a person skilled in the art by using phenotypic techniques such as flow cytometry. Phenotypic markers used to identify T cells are generally conserved in mammals and include CD3, TCRα, TCRβ, TCRδ, CD4, and CD8. Phenotypic markers used to identify memory T cells can vary by species and by tissue, but may include cell surface markers such as CD45RO, LY6C, CD44, and CD95.

As used herein the expression “cell therapy” refers to the infusion or transplantation of modified human cells into a patient for the treatment of a disease. The origin of the cells can be from the patient (autologous) or from a healthy donor (allogeneic).

As used herein the term “reversible”, “reversibly”, “degradable” or “degradably” with regard to the attachment of a first moiety to a second moiety means that the linkage that connects said first and second moiety is cleavable under physiological conditions, which are aqueous buffer at pH 7.4, 37° C., with a half-life ranging from one hour to three months, such as from one hour to two months, from three hours to one month, from six hours to 28 days, from 12 hours to 21 days, from 24 hours to 14 days or from 48 hours to 7 days. Cleavage may be enzymatically or non-enzymatically and is in certain embodiments non-enzymatically. Accordingly, the term “stable” or “permanent” with regard to the attachment of a first moiety to a second moiety means that the linkage that connects said first and second moiety is cleavable with a half-life of more than three months under physiological conditions.

As used herein the term “reagent” means a chemical compound, which comprises at least one functional group for reaction with the functional group of another chemical compound or drug. It is understood that a drug comprising a functional group (such as a primary or secondary amine or hydroxyl functional group) is also a reagent.

As used herein the term “moiety” means a part of a molecule, which lacks one or more atom(s) compared to the corresponding reagent. If, for example, a reagent of the formula “H—X—H″ reacts with another reagent and becomes part of the reaction product, the corresponding moiety of the reaction product has the structure “H—X—” or “—X—”, whereas each “-” indicates attachment to another moiety. Accordingly, a drug moiety is released from a reversible linkage as a drug.

As used herein the term “tag moiety” refers to a peptide or protein sequence which is translationally fused to the IL-2 protein. It may fulfill various functions, such as for example the stabilization or half-life extension of the IL-2 protein (“stabilization tag”), aid in the purification of the IL-2 protein (“purification tag”) or target the IL-2 protein towards a particular cell type or tissue (“targeting tag”).

It is understood that if the sequence or chemical structure of a group of atoms is provided which group of atoms is attached to two moieties or is interrupting a moiety, said sequence or chemical structure can be attached to the two moieties in either orientation, unless explicitly stated otherwise. For example, a moiety “—C(O)N(R¹)—” can be attached to two moieties or interrupting a moiety either as “—C(O)N(R¹)—” or as “—N(R¹)C(O)—”. Similarly, a moiety

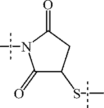

can be attached to two moieties or can interrupt a moiety either as

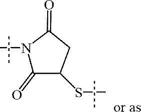

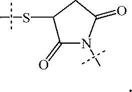

The term “substituted” as used herein means that one or more —H atom(s) of a molecule or moiety are replaced by a different atom or a group of atoms, which are referred to as “substituent”. In certain embodiments no more than 6 —H atoms of a moiety or molecule are substituted. In certain embodiments no more than 5 —H atoms of a moiety or molecule are substituted. In certain embodiments no more than 4 —H atoms of a moiety or molecule are substituted. In certain embodiments no more than 3 —H atoms of a moiety or molecule are substituted. In certain embodiments no more than 2 —H atoms of a moiety or molecule are substituted. In certain embodiments 1 —H atom of a moiety or molecule are substituted.

As used herein the term “substituent” refers in certain embodiments to a moiety selected from the group consisting of halogen, —CN, -COOR^{x1}, —OR^{x1}, —C(O)R^{x1}, -C(O)N(R^{x1}R^{x1a}), -S(O)₂N(R^{x1}R^{x1a}), -S(O)N(R^{x1}R^{x1a}), —S(O)₂R^{x1}, —S(O)R^{x1}, -N(R^{x1})S(O)₂N(R^{x1a}R^{x1b}), —SR^{x1}, -N(R^{x1}R^{x1a}), —NO₂, —OC(O)R^{x1}, -N(R^{x1})C(O)R^{x1a}, -N(R^{x1})S(O)₂R^{x1a}, -N(R^{x1})S(O)R^{x1a}, -N(R^{x1})C(O)OR^{x1a}, -N(R^{x1})C(O)N(R^{x1a}R^{x1b}), -OC(O)N(R^{x1}R^{x1a}), -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more -R^{x2}, which are the same or different and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, -N(R^{x3})S(O)₂N(R^{x3a})-, —S—, —N(R^{x3})—, -OC(OR^{x3})(R^{x3a})-, -N(R^{x3})C(O)N(R^{x3a})-, and —OC(O)N(R^{x3})—;
-R^{x1}, -R^{x1a}, -R^{x1b} are independently of each other selected from the group consisting of —H, -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more -R^{x2}, which are the same or different and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—; —S(O)₂—, —S(O)—, -N(R^{x3})S(O)₂N(R^{x3a})-, —S—, —N(R^{x3})—, -OC(OR^{x3})(R^{x3a})-, -N(R^{x3})C(O)N(R^{x3a})-, and —OC(O)N(R^{x3})—;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more -R^{x2}, which are the same or different;
each -R^{x2} is independently selected from the group consisting of halogen, —CN, oxo (=O), -COOR^{x4}, —OR^{x4}, —C(O)R^{x4}, -C(O)N(R^{x4}R^{x4a}), -S(O)₂N(R^{x4}R^{x4a}), -S(O)N(R^{x4}R^{x4a}), —S(O)₂R^{x4}, —S(O)R^{x4}, -N(R^{x4})S(O)₂N(R^{x4a}R^{x4b}), —SR^{x4}, -N(R^{x4}R^{x4a}), —NO₂, -OC(O)R^{x4} -N(R^{x4})C(O)R^{x4a}, -N(R^{x4})S(O)₂R^{x4a}, -N(R^{x4})S(O)R^{x4a}, -N(R^{x4})C(O)OR^{x4a}, -N(R^{x4})C(O)N(R^{x4a}R^{x46}), -OC(O)N(R^{x4}R^{x4a}), and C₁₋₆ alkyl; wherein C₁₋₆ alkyl is optionally substituted with one or more halogen, which are the same or different;
each -R^{x3}, -R^{x3a}, -R^{x4}, -R^{x4a}, -R^{x4b} is independently selected from the group consisting of —H and C₁₋₆ alkyl; wherein C₁₋₆ alkyl is optionally substituted with one or more halogen, which are the same or different.

In certain embodiments a maximum of 6 —H atoms of an optionally substituted molecule are independently replaced by a substituent, e.g. 5 —H atoms are independently replaced by a substituent, 4 —H atoms are independently replaced by a substituent, 3 —H atoms are independently replaced by a substituent, 2 —H atoms are independently replaced by a substituent, or 1 —H atom is replaced by a substituent.

As used herein the term “fatty acid” refers to a saturated or unsaturated monocarboxylic acid having an aliphatic tail, which may include from 4 to 28 carbon atoms. The fatty acid may be saturated or unsaturated, linear or branched. The term “fatty acid variant” refers to a modified fatty acid in which certain carbon atoms may be replaced by other atoms or groups of atoms and which may be substituted.

The term “peptide” as used herein refers to a chain of at least 2 and up to and including 50 amino acid monomer moieties linked by peptide (amide) linkages. The term “peptide” also includes peptidomimetics, such as D-peptides, peptoids or beta-peptides, and covers such peptidomimetic chains with up to and including 50 monomer moieties.

As used herein the term “protein” refers to a chain of more than 50 amino acid monomer moieties, which may also be referred to as “amino acid residues”, linked by peptide linkages, in which in certain embodiments no more than 12000 amino acid monomers are linked by peptide linkages, such as no more than 10000 amino acid monomer moieties, no more than 8000 amino acid monomer moieties, no more than 5000 amino acid monomer moieties or no more than 2000 amino acid monomer moieties.

As used herein the term “about” in combination with a numerical value is used to indicate a range ranging from and including the numerical value plus and minus no more than 25% of said numerical value, in certain embodiments plus and minus no more than 20% of said numerical value and in certain embodiments plus and minus no more than 10% of said numerical value. For example, the phrase “about 200” is used to mean a range ranging from and including 200 +/- 25%, i.e. ranging from and including 150 to 250; in certain embodiments 200 +/- 20%, i.e. ranging from and including 160 to 240; and in certain embodiments from and including 200 +/-10%, i.e. ranging from and including 180 to 220. It is understood that a percentage given as “about 50%” does not mean “50% +/- 25%”, i.e. ranging from and including 25 to 75%, but “about 50%” means ranging from and including 37.5 to 62.5%, i.e. plus and minus 25% of the numerical value which is 50.

As used herein the term “polymer” means a molecule comprising repeating structural units, i.e. the monomers, connected by chemical bonds in a linear, circular, branched, crosslinked or dendrimeric way or a combination thereof, which may be of synthetic or biological origin or a combination of both. It is understood that a polymer may also comprise one or more other chemical group(s) and/or moiety/moieties, such as, for example, one or more functional group(s). Likewise, it is understood that also a peptide or protein is a polymer, even though the side chains of individual amino acid residues may be different. In certain embodiments a soluble polymer has a molecular weight of at least 0.5 kDa, e.g. a molecular weight of at least 1 kDa, a molecular weight of at least 2 kDa, a molecular weight of at least 3 kDa or a molecular weight of at least 5 kDa. If the polymer is soluble, it in certain embodiments has a molecular weight of at most 1000 kDa, such as at most 750 kDa, such as at most 500 kDa, such as at most 300 kDa, such as at most 200 kDa, such as at most 100 kDa. It is understood that for insoluble polymers, such as hydrogels, no meaningful molecular weight ranges can be provided.

As used herein the term “polymeric” means a reagent or a moiety comprising one or more polymer(s) or polymer moiety/moieties. A polymeric reagent or moiety may optionally also comprise one or more other moiety/moieties, which are in certain embodiments selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group comprising

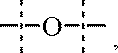

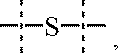

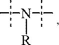

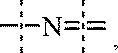

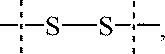

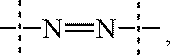

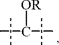

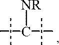

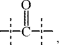

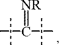

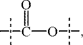

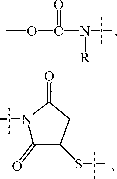

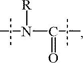

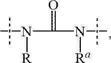

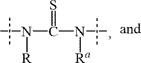

wherein
   dashed lines indicate attachment to the remainder of the moiety or reagent, and -R and -R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

The person skilled in the art understands that the polymerization products obtained from a polymerization reaction do not all have the same molecular weight, but rather exhibit a molecular weight distribution. Consequently, the molecular weight ranges, molecular weights, ranges of numbers of monomers in a polymer and numbers of monomers in a polymer as used herein, refer to the number average molecular weight and number average of monomers, i.e. to the arithmetic mean of the molecular weight of the polymer or polymeric moiety and the arithmetic mean of the number of monomers of the polymer or polymeric moiety.

Accordingly, in a polymeric moiety comprising “x” monomer units any integer given for “x” therefore corresponds to the arithmetic mean number of monomers. Any range of integers given for “x” provides the range of integers in which the arithmetic mean numbers of monomers lies. An integer for “x” given as “about x” means that the arithmetic mean numbers of monomers lies in a range of integers of x +/- 25%, preferably x+/- 20% and more preferably x +/- 10%.

As used herein the term “number average molecular weight” means the ordinary arithmetic mean of the molecular weights of the individual polymers.

As used herein the term “PEG-based” in relation to a moiety or reagent means that said moiety or reagent comprises PEG. In certain embodiments a PEG-based moiety or reagent comprises at least 10% (w/w) PEG, such as at least 20% (w/w) PEG, such as at least 30% (w/w) PEG, such as at least 40% (w/w) PEG, such as at least 50% (w/w), such as at least 60% (w/w) PEG, such as at least 70% (w/w) PEG, such as at least 80% (w/w) PEG, such as at least 90% (w/w) PEG, such as at least 95%. The remaining weight percentage of the PEG-based moiety or reagent are other moieties that in certain embodiments are selected from the following moieties and linkages:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group comprising

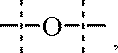

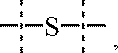

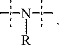

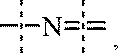

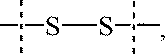

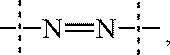

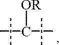

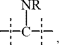

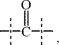

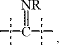

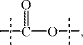

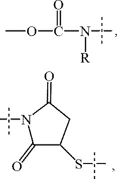

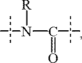

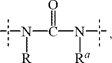

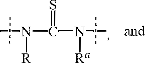

wherein
   dashed lines indicate attachment to the remainder of the moiety or reagent, and -R and -R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

The term “hyaluronic acid-based” is used accordingly.

As used herein the term “PEG-based comprising at least X% PEG” in relation to a moiety or reagent means that said moiety or reagent comprises at least X% (w/w) ethylene glycol units (—CH₂CH₂O—), wherein the ethylene glycol units may be arranged blockwise, alternating or may be randomly distributed within the moiety or reagent and in certain embodiments all ethylene glycol units of said moiety or reagent are present in one block; the remaining weight percentage of the PEG-based moiety or reagent are other moieties that in certain embodiments are selected from the following moieties and linkages:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group comprising

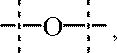

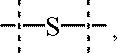

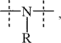

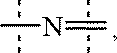

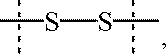

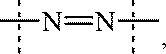

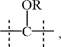

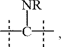

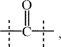

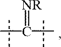

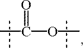

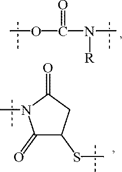

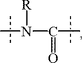

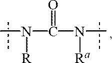

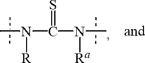

wherein
   dashed lines indicate attachment to the remainder of the moiety or reagent, and -R and -R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

The term “hyaluronic acid-based comprising at least X% hyaluronic acid” is used accordingly.

As used herein the term “hydrogel” means a hydrophilic or amphiphilic polymeric network composed of homopolymers or copolymers, which is insoluble due to the presence of hydrophobic interactions, hydrogen bonds, ionic interactions and/or covalent chemical crosslinks. In certain embodiments a hydrogel is insoluble due to the presence of covalent chemical crosslinks. In general, the crosslinks provide the network structure and physical integrity.

The term “interrupted” means that a moiety is inserted between two carbon atoms or - if the insertion is at one of the moiety’s ends - between a carbon or heteroatom and a hydrogen atom.

As used herein the term “C₁₋₄ alkyl” alone or in combination means a straight-chain or branched alkyl moiety having 1 to 4 carbon atoms. If present at the end of a molecule, examples of straight-chain or branched C₁₋₄ alkyl are methyl, ethyl, n-propyl, isopropyl, n-butyl, isobutyl, sec-butyl and tert-butyl. When two moieties of a molecule are linked by the C₁₋₄ alkyl, then examples for such C₁₋₄ alkyl groups are —CH₂—, —CH₂—CH₂—, —CH(CH₃)—, —CH₂—CH₂—CH₂—, —CH(C₂H₅)—, —C(CH₃)₂—. Each hydrogen of a C₁₋₄ alkyl carbon may optionally be replaced by a substituent as defined above. Optionally, a C₁₋₄ alkyl may be interrupted by one or more moieties as defined below.

As used herein the term “C₁₋₆ alkyl” alone or in combination means a straight-chain or branched alkyl moiety having 1 to 6 carbon atoms. If present at the end of a molecule, examples of straight-chain and branched C₁₋₆ alkyl groups are methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. When two moieties of a molecule are linked by the C₁₋₆ alkyl group, then examples for such C₁₋₆ alkyl groups are —CH₂—, —CH₂—CH₂—, —CH(CH₃)—, —CH₂—CH₂—CH₂—, —CH(C₂H₅)— and —C(CH₃)₂—. Each hydrogen atom of a C₁₋₆ carbon may optionally be replaced by a substituent as defined above. Optionally, a C₁₋₆ alkyl may be interrupted by one or more moieties as defined below.

Accordingly, “C₁₋₁₀ alkyl”, “C₁₋₂₀ alkyl” or “C₁₋₅₀ alkyl” means an alkyl chain having 1 to 10, 1 to 20 or 1 to 50 carbon atoms, respectively, wherein each hydrogen atom of the C₁₋₁₀, C₁₋₂₀ or C₁₋₅₀ carbon may optionally be replaced by a substituent as defined above. Optionally, a C₁₋₁₀ or C₁₋₅₀ alkyl may be interrupted by one or more moieties as defined below.

As used herein the term “C₂₋₆ alkenyl” alone or in combination means a straight-chain or branched hydrocarbon moiety comprising at least one carbon-carbon double bond having 2 to 6 carbon atoms. If present at the end of a molecule, examples are —CH═CH₂, —CH═CH—CH₃, —CH₂—CH═CH₂, —CH═CHCH₂—CH₃ and —CH═CH—CH═CH₂. When two moieties of a molecule are linked by the C₂₋₆ alkenyl group, then an example for such C₂₋₆ alkenyl is —CH═CH—. Each hydrogen atom of a C₂₋₆ alkenyl moiety may optionally be replaced by a substituent as defined above. Optionally, a C₂₋₆ alkenyl may be interrupted by one or more moieties as defined below.

Accordingly, the term “C₂₋₁₀ alkenyl”, “C₂₋₂₀ alkenyl” or “C₂₋₅₀ alkenyl” alone or in combination means a straight-chain or branched hydrocarbon moiety comprising at least one carbon-carbon double bond having 2 to 10, 2 to 20 or 2 to 50 carbon atoms. Each hydrogen atom of a C₂₋₁₀ alkenyl, C₂₋₂₀ alkenyl or C₂₋₅₀ alkenyl group may optionally be replaced by a substituent as defined above. Optionally, a C₂₋₁₀ alkenyl, C₂₋₂₀ alkenyl or C₂₋₅₀ alkenyl may be interrupted by one or more moieties as defined below.

As used herein the term “C₂₋₆ alkynyl” alone or in combination means a straight-chain or branched hydrocarbon moiety comprising at least one carbon-carbon triple bond having 2 to 6 carbon atoms. If present at the end of a molecule, examples are —C≡CH, —CH₂—C≡CH, —CH₂—CH₂—C≡CH and CH₂—C≡C—CH₃. When two moieties of a molecule are linked by the alkynyl group, then an example is —C≡C—. Each hydrogen atom of a C₂₋₆ alkynyl group may optionally be replaced by a substituent as defined above. Optionally, one or more double bond(s) may occur. Optionally, a C₂₋₆ alkynyl may be interrupted by one or more moieties as defined below.

Accordingly, as used herein, the term “C₂₋₁₀ alkynyl”, “C₂₋₂₀ alkynyl” and “C₂₋₅₀ alkynyl” alone or in combination means a straight-chain or branched hydrocarbon moiety comprising at least one carbon-carbon triple bond having 2 to 10, 2 to 20 or 2 to 50 carbon atoms, respectively. Each hydrogen atom of a C₂₋₁₀ alkynyl, C₂₋₂₀ alkynyl or C₂₋₅₀ alkynyl group may optionally be replaced by a substituent as defined above. Optionally, one or more double bond(s) may occur. Optionally, a C₂₋₁₀ alkynyl, C₂₋₂₀ alkynyl or C₂₋₅₀ alkynyl may be interrupted by one or more moieties as defined below.

As mentioned above, a C₁₋₄ alkyl, C₁₋₆ alkyl, C₁₋₁₀ alkyl, C₁₋₂₀ alkyl, C₁₋₅₀ alkyl, C₂₋₆ alkenyl, C₂₋₁₀ alkenyl, C₂₋₂₀ alkenyl, C₂₋₅₀ alkenyl, C₂₋₆ alkynyl, C₂₋₁₀ alkynyl, C₂₋₂₀ alkenyl or C₂₋₅₀ alkynyl may optionally be interrupted by one or more moieties which are preferably selected from the group consisting of

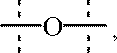

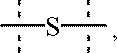

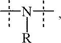

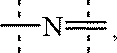

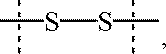

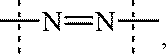

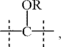

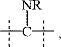

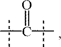

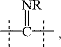

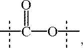

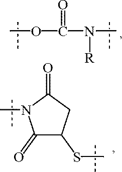

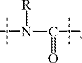

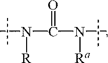

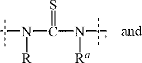

wherein
dashed lines indicate attachment to the remainder of the moiety or reagent; and
-R and -R^{a} are independently of each other selected from the group consisting of —H, and methyl, ethyl, propyl, butyl, pentyl and hexyl.

As used herein the term “C₃₋₁₀ cycloalkyl” means a cyclic alkyl chain having 3 to 10 carbon atoms, which may be saturated or unsaturated, e.g. cyclopropyl, cyclobutyl, cyclopentyl, cyclohexyl, cyclohexenyl, cycloheptyl, cyclooctyl, cyclononyl or cyclodecyl. Each hydrogen atom of a C₃₋₁₀ cycloalkyl carbon may be replaced by a substituent as defined above. The term “C₃₋₁₀ cycloalkyl” also includes bridged bicycles like norbornane or norbornene.

As used herein the term “8- to 30-membered carbopolycyclyl” or “8- to 30-membered carbopolycycle” means a cyclic moiety of two or more rings with 8 to 30 ring atoms, where two neighboring rings share at least one ring atom and that may contain up to the maximum number of double bonds (aromatic or non-aromatic ring which is fully, partially or un-saturated). Preferably a 8- to 30-membered carbopolycyclyl means a cyclic moiety of two, three, four or five rings, more preferably of two, three or four rings.

As used herein the term “3- to 10-membered heterocyclyl” or “3- to 10-membered heterocycle” means a ring with 3, 4, 5, 6, 7, 8, 9 or 10 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for 3- to 10-membered heterocycles include but are not limited to aziridine, oxirane, thiirane, azirine, oxirene, thiirene, azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine and homopiperazine. Each hydrogen atom of a 3- to 10-membered heterocyclyl or 3- to 10-membered heterocyclic group may be replaced by a substituent as defined below.

As used herein the term “8- to 11-membered heterobicyclyl” or “8- to 11-membered heterobicycle” means a heterocyclic moiety of two rings with 8 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for an 8- to 11-membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, benzazepine, purine and pteridine. The term 8- to 11-membered heterobicycle also includes spiro structures of two rings like 1,4-dioxa-8-azaspiro[4.5]decane or bridged heterocycles like 8-aza-bicyclo[3.2.1]octane. Each hydrogen atom of an 8- to 11-membered heterobicyclyl or 8- to 11-membered heterobicycle carbon may be replaced by a substituent as defined below.

Similary, the term “8- to 30-membered heteropolycyclyl” or “8- to 30-membered heteropolycycle” means a heterocyclic moiety of more than two rings with 8 to 30 ring atoms, preferably of three, four or five rings, where two neighboring rings share at least one ring atom and that may contain up to the maximum number of double bonds (aromatic or non-aromatic ring which is fully, partially or unsaturated), wherein at least one ring atom up to 10 ring atoms are replaced by a heteroatom selected from the group of sulfur (including —S(O)—, —S(O)₂—), oxygen and nitrogen (including ═N(O)—) and wherein the ring is linked to the rest of a molecule via a carbon or nitrogen atom.

It is understood that the phrase “the pair R^{x}/R^{y} is joined together with the atom to which they are attached to form a C₃₋₁₀ cycloalkyl or a 3- to 10-membered heterocyclyl” in relation with a moiety of the structure

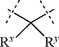

means that R^{x} and R^{y} form the following structure:

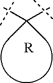

wherein R is C₃₋₁₀ cycloalkyl or 3- to 10-membered heterocyclyl.

It is also understood that the phrase “the pair R^{x}/R^{y} is joined together with the atoms to which they are attached to form a ring A” in relation with a moiety of the structure

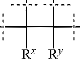

means that R^{x} and R^{y} form the following structure:

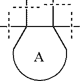

As used herein “halogen” means fluoro, chloro, bromo or iodo. It is generally preferred that halogen is fluoro or chloro.

As used herein the term “functional group” means a group of atoms which can react with other groups of atoms. Exemplary functional groups are, for example, carboxylic acid (—(C═O)OH), primary or secondary amine (—NH₂, —NH—), maleimide, thiol (—SH), sulfonic acid (—(O═S═O)OH), carbonate, carbamate (—O(C═O)N<), hydroxyl (—OH), aldehyde (—(C═O)H), ketone (—(C═O)—), hydrazine (>N—N<), isocyanate, isothiocyanate, phosphoric acid (—O(P═O)OHOH), phosphonic acid (—O(P═O)OHH), haloacetyl, alkyl halide, acryloyl, aryl fluoride, hydroxylamine, disulfide, sulfonamides, sulfuric acid, vinyl sulfone, vinyl ketone, diazoalkane, oxirane, and aziridine.

In case the IL-2 proteins or conjugates of the present invention comprise one or more acidic or basic groups, the invention also comprises their corresponding pharmaceutically or toxicologically acceptable salts, in particular their pharmaceutically utilizable salts. Thus, the IL-2 proteins or conjugates of the present invention comprising acidic groups can be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids. IL-2 proteins or conjugates of the present invention comprising one or more basic groups, i.e. groups which can be protonated, can be present and can be used according to the invention in the form of their addition salts with inorganic or organic acids. Examples for suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. For the person skilled in the art further methods are known for converting the basic group into a cation like the alkylation of an amine group resulting in a positively-charge ammonium group and an appropriate counterion of the salt. If the IL-2 proteins or conjugates of the present invention simultaneously comprise acidic and basic groups, the invention also includes, in addition to the salt forms mentioned, inner salts or betaines (zwitterions). The respective salts can be obtained by customary methods, which are known to the person skilled in the art like, for example by contacting these prodrugs with an organic or inorganic acid or base in a solvent or dispersant, or by anion exchange or cation exchange with other salts. The present invention also includes all salts of the IL-2 proteins or conjugates of the present invention which, owing to low physiological compatibility, are not directly suitable for use in pharmaceuticals but which can be used, for example, as intermediates for chemical reactions or for the preparation of pharmaceutically acceptable salts.

As used herein the term “pharmaceutically acceptable” means a substance that does not cause harm when administered to a patient and preferably means approved by a regulatory agency, such as the EMA (Europe) and/or the FDA (US) and/or any other national regulatory agency for use in animals, such as for use in humans.

As used herein the term “excipient” refers to a diluent, adjuvant, or vehicle with which the therapeutic, such as a drug or prodrug, is administered. Such pharmaceutical excipient can be sterile liquids, such as water and oils, including those of petroleum, animal, vegetable or synthetic origin, including but not limited to peanut oil, soybean oil, mineral oil, sesame oil and the like. Water is a preferred excipient when the pharmaceutical composition is administered orally. Saline and aqueous dextrose are preferred excipients when the pharmaceutical composition is administered intravenously. Saline solutions and aqueous dextrose and glycerol solutions are preferably employed as liquid excipients for injectable solutions. Suitable pharmaceutical excipients include starch, glucose, lactose, sucrose, mannitol, trehalose, gelatin, malt, rice, flour, chalk, silica gel, sodium stearate, glycerol monostearate, talc, sodium chloride, dried skim milk, glycerol, propylene, glycol, water, ethanol and the like. The pharmaceutical composition, if desired, can also contain minor amounts of wetting or emulsifying agents, pH buffering agents, like, for example, acetate, succinate, tris, carbonate, phosphate, HEPES (4-(2-hydroxyethyl)-1-piperazineethanesulfonic acid), MES (2-(N-morpholino)ethanesulfonic acid), or can contain detergents, like Tween, poloxamers, poloxamines, CHAPS, Igepal, or amino acids like, for example, glycine, lysine, or histidine. These pharmaceutical compositions can take the form of solutions, suspensions, emulsions, tablets, pills, capsules, powders, sustained-release formulations and the like. The pharmaceutical composition can be formulated as a suppository, with traditional binders and excipients such as triglycerides. Oral formulation can include standard excipients such as pharmaceutical grades of mannitol, lactose, starch, magnesium stearate, sodium saccharine, cellulose, magnesium carbonate, etc. Such compositions will contain a therapeutically effective amount of the drug or biologically active moiety, together with a suitable amount of excipient so as to provide the form for proper administration to the patient. The formulation should suit the mode of administration.

In general, the term “comprise” or “comprising” also encompasses “consist of” or “consisting of”.

SEQ A of formula (I) has at least 89% sequence identity to SEQ ID NO:1. SEQ ID NO:1 has the following sequence:

PTSSSTKKTQLQLEHLLLDLQMILNGINN

SEQ B of formula (I) has at least 76% sequence identity to SEQ ID NO:2. SEQ ID NO:2 has the following sequence:

YKNPKLTRMLTFKFYMPKKATELKHLQCLEEELKPLEEVLNLAQSK

SEQ C of formula (I) has at least 91% sequence identity to SEQ ID NO:4. SEQ ID NO:4 has the following sequence:

NFHLRPRDLISNINVIVLELKGSETTFMCEYADETATIVEFLNRWITFCQ
SIISTLT

Unless stated otherwise all amino acid positions given herein are based on SEQ ID NO:1; SEQ ID NO:2 or SEQ ID NO:4, respectively.

In certain embodiments SEQ A of formula (I) has at least 93% sequence identity to SEQ ID NO:1. In certain embodiments SEQ A of formula (I) has at least 96% sequence identity to SEQ ID NO:1.

In certain embodiments SEQ A comprises three amino acid changes compared to SEQ ID NO:1. In certain embodiments SEQ A comprises two amino acid changes compared to SEQ ID NO:1. In certain embodiments SEQ A comprises one amino acid change compared to SEQ ID NO:1. Such amino acid change may be an amino acid deletion, amino acid addition or the exchange of one amino acid for another amino acid, i.e. a mutation. Such mutation may also be the exchange of a proteinogenic amino acid for a non-proteinogenic amino acid. In certain embodiments SEQ A comprises no amino acid change compared to SEQ ID NO:1, i.e. SEQ A has the sequence of SEQ ID NO:1. In certain embodiments SEQ A has the sequence of SEQ ID NO:36: PASSSTKKTQLQLEHLLLDLQMILNGINN.

In certain embodiments, SEQ A comprises an amino acid mutation, which eliminates the endogenous O-glycosylation motif. Preferably, such amino acid mutation is at position 2 of SEQ ID NO:1, even more preferably such amino acid mutation is selected from the group consisting of T2A, T2G, T2Q, T2E, T2N, T2D, T2R, T2K and T2P. In certain embodiments such amino acid mutation is T2A, based on SEQ ID NO:1 or the corresponding positions of homologs or variants thereof.

In certain embodiments SEQ B of formula (I) has at least 78% sequence identity to SEQ ID NO:2. In certain embodiments SEQ B of formula (I) has at least 80% sequence identity to SEQ ID NO:2. In certain embodiments SEQ B of formula (I) has at least 82% sequence identity to SEQ ID NO:2. In certain embodiments SEQ B of formula (I) has at least 84% sequence identity to SEQ ID NO:2. In certain embodiments SEQ B of formula (I) has at least 87% sequence identity to SEQ ID NO:2. In certain embodiments SEQ B of formula (I) has at least 89% sequence identity to SEQ ID NO:2. In certain embodiments SEQ B of formula (I) has at least 91% sequence identity to SEQ ID NO:2. In certain embodiments SEQ B of formula (I) has at least 93% sequence identity to SEQ ID NO:2. In certain embodiments SEQ B of formula (I) has at least 95% sequence identity to SEQ ID NO:2.

In certain embodiments SEQ B comprises eleven amino acid changes compared to SEQ ID NO:2. In certain embodiments SEQ B comprises ten amino acid changes compared to SEQ ID NO:2. In certain embodiments SEQ B comprises nine amino acid changes compared to SEQ ID NO:2. In certain embodiments SEQ B comprises eight amino acid changes compared to SEQ ID NO:2. In certain embodiments SEQ B comprises seven amino acid changes compared to SEQ ID NO:2. In certain embodiments SEQ B comprises six amino acid changes compared to SEQ ID NO:2. In certain embodiments SEQ B comprises five amino acid changes compared to SEQ ID NO:2. In certain embodiments SEQ B comprises four amino acid changes compared to SEQ ID NO:2. In certain embodiments SEQ B comprises three amino acid changes compared to SEQ ID NO:2. In certain embodiments SEQ B comprises two amino acid changes compared to SEQ ID NO:2.

SEQ B comprises at least one glycosylation motif. In certain embodiments SEQ B comprises one glycosylation motif. In certain embodiments SEQ B comprises two glycosylation motifs, which are the same or different. In certain embodiments SEQ B comprises three glycosylation motifs, which are the same or different.

In certain embodiments the at least one glycosylation motif is selected from the group consisting of N-glycosylation motifs, O-glycosylation motifs, phosphoglycosylation motifs, and C-glycosylation motifs.

In certain embodiments the at least one glycosylation motif is an N-glycosylation motif.

Such N-glycosylation occurs at the amine functional group of an asparagine amino acid. The asparagine having the amine functional group may either be naturally occurring in the sequence of SEQ ID NO:2 or may be introduced by substituting an amino acid for an asparagine residue.

In certain embodiments the N-glycosylation motif comprises the amino acid sequence X₁X₂NX₃X₄ (SEQ ID NO:254), wherein
X₁ is any proteinogenic or non-proteinogenic amino acid or is absent;
X₂ is any proteinogenic or non-proteinogenic amino acid or is absent;
N is asparagine;
X₃ is any proteinogenic or non-proteinogenic amino acid except proline; and
X₄ is selected from the group consisting of threonine, serine and cysteine.

In certain embodiments X₁ is a proteinogenic amino acid, a non-proteinogenic amino acid or is absent. In certain embodiments X₁ is a proteinogenic amino acid. In certain embodiments X₁ is a non-proteinogenic amino acid. In certain embodiments X₁ is absent. In certain embodiments X₁ is phenylalanine, glutamic acid or aspartic acid. In certain embodiments X₁ is phenylalanine. In certain embodiments X₁ is glutamic acid. In certain embodiments X₁ is aspartic acid.

In certain embodiments X₂ is a proteinogenic amino acid, a non-proteinogenic amino acid or is absent. In certain embodiments X₂ is a proteinogenic amino acid. In certain embodiments X₂ is a non-proteinogenic amino acid. In certain embodiments X₂ is absent. In certain embodiments X₂ is glycine or alanine. In certain embodiments X₂ is glycine. In certain embodiments X₂ is alanine.

In certain embodiments X₃ is a proteinogenic amino acid except proline. In certain embodiments X₃ is a non-proteinogenic amino acid. In certain embodiments X₃ is serine.

In certain embodiments X₄ is threonine, serine or cysteine. In certain embodiments X₄ is threonine. In certain embodiments X₄ is serine. In certain embodiments X₄ is cysteine.

In certain embodiments the N-glycosylation motif has the amino acid sequence NX₃X₄, wherein X₃ and X₄ are as described elsewhere herein. In certain embodiments the N-glycosylation motif is selected from the group consisting of NX₃T, NX₃S and NX₃C. In certain embodiments the N-glycosylation motif is NX₃T. In certain embodiments the N-glycosylation motif is NX₃S. In certain embodiments the N-glycosylation motif is NX₃C. In certain embodiments the N-glycosylation motif is NSX₄. In certain embodiments the N-glycosylation motif is selected from the group consisting of NST, NSS and NSC. In certain embodiments the N-glycosylation motif is NST. In certain embodiments the N-glycosylation motif is NSS. In certain embodiments the N-glycosylation motif is NSC.

In certain embodiments the N-glycosylation motif is X₂NX₃X₄ (SEQ ID NO:255), wherein X₂, X₃ and X₄ are defined as elsewhere herein. In certain embodiments the N-glycosylation motif is selected from the group consisting of X₂NX₃T (SEQ ID NO:256), X₂NX₃S (SEQ ID NO:257) and X₂NX₃C (SEQ ID NO:258). In certain embodiments the N-glycosylation motif is X₂NX₃T (SEQ ID NO:256). In certain embodiments the N-glycosylation motif X₂NX₃S (SEQ ID NO:257). In certain embodiments the N-glycosylation motif is X₂NX₃C (SEQ ID NO:258). In certain embodiments the N-glycosylation motif is X₂NSX₄ (SEQ ID NO:259). In certain embodiments the N-glycosylation motif is selected from the group consisting of X₂NST (SEQ ID NO:260), X₂NSS (SEQ ID NO:261) and X₂NSC (SEQ ID NO:262). In certain embodiments the N-glycosylation motif is X₂NST (SEQ ID NO:260). In certain embodiments the N-glycosylation motif is X₂NSS (SEQ ID NO:261). In certain embodiments the N-glycosylation motif is X₂NSC (SEQ ID NO:262). In certain embodiments the N-glycosylation motif is GNX₃X₄ (SEQ ID NO:263). In certain embodiments the N-glycosylation motif is selected from the group consisting of GNX₃T (SEQ ID NO:264), GNX₃S (SEQ ID NO:265) and GNX₃C (SEQ ID NO:266). In certain embodiments the N-glycosylation motif is GNX₃T (SEQ ID NO:264). In certain embodiments the N-glycosylation motif is GNX₃S (SEQ ID NO:265). In certain embodiments the N-glycosylation motif is GNX₃C (SEQ ID NO:266). In certain embodiments the N-glycosylation motif is GNSX₄ (SEQ ID NO:267). In certain embodiments the N-glycosylation motif is selected from the group consisting of GNST (SEQ ID NO:268), GNSS (SEQ ID NO:269) and GNSC (SEQ ID NO:270). In certain embodiments the N-glycosylation motif is GNST (SEQ ID NO:268). In certain embodiments the N-glycosylation motif is GNSS (SEQ ID NO:269. In certain embodiments the N-glycosylation motif is GNSC (SEQ ID NO:270). In certain embodiments the N-glycosylation motif is ANX₃X₄ (SEQ ID NO:271). In certain embodiments the N-glycosylation motif is selected from the group consisting of ANX₃T (SEQ ID NO:272), ANX₃S (SEQ ID NO:273) and ANX₃C (SEQ ID NO:274). In certain embodiments the N-glycosylation motif is ANX₃T (SEQ ID NO:272). In certain embodiments the N-glycosylation motif is ANX₃S (SEQ ID NO:273). In certain embodiments the N-glycosylation motif is ANX₃C (SEQ ID NO:274). In certain embodiments the N-glycosylation motif is ANSX₄ (SEQ ID NO:275). In certain embodiments the N-glycosylation motif is selected from the group consisting of ANST (SEQ ID NO:276), ANSS (SEQ ID NO:277) and ANSC (SEQ ID NO:278). In certain embodiments the N-glycosylation motif is ANST (SEQ ID NO:276). In certain embodiments the N-glycosylation motif is ANSS (SEQ ID NO:277). In certain embodiments the N-glycosylation motif is ANSC (SEQ ID NO:278).

In certain embodiments the N-glycosylation motif is X₁X₂NX₃X₄ (SEQ ID NO:254), wherein X₁, X₂, X₃ and X₄ are defined as elsewhere herein. In certain embodiments the N-glycosylation motif is selected from the group consisting of X₁X₂NX₃T (SEQ ID NO:279), X₁X₂NX₃S (SEQ ID NO:280) and X₁X₂NX₃C (SEQ ID NO:281). In certain embodiments the N-glycosylation motif is of X₁X₂NX₃T (SEQ ID NO:279). In certain embodiments the N-glycosylation motif is X₁X₂NX₃S (SEQ ID NO:280). In certain embodiments the N-glycosylation motif is X₁X₂NX₃C (SEQ ID NO:281). In certain embodiments the N-glycosylation motif is X₁X₂NSX₄ (SEQ ID NO:282). In certain embodiments the N-glycosylation motif is selected from the group consisting of X₁X₂NST (SEQ ID NO:283), X₁X₂NSS (SEQ ID NO:284) and X₁X₂NSC (SEQ ID NO:285). In certain embodiments the N-glycosylation motif is X₁X₂NST (SEQ ID NO:283). In certain embodiments the N-glycosylation motif is X₁X₂NSS (SEQ ID NO:284). In certain embodiments the N-glycosylation motif is X₁X₂NSC (SEQ ID NO:285). In certain embodiments the N-glycosylation motif is X₁GNX₃X₄ (SEQ ID NO:286). In certain embodiments the N-glycosylation motif is selected from the group consisting of X₁GNX₃T (SEQ ID NO:287), X₁GNX₃S (SEQ ID NO:288) and X₁GNX₃C (SEQ ID NO:289). In certain embodiments the N-glycosylation motif is X₁GNX₃T (SEQ ID NO:287). In certain embodiments the N-glycosylation motif is X₁GNX₃S (SEQ ID NO:288). In certain embodiments the N-glycosylation motif is X₁GNX₃C (SEQ ID NO:289). In certain embodiments the N-glycosylation motif is X₁GNSX₄ (SEQ ID NO:290). In certain embodiments the N-glycosylation motif is selected from the group consisting of X₁ GNST (SEQ ID NO:291), X₁GNSS (SEQ ID NO:292) and X₁GNSC (SEQ ID NO:293). In certain embodiments the N-glycosylation motif is X₁GNST (SEQ ID NO:291). In certain embodiments the N-glycosylation motif is X₁GNSS (SEQ ID NO:292). In certain embodiments the N-glycosylation motif is X₁GNSC (SEQ ID NO:293). In certain embodiments the N-glycosylation motif is FX₂NX₃X₄ (SEQ ID NO:294). In certain embodiments the N-glycosylation motif is selected from the group consisting of FX₂NX₃T (SEQ ID NO:295), FX₂NX₃S (SEQ ID NO:296) and FX₂NX₃C (SEQ ID NO:297). In certain embodiments the N-glycosylation motif is FX₂NX₃T (SEQ ID NO:295). In certain embodiments the N-glycosylation motif is FX₂NX₃S (SEQ ID NO:296). In certain embodiments the N-glycosylation motif is FX₂NX₃C (SEQ ID NO:297). In certain embodiments the N-glycosylation motif is FX₂NSX₄ (SEQ ID NO:298). In certain embodiments the N-glycosylation motif is selected from the group consisting of FX₂NST (SEQ ID NO:299), FX₂NSS (SEQ ID NO:300) and FX₂NSC (SEQ ID NO:301). In certain embodiments the N-glycosylation motif is FX₂NST (SEQ ID NO:299). In certain embodiments the N-glycosylation motif is FX₂NSS (SEQ ID NO:300). In certain embodiments the N-glycosylation motif is FX₂NSC (SEQ ID NO:301). In certain embodiments the N-glycosylation motif is FGNX₃X₄ (SEQ ID NO:302). In certain embodiments the N-glycosylation motif is selected from the group consisting of FGNX₃T (SEQ ID NO:303), FGNX₃S (SEQ ID NO:304) and FGNX₃C (SEQ ID NO:305). In certain embodiments the N-glycosylation motif is FGNX₃T (SEQ ID NO:303). In certain embodiments the N-glycosylation motif is FGNX₃S (SEQ ID NO:304). In certain embodiments the N-glycosylation motif is FGNX₃C (SEQ ID NO:305). In certain embodiments the N-glycosylation motif is FGNSX₄ (SEQ ID NO:306). In certain embodiments the N-glycosylation motif is selected from the group consisting of FGNST (SEQ ID NO:307), FGNSS (SEQ ID NO:308) and FGNSC (SEQ ID NO:309). In certain embodiments the N-glycosylation motif is FGNST (SEQ ID NO:307). In certain embodiments the N-glycosylation motif is FGNSS (SEQ ID NO:308). In certain embodiments the N-glycosylation motif is FGNSC (SEQ ID NO:309). In certain embodiments the N-glycosylation motif is X₁ANX₃X₄ (SEQ ID NO:310). In certain embodiments the N-glycosylation motif is selected from the group consisting of X₁ANX₃T (SEQ ID NO:311), X₁ANX₃S (SEQ ID NO:312) and X₁ANX₃C (SEQ ID NO:313). In certain embodiments the N-glycosylation motif is X₁ANX₃T (SEQ ID NO:311). In certain embodiments the N-glycosylation motif is X₁ANX₃S (SEQ ID NO:312). In certain embodiments the N-glycosylation motif is X₁ANX₃C (SEQ ID NO:313). In certain embodiments the N-glycosylation motif is XiANSX₄ (SEQ ID NO:314). In certain embodiments the N-glycosylation motif is selected from the group consisting of X₁ANST (SEQ ID NO:315), X₁ANSS (SEQ ID NO:316) and X₁ANSC (SEQ ID NO:317). In certain embodiments the N-glycosylation motif is X₁ANST (SEQ ID NO:315). In certain embodiments the N-glycosylation motif is X₁ANSS (SEQ ID NO:316). In certain embodiments the N-glycosylation motif is X₁ANSC (SEQ ID NO:317). In certain embodiments the N-glycosylation motif is FX₂NX₃X₄ (SEQ ID NO:318). In certain embodiments the N-glycosylation motif is selected from the group consisting of FX₂NX₃T (SEQ ID NO:319), FX₂NX₃S (SEQ ID NO:320) and FX₂NX₃C (SEQ ID NO:321). In certain embodiments the N-glycosylation motif is FX₂NX₃T (SEQ ID NO:319).

In certain embodiments the N-glycosylation motif is FX₂NX₃S (SEQ ID NO:320). In certain embodiments the N-glycosylation motif is FX₂NX₃C (SEQ ID NO:321). In certain embodiments the N-glycosylation motif is FX₂NSX₄ (SEQ ID NO:322). In certain embodiments the N-glycosylation motif is selected from the group consisting of FX₂NST (SEQ ID NO:323), FX₂NSS (SEQ ID NO:324) and FX₂NSC (SEQ ID NO:325). In certain embodiments the N-glycosylation motif is FX₂NST (SEQ ID NO:323). In certain embodiments the N-glycosylation motif is FX₂NSS (SEQ ID NO:324). In certain embodiments the N-glycosylation motif is FX₂NSC (SEQ ID NO:325). In certain embodiments the N-glycosylation motif is FANX₃X₄ (SEQ ID NO:326). In certain embodiments the N-glycosylation motif is selected from the group consisting of FANX₃T (SEQ ID NO:327), FANX₃S (SEQ ID NO:328) and FANX₃C (SEQ ID NO:329). In certain embodiments the N-glycosylation motif is FANX₃T (SEQ ID NO:327). In certain embodiments the N-glycosylation motif is FANX₃S (SEQ ID NO:328). In certain embodiments the N-glycosylation motif is FANX₃C (SEQ ID NO:329). In certain embodiments the N-glycosylation motif is FANSX₄ (SEQ ID NO:330). In certain embodiments the N-glycosylation motif is selected from the group consisting of FANST (SEQ ID NO:331), FANSS (SEQ ID NO:332) and FANSC (SEQ ID NO:333). In certain embodiments the N-glycosylation motif is FANST (SEQ ID NO:331). In certain embodiments the N-glycosylation motif is FANSS (SEQ ID NO:332). In certain embodiments the N-glycosylation motif is FANSC (SEQ ID NO:333).

In certain embodiments the N-glycosylation motif is FGNST (SEQ ID NO:307) or FANST (SEQ ID NO:331).

In certain embodiments the at least one glycosylation motif is an O-glycosylation motif.

Such O-glycosylation occurs at an amino acid having a hydroxyl functional group. The amino acid having a hydroxyl functional group may either be naturally occurring in the sequence of SEQ ID NO:2 or may be introduced by substituting an endogenous amino acid for an amino acid comprising a hydroxyl functional group. The amino acid used for O-glycosylation may either be a proteinogenic or a non-proteinogenic amino acid having a hydroxyl functional group. In certain embodiments the amino acid used for O-glycosylation is a proteinogenic amino acid. In certain embodiments the proteinogenic amino acid with a hydroxyl functional group is selected from the group consisting of serine, threonine, tyrosine, hydroxylysine and hydroxyproline. In certain embodiments an amino acid in -6 to -1 or +1 to +4 position to the amino acid with a hydroxyl functional group used for O-glycosylation is substituted by proline. In certain embodiments an amino acid in -6 position to the amino acid with a hydroxyl functional group used for O-glycosylation is substituted by proline. In certain embodiments an amino acid in -5 position to the amino acid with a hydroxyl functional group used for O-glycosylation is substituted by proline. In certain embodiments an amino acid in -4 position to the amino acid with a hydroxyl functional group used for O-glycosylation is substituted by proline. In certain embodiments an amino acid in -3 position to the amino acid with a hydroxyl functional group used for O-glycosylation is substituted by proline. In certain embodiments an amino acid in -2 position to the amino acid with a hydroxyl functional group used for O-glycosylation is substituted by proline. In certain embodiments an amino acid in -1 position to the amino acid with a hydroxyl functional group used for O-glycosylation is substituted by proline. In certain embodiments an amino acid in +1 position to the amino acid with a hydroxyl functional group used for O-glycosylation is substituted by proline. In certain embodiments an amino acid in +2 position to the amino acid with a hydroxyl functional group used for O-glycosylation is substituted by proline. In certain embodiments an amino acid in +3 position to the amino acid with a hydroxyl functional group used for O-glycosylation is substituted by proline. In certain embodiments an amino acid in +4 position to the amino acid with a hydroxyl functional group used for O-glycosylation is substituted by proline.

In certain embodiments the at least one glycosylation motif is obtained by mutating certain consecutive amino acids of SEQ ID NO:2. In certain embodiments one such glycosylation motif is introduced into SEQ ID NO:2 by mutating certain amino acids of SEQ ID NO:2. In certain embodiments two such glycosylation motifs are introduced into SEQ ID NO:2 by mutating certain amino acids of SEQ ID NO:2, wherein the two glycosylation motifs may be the same or different.

In certain embodiments SEQ B comprises an N-glycosylation site at an amino acid position selected from the group consisting of position 3, 4, 5, 6, 7, 8, 9, 10, 11, 12, 13, 14, 15, 16, 17, 29, 30, 31, 32, 33, 34, 40, 41, 42, 43 and 44, based on SEQ ID NO:2 or at the corresponding positions of homologs or variants thereof. In certain embodiments SEQ B comprises an N-glycosylation site at position 3 based on SEQ ID NO:2 or at the corresponding positions of homologs or variants thereof. In certain embodiments SEQ B comprises an N-glycosylation site at position 4 based on SEQ ID NO:2 or at the corresponding positions of homologs or variants thereof. In certain embodiments SEQ B comprises an N-glycosylation site at position 5 based on SEQ ID NO:2 or at the corresponding positions of homologs or variants thereof. In certain embodiments SEQ B comprises an N-glycosylation site at position 6 based on SEQ ID NO:2 or at the corresponding positions of homologs or variants thereof. In certain embodiments SEQ B comprises an N-glycosylation site at position 7 based on SEQ ID NO:2 or at the corresponding positions of homologs or variants thereof. In certain embodiments SEQ B comprises an N-glycosylation site at position 8 based on SEQ ID NO:2 or at the corresponding positions of homologs or variants thereof. In certain embodiments SEQ B comprises an N-glycosylation site at position 9 based on SEQ ID NO:2 or at the corresponding positions of homologs or variants thereof. In certain embodiments SEQ B comprises an N-glycosylation site at position 10 based on SEQ ID NO:2 or at the corresponding positions of homologs or variants thereof. In certain embodiments SEQ B comprises an N-glycosylation site at position 11 based on SEQ ID NO:2 or at the corresponding positions of homologs or variants thereof. In certain embodiments SEQ B comprises an N-glycosylation site at position 12 based on SEQ ID NO:2 or at the corresponding positions of homologs or variants thereof. In certain embodiments SEQ B comprises an N-glycosylation site at position 13 based on SEQ ID NO:2 or at the corresponding positions of homologs or variants thereof. In certain embodiments SEQ B comprises an N-glycosylation site at position 14 based on SEQ ID NO:2 or at the corresponding positions of homologs or variants thereof. In certain embodiments SEQ B comprises an N-glycosylation site at position 15 based on SEQ ID NO:2 or at the corresponding positions of homologs or variants thereof. In certain embodiments SEQ B comprises an N-glycosylation site at position 16 based on SEQ ID NO:2 or at the corresponding positions of homologs or variants thereof. In certain embodiments SEQ B comprises an N-glycosylation site at position 17 based on SEQ ID NO:2 or at the corresponding positions of homologs or variants thereof. In certain embodiments SEQ B comprises an N-glycosylation site at position 29 based on SEQ ID NO:2 or at the corresponding positions of homologs or variants thereof. In certain embodiments SEQ B comprises an N-glycosylation site at position 30 based on SEQ ID NO:2 or at the corresponding positions of homologs or variants thereof. In certain embodiments SEQ B comprises an N-glycosylation site at position 31 based on SEQ ID NO:2 or at the corresponding positions of homologs or variants thereof. In certain embodiments SEQ B comprises an N-glycosylation site at position 32 based on SEQ ID NO:2 or at the corresponding positions of homologs or variants thereof. In certain embodiments SEQ B comprises an N-glycosylation site at position 33 based on SEQ ID NO:2 or at the corresponding positions of homologs or variants thereof. In certain embodiments SEQ B comprises an N-glycosylation site at position 34 based on SEQ ID NO:2 or at the corresponding positions of homologs or variants thereof. In certain embodiments SEQ B comprises an N-glycosylation site at position 40 based on SEQ ID NO:2 or at the corresponding positions of homologs or variants thereof. In certain embodiments SEQ B comprises an N-glycosylation site at position 41 based on SEQ ID NO:2 or at the corresponding positions of homologs or variants thereof. In certain embodiments SEQ B comprises an N-glycosylation site at position 42 based on SEQ ID NO:2 or at the corresponding positions of homologs or variants thereof. In certain embodiments SEQ B comprises an N-glycosylation site at position 43 based on SEQ ID NO:2 or at the corresponding positions of homologs or variants thereof. In certain embodiments SEQ B comprises an N-glycosylation site at position 44 based on SEQ ID NO:2 or at the corresponding positions of homologs or variants thereof.

In certain embodiments SEQ B has a sequence selected from the group consisting of

NX₃X₄PKLTRMLTFKFYMPKKATELKHLQCLEEELKPLEEVLNLAQSK (
SEQ IDNO:85);

YNX₃X₄KLTRMLTFKFYMPKKATELKHLQCLEEELKPLEEVLNLAQSK (
SEQ IDNO:86);

YKNX₃X₄LTRMLTFKFYMPKKATELKHLQCLEEELKPLEEVLNLAQSK (
SEQ IDNO:87);

YKNNX₃X₄TRMLTFKFYMPKKATELKHLQCLEEELKPLEEVLNLAQSK (
SEQ IDNO:88);

YKNPNX₃X₄RMLTFKFYMPKKATELKHLQCLEEELKPLEEVLNLAQSK (
SEQ IDNO:89);

YKNPKNX₃X₄MLTFKFYMPKKATELKHLQCLEEELKPLEEVLNLAQSK (
SEQ IDNO:90);

YKNPKLNX₃X₄LTFKFYMPKKATELKHLQCLEEELKPLEEVLNLAQSK (
SEQ IDNO:91);

YKNPKLTNX₃X₄TFKFYMPKKATELKHLQCLEEELKPLEEVLNLAQSK (
SEQ IDNO:92);

YKNPKLTRNX₃X₄FKFYMPKKATELKHLQCLEEELKPLEEVLNLAQSK (
SEQ IDNO:93);

YKNPKLTRMNX₃X₄KFYMPKKATELKHLQCLEEELKPLEEVLNLAQSK (
SEQ IDNO:94);

YKNPKLTRMLNX₃X₄FYMPKKATELKHLQCLEEELKPLEEVLNLAQSK (
SEQ IDNO:95);

YKNPKLTRMLTNX₃X₄YMPKKATELKHLQCLEEELKPLEEVLNLAQSK (
SEQ IDNO:96);

YKNPKLTRMLTFNX₃X₄MPKKATELKHLQCLEEELKPLEEVLNLAQSK (
SEQ IDNO:97);

YKNPKLTRMLTFKNX₃X₄PKKATELKHLQCLEEELKPLEEVLNLAQSK (
SEQ IDNO:98);

YKNPKLTRMLTFKFNX₃X₄KKATELKHLQCLEEELKPLEEVLNLAQSK (
SEQ IDNO:99);

YKNPKLTRMLTFKFYNX₃X₄KATELKHLQCLEEELKPLEEVLNLAQSK (
SEQ IDNO:100);

YKNPKLTRMLTFKFYMNX₃X₄ATELKHLQCLEEELKPLEEVLNLAQSK (
SEQ IDNO:101);

YKNPKLTRMLTFKFYMPNX₃X₄TELKHLQCLEEELKPLEEVLNLAQSK (
SEQ IDNO:102);

YKNPKLTRMLTFKFYMPKNX₃X₄ELKHLQCLEEELKPLEEVLNLAQSK (
SEQ IDNO:103);

YKNPKLTRMLTFKFYMPIG<.NX₃X₄LKHLQCLEEELKPLEEVLNLAQSK
(SEQ IDNO:104);

YKNPKLTRMLTFKFYMPKKANX₃X₄KHLQCLEEELKPLEEVLNLAQSK (
SEQ IDNO:105);

YKNPKLTRMLTFKFYMPKKATNX₃X₄HLQCLEEELKPLEEVLNLAQSK (
SEQ IDNO:106);

YKNPKLTRMLTFKFYMPKKATENX₃X₄LQCLEEELKPLEEVLNLAQSK (
SEQ IDNO:107);

YKNPKLTRMLTFKFYMPKKATELNX₃X₄QCLEEELKPLEEVLNLAQSK (
SEQ IDNO:108);

YKNPKLTRMLTFKFYMPKKATELKNX₃X₄CLEEELKPLEEVLNLAQSK (
SEQ IDNO:109);

YKNPKLTRMLTFKFYMPIGTELKHNX3X4LEEELKPLEEVLNLAQSK (S
EQ IDNO:110);

YKNPKLTRMLTFKFYMPKKATELKHLNX₃X₄EEELKPLEEVLNLAQSK (
SEQ IDNO: 111);

YKNPKLTRMLTFKFYMPKKATELIELQNX₃X₄EELKPLEEVLNLAQSK (
SEQ IDNO:113);

YKNPKLTRMLTFKFYMPIGTELKHLQCNX₃X₄ELKPLEEVLNLAQSK (S
EQ IDNO:114);

YKNPKLTRMLTFKFYMPKKATELKHLQCLNX₃X₄LKPLEEVLNLAQSK (
SEQ IDNO:115);

YKNPKLTRMLTFKFYMPIGTELKHLQCLENX₃X₄KPLEEVLNLAQSK (S
EQ IDNO:116);

YKNPKLTRMLTFKFYMPKKATELKHLQCLEENX₃X₄PLEEVLNLAQSK (
SEQ IDNO:117);

YKNPKLTRMLTFKFYMPKKATELIELQCLEEENX₃X₄LEEVLNLAQSK (
SEQ IDNO:118);

YKNPKLTRMLTFKFYMPKKATELKHLQCLEEELNX₃X₄EEVLNLAQSK (
SEQ IDNO:119);

YKNPKLTRMLTFKFYMPKKATELIELQCLEEELKNX₃X₄EVLNLAQSK (
SEQ IDNO:120);

YKNPKLTRMLTFKFYMPKKATELKHLQCLEEELKPNX₃X₄VLNLAQSK (
SEQ IDNO:121);

YKNPKLTRMLTFKFYMPIGTELKHLQCLEEELKPLNX₃X₄LNLAQSK (S
EQ IDNO:122);

YKNPKLTRMLTFKFYMPKKATELKHLQCLEEELKPLENX₃X₄NLAQSK (
SEQ IDNO:123);

YKNPKLTRMLTFKFYMPKKATELIELQCLEEELKPLEENX₃X₄LAQSK (
SEQ IDNO:124);

YKNPKLTRMLTFKFYMPKKATELKHLQCLEEELKPLEEVNX₃X₄AQSK (
SEQ IDNO:125);

YKNPKLTRMLTFKFYMPIGTELKHLQCLEEELKPLEEVLNX₃X₄QSK (S
EQ IDNO:126);

YKNPKLTRMLTFKFYMPKKATELKHLQCLEEELKPLEEVLNNX₃X₄SK (
SEQ IDNO:127);

YKNPKLTRMLTFKFYMPKKATELIELQCLEEELKPLEEVLNLNX₃X₄K (
SEQ IDNO:128); and

YKNPKLTRMLTFKFYMPKKATELKHLQCLEEELKPLEEVLNLANX₃X₄ (
SEQ IDNO:129), wherein X₃ and X₄ are used as defin
ed elsewhere

In certain embodiments SEQ B has a sequence selected from the group consisting of

X₁X₂NX₃X₄LTRMLTFKFYMPKKATELKHLQCLEEELKPLEEVLNLAQSK
(SEQ IDNO:130);

YX₁X₂NX₃X₄TRMLTFKFYMPKKATELKHLQCLEEELKPLEEVLNLAQSK
(SEQ IDNO: 131);

YKX₁X₂NX₃X₄RMLTFKFYMPKKATELKHLQCLEEELKPLEEVLNLAQSK
(SEQ IDNO:132);

YKNX₁X₂NX₃X₄MLTFIUYMPICATELKHLQCLEEELIULEEVLNLAQSK
(SEQ IDNO:133);

YKNPX₁X₂NX₃X₄LTFKFYMPKKATELKHLQCLEEELKPLEEVLNLAQSK
(SEQ IDNO:134);

YKNPKX₁X₂NX₃X₄TFKFYMPKKATELKHLQCLEEELKPLEEVLNLAQSK
(SEQ IDNO:135);

YKNPKLX₁X₂NX₃X₄FKFYMPKKATELKHLQCLEEELKPLEEVLNLAQSK
(SEQ IDNO:136);

YKNPKLTX_{I}X₂NX₃X₄KFYMPKKATELKHLQCLEEELKPLEEVLNLAQSK
(SEQ IDNO:137);

YKNPKLTRX₁X₂NX₃X₄FYMPKKATELKHLQCLEEELKPLEEVLNLAQSK
(SEQ IDNO:138);

YKNPKLTRMX₁X₂NX₃X₄YMPICATELKHLQCLEEELKPLEEVLNLAQSK
(SEQ IDNO:139);

YKNPKLTRMLX₁X₂NX₃X₄MPKKATELKHLQCLEEELKPLEEVLNLAQSK
(SEQ IDNO: 140);

YKNPKLTRMLTX₁X₂NX₃X₄PICATELKHLQCLEEELIULEEVLNLAQSK
(SEQ IDNO:141);

YKNPKLTRMLTFX₁X₂NX₃X₄KKATELKHLQCLEEELKPLEEVLNLAQSK
(SEQ IDNO:142);

YKNPKLTRMLTFKX₁X₂NX₃X₄KATELKHLQCLEEELKPLEEVLNLAQSK
(SEQ IDNO:143);

YKNPKLTRMLTFKFX₁X₂NX₃X₄ATELKHLQCLEEELKPLEEVLNLAQSK
(SEQ IDNO:144);

YKNPKLTRMLTFKFYX₁X₂NX₃X₄TELKHLQCLEEELKPLEEVLNLAQSK
(SEQ IDNO:145);

YKNPKLTRMLTFKFYMX_{I}X₂NX₃X₄ELKHLQCLEEELKPLEEVLNLAQSK
(SEQ IDNO:146);

YKNPKLTRMLTFKFYMPX₁X₂NX₃X₄LKHLQCLEEELKPLEEVLNLAQSK
(SEQ IDNO:147);

YKNPKLTRMLTFKFYMPKX₁X₂NX₃X₄KHLQCLEEELKPLEEVLNLAQSK
(SEQ IDNO:148);

YKNPKLTRMLTFKFYMPKKX₁X₂NX₃X₄HLQCLEEELKPLEEVLNLAQSK
(SEQ IDNO: 149);

YKNPKLTRMLTFKFYMPKKAX₁X₂NX₃X₄LQCLEEELKPLEEVLNLAQSK
(SEQ IDNO:150);

YKNPKLTRMLTFKFYMPKKATX₁X₂NX₃X₄QCLEEELKPLEEVLNLAQSK
(SEQ IDNO:151);

YKNPKLTRMLTFKFYMPIGTEX₁X₂NX₃X₄CLEEELKPLEEVLNLAQSK
(SEQ IDNO:152);

YKNPKLTRMLTFKFYMPKKATELX₁X₂NX₃X₄LEEELKPLEEVLNLAQSK
(SEQ IDNO:153);

YKNPKLTRMLTFKFYMPICATELIX₁X₂NX₃X₄EEELKPLEEVLNLAQSK
(SEQ IDNO:154);

YKNPKLTRMLTFKFYMPKKATELKHX₁X₂NX₃X₄EELKPLEEVLNLAQSK
(SEQ IDNO:155);

YKNPKLTRMLTFKFYMPKKATELKHLX₁X₂NX₃X₄ELIPLEEVLNLAQSK
(SEQ IDNO:156);

YKNPKLTRMLTFKFYMPKKATELKHLQX₁X₂NX₃X₄LKPLEEVLNLAQSK
(SEQ IDNO:157);

YKNPKLTRMLTFKFYMPKKATELKHLQCX₁X₂NX₃X₄KPLEEVLNLAQSK
(SEQ IDNO:158);

YKNPKLTRMLTFKFYMPKKATELKHLQCLX₁X₂NX₃X₄PLEEVLNLAQSK
(SEQ IDNO:159);

YKNPKLTRMLTFKFYMPIGTELKHLQCLEX₁X₂NX₃X₄LEEVLNLAQSK
(SEQ IDNO:160);

YKNPKLTRMLTFKFYMPKKATELKHLQCLEEX₁X₂NX₃X₄EEVLNLAQSK
(SEQ IDNO:161);

YKNPKLTRMLTFKFYMPICATELIHLQCLEEEX₁X₂NX₃X₄EVLNLAQSK
(SEQ IDNO:162);

YKNPKLTRMLTFKFYMPIGTELKHLQCLEEELX₁X₂NX₃X₄VLNLAQSK
(SEQ IDNO:163);

YKNPKLTRMLTFKFYMPKKATELKHLQCLEEELKX₁X₂NX₃X₄LNLAQSK
(SEQ IDNO:164);

YKNPKLTRMLTFKFYMPKKATELKHLQCLEEELKPX₁X₂NX₃X₄NLAQSK
(SEQ IDNO:165);

YKNPKLTRMLTFKFYMPKKATELKHLQCLEEELKPLX₁X₂NX₃X₄LAQSK
(SEQ IDNO:166);

YKNPKLTRMLTFKFYMPIGTELKHLQCLEEELKPLEX₁X₂NX₃X₄AQSK
(SEQ IDNO:167);

YKNPKLTRMLTFKFYMPKKATELKHLQCLEEELKPLEEX₁X₂NX₃X₄QSK
(SEQ IDNO:168);

YKNPKLTRMLTFKFYMPIGTELKHLQCLEEELKPLEEVX₁X₂NX₃X₄SK
(SEQ IDNO:169);

YKNPKLTRMLTFKFYMPKKATELKHLQCLEEELKPLEEVLX₁X₂NX₃X₄K
(SEQ IDNO: 170); and

YKNPKLTRMLTFKFYMPKKATELKHLQCLEEELKPLEEVLNX₁X₂NX₃X₄
(SEQ IDNO:171).

In certain embodiments SEQ B has a sequence selected from the group consisting of SEQ ID NO:87; SEQ ID NO:88; SEQ ID NO:89; SEQ ID NO:90; SEQ ID NO:91; SEQ ID NO:92; SEQ ID NO:93; SEQ ID NO:94; SEQ ID NO:95; SEQ ID NO:96; SEQ ID NO:97; SEQ ID NO:98; SEQ ID NO:99; SEQ ID NO:100; SEQ ID NO:114; SEQ ID NO:115; SEQ ID NO:116; SEQ ID NO:117; SEQ ID NO:125; SEQ ID NO:126; and SEQ ID NO:127:

In certain embodiments SEQ B has a sequence selected from the group consisting of SEQ ID NO:130; SEQ ID NO:131; SEQ ID NO:132; SEQ ID NO:133; SEQ ID NO:134; SEQ ID NO:135; SEQ ID NO:136; SEQ ID NO:137; SEQ ID NO:138; SEQ ID NO:139; SEQ ID NO:140; SEQ ID NO:141; SEQ ID NO:142; SEQ ID NO:143; SEQ ID NO:144; SEQ ID NO:156; SEQ ID NO:157; SEQ ID NO:158; SEQ ID NO:159; SEQ ID NO:160; SEQ ID NO:161; SEQ ID NO:167; SEQ ID NO:168; SEQ ID NO:169; SEQ ID NO:170; and SEQ ID NO:171.

In certain embodiments SEQ B has a sequence selected from the group consisting of SEQ ID NO:89; SEQ ID NO:92; SEQ ID NO:93; SEQ ID NO:95; SEQ ID NO:96; SEQ ID NO:97; SEQ ID NO:98; SEQ ID NO:99; SEQ ID NO:116; SEQ ID NO:117; and SEQ ID NO:127.

In certain embodiments SEQ B has a sequence selected from the group consisting of SEQ ID NO:134; SEQ ID NO:137; SEQ ID NO:138; SEQ ID NO:140; SEQ ID NO:141; SEQ ID NO:142; SEQ ID NO:143; SEQ ID NO:144; SEQ ID NO:160; SEQ ID NO:161; and SEQ ID NO:171.

In certain embodiments SEQ B has a sequence selected from the group consisting of

YKNPNSTRMLTFKFYMPKKATELKHLQCLEEELKPLEEVLNLAQSK (SE
Q IDNO: 172);

YKNPKLTNSTTFKFYMPKKATELKHLQCLEEELKPLEEVLNLAQSK (SE
Q IDNO:173);

YKNPKLTRNSTFKFYMPKKATELKHLQCLEEELKPLEEVLNLAQSK (SE
Q IDNO:174);

YKNPKLTRMLNSTFYMPKKATELKHLQCLEEELKPLEEVLNLAQSK (SE
Q IDNO:175);

YKNPKLTRMLTNSTYMPKKATELKHLQCLEEELKPLEEVLNLAQSK (SE
Q IDNO:176);

YKNPKLTRMLTFNSTMPKKATELKHLQCLEEELKPLEEVLNLAQSK (SE
Q IDNO: 177);

YKNPKLTRMLTFKNSTPKKATELKHLQCLEEELKPLEEVLNLAQSK (SE
Q IDNO: 178);

YKNPKLTRMLTFKFNSTKKATELKHLQCLEEELKPLEEVLNLAQSK (SE
Q IDNO: 179);

YKNPKLTRMLTFKFYMPKKATELKHLQCLENSTKPLEEVLNLAQSK (SE
Q IDNO:112);

YKNPKLTRMLTFKFYMPKKATELKHLQCLEENSTPLEEVLNLAQSK (SE
Q IDNO: 180); and

YKNPKLTRMLTFKFYMPKKATELKHLQCLEEELKPLEEVLNNSTSK (SE
Q IDNO:181).

In certain embodiments SEQ B has a sequence selected from the group consisting of

YKNPFANSTLTFKFYMPKKATELKHLQCLEEELKPLEEVLNLAQSK (SE
Q ID NO: 182);

YKNPKLTFANSTKFYMPKKATELKHLQCLEEELKPLEEVLNLAQSK (SE
Q ID NO:14);

YKNPKLTRFANSTFYMPKKATELKHLQCLEEELKPLEEVLNLAQSK (SE
Q IDNO:183);

YKNPKLTRMLFANSTMPKKATELKHLQCLEEELKPLEEVLNLAQSK (SE
Q ID NO:20);

YKNPKLTRMLTFANSTPKKATELKHLQCLEEELKPLEEVLNLAQSK (SE
Q IDNO:184);

YKNPKLTRMLTFFANSTKKATELKHLQCLEEELKPLEEVLNLAQSK (SE
Q IDNO: 185);

YKNPKLTRMLTFKFANSTKATELKHLQCLEEELKPLEEVLNLAQSK (SE
Q IDNO: 186);

YKNPKLTRMLTFKFFANSTATELKHLQCLEEELKPLEEVLNLAQSK (SE
Q ID NO:187);

YKNPKLTRMLTFKFYMPKKATELKHLQCLEFANSTLEEVLNLAQSK (SE
Q ID NO:26);

YKNPKLTRMLTFKFYMPKKATELKHLQCLEEFANSTEEVLNLAQSK (SE
Q IDNO:188);

and YKNPKLTRMLTFKFYMPKKATELKHLQCLEEELKPLEEVLNFANST
(SEQ IDNO: 189).

In certain embodiments SEQ B has a sequence selected from the group consisting of

YKNPFGNSTLTFKFYMPKKATELKHLQCLEEELKPLEEVLNLAQSK (SE
Q ID NO: 190);

YKNPKLTFGNSTIUYMPIC<-ATELKHLQCLEEELIULEEVLNLAQSK (
SEQ ID NO: 11);

YKNPKLTRFGNSTFYMPKKATELKHLQCLEEELKPLEEVLNLAQSK (SE
Q IDNO:191);

YKNPKLTRMLFGNSTMPKKATELKHLQCLEEELKPLEEVLNLAQSK (SE
Q ID NO: 17);

YKNPKLTRMLTFGNSTPKKATELKHLQCLEEELKPLEEVLNLAQSK (SE
Q IDNO:192);

YKNPKLTRMLTFFGNSTKKATELKHLQCLEEELKPLEEVLNLAQSK (SE
Q IDNO:193);

YKNPKLTRMLTFKFGNSTKATELKHLQCLEEELKPLEEVLNLAQSK (SE
Q IDNO:194);

YKNPKLTRMLTFKFFGNSTATELKHLQCLEEELKPLEEVLNLAQSK (SE
Q ID NO: 195);

YKNPKLTRMLTFKFYMPKKATELKHLQCLEFGNSTLEEVLNLAQSK (SE
Q ID NO:23);

YKNPKLTRMLTFKFYMPKKATELKHLQCLEEFGNSTEEVLNLAQSK (SE
Q IDNO: 196);

and

YKNPKLTRMLTFKFYMPKKATELKHLQCLEEELKPLEEVLNFGNST (SE
Q IDNO:197).

In certain embodiments the N-glycosylation motif is introduced in a way so that an endogenous asparagine at position 3 or 41 of sequence SEQ ID NO:2 becomes part of an N-glycosylation motif, in which case the asparagine does not need to be substituted, resulting in SEQ B of one of the sequences selected from the group of sequences SEQ ID NO:87; SEQ ID NO:130; SEQ ID NO:127; SEQ ID NO:168;

YKNSTLTRMLTFKFYMPKKATELKHLQCLEEELKPLEEVLNLAQSK (SE
Q IDNO: 198);

FANSTLTRMLTFKFYMPKKATELKHLQCLEEELKPLEEVLNLAQSK (SE
Q IDNO: 199);

FGNSTLTRMLTFKFYMPKKATELKHLQCLEEELKPLEEVLNLAQSK (SE
Q IDNO:200);

YKNPKLTRMLTFKFYMPKKATELKHLQCLEEELKPLEEVLNSTQSK (SE
Q IDNO:201);

YKNPKLTRMLTFKFYMPKKATELKHLQCLEEELKPLEEFANSTQSK (SE
Q ID NO:32);

and

YKNPKLTRMLTFKFYMPKKATELKHLQCLEEELKPLEEFGNSTQSK (SE
Q ID NO:35).

In certain embodiments the O-glycosylation motif is introduced by substituting any amino acid in sequence SEQ B with an amino acid selected from the group consisting of serine, threonine, tyrosine, hydroxylysine, and hydroxyproline.

In certain embodiments the O-glycosylation motif, i.e. the mutation of an amino acid of SEQ ID NO:2 to an amino acid selected from the group consisting of serine, threonine, tyrosine, hydroxylysine, and hydroxyproline, occurs at positions K5, R8, M9, T11, F12, K13, F14, Y15, E32, or L42 of sequence SEQ B.

In certain embodiments the O-glycosylation motif is introduced by an amino acid substitution in sequence SEQ B selected from the group consisting of K5S, K5T, K5Y, K5Hyl, K5Hyp, R8S, R8T, R8Y, R8Hyl, R8Hy, M9S, M9T, M9Y, M9Hyl, M9Hyp, T11S, T11Y, T11Hyl, T11Hyp, F12S, F12T, F12Y, F12Hyl, F12Hyp, K13S, K13T, K13Y, K13Hyl, K13Hyp, Y15S, Y15T, Y15Hyl, Y15Hyp, E32S, E32T, E32Y, E32Hyl, E32Hyp, L42S, L42T, L42Y, L42Hyl and L42Hyp.

In certain embodiments SEQ B is selected from the group consisting of SEQ ID NO: 11, SEQ ID NO:14, SEQ ID NO:17, SEQ ID NO:20, SEQ ID NO:23, SEQ ID NO:26, SEQ ID NO:32, SEQ ID NO:35, SEQ ID NO:214 and SEQ ID NO:215.

In certain embodiments SEQ B has the sequence of SEQ ID NO: 11. In certain embodiments SEQ B has the sequence of SEQ ID NO:14. In certain embodiments SEQ B has the sequence of SEQ ID NO: 17. In certain embodiments SEQ B has the sequence of SEQ ID NO:20. In certain embodiments SEQ B has the sequence of SEQ ID NO:23. In certain embodiments SEQ B has the sequence of SEQ ID NO:26. In certain embodiments SEQ B has the sequence of SEQ ID NO:32. In certain embodiments SEQ B has the sequence of SEQ ID NO:35. In certain embodiments SEQ B has the sequence of SEQ ID NO:214. In certain embodiments SEQ B has the sequence of SEQ ID NO:215.

In certain embodiments the insertion of a glycosylation motif into SEQ B results in at least 50% site occupancy. In certain embodiments the insertion of a glycosylation motif into SEQ B results in at least 55% site occupancy. In certain embodiments the insertion of a glycosylation motif into SEQ B results in at least 60% site occupancy. In certain embodiments the insertion of a glycosylation motif into SEQ B results in at least 65% site occupancy. In certain embodiments the insertion of a glycosylation motif into SEQ B results in at least 70% site occupancy. In certain embodiments the insertion of a glycosylation motif into SEQ B results in at least 75% site occupancy. In certain embodiments the insertion of a glycosylation motif into SEQ B results in at least 80% site occupancy. In certain embodiments the insertion of a glycosylation motif into SEQ B results in at least 85% site occupancy. In certain embodiments the insertion of a glycosylation motif into SEQ B results in at least 90% site occupancy. In certain embodiments the insertion of a glycosylation motif into SEQ B results in at least 95% site occupancy.

In certain embodiments SEQ B further comprises at least one amino acid mutation occurring at an amino acid position selected from the group consisting of K5, R8, M9, T11, F12, K13, F14, Y15, E31, E32 and L42, based on SEQ ID NO:2 or at the corresponding positions of homologs or variants thereof. Even more preferably the at least one amino acid mutation comprises a mutation at an amino acid position selected from the group consisting of F12, Y15, E31, E32 and L42 based on SEQ ID NO:2 or at the corresponding positions of homologs or variants thereof. In certain embodiments the at least one amino acid mutation comprises a mutation at amino acid position K5 based on SEQ ID NO:2 or at the corresponding positions of homologs or variants thereof. In certain embodiments the at least one amino acid mutation comprises a mutation at amino acid position R8 based on SEQ ID NO:2 or at the corresponding positions of homologs or variants thereof. In certain embodiments the at least one amino acid mutation comprises a mutation at amino acid position M9 based on SEQ ID NO:2 or at the corresponding positions of homologs or variants thereof. In certain embodiments the at least one amino acid mutation comprises a mutation at amino acid position T11 based on SEQ ID NO:2 or at the corresponding positions of homologs or variants thereof. In certain embodiments the at least one amino acid mutation comprises a mutation at amino acid position F12 based on SEQ ID NO:2 or at the corresponding positions of homologs or variants thereof. In certain embodiments the at least one amino acid mutation comprises a mutation at amino acid position K13 based on SEQ ID NO:2 or at the corresponding positions of homologs or variants thereof. In certain embodiments the at least one amino acid mutation comprises a mutation at amino acid position F14 based on SEQ ID NO:2 or at the corresponding positions of homologs or variants thereof. In certain embodiments the at least one amino acid mutation comprises a mutation at amino acid position Y15 based on SEQ ID NO:2 or at the corresponding positions of homologs or variants thereof. In certain embodiments the at least one amino acid mutation comprises a mutation at amino acid position E31 based on SEQ ID NO:2 or at the corresponding positions of homologs or variants thereof. In certain embodiments the at least one amino acid mutation comprises a mutation at amino acid position E32 based on SEQ ID NO:2 or at the corresponding positions of homologs or variants thereof. In certain embodiments the at least one amino acid mutation comprises a mutation at amino acid position L42 based on SEQ ID NO:2 or at the corresponding positions of homologs or variants thereof.

In certain embodiments such mutations are a replacement of a proteinogenic naturally occurring amino acid with an amino acid residue selected from the group consisting of alanine, arginine, asparagine, aspartic acid, cysteine, glutamine, glutamic acid, glycine, histidine, lysine, serine, threonine, tryptophan and tyrosine. In certain embodiments the naturally occurring amino acid is replaced with alanine. In certain embodiments the naturally occurring amino acid is replaced with arginine. In certain embodiments the naturally occurring amino acid is replaced with asparagine. In certain embodiments the naturally occurring amino acid is replaced with aspartic acid. In certain embodiments the naturally occurring amino acid is replaced with cysteine. In certain embodiments the naturally occurring amino acid is replaced with glutamine. In certain embodiments the naturally occurring amino acid is replaced with glutamic acid. In certain embodiments the naturally occurring amino acid is replaced with glycine. In certain embodiments the naturally occurring amino acid is replaced with histidine. In certain embodiments the naturally occurring amino acid is replaced with lysine. In certain embodiments the naturally occurring amino acid is replaced with serine. In certain embodiments the naturally occurring amino acid is replaced with threonine. In certain embodiments the naturally occurring amino acid is replaced with tryptophan. In certain embodiments the naturally occurring amino acid is replaced with tyrosine. In certain embodiments such mutations are a replacement of a naturally occurring amino acid with an amino acid residue selected from the group consisting of arginine, aspartic acid, cysteine, glutamine, glutamic acid, histidine, lysine, serine, threonine, tryptophan and tyrosine. In certain embodiments such mutations are a replacement of a naturally occurring amino acid with an amino acid residue selected from the group consisting of cysteine, glutamic acid, lysine, serine, threonine and tyrosine. In certain embodiments the naturally occurring amino acid is replaced by a non-proteinogenic amino acid. Embodiments for such non-proteinogenic amino acids are as described above.

In certain embodiments SEQ B comprises an amino acid mutation selected from the group consisting of K5A, K5C, K5G, K5S, K5T, K5Q, K5E, K5N, K5D, K5H, K5W, K5Y and K5R, based on SEQ ID NO:2 or the corresponding positions of homologs or variants thereof. In certain embodiments SEQ B comprises the K5A mutation. In certain embodiments the SEQ B comprises the K5C mutation. In certain embodiments the SEQ B comprises the K5G mutation. In certain embodiments the SEQ B comprises the K5S mutation. In certain embodiments the SEQ B comprises the K5T mutation. In certain embodiments the SEQ B comprises the K5Q mutation. In certain embodiments the SEQ B comprises the K5E mutation. In certain embodiments the SEQ B comprises the K5D mutation. In certain embodiments the SEQ B comprises the K5H mutation. In certain embodiments the SEQ B comprises the K5W mutation. In certain embodiments the SEQ B comprises the K5Y mutation. In certain embodiments the SEQ B comprises the K5R mutation.

In certain embodiments SEQ B has a sequence selected from the group consisting of YKNPDLTFGNSTIUYMPICATELKHLQCLEEELIULEEVLNLAQSK (SEQ ID NO:214), YKNPELTFGNSTIUYMPICATELKHLQCLEEELIULEEVLNLAQSK (SEQ ID NO:215), YKNPDLTFANSTKFYMPKKATELKHLQCLEEELKPLEEVLNLAQSK (SEQ ID NO:235) and YKNPELTFANSTKFYMPKKATELKHLQCLEEELKPLEEVLNLAQSK (SEQ ID NO:236). In certain embodiments SEQ B has the sequence of SEQ ID NO:214. In certain embodiments SEQ B has the sequence of SEQ ID NO:215. In certain embodiments SEQ B has the sequence of SEQ ID NO:235. In certain embodiments SEQ B has the sequence of SEQ ID NO:236.

In certain embodiments SEQ B comprises an amino acid mutation selected from the group consisting of R8A, R8C, R8G, R8S, R8T, R8Q, R8E, R8N, R8D, R8H, R8W, R8Y and R8K, based on SEQ ID NO:2 or the corresponding positions of homologs or variants thereof. In certain embodiments SEQ B comprises the R8A mutation. In certain embodiments the SEQ B comprises the R8C mutation. In certain embodiments the SEQ B comprises the R8G mutation. In certain embodiments the SEQ B comprises the R8S mutation. In certain embodiments the SEQ B comprises the R8T mutation. In certain embodiments the SEQ B comprises the R8Q mutation. In certain embodiments the SEQ B comprises the R8E mutation. In certain embodiments the SEQ B comprises the R8N mutation. In certain embodiments the SEQ B comprises the R8D mutation. In certain embodiments the SEQ B comprises the R8H mutation. In certain embodiments the SEQ B comprises the R8K mutation. In certain embodiments the SEQ B comprises the R8W mutation. In certain embodiments the SEQ B comprises the R8Y mutation. In certain embodiments the SEQ B comprises the R8K mutation.

In certain embodiments SEQ B comprises an amino acid mutation selected from the group consisting of F12A, F12C, F12G, F12S, F12T, F12Q, F12E, F12N, F12D, F12R, F12H, F12W, F 12Y and F 12K, based on SEQ ID NO:2 or the corresponding positions of homologs or variants thereof. In certain embodiments the SEQ B comprises an amino acid mutation selected from the group consisting of F12A, F12C, F12G, F12S, F12T, F12Q, F12E, F12N, F12D, F12R and F12K, based on SEQ ID NO:2 or the corresponding positions of homologs or variants thereof. In certain embodiments the SEQ B comprises the F12A mutation. In certain embodiments the SEQ B comprises the F12C mutation. In certain embodiments the SEQ B comprises the F12G mutation. In certain embodiments the SEQ B comprises the F12S mutation. In certain embodiments the SEQ B comprises the F12T mutation. In certain embodiments the SEQ B comprises the F12Q mutation. In certain embodiments the SEQ B comprises the F12E mutation. In certain embodiments the SEQ B comprises the F12N mutation. In certain embodiments the SEQ B comprises the F12D mutation. In certain embodiments the SEQ B comprises the F12R mutation. In certain embodiments the SEQ B comprises the F12H mutation. In certain embodiments the SEQ B comprises the F12W mutation. In certain embodiments the SEQ B comprises the F12Y mutation. In certain embodiments the SEQ B comprises the F12K mutation.

In certain embodiments SEQ B comprises an amino acid mutation selected from the group consisting of Y15A, Y15C, Y15G, Y15S, Y15T, Y15Q, Y15E, Y15N, Y15D, Y15R, Y15H, Y15W and Y15K, based on SEQ ID NO:2 or the corresponding positions of homologs or variants thereof. In certain embodiments the SEQ B comprises an amino acid mutation selected from the group consisting of Y15A, Y15C, Y15G, Y15S, Y15T, Y15Q, Y15E, Y15N, Y15D, Y15R and Y15K, based on SEQ ID NO:2or the corresponding positions of homologs or variants thereof. In certain embodiments the SEQ B comprises the Y15A mutation. In certain embodiments the SEQ B comprises the Y15C mutation. In certain embodiments the SEQ B comprises the Y15G mutation. In certain embodiments the SEQ B comprises the Y15S mutation. In certain embodiments the SEQ B comprises the Y15T mutation. In certain embodiments the SEQ B comprises the Y15Q mutation. In certain embodiments the SEQ B comprises the Y15E mutation. In certain embodiments the SEQ B comprises the Y15N mutation. In certain embodiments the SEQ B comprises the Y15D mutation. In certain embodiments the SEQ B comprises the Y15R mutation. In certain embodiments the SEQ B comprises the Y15H mutation. In certain embodiments the SEQ B comprises the Y15W mutation. In certain embodiments the SEQ B comprises the Y15K mutation.

In certain embodiments SEQ B comprises an amino acid mutation selected from the group consisting of L42G, L42C, L42A, L42S, L42T, L42Q, L42E, L42N, L42D, L42R, L42H, L42W, L42Y and L42K, based on SEQ ID NO:2 or the corresponding positions of homologs or variants thereof. In certain embodiments the SEQ B comprises an amino acid mutation selected from the group consisting of L42G, L42C, L42A, L42S, L42T, L42Q, L42E, L42N, L42D, L42R and L42K, based on SEQ ID NO:2 or the corresponding positions of homologs or variants thereof. In certain embodiments the SEQ B comprises the L42G mutation. In certain embodiments the SEQ B comprises the L42C mutation. In certain embodiments the SEQ B comprises the L42A mutation. In certain embodiments the SEQ B comprises the L42S mutation. In certain embodiments the SEQ B comprises the L42T mutation. In certain embodiments the SEQ B comprises the L42Q mutation. In certain embodiments the SEQ B comprises the L42E mutation. In certain embodiments the SEQ B comprises the L42N mutation. In certain embodiments the SEQ B comprises the L42D mutation. In certain embodiments the SEQ B comprises the L42R mutation. In certain embodiments the SEQ B comprises the L42H mutation. In certain embodiments the SEQ B comprises the L42W mutation. In certain embodiments the SEQ B comprises the L42Y mutation. In certain embodiments the SEQ B comprises the L42K mutation.

In certain embodiments SEQ B comprises an amino acid mutation selected from the group consisting of F12A, F12C, F12G, F12S, F12T, F12Q, F12E, F12N, F12D, F12R and F12K and a further amino acid mutation selected from the group consisting of Y15A, Y15C, Y15G, Y15S, Y15T, Y15Q, Y15E, Y15N, Y15D, Y15R and Y15K, based on SEQ ID NO:2 or the corresponding positions of homologs or variants thereof. In certain embodiments SEQ B comprises the F12A and Y15A mutation. In certain embodiments the SEQ B comprises the F12C mutation and the Y15A mutation. In certain embodiments the SEQ B comprises the F12A and the Y15C mutation.

In certain embodiments SEQ B comprises an amino acid mutation selected from the group consisting of F12A, F12C, F12G, F12S, F12T, F12Q, F12E, F12N, F12D, F12R and F12K and a further amino acid mutation selected from the group consisting of L42G, L42C, L42A, L42S, L42T, L42Q, L42E, L42N, L42D, L42R and L42K, based on SEQ ID NO:2 or the corresponding positions of homologs or variants thereof. In certain embodiments SEQ B comprises the F12A and L42G mutations. In certain embodiments SEQ B comprises the F42C and L42G mutations. In certain embodiments SEQ B comprises the F42A and L42C mutations.

In certain embodiments SEQ B comprises an amino acid mutation selected from the group consisting of Y15A, Y15C, Y15G, Y15S, Y15T, Y15Q, Y15E, Y15N, Y15D, Y15R and Y15K and a further amino acid mutation selected from the group consisting of L42G, L42C, L42A, L42S, L42T, L42Q, L42E, L42N, L42D, L42R and L42K, based on SEQ ID NO:2 or the corresponding positions of homologs or variants thereof. In certain embodiments SEQ B comprises the Y15A and L42G mutations. In certain embodiments SEQ B comprises the Y15C and L42G mutations. In certain embodiments SEQ B comprises the Y15A and L42C mutations.

In certain embodiments SEQ B comprises an amino acid mutation selected from the group consisting of F12A, F12C, F12G, F12S, F12T, F12Q, F12E, F12N, F12D, F12R and F12K; a further amino acid mutation selected from the group consisting of Y15A, Y15C, Y15G, Y15S, Y15T, Y15Q, Y15E, Y15N, Y15D, Y15R and Y15K and a further amino acid mutation selected from the group consisting of L42G, L42C, L42A, L42S, L42T, L42Q, L42E, L42N, L42D, L42R and L42K, based on SEQ ID NO:2 or the corresponding positions of homologs or variants thereof. In certain embodiments SEQ B comprises the F12A, Y15A and L42G mutations. In certain embodiments SEQ B comprises the F12C, Y15A and L42G mutations. In certain embodiments SEQ B comprises the F12A, Y15C and the L42G mutations. In certain embodiments SEQ B comprises the F12A, Y15C and L42C mutations.

In certain embodiments SEQ C of formula (I) has at least 93% sequence identity to SEQ ID NO:4. In certain embodiments SEQ C of formula (I) has at least 94% sequence identity to SEQ ID NO:4. In certain embodiments SEQ C of formula (I) has at least 96% sequence identity to SEQ ID NO:4. In certain embodiments SEQ C of formula (I) has at least 98% sequence identity to SEQ ID NO:4.

In certain embodiments SEQ C comprises five amino acid changes compared to SEQ ID NO:4. In certain embodiments SEQ C comprises four amino acid changes compared to SEQ ID NO:4. In certain embodiments SEQ C comprises three amino acid changes compared to SEQ ID NO:4. In certain embodiments SEQ C comprises two amino acid changes compared to SEQ ID NO:4. In certain embodiments SEQ C comprises one amino acid change compared to SEQ ID NO:4. Such amino acid change may be an amino acid deletion, amino acid addition or the exchange of one amino acid for another amino acid, i.e. a mutation. Such mutation may also be the exchange of a proteinogenic amino acid for a non-proteinogenic amino acid and for the D-stereoisomers of proteinogenic amino acids. In certain embodiments SEQ C comprises no amino acid change compared to SEQ ID NO:4, meaning that it has the sequence of SEQ ID NO:4.

In certain embodiments SEQ C comprises an amino acid mutation selected from the group consisting of C49A, C49G, C49S, C49T, C49Q, C49E, C49N, C49D, C49H, C49W, C49Y, C49R, C49I, C49L, C49K, C49M, C49F, C49P, and C49V based on SEQ ID NO:4 or the corresponding positions of homologs or variants thereof. In certain embodiments SEQ C comprises the C49A mutation. In certain embodiments SEQ C comprises the C49G mutation. In certain embodiments SEQ C comprises the C49S mutation. In certain embodiments SEQ C comprises the C49T mutation. In certain embodiments SEQ C comprises the C49Q mutation.

In certain embodiments SEQ C comprises the C49E mutation. In certain embodiments SEQ C comprises the C49D mutation. In certain embodiments SEQ C comprises the C49H mutation. In certain embodiments SEQ C comprises the C49W mutation. In certain embodiments the SEQ C comprises the C49Y mutation. In certain embodiments SEQ C comprises the C49R mutation. In certain embodiments SEQ C comprises the C49I mutation. In certain embodiments the SEQ C comprises the C49L mutation. In certain embodiments SEQ C comprises the C49K mutation. In certain embodiments SEQ C comprises the C49M mutation. In certain embodiments SEQ C comprises the C49F mutation. In certain embodiments SEQ C comprises the C49P mutation. In certain embodiments SEQ C comprises the C49V mutation.

In certain embodiments SEQ C is selected from the group consisting of SEQ ID NO:3, SEQ ID NO:4 and SEQ ID NO:212.

In certain embodiments SEQ C has the sequence of SEQ ID NO:3: NFHLRPRDLISNINVIVLELKGSETTFMCEYADETATIVEFLNRWITFSQSIISTLT (SEQ ID NO:3). In certain embodiments SEQ C has the sequence of SEQ ID NO:4: NFHLRPRDLISNINVIVLELKGSETTFMCEYADETATIVEFLNRWITFCQSIISTLT (SEQ ID NO:4). In certain embodiments SEQ C has the sequence of SEQ ID NO:212: NFHLRPRDLISNINVIVLELKGSETTFMCEYADETATIVEFLNRWITFAQSIISTLT (SEQ ID NO:212).

In certain embodiments the alanine at the N-terminus of SEQ A of formula (I) is absent, i.e. x is 0. In certain embodiments the alanine at the N-terminus of SEQ A of formula (I) is present, i.e. x is 1.

In certain embodiments Tag¹ of formula (I) is a purification tag which in certain embodiments is selected from the group consisting of albumin-binding protein, alkaline phosphatase, AU1 epitope, AU5 epitope, bacteriophage T7 epitope (T7-tag), bacteriophage V5 epitope (V5-tag), biotin-carboxy carrier protein, bluetongue virus tag (B-tag), calmodulin binding peptide, chloramphenicol acetyl transferase, cellulose binding domain, chitin binding domain, choline-binding domain, dihydrofolate reductase, E2 epitope, FLAG epitope, galactose-binding protein, green fluorescent protein, Glu-Glu (EE-tag), glutathione S-transferase, human influenza hemagglutinin, HaloTag®, histidine affinity tag, horseradish peroxidase, HSV epitope, ketosteroid isomerase, KT3 epitope, lacz, luciferase, maltose-binding protein, myc epitope,

NusA, PDZ domain, PDZ ligand, polyarginine (Arg-tag), polyaspartate (Asp-tag), polycysteine (Cys-tag), polyhistidine (His-tag), polyphenylalanine (Phe-tag), profinity eXact, protein C, S 1-tag, S-tag, streptavadin-binding peptide, staphylococcal protein A, staphylococcal protein G, strep-tag, streptavadin, small ubiquitin-like modifier, tandem affinity purification, T7 epitope, thioredoxin, TrpE, ubiquitin, universal, and VSV-G.

In certain embodiments Tag¹ of formula (I) is a purification tag which in certain embodiments is selected from the group consisting of albumin-binding protein, alkaline phosphatase, AU1 epitope, AU5 epitope, bacteriophage T7 epitope (T7-tag), bacteriophage V5 epitope (V5-tag), biotin-carboxy carrier protein, bluetongue virus tag (B-tag), calmodulin binding peptide, chloramphenicol acetyl transferase, cellulose binding domain, chitin binding domain, choline-binding domain, dihydrofolate reductase, E2 epitope, FLAG epitope, galactose-binding protein, green fluorescent protein, Glu-Glu (EE-tag), glutathione S-transferase, human influenza hemagglutinin, histidine affinity tag, horseradish peroxidase, HSV epitope, ketosteroid isomerase, KT3 epitope, lacz, luciferase, maltose-binding protein, myc epitope, NusA, PDZ domain, PDZ ligand, polyarginine (Arg-tag), polyaspartate (Asp-tag), polycysteine (Cys-tag), polyhistidine (His-tag), polyphenylalanine (Phe-tag), profinity eXact, protein C, S1-tag, S-tag, streptavadin-binding peptide, staphylococcal protein A, staphylococcal protein G, strep-tag, streptavadin, small ubiquitin-like modifier, tandem affinity purification, T7 epitope, thioredoxin, TrpE, ubiquitin, universal, and VSV-G.

In certain embodiments Tag¹of formula (I) is a polyhistidine (His-tag), such as a His-tag comprising 5 histidines, 6 histidines, 7 histidines, 8 histidines, 9 histidines, 10 histidines, 11 histidines, 12 histidines, 13 histidines, 14 histidines, or 15 histidines. In certain embodiments Tag¹of formula (I) is a His-tag of the sequence: AHHHHHHGSDDDDK (SEQ ID NO:234).

In certain embodiments Tag² of formula (I) is a purification tag which in certain embodiments is selected from the group consisting of albumin-binding protein, alkaline phosphatase, AU1 epitope, AU5 epitope, bacteriophage T7 epitope (T7-tag), bacteriophage V5 epitope (V5-tag), biotin-carboxy carrier protein, bluetongue virus tag (B-tag), calmodulin binding peptide, chloramphenicol acetyl transferase, cellulose binding domain, chitin binding domain, choline-binding domain, dihydrofolate reductase, E2 epitope, FLAG epitope, galactose-binding protein, green fluorescent protein, Glu-Glu (EE-tag), glutathione S-transferase, human influenza hemagglutinin, HaloTag®, histidine affinity tag, horseradish peroxidase, HSV epitope, ketosteroid isomerase, KT3 epitope, lacz, luciferase, maltose-binding protein, myc epitope, NusA, PDZ domain, PDZ ligand, polyarginine (Arg-tag), polyaspartate (Asp-tag), polycysteine (Cys-tag), polyhistidine (His-tag), polyphenylalanine (Phe-tag), profinity eXact, protein C, S 1-tag, S-tag, streptavadin-binding peptide, staphylococcal protein A, staphylococcal protein G, strep-tag, streptavadin, small ubiquitin-like modifier, tandem affinity purification, T7 epitope, thioredoxin, TrpE, ubiquitin, universal, and VSV-G.

In certain embodiments Tag² of formula (I) is a purification tag which in certain embodiments is selected from the group consisting of albumin-binding protein, alkaline phosphatase, AU1 epitope, AU5 epitope, bacteriophage T7 epitope (T7-tag), bacteriophage V5 epitope (V5-tag), biotin-carboxy carrier protein, bluetongue virus tag (B-tag), calmodulin binding peptide, chloramphenicol acetyl transferase, cellulose binding domain, chitin binding domain, choline-binding domain, dihydrofolate reductase, E2 epitope, FLAG epitope, galactose-binding protein, green fluorescent protein, Glu-Glu (EE-tag), glutathione S-transferase, human influenza hemagglutinin, histidine affinity tag, horseradish peroxidase, HSV epitope, ketosteroid isomerase, KT3 epitope, lacz, luciferase, maltose-binding protein, myc epitope, NusA, PDZ domain, PDZ ligand, polyarginine (Arg-tag), polyaspartate (Asp-tag), polycysteine (Cys-tag), polyhistidine (His-tag), polyphenylalanine (Phe-tag), profinity eXact, protein C, S1-tag, S-tag, streptavadin-binding peptide, staphylococcal protein A, staphylococcal protein G, strep-tag, streptavadin, small ubiquitin-like modifier, tandem affinity purification, T7 epitope, thioredoxin, TrpE, ubiquitin, universal, and VSV-G.

In certain embodiments Tag² of formula (I) is a polyhistidine (His-tag), such as a His-tag comprising 5 histidines, 6 histidines, 7 histidines, 8 histidines, 9 histidines, 10 histidines, 11 histidines, 12 histidines, 13 histidines, 14 histidines, or 15 histidines. In certain embodiments Tag¹ of formula (I) is a His-tag of the sequence: AHHHHHHGSDDDDK (SEQ ID NO:234).

In certain embodiments one of Tag¹ and Tag² of formula (I) is a purification tag, such as Tag¹ of formula (I) is a purification tag and Tag² of formula (I) is a different type of tag or is absent. In certain embodiments Tag² of formula (I) is a purification tag and Tag¹is a different type of tag or is absent. In certain embodiments both Tag¹ and Tag² of formula (I) are a purification tag, which may be the same or different.

In certain embodiments Tag¹of formula (I) is a stabilization tag which in certain embodiments is selected from the group consisting of an Fc, Fc fragment, IgG, IgG fragment, antibody, antibody fragment, human serum albumin, albumin binding fragment, transferrin, extended recombinant polypeptide (XTEN), proline-alanine-serine polymer (PAS), proline-alanine polymer (PA), elastin-like peptide (ELP), homo-amino acid polymer (HAP), gelatin-like protein (GLK) or CG β-subunit to antibody fragment.

In certain embodiments Tag² of formula (I) is a stabilization tag which in certain embodiments is selected from the group consisting of an Fc, Fc fragment, IgG, IgG fragment, antibody, antibody fragment, human serum albumin, albumin binding fragment, transferrin, extended recombinant polypeptide (XTEN), proline-alanine-serine polymer (PAS), proline-alanine polymer (PA), elastin-like peptide (ELP), homo-amino acid polymer (HAP), gelatin-like protein (GLK) or CG β-subunit to antibody fragment.

In certain embodiments one of Tag¹ and Tag² of formula (I) is a stabilization tag, such as Tag¹ of formula (I) is a stabilization tag and Tag² of formula (1) is a different type of tag or is absent. In certain embodiments Tag² of formula (I) is a stabilization tag and Tag¹is a different type of tag or is absent. In certain embodiments both Tag¹ and Tag² of formula (I) are a stabilization tag, which may be the same or different.

In certain embodiments Tag¹ of formula (I) is a targeting tag which in certain embodiments is selected from the group consisting of antibodies, antibody fragments, Fabs, affibodies, affilins, affimers, affitins, alphamabs, alphabodies, anticalins, avimers, DARPins, Fynomers®, Kunitz domain peptides, monobodies, nanoCLAMPs, cyclic peptides, peptides, heavy chain only antibodies, VHH antibodies or Nanobodies®, single chain variable Fragments (scFvs), and natural or modified peptide or protein receptor ligands.

In certain embodiments Tag¹ of formula (I) is a targeting tag which in certain embodiments is selected from the group consisting of antibodies, antibody fragments, Fabs, affibodies, affilins, affimers, affitins, alphamabs, alphabodies, anticalins, avimers, DARPins, Kunitz domain peptides, monobodies, nanoCLAMPs, cyclic peptides, peptides, heavy chain only antibodies, VHH antibodies, single chain variable Fragments (scFvs), and natural or modified peptide or protein receptor ligands.

In certain embodiments Tag² of formula (I) is a targeting tag which in certain embodiments is selected from the group consisting of antibodies, antibody fragments, affibodies, affilins, affimers, affitins, alphamabs, alphabodies, anticalins, avimers, DARPins, Fynomers®, Kunitz domain peptides, monobodies, nanoCLAMPs, cyclic peptides, peptides, heavy chain only antibodies, VHH antibodies or Nanobodies®, single chain variable Fragments (scFvs), and natural or modified peptide or protein receptor ligands.

In certain embodiments Tag² of formula (I) is a targeting tag which in certain embodiments is selected from the group consisting of antibodies, antibody fragments, affibodies, affilins, affimers, affitins, alphamabs, alphabodies, anticalins, avimers, DARPins, Kunitz domain peptides, monobodies, nanoCLAMPs, cyclic peptides, peptides, heavy chain only antibodies, VHH antibodies, single chain variable Fragments (scFvs), and natural or modified peptide or protein receptor ligands.

In certain embodiments one of Tag¹ and Tag² of formula (I) is a targeting tag, such as Tag¹of formula (I) is a targeting tag and Tag² of formula (I) is a different type of tag or is absent. In certain embodiments Tag² of formula (I) is a targeting tag and Tag¹is a different type of tag or is absent. In certain embodiments both Tag¹ and Tag² of formula (I) are a targeting tag, which may be the same or different.

In certain embodiments “Tag¹” of formula (I) is absent, i.e. y is 0. In certain embodiments “Tag^{l}” of formula (I) is present, i.e. y is 1. In certain embodiments “Tag²” of formula (I) is absent, i.e. z is 0. In certain embodiments “Tag²” of formula (I) is present, i.e. z is 1.

In certain embodiments x of formula (I) is 0; SEQ A of formula (I) has the sequence of SEQ ID NO: 1; SEQ B of formula (I) has the sequence of SEQ ID NO: 11; SEQ C of formula (I) has the sequence of SEQ ID NO:3; y of formula (I) is 0; and z of formula (I) is 0. Accordingly, the IL-2 protein of formula (I) has the sequence of SEQ ID NO:9:

PTSSSTKKTQLQLEHLLLDLQMILNGINNYKNPKLTFGNSTKFYMPKKAT
ELKHLQCLEEELKPLEEVLNLAQSKNFHLRPRDLISNINVIVLELKGSET
TFMCEYADETATIVEFLNRWITFSQSIISTLT (SEQ ID NO:9)

In certain embodiments x of formula (I) is 1; SEQ A of formula (I) has the sequence of SEQ ID NO: 1; SEQ B of formula (I) has the sequence of SEQ ID NO: 11; SEQ C of formula (I) has the sequence of SEQ ID NO:3; y of formula (I) is 0 and z of formula (I) is 0. Accordingly, the IL-2 protein of formula (I) has the sequence of SEQ ID NO:10:

APTSSSTKKTQLQLEHLLLDLQMILNGINNYKNPKLTFGNSTKFYMPKKA
TELKHLQCLEEELKPLEEVLNLAQSKNFHLRPRDLISNINVIVLELKGSE
TTFMCEYADETATIVEFLNRWITFSQSIISTLT (SEQ ID NO: 10)

In certain embodiments x of formula (I) is 0; SEQ A of formula (I) has the sequence of SEQ ID NO:36; SEQ B of formula (I) has the sequence of SEQ ID NO: 11; SEQ C of formula (I) has the sequence of SEQ ID NO:3; y of formula (I) is 0 and z of formula (I) is 0. Accordingly, the IL-2 protein of formula (I) has the sequence of SEQ ID NO:37:

PASSSTKKTQLQLEHLLLDLQMILNGINNYKNPKLTFGNSTKFYMPKKAT
ELKHLQCLEEELKPLEEVLNLAQSKNFHLRPRDLISNINVIVLELKGSET
TFMCEYADETATIVEFLNRWITFSQSIISTLT (SEQ ID NO:37)

In certain embodiments x of formula (I) is 1; SEQ A of formula (I) has the sequence of SEQ ID NO:36; SEQ B of formula (I) has the sequence of SEQ ID NO: 11; SEQ C of formula (I) has the sequence of SEQ ID NO:3; y of formula (I) is 0 and z of formula (I) is 0. Accordingly, the IL-2 protein of formula (I) has the sequence of SEQ ID NO:38:

APASSSTKKTQLQLEHLLLDLQMILNGINNYKNPKLTFGNSTKFYMPKKA
TELKHLQCLEEELKPLEEVLNLAQSKNFHLRPRDLISNINVIVLELKGSE
TTFMCEYADETATIVEFLNRWITFSQSIISTLT (SEQ ID NO:38)

In certain embodiments x of formula (I) is 0; SEQ A of formula (I) has the sequence of SEQ ID NO: 1; SEQ B of formula (I) has the sequence of SEQ ID NO: 11; SEQ C of formula (I) has the sequence of SEQ ID NO:4; y of formula (I) is 0 and z of formula (I) is 0. Accordingly, the IL-2 protein of formula (I) has the sequence of SEQ ID NO:39:

PTSSSTKKTQLQLEHLLLDLQMILNGINNYKNPKL TFGNSTKFYMPKKA
TELKHLQCLEEELKPLEEVLNLAQSKNFHLRPRDLISNINVIVLELKGS
ETTFMCEYADETATIVEFLNRWITFCQSIISTLT (SEQ ID NO:39)

In certain embodiments x of formula (I) is 1; SEQ A of formula (I) has the sequence of SEQ ID NO: 1; SEQ B of formula (I) has the sequence of SEQ ID NO: 11; SEQ C of formula (I) has the sequence of SEQ ID NO:4; y of formula (I) is 0 and z of formula (I) is 0. Accordingly, the IL-2 protein of formula (I) has the sequence of SEQ ID NO:40:

APTSSSTKKTQLQLEHLLLDLQMILNGINNYKNPKLTFGNSTKFYMPKKA
TELKHLQCLEEELKPLEEVLNLAQSKNFHLRPRDLISNINVIVLELKGSE
TTFMCEYADETATIVEFLNRWITFCQSIISTLT (SEQ ID NO:40)

In certain embodiments x of formula (I) is 0; SEQ A of formula (I) has the sequence of SEQ ID NO:36; SEQ B of formula (I) has the sequence of SEQ ID NO: 11; SEQ C of formula (I) has the sequence of SEQ ID NO:4; y of formula (I) is 0 and z of formula (I) is 0. Accordingly, the IL-2 protein of formula (I) has the sequence of SEQ ID NO:41:

PASSSTKKTQLQLEHLLLDLQMILNGINNYKNPKLTFGNSTKFYMPKKAT
ELKHLQCLEEELKPLEEVLNLAQSKNFHLRPRDLISNINVIVLELKGSET
TFMCEYADETATIVEFLNRWITFCQSIISTLT (SEQ ID NO:41)

In certain embodiments x of formula (I) is 1; SEQ A of formula (I) has the sequence of SEQ ID NO:36; SEQ B of formula (I) has the sequence of SEQ ID NO: 11; SEQ C of formula (I) has the sequence of SEQ ID NO:4; y of formula (I) is 0 and z of formula (I) is 0. Accordingly, the IL-2 protein of formula (I) has the sequence of SEQ ID NO:42:

APASSSTKKTQLQLEHLLLDLQMILNGINNYKNPKLTFGNSTKFYMPKKA
TELKHLQCLEEELKPLEEVLNLAQSKNFHLRPRDLISNINVIVLELKGSE
TTFMCEYADETATIVEFLNRWITFCQSIISTLT (SEQ ID NO:42)

In certain embodiments x of formula (I) is 0; SEQ A of formula (I) has the sequence of SEQ ID NO: 1; SEQ B of formula (I) has the sequence of SEQ ID NO:214; SEQ C of formula (I) has the sequence of SEQ ID NO:3; y of formula (I) is 0; and z of formula (I) is 0. Accordingly, the IL-2 protein of formula (I) has the sequence of SEQ ID NO:216:

PTSSSTKKTQLQLEHLLLDLQMILNGINNYKNPDLTFGNSTKFYMPKKAT
ELKHLQCLEEELKPLEEVLNLAQSKNFHLRPRDLISNINVIVLELKGSET
TFMCEYADETATIVEFLNRWITFSQSIISTLT (SEQ ID NO:216)

In certain embodiments x of formula (I) is 1; SEQ A of formula (I) has the sequence of SEQ ID NO: 1; SEQ B of formula (I) has the sequence of SEQ ID NO:214; SEQ C of formula (I) has the sequence of SEQ ID NO:3; y of formula (I) is 0 and z of formula (I) is 0. Accordingly, the IL-2 protein of formula (I) has the sequence of SEQ ID NO:217:

APTSSSTKKTQLQLEHLLLDLQMILNGINNYKNPDLTFGNSTKFYMPKKA
TELKHLQCLEEELKPLEEVLNLAQSKNFHLRPRDLISNINVIVLELKGSE
TTFMCEYADETATIVEFLNRWITFSQSIISTLT (SEQ ID NO:217)

In certain embodiments x of formula (I) is 0; SEQ A of formula (I) has the sequence of SEQ ID NO:36; SEQ B of formula (I) has the sequence of SEQ ID NO:214; SEQ C of formula (I) has the sequence of SEQ ID NO:3; y of formula (I) is 0 and z of formula (I) is 0. Accordingly, the IL-2 protein of formula (I) has the sequence of SEQ ID NO:218:

PASSSTKKTQLQLEHLLLDLQMILNGINNYKNPDLTFGNSTKFYMPKKAT
ELKHLQCLEEELKPLEEVLNLAQSKNFHLRPRDLISNINVIVLELKGSET
TFMCEYADETATIVEFLNRWITFSQSIISTLT (SEQ ID NO:218)

In certain embodiments x of formula (I) is 1; SEQ A of formula (I) has the sequence of SEQ ID NO:36; SEQ B of formula (I) has the sequence of SEQ ID NO:214; SEQ C of formula (I) has the sequence of SEQ ID NO:3; y of formula (I) is 0 and z of formula (I) is 0. Accordingly, the IL-2 protein of formula (I) has the sequence of SEQ ID NO:219:

APASSSTKKTQLQLEHLLLDLQMILNGINNYKNPDLTFGNSTKFYMPKKA
TELKHLQCLEEELKPLEEVLNLAQSKNFHLRPRDLISNINVIVLELKGSE
TTFMCEYADETATIVEFLNRWITFSQSIISTLT (SEQ ID NO:219)

In certain embodiments x of formula (I) is 0; SEQ A of formula (I) has the sequence of SEQ ID NO: 1; SEQ B of formula (I) has the sequence of SEQ ID NO:214; SEQ C of formula (I) has the sequence of SEQ ID NO:4; y of formula (I) is 0 and z of formula (I) is 0. Accordingly, the IL-2 protein of formula (I) has the sequence of SEQ ID NO:220:

PTSSSTKKTQLQLEHLLLDLQMILNGINNYKNPDLTFGNSTKFYMPKKAT
ELKHLQCLEEELKPLEEVLNLAQSKNFHLRPRDLISNINVIVLELKGSET
TFMCEYADETATIVEFLNRWITFCQSIISTLT (SEQ ID NO:220)

In certain embodiments x of formula (I) is 1; SEQ A of formula (I) has the sequence of SEQ ID NO: 1; SEQ B of formula (I) has the sequence of SEQ ID NO:214; SEQ C of formula (I) has the sequence of SEQ ID NO:4; y of formula (I) is 0 and z of formula (I) is 0. Accordingly, the IL-2 protein of formula (I) has the sequence of SEQ ID NO:221:

APTSSSTKKTQLQLEHLLLDLQMILNGINNYKNPDLTFGNSTKFYMPKKA
TELKHLQCLEEELKPLEEVLNLAQSKNFHLRPRDLISNINVIVLELKGSE
TTFMCEYADETATIVEFLNRWITFCQSIISTLT (SEQ ID NO:221)

In certain embodiments x of formula (I) is 0; SEQ A of formula (I) has the sequence of SEQ ID NO:36; SEQ B of formula (I) has the sequence of SEQ ID NO:214; SEQ C of formula (I) has the sequence of SEQ ID NO:4; y of formula (I) is 0 and z of formula (I) is 0. Accordingly, the IL-2 protein of formula (I) has the sequence of SEQ ID NO:222:

PASSSTKKTQLQLEHLLLDLQMILNGINNYKNPDLTFGNSTKFYMPKKAT
ELKHLQCLEEELKPLEEVLNLAQSKNFHLRPRDLISNINVIVLELKGSET
TFMCEYADETATIVEFLNRWITFCQSIISTLT (SEQ ID NO:222)

In certain embodiments x of formula (I) is 1; SEQ A of formula (I) has the sequence of SEQ ID NO:36; SEQ B of formula (I) has the sequence of SEQ ID NO:214; SEQ C of formula (I) has the sequence of SEQ ID NO:4; y of formula (I) is 0 and z of formula (I) is 0. Accordingly, the IL-2 protein of formula (I) has the sequence of SEQ ID NO:223:

APASSSTKKTQLQLEHLLLDLQMILNGINNYKNPDLTFGNSTKFYMPKKA
TELKHLQCLEEELKPLEEVLNLAQSKNFHLRPRDLISNINVIVLELKGSE
TTFMCEYADETATIVEFLNRWITFCQSIISTLT (SEQ ID NO:223)

In certain embodiments x of formula (I) is 0; SEQ A of formula (I) has the sequence of SEQ ID NO:1; SEQ B of formula (I) has the sequence of SEQ ID NO:215; SEQ C of formula (I) has the sequence of SEQ ID NO:3; y of formula (I) is 0; and z of formula (I) is 0. Accordingly, the IL-2 protein of formula (I) has the sequence of SEQ ID NO:224:

PTSSSTKKTQLQLEHLLLDLQMILNGINNYKNPELTFGNSTKFYMPKKAT
ELKHLQCLEEELKPLEEVLNLAQSKNFHLRPRDLISNINVIVLELKGSET
TFMCEYADETATIVEFLNRWITFSQSIISTLT (SEQ ID NO:224)

In certain embodiments x of formula (I) is 1; SEQ A of formula (I) has the sequence of SEQ ID NO:1; SEQ B of formula (I) has the sequence of SEQ ID NO:215; SEQ C of formula (I) has the sequence of SEQ ID NO:3; y of formula (I) is 0 and z of formula (I) is 0. Accordingly, the IL-2 protein of formula (I) has the sequence of SEQ ID NO:225:

APTSSSTKKTQLQLEHLLLDLQMILNGINNYKNPELTFGNSTKFYMPKKA
TELKHLQCLEEELKPLEEVLNLAQSKNFHLRPRDLISNINVIVLELKGSE
TTFMCEYADETATIVEFLNRWITFSQSIISTLT (SEQ ID NO:225)

In certain embodiments x of formula (I) is 0; SEQ A of formula (I) has the sequence of SEQ ID NO:36; SEQ B of formula (I) has the sequence of SEQ ID NO:215; SEQ C of formula (I) has the sequence of SEQ ID NO:3; y of formula (I) is 0 and z of formula (I) is 0. Accordingly, the IL-2 protein of formula (I) has the sequence of SEQ ID NO:226:

PASSSTKKTQLQLEHLLLDLQMILNGINNYKNPELTFGNSTKFYMPKKAT
ELKHLQCLEEELKPLEEVLNLAQSKNFHLRPRDLISNINVIVLELKGSET
TFMCEYADETATIVEFLNRWITFSQSIISTLT (SEQ ID NO:226)

In certain embodiments x of formula (I) is 1; SEQ A of formula (I) has the sequence of SEQ ID NO:36; SEQ B of formula (I) has the sequence of SEQ ID NO:215; SEQ C of formula (I) has the sequence of SEQ ID NO:3; y of formula (I) is 0 and z of formula (I) is 0. Accordingly, the IL-2 protein of formula (I) has the sequence of SEQ ID NO:227:

APASSSTKKTQLQLEHLLLDLQMILNGINNYKNPELTFGNSTKFYMPKKA
TELKHLQCLEEELKPLEEVLNLAQSKNFHLRPRDLISNINVIVLELKGSE
TTFMCEYADETATIVEFLNRWITFSQSIISTLT (SEQ ID NO:227)

In certain embodiments x of formula (I) is 0; SEQ A of formula (I) has the sequence of SEQ ID NO:1; SEQ B of formula (I) has the sequence of SEQ ID NO:215; SEQ C of formula (I) has the sequence of SEQ ID NO:4; y of formula (I) is 0 and z of formula (I) is 0. Accordingly, the IL-2 protein of formula (I) has the sequence of SEQ ID NO:228:

PTSSSTKKTQLQLEHLLLDLQMILNGINNYKNPELTFGNSTKFYMPKKAT
ELKHLQCLEEELKPLEEVLNLAQSKNFHLRPRDLISNINVIVLELKGSET
TFMCEYADETATIVEFLNRWITFCQSIISTLT (SEQ ID NO:228)

In certain embodiments x of formula (I) is 1; SEQ A of formula (I) has the sequence of SEQ ID NO:1; SEQ B of formula (I) has the sequence of SEQ ID NO:215; SEQ C of formula (I) has the sequence of SEQ ID NO:4; y of formula (I) is 0 and z of formula (I) is 0. Accordingly, the IL-2 protein of formula (I) has the sequence of SEQ ID NO:27:

APTSSSTKKTQLQLEHLLLDLQMILNGINNYKNPELTFGNSTKFYMPKKA
TELKHLQCLEEELKPLEEVLNLAQSKNFHLRPRDLISNINVIVLELKGSE
TTFMCEYADETATIVEFLNRWITFCQSIISTLT (SEQ ID NO:27)

In certain embodiments x of formula (I) is 0; SEQ A of formula (I) has the sequence of SEQ ID NO:36; SEQ B of formula (I) has the sequence of SEQ ID NO:215; SEQ C of formula (I) has the sequence of SEQ ID NO:4; y of formula (I) is 0 and z of formula (I) is 0. Accordingly, the IL-2 protein of formula (I) has the sequence of SEQ ID NO:28:

PASSSTKKTQLQLEHLLLDLQMILNGINNYKNPELTFGNSTKFYMPKKAT
ELKHLQCLEEELKPLEEVLNLAQSKNFHLRPRDLISNINVIVLELKGSET
TFMCEYADETATIVEFLNRWITFCQSIISTLT (SEQ ID NO:28)

In certain embodiments x of formula (I) is 1; SEQ A of formula (I) has the sequence of SEQ ID NO:36; SEQ B of formula (I) has the sequence of SEQ ID NO:215; SEQ C of formula (I) has the sequence of SEQ ID NO:4; y of formula (I) is 0 and z of formula (I) is 0. Accordingly, the IL-2 protein of formula (I) has the sequence of SEQ ID NO:29:

APASSSTKKTQLQLEHLLLDLQMILNGINNYKNPELTFGNSTKFYMPKKA
TELKHLQCLEEELKPLEEVLNLAQSKNFHLRPRDLISNINVIVLELKGSE
TTFMCEYADETATIVEFLNRWITFCQSIISTLT (SEQ ID NO:29)

In certain embodiments x of formula (I) is 0; SEQ A of formula (I) has the sequence of SEQ ID NO: 1; SEQ B of formula (I) has the sequence of SEQ ID NO: 14; SEQ C of formula (I) has the sequence of SEQ ID NO:3; y of formula (I) is 0 and z of formula (I) is 0. Accordingly, the IL-2 protein of formula (1) has the sequence of SEQ ID NO:12:

PTSSSTKKTQLQLEHLLLDLQMILNGINNYKNPKLTFANSTKFYMPKKAT
ELKHLQCLEEELKPLEEVLNLAQSKNFHLRPRDLISNINVIVLELKGSET
TFMCEYADETATIVEFLNRWITFSQSIISTLT (SEQ ID NO:12)

In certain embodiments x of formula (I) is 1; SEQ A of formula (I) has the sequence of SEQ ID NO: 1; SEQ B of formula (I) has the sequence of SEQ ID NO: 14; SEQ C of formula (I) has the sequence of SEQ ID NO:3; y of formula (I) is 0 and z of formula (I) is 0. Accordingly, the IL-2 protein of formula (I) has the sequence of SEQ ID NO: 13:

APTSSSTKKTQLQLEHLLLDLQMILNGINNYKNPKLTFANSTKFYMPKKA
TELKHLQCLEEELKPLEEVLNLAQSKNFHLRPRDLISNINVIVLELKGSE
TTFMCEYADETATIVEFLNRWITFSQSIISTLT (SEQ ID NO:13)

In certain embodiments x of formula (I) is 0; SEQ A of formula (I) has the sequence of SEQ ID NO:36; SEQ B of formula (I) has the sequence of SEQ ID NO:14; SEQ C of formula (I) has the sequence of SEQ ID NO:3; y of formula (I) is 0 and z of formula (I) is 0. Accordingly, the IL-2 protein of formula (I) has the sequence of SEQ ID NO:43:

PASSSTKKTQLQLEHLLLDLQMILNGINNYKNPKLTFANSTKFYMPKKAT
ELKHLQCLEEELKPLEEVLNLAQSKNFHLRPRDLISNINVIVLELKGSET
TFMCEYADETATIVEFLNRWITFSQSIISTLT (SEQ ID NO:43)

In certain embodiments x of formula (I) is 1; SEQ A of formula (I) has the sequence of SEQ ID NO:36; SEQ B of formula (I) has the sequence of SEQ ID NO:14; SEQ C of formula (I) has the sequence of SEQ ID NO:3; y of formula (I) is 0 and z of formula (I) is 0. Accordingly, the IL-2 protein of formula (I) has the sequence of SEQ ID NO:44:

APASSSTKKTQLQLEHLLLDLQMILNGINNYKNPKLTFANSTKFYMPKKA
TELKHLQCLEEELKPLEEVLNLAQSKNFHLRPRDLISNINVIVLELKGSE
TTFMCEYADETATIVEFLNRWITFSQSIISTLT (SEQ ID NO:44)

In certain embodiments x of formula (I) is 0; SEQ A of formula (I) has the sequence of SEQ ID NO:1; SEQ B of formula (I) has the sequence of SEQ ID NO:14; SEQ C of formula (I) has the sequence of SEQ ID NO:4; y of formula (I) is 0 and z of formula (I) is 0. Accordingly, the IL-2 protein of formula (I) has the sequence of SEQ ID NO:45:

PTSSSTKKTQLQLEHLLLDLQMILNGINNYKNPKLTFANSTKFYMPKKAT
ELKHLQCLEEELKPLEEVLNLAQSKNFHLRPRDLISNINVIVLELKGSET
TFMCEYADETATIVEFLNRWITFCQSIISTLT (SEQ ID NO:45)

In certain embodiments x of formula (I) is 1; SEQ A of formula (I) has the sequence of SEQ ID NO:1; SEQ B of formula (I) has the sequence of SEQ ID NO: 14; SEQ C of formula (I) has the sequence of SEQ ID NO:4; y of formula (I) is 0 and z of formula (I) is 0. Accordingly, the IL-2 protein of formula (I) has the sequence of SEQ ID NO:46:

APTSSSTKKTQLQLEHLLLDLQMILNGINNYKNPKLTFANSTKFYMPKKA
TELKHLQCLEEELKPLEEVLNLAQSKNFHLRPRDLISNINVIVLELKGSE
TTFMCEYADETATIVEFLNRWITFCQSIISTLT (SEQ ID NO:46)

In certain embodiments x of formula (I) is 0; SEQ A of formula (I) has the sequence of SEQ ID NO:36; SEQ B of formula (I) has the sequence of SEQ ID NO:14; SEQ C of formula (I) has the sequence of SEQ ID NO:4; y of formula (I) is 0 and z of formula (I) is 0. Accordingly, the IL-2 protein of formula (I) has the sequence of SEQ ID NO:47:

PASSSTKKTQLQLEHLLLDLQMILNGINNYKNPKLTFANSTKFYMPKKAT
ELKHLQCLEEELKPLEEVLNLAQSKNFHLRPRDLISNINVIVLELKGSET
TFMCEYADETATIVEFLNRWITFCQSIISTLT (SEQ ID NO:47)

In certain embodiments x of formula (I) is 1; SEQ A of formula (I) has the sequence of SEQ ID NO:36; SEQ B of formula (I) has the sequence of SEQ ID NO:14; SEQ C of formula (I) has the sequence of SEQ ID NO:4; y of formula (I) is 0 and z of formula (I) is 0. Accordingly, the IL-2 protein of formula (I) has the sequence of SEQ ID NO:48:

APASSSTKKTQLQLEHLLLDLQMILNGINNYKNPKLTFANSTKFYMPKKA
TELKHLQCLEEELKPLEEVLNLAQSKNFHLRPRDLISNINVIVLELKGSE
TTFMCEYADETATIVEFLNRWITFCQSIISTLT (SEQ ID NO:48)

In certain embodiments x of formula (I) is 0; SEQ A of formula (I) has the sequence of SEQ ID NO:1; SEQ B of formula (I) has the sequence of SEQ ID NO:17; SEQ C of formula (I) has the sequence of SEQ ID NO:3; y of formula (I) is 0 and z of formula (I) is 0. Accordingly, the IL-2 protein of formula (I) has the sequence of SEQ ID NO:15:

PTSSSTKKTQLQLEHLLLDLQMILNGINNYKNPKLTRMLFGNSTMPKKAT
ELKHLQCLEEELKPLEEVLNLAQSKNFHLRPRDLISNINVIVLELKGSET
TFMCEYADETATIVEFLNRWITFSQSIISTLT (SEQ ID NO:15)

In certain embodiments x of formula (I) is 1; SEQ A of formula (I) has the sequence of SEQ ID NO:1; SEQ B of formula (I) has the sequence of SEQ ID NO:17; SEQ C of formula (I) has the sequence of SEQ ID NO:3; y of formula (I) is 0 and z of formula (I) is 0. Accordingly, the IL-2 protein of formula (I) has the sequence of SEQ ID NO:16:

APTSSSTKKTQLQLEHLLLDLQMILNGINNYKNPKLTRMLFGNSTMPKKA
TELKHLQCLEEELKPLEEVLNLAQSKNFHLRPRDLISNINVIVLELKGSE
TTFMCEYADETATIVEFLNRWITFSQSIISTLT (SEQ ID NO:16)

In certain embodiments x of formula (I) is 0; SEQ A of formula (I) has the sequence of SEQ ID NO:36; SEQ B of formula (I) has the sequence of SEQ ID NO:17; SEQ C of formula (I) has the sequence of SEQ ID NO:3; y of formula (I) is 0 and z of formula (I) is 0. Accordingly, the IL-2 protein of formula (I) has the sequence of SEQ ID NO:49:

PASSSTKKTQLQLEHLLLDLQMILNGINNYKNPKLTRMLFGNSTMPKKAT
ELKHLQCLEEELKPLEEVLNLAQSKNFHLRPRDLISNINVIVLELKGSET
TFMCEYADETATIVEFLNRWITFSQSIISTLT (SEQ ID NO:49)

In certain embodiments x of formula (I) is 1; SEQ A of formula (I) has the sequence of SEQ ID NO:36; SEQ B of formula (I) has the sequence of SEQ ID NO:17; SEQ C of formula (I) has the sequence of SEQ ID NO:3; y of formula (I) is 0 and z of formula (I) is 0. Accordingly, the IL-2 protein of formula (I) has the sequence of SEQ ID NO:50:

APASSSTKKTQLQLEHLLLDLQMILNGINNYKNPKLTRMLFGNSTMPKKA
TELKHLQCLEEELKPLEEVLNLAQSKNFHLRPRDLISNINVIVLELKGSE
TTFMCEYADETATIVEFLNRWITFSQSIISTLT (SEQ ID NO:50)

In certain embodiments x of formula (I) is 0; SEQ A of formula (I) has the sequence of SEQ ID NO:1; SEQ B of formula (I) has the sequence of SEQ ID NO:17; SEQ C of formula (I) has the sequence of SEQ ID NO:4; y of formula (I) is 0 and z of formula (I) is 0. Accordingly, the IL-2 protein of formula (I) has the sequence of SEQ ID NO:51:

PTSSSTKKTQLQLEHLLLDLQMILNGINNYKNPKLTRMLFGNSTMPKKAT
ELKHLQCLEEELKPLEEVLNLAQSKNFHLRPRDLISNINVIVLELKGSET
TFMCEYADETATIVEFLNRWITFCQSIISTLT (SEQ ID NO:51)

In certain embodiments x of formula (I) is 1; SEQ A of formula (I) has the sequence of SEQ ID NO:1; SEQ B of formula (I) has the sequence of SEQ ID NO:17; SEQ C of formula (I) has the sequence of SEQ ID NO:4; y of formula (I) is 0 and z of formula (I) is 0. Accordingly, the IL-2 protein of formula (I) has the sequence of SEQ ID NO:52:

APTSSSTKKTQLQLEHLLLDLQMILNGINNYKNPKLTRMLFGNSTMPKKA
TELKHLQCLEEELKPLEEVLNLAQSKNFHLRPRDLISNINVIVLELKGSE
TTFMCEYADETATIVEFLNRWITFCQSIISTLT (SEQ ID NO:52)

In certain embodiments x of formula (I) is 0; SEQ A of formula (I) has the sequence of SEQ ID NO:36; SEQ B of formula (I) has the sequence of SEQ ID NO:17; SEQ C of formula (I) has the sequence of SEQ ID NO:4; y of formula (I) is 0 and z of formula (I) is 0. Accordingly, the IL-2 protein of formula (I) has the sequence of SEQ ID NO:53:

PASSSTKKTQLQLEHLLLDLQMILNGINNYKNPKLTRMLFGNSTMPKKAT
ELKHLQCLEEELKPLEEVLNLAQSKNFHLRPRDLISNINVIVLELKGSET
TFMCEYADETATIVEFLNRWITFCQSIISTLT (SEQ ID NO:53)

In certain embodiments x of formula (I) is 1; SEQ A of formula (I) has the sequence of SEQ ID NO:36; SEQ B of formula (I) has the sequence of SEQ ID NO:17; SEQ C of formula (I) has the sequence of SEQ ID NO:4; y of formula (I) is 0 and z of formula (I) is 0. Accordingly, the IL-2 protein of formula (I) has the sequence of SEQ ID NO:54:

APASSSTKKTQLQLEHLLLDLQMILNGINNYKNPKLTRMLFGNSTMPKKA
TELKHLQCLEEELKPLEEVLNLAQSKNFHLRPRDLISNINVIVLELKGSE
TTFMCEYADETATIVEFLNRWITFCQSIISTLT (SEQ ID NO:54)

In certain embodiments x of formula (I) is 0; SEQ A of formula (I) has the sequence of SEQ ID NO:1; SEQ B of formula (I) has the sequence of SEQ ID NO:20; SEQ C of formula (I) has the sequence of SEQ ID NO:3; y of formula (I) is 0 and z of formula (I) is 0. Accordingly, the IL-2 protein of formula (I) has the sequence of SEQ ID NO:18:

PTSSSTKKTQLQLEHLLLDLQMILNGINNYKNPKLTRMLFANSTMPKKAT
ELKHLQCLEEELKPLEEVLNLAQSKNFHLRPRDLISNINVIVLELKGSET
TFMCEYADETATIVEFLNRWITFSQSIISTLT (SEQ ID NO:18)

In certain embodiments x of formula (I) is 1; SEQ A of formula (I) has the sequence of SEQ ID NO:1; SEQ B of formula (I) has the sequence of SEQ ID NO:20; SEQ C of formula (I) has the sequence of SEQ ID NO:3; y of formula (I) is 0 and z of formula (I) is 0. Accordingly, the IL-2 protein of formula (I) has the sequence of SEQ ID NO:19:

APTSSSTKKTQLQLEHLLLDLQMILNGINNYKNPKLTRMLFANSTMPKKA
TELKHLQCLEEELKPLEEVLNLAQSKNFHLRPRDLISNINVIVLELKGSE
TTFMCEYADETATIVEFLNRWITFSQSIISTLT (SEQ ID NO:19)

In certain embodiments x of formula (I) is 0; SEQ A of formula (I) has the sequence of SEQ ID NO:36; SEQ B of formula (I) has the sequence of SEQ ID NO:20; SEQ C of formula (I) has the sequence of SEQ ID NO:3; y of formula (I) is 0 and z of formula (I) is 0. Accordingly, the IL-2 protein of formula (I) has the sequence of SEQ ID NO:55:

PASSSTKKTQLQLEHLLLDLQMILNGINNYKNPKLTRMLFANSTMPKKAT
ELKHLQCLEEELKPLEEVLNLAQSKNFHLRPRDLISNINVIVLELKGSET
TFMCEYADETATIVEFLNRWITFSQSIISTLT (SEQ ID NO:55)

In certain embodiments x of formula (I) is 1; SEQ A of formula (I) has the sequence of SEQ ID NO:36; SEQ B of formula (I) has the sequence of SEQ ID NO:20; SEQ C of formula (I) has the sequence of SEQ ID NO:3; y of formula (I) is 0 and z of formula (I) is 0. Accordingly, the IL-2 protein of formula (I) has the sequence of SEQ ID NO:56:

APASSSTKKTQLQLEHLLLDLQMILNGINNYKNPKLTRMLFANSTMPKKA
TELKHLQCLEEELKPLEEVLNLAQSKNFHLRPRDLISNINVIVLELKGSE
TTFMCEYADETATIVEFLNRWITFSQSIISTLT (SEQ ID NO:56)

In certain embodiments x of formula (I) is 0; SEQ A of formula (I) has the sequence of SEQ ID NO:1; SEQ B of formula (I) has the sequence of SEQ ID NO:20; SEQ C of formula (I) has the sequence of SEQ ID NO:4; y of formula (I) is 0 and z of formula (I) is 0. Accordingly, the IL-2 protein of formula (I) has the sequence of SEQ ID NO:57:

PTSSSTKKTQLQLEHLLLDLQMILNGINNYKNPKLTRMLFANSTMPKKAT
ELKHLQCLEEELKPLEEVLNLAQSKNFHLRPRDLISNINVIVLELKGSET
TFMCEYADETATIVEFLNRWITFCQSIISTLT (SEQ ID NO:57)

In certain embodiments x of formula (I) is 1; SEQ A of formula (I) has the sequence of SEQ ID NO:1; SEQ B of formula (I) has the sequence of SEQ ID NO:20; SEQ C of formula (I) has the sequence of SEQ ID NO:4; y of formula (I) is 0 and z of formula (I) is 0. Accordingly, the IL-2 protein of formula (I) has the sequence of SEQ ID NO:58:

APTSSSTKKTQLQLEHLLLDLQMILNGINNYKNPKLTRMLFANSTMPKKA
TELKHLQCLEEELKPLEEVLNLAQSKNFHLRPRDLISNINVIVLELKGSE
TTFMCEYADETATIVEFLNRWITFCQSIISTLT (SEQ ID NO:58)

In certain embodiments x of formula (I) is 0; SEQ A of formula (I) has the sequence of SEQ ID NO:36; SEQ B of formula (I) has the sequence of SEQ ID NO:20; SEQ C of formula (I) has the sequence of SEQ ID NO:4; y of formula (I) is 0 and z of formula (I) is 0. Accordingly, the IL-2 protein of formula (I) has the sequence of SEQ ID NO:59:

PASSSTKKTQLQLEHLLLDLQMILNGINNYKNPKLTRMLFANSTMPKKAT
ELKHLQCLEEELKPLEEVLNLAQSKNFHLRPRDLISNINVIVLELKGSET
TFMCEYADETATIVEFLNRWITFCQSIISTLT (SEQ ID NO:59)

In certain embodiments x of formula (I) is 1; SEQ A of formula (I) has the sequence of SEQ ID NO:36; SEQ B of formula (I) has the sequence of SEQ ID NO:20; SEQ C of formula (I) has the sequence of SEQ ID NO:4; y of formula (I) is 0 and z of formula (I) is 0. Accordingly, the IL-2 protein of formula (I) has the sequence of SEQ ID NO:60:

APASSSTKKTQLQLEHLLLDLQMILNGINNYKNPKLTRMLFANSTMPKKA
TELKHLQCLEEELKPLEEVLNLAQSKNFHLRPRDLISNINVIVLELKGSE
TTFMCEYADETATIVEFLNRWITFCQSIISTLT (SEQ ID NO:60)

In certain embodiments x of formula (I) is 0; SEQ A of formula (I) has the sequence of SEQ ID NO:1; SEQ B of formula (I) has the sequence of SEQ ID NO:23; SEQ C of formula (I) has the sequence of SEQ ID NO:3; y of formula (I) is 0 and z of formula (I) is 0. Accordingly, the IL-2 protein of formula (I) has the sequence of SEQ ID NO:21:

PTSSSTKKTQLQLEHLLLDLQMILNGINNYKNPKLTRMLTFKFYMPKKAT
ELKHLQCLEFGNSTLEEVLNLAQSKNFHLRPRDLISNINVIVLELKGSET
TFMCEYADETATIVEFLNRWITFSQSIISTLT (SEQ ID NO:21)

In certain embodiments x of formula (I) is 1; SEQ A of formula (I) has the sequence of SEQ ID NO:1; SEQ B of formula (I) has the sequence of SEQ ID NO:23; SEQ C of formula (I) has the sequence of SEQ ID NO:3; y of formula (I) is 0 and z of formula (I) is 0. Accordingly, the IL-2 protein of formula (I) has the sequence of SEQ ID NO:22:

APTSSSTKKTQLQLEHLLLDLQMILNGINNYKNPKLTRMLTFKFYMPKKA
TELKHLQCLEFGNSTLEEVLNLAQSKNFHLRPRDLISNINVIVLELKGSE
TTFMCEYADETATIVEFLNRWITFSQSIISTLT (SEQ ID NO:22)

In certain embodiments x of formula (I) is 0; SEQ A of formula (I) has the sequence of SEQ ID NO:36; SEQ B of formula (I) has the sequence of SEQ ID NO:23; SEQ C of formula (I) has the sequence of SEQ ID NO:3; y of formula (I) is 0 and z of formula (I) is 0. Accordingly, the IL-2 protein of formula (I) has the sequence of SEQ ID NO:61:

PASSSTKKTQLQLEHLLLDLQMILNGINNYKNPKLTRMLTFKFYMPKKAT
ELKHLQCLEFGNSTLEEVLNLAQSKNFHLRPRDLISNINVIVLELKGSET
TFMCEYADETATIVEFLNRWITFSQSIISTLT (SEQ ID NO:61)

In certain embodiments x of formula (I) is 1; SEQ A of formula (I) has the sequence of SEQ ID NO:36; SEQ B of formula (I) has the sequence of SEQ ID NO:23; SEQ C of formula (I) has the sequence of SEQ ID NO:3; y of formula (I) is 0 and z of formula (I) is 0. Accordingly, the IL-2 protein of formula (I) has the sequence of SEQ ID NO:62:

APASSSTKKTQLQLEHLLLDLQMILNGINNYKNPKLTRMLTFKFYMPKKA
TELKHLQCLEFGNSTLEEVLNLAQSKNFHLRPRDLISNINVIVLELKGSE
TTFMCEYADETATIVEFLNRWITFSQSIISTLT (SEQ ID NO:62)

In certain embodiments x of formula (I) is 0; SEQ A of formula (I) has the sequence of SEQ ID NO:1; SEQ B of formula (I) has the sequence of SEQ ID NO:23; SEQ C of formula (I) has the sequence of SEQ ID NO:4; y of formula (I) is 0 and z of formula (I) is 0. Accordingly, the IL-2 protein of formula (I) has the sequence of SEQ ID NO:63:

PTSSSTKKTQLQLEHLLLDLQMILNGINNYKNPKLTRMLTFKFYMPKKAT
ELKHLQCLEFGNSTLEEVLNLAQSKNFHLRPRDLISNINVIVLELKGSET
TFMCEYADETATIVEFLNRWITFCQSIISTLT (SEQ ID NO:63)

In certain embodiments x of formula (I) is 1; SEQ A of formula (I) has the sequence of SEQ ID NO:1; SEQ B of formula (I) has the sequence of SEQ ID NO:23; SEQ C of formula (I) has the sequence of SEQ ID NO:4; y of formula (I) is 0 and z of formula (I) is 0. Accordingly, the IL-2 protein of formula (I) has the sequence of SEQ ID NO:64:

APTSSSTKKTQLQLEHLLLDLQMILNGINNYKNPKLTRMLTFKFYMPKKA
TELKHLQCLEFGNSTLEEVLNLAQSKNFHLRPRDLISNINVIVLELKGSE
TTFMCEYADETATIVEFLNRWITFCQSIISTLT (SEQ ID NO:64)

In certain embodiments x of formula (I) is 0; SEQ A of formula (I) has the sequence of SEQ ID NO:36; SEQ B of formula (I) has the sequence of SEQ ID NO:23; SEQ C of formula (I) has the sequence of SEQ ID NO:4; y of formula (I) is 0 and z of formula (I) is 0. Accordingly, the IL-2 protein of formula (I) has the sequence of SEQ ID NO:65:

PASSSTKKTQLQLEHLLLDLQMILNGINNYKNPKLTRMLTFKFYMPKKAT
ELKHLQCLEFGNSTLEEVLNLAQSKNFHLRPRDLISNINVIVLELKGSET
TFMCEYADETATIVEFLNRWITFCQSIISTLT (SEQ ID NO:65)

In certain embodiments x of formula (I) is 1; SEQ A of formula (I) has the sequence of SEQ ID NO:36; SEQ B of formula (I) has the sequence of SEQ ID NO:23; SEQ C of formula (I) has the sequence of SEQ ID NO:4; y of formula (I) is 0 and z of formula (I) is 0. Accordingly, the IL-2 protein of formula (I) has the sequence of SEQ ID NO:66:

APASSSTKKTQLQLEHLLLDLQMILNGINNYKNPKLTRMLTFKFYMPKKA
TELKHLQCLEFGNSTLEEVLNLAQSKNFHLRPRDLISNINVIVLELKGSE
TTFMCEYADETATIVEFLNRWITFCQSIISTLT (SEQ ID NO:66)

In certain embodiments x of formula (I) is 0; SEQ A of formula (I) has the sequence of SEQ ID NO:1; SEQ B of formula (I) has the sequence of SEQ ID NO:26; SEQ C of formula (I) has the sequence of SEQ ID NO:3; y of formula (I) is 0 and z of formula (I) is 0. Accordingly, the IL-2 protein of formula (I) has the sequence of SEQ ID NO:24:

PTSSSTKKTQLQLEHLLLDLQMILNGINNYKNPKLTRMLTFKFYMPKKAT
ELKHLQCLEFANSTLEEVLNLAQSKNFHLRPRDLISNINVIVLELKGSET
TFMCEYADETATIVEFLNRWITFSQSIISTLT (SEQ ID NO:24)

In certain embodiments x of formula (I) is 1; SEQ A of formula (I) has the sequence of SEQ ID NO:1; SEQ B of formula (I) has the sequence of SEQ ID NO:26; SEQ C of formula (I) has the sequence of SEQ ID NO:3; y of formula (I) is 0 and z of formula (I) is 0. Accordingly, the IL-2 protein of formula (I) has the sequence of SEQ ID NO:25:

APTSSSTKKTQLQLEHLLLDLQMILNGINNYKNPKLTRMLTFKFYMPKKA
TELKHLQCLEFANSTLEEVLNLAQSKNFHLRPRDLISNINVIVLELKGSE
TTFMCEYADETATIVEFLNRWITFSQSIISTLT (SEQ ID NO:25)

In certain embodiments x of formula (I) is 0; SEQ A of formula (I) has the sequence of SEQ ID NO:36; SEQ B has the sequence of SEQ ID NO:26; SEQ C of formula (I) has the sequence of SEQ ID NO:3; y of formula (I) is 0 and z of formula (I) is 0. Accordingly, the IL-2 protein of formula (I) has the sequence of SEQ ID NO:67:

PASSSTKKTQLQLEHLLLDLQMILNGINNYKNPKLTRMLTFKFYMPKKAT
ELKHLQCLEFANSTLEEVLNLAQSKNFHLRPRDLISNINVIVLELKGSET
TFMCEYADETATIVEFLNRWITFSQSIISTLT (SEQ ID NO:67)

In certain embodiments x of formula (I) is 1; SEQ A of formula (I) has the sequence of SEQ ID NO:36; SEQ B of formula (I) has the sequence of SEQ ID NO:26; SEQ C of formula (I) has the sequence of SEQ ID NO:3; y of formula (I) is 0 and z of formula (I) is 0. Accordingly, the IL-2 protein of formula (I) has the sequence of SEQ ID NO:68:

APASSSTKKTQLQLEHLLLDLQMILNGINNYKNPKLTRMLTFKFYMPKKA
TELKHLQCLEFANSTLEEVLNLAQSKNFHLRPRDLISNINVIVLELKGSE
TTFMCEYADETATIVEFLNRWITFSQSIISTLT (SEQ ID NO:68)

In certain embodiments x of formula (I) is 0; SEQ A of formula (I) has the sequence of SEQ ID NO:1; SEQ B of formula (I) has the sequence of SEQ ID NO:26; SEQ C of formula (I) has the sequence of SEQ ID NO:4; y of formula (I) is 0 and z of formula (I) is 0. Accordingly, the IL-2 protein of formula (I) has the sequence of SEQ ID NO:69:

PTSSSTKKTQLQLEHLLLDLQMILNGINNYKNPKLTRMLTFKFYMPKKAT
ELKHLQCLEFANSTLEEVLNLAQSKNFHLRPRDLISNINVIVLELKGSET
TFMCEYADETATIVEFLNRWITFCQSIISTLT (SEQ ID NO:69)

In certain embodiments x of formula (I) is 1; SEQ A of formula (I) has the sequence of SEQ ID NO:1; SEQ B of formula (I) has the sequence of SEQ ID NO:26; SEQ C of formula (I) has the sequence of SEQ ID NO:4; y of formula (I) is 0 and z of formula (I) is 0. Accordingly, the IL-2 protein of formula (I) has the sequence of SEQ ID NO:70:

APTSSSTKKTQLQLEHLLLDLQMILNGINNYKNPKLTRMLTFKFYMPKKA
TELKHLQCLEFANSTLEEVLNLAQSKNFHLRPRDLISNINVIVLELKGSE
TTFMCEYADETATIVEFLNRWITFCQSIISTLT (SEQ ID NO:70)

In certain embodiments x of formula (I) is 0; SEQ A of formula (I) has the sequence of SEQ ID NO:36; SEQ B of formula (I) has the sequence of SEQ ID NO:26; SEQ C of formula (I) has the sequence of SEQ ID NO:4; y of formula (I) is 0 and z of formula (I) is 0. Accordingly, the IL-2 protein of formula (I) has the sequence of SEQ ID NO:71:

PASSSTKKTQLQLEHLLLDLQMILNGINNYKNPKLTRMLTFKFYMPKKAT
ELKHLQCLEFANSTLEEVLNLAQSKNFHLRPRDLISNINVIVLELKGSET
TFMCEYADETATIVEFLNRWITFCQSIISTLT (SEQ ID NO:71)

In certain embodiments x of formula (I) is 1; SEQ A of formula (I) has the sequence of SEQ ID NO:36; SEQ B of formula (I) has the sequence of SEQ ID NO:26; SEQ C of formula (I) has the sequence of SEQ ID NO:4; y of formula (I) is 0 and z of formula (I) is 0. Accordingly, the IL-2 protein of formula (I) has the sequence of SEQ ID NO:72:

APASSSTKKTQLQLEHLLLDLQMILNGINNYKNPKLTRMLTFKFYMPKKA
TELKHLQCLEFANSTLEEVLNLAQSKNFHLRPRDLISNINVIVLELKGSE
TTFMCEYADETATIVEFLNRWITFCQSIISTLT (SEQ ID NO:72)

In certain embodiments x of formula (I) is 0; SEQ A of formula (I) has the sequence of SEQ ID NO:1; SEQ B of formula (I) has the sequence of SEQ ID NO:32; SEQ C of formula (I) has the sequence of SEQ ID NO:3; y of formula (I) is 0 and z of formula (I) is 0. Accordingly, the IL-2 protein of formula (I) has the sequence of SEQ ID NO:30:

PTSSSTKKTQLQLEHLLLDLQMILNGINNYKNPKLTRMLTFKFYMPKKAT
ELKHLQCLEEELKPLEEFANSTQSKNFHLRPRDLISNINVIVLELKGSET
TFMCEYADETATIVEFLNRWITFSQSIISTLT (SEQ ID NO:30)

In certain embodiments x of formula (I) is 1; SEQ A of formula (I) has the sequence of SEQ ID NO:1; SEQ B of formula (I) has the sequence of SEQ ID NO:32; SEQ C of formula (I) has the sequence of SEQ ID NO:3; y of formula (I) is 0 and z of formula (I) is 0. Accordingly, the IL-2 protein of formula (I) has the sequence of SEQ ID NO:31:

APTSSSTKKTQLQLEHLLLDLQMILNGINNYKNPKLTRMLTFKFYMPKKA
TELKHLQCLEEELKPLEEFANSTQSKNFHLRPRDLISNINVIVLELKGSE
TTFMCEYADETATIVEFLNRWITFSQSIISTLT (SEQ ID NO:31)

In certain embodiments x of formula (I) is 0; SEQ A of formula (I) has the sequence of SEQ ID NO:36; SEQ B of formula (I) has the sequence of SEQ ID NO:32; SEQ C of formula (I) has the sequence of SEQ ID NO:3; y of formula (I) is 0 and z of formula (I) is 0. Accordingly, the IL-2 protein of formula (I) has the sequence of SEQ ID NO:73:

PASSSTKKTQLQLEHLLLDLQMILNGINNYKNPKLTRMLTFKFYMPKKAT
ELKHLQCLEEELKPLEEFANSTQSKNFHLRPRDLISNINVIVLELKGSET
TFMCEYADETATIVEFLNRWITFSQSIISTLT (SEQ ID NO:73)

In certain embodiments x of formula (I) is 1; SEQ A of formula (I) has the sequence of SEQ ID NO:36; SEQ B of formula (I) has the sequence of SEQ ID NO:32; SEQ C of formula (I) has the sequence of SEQ ID NO:3; y of formula (I) is 0 and z of formula (I) is 0. Accordingly, the IL-2 protein of formula (I) has the sequence of SEQ ID NO:74:

APASSSTKKTQLQLEHLLLDLQMILNGINNYKNPKLTRMLTFKFYMPKKA
TELKHLQCLEEELKPLEEFANSTQSKNFHLRPRDLISNINVIVLELKGSE
TTFMCEYADETATIVEFLNRWITFSQSIISTLT (SEQ ID NO:74)

In certain embodiments x of formula (I) is 0; SEQ A of formula (I) has the sequence of SEQ ID NO:1; SEQ B of formula (I) has the sequence of SEQ ID NO:32; SEQ C of formula (I) has the sequence of SEQ ID NO:4; y of formula (I) is 0 and z of formula (I) is 0. Accordingly, the IL-2 protein of formula (I) has the sequence of SEQ ID NO:75:

PTSSSTKKTQLQLEHLLLDLQMILNGINNYKNPKLTRMLTFKFYMPKKAT
ELKHLQCLEEELKPLEEFANSTQSKNFHLRPRDLISNINVIVLELKGSET
TFMCEYADETATIVEFLNRWITFCQSIISTLT (SEQ ID NO:75)

In certain embodiments x of formula (I) is 1; SEQ A of formula (I) has the sequence of SEQ ID NO:1; SEQ B of formula (I) has the sequence of SEQ ID NO:32; SEQ C of formula (I) has the sequence of SEQ ID NO:4; y of formula (I) is 0 and z of formula (I) is 0. Accordingly, the IL-2 protein of formula (I) has the sequence of SEQ ID NO:76:

APTSSSTKKTQLQLEHLLLDLQMILNGINNYKNPKLTRMLTFKFYMPKKA
TELKHLQCLEEELKPLEEFANSTQSKNFHLRPRDLISNINVIVLELKGSE
TTFMCEYADETATIVEFLNRWITFCQSIISTLT (SEQ ID NO:76)

In certain embodiments x of formula (I) is 0; SEQ A of formula (I) has the sequence of SEQ ID NO:36; SEQ B of formula (I) has the sequence of SEQ ID NO:32; SEQ C of formula (I) has the sequence of SEQ ID NO:4; y of formula (I) is 0 and z of formula (I) is 0. Accordingly, the IL-2 protein of formula (I) has the sequence of SEQ ID NO:77:

PASSSTKKTQLQLEHLLLDLQMILNGINNYKNPKLTRMLTFKFYMPKKAT
ELKHLQCLEEELKPLEEFANSTQSKNFHLRPRDLISNINVIVLELKGSET
TFMCEYADETATIVEFLNRWITFCQSIISTLT (SEQ ID NO:77)

In certain embodiments x of formula (I) is 1; SEQ A of formula (I) has the sequence of SEQ ID NO:36; SEQ B of formula (I) has the sequence of SEQ ID NO:32; SEQ C of formula (I) has the sequence of SEQ ID NO:4; y of formula (I) is 0 and z of formula (I) is 0. Accordingly, the IL-2 protein of formula (I) has the sequence of SEQ ID NO:78:

APASSSTKKTQLQLEHLLLDLQMILNGINNYKNPKLTRMLTFKFYMPKKA
TELKHLQCLEEELKPLEEFANSTQSKNFHLRPRDLISNINVIVLELKGSE
TTFMCEYADETATIVEFLNRWITFCQSIISTLT (SEQ ID NO:78)

In certain embodiments x of formula (I) is 0; SEQ A of formula (I) has the sequence of SEQ ID NO:1; SEQ B of formula (I) has the sequence of SEQ ID NO:35; SEQ C of formula (I) has the sequence of SEQ ID NO:3; y of formula (I) is 0 and z of formula (I) is 0. Accordingly, the IL-2 protein of formula (I) has the sequence of SEQ ID NO:33:

PTSSSTKKTQLQLEHLLLDLQMILNGINNYKNPKLTRMLTFKFYMPKKAT
ELKHLQCLEEELKPLEEFGNSTQSKNFHLRPRDLISNINVIVLELKGSET
TFMCEYADETATIVEFLNRWITFSQSIISTLT (SEQ ID NO:33)

In certain embodiments x of formula (I) is 1; SEQ A of formula (I) has the sequence of SEQ ID NO:1; SEQ B of formula (I) has the sequence of SEQ ID NO:35; SEQ C of formula (I) has the sequence of SEQ ID NO:3; y of formula (I) is 0 and z of formula (I) is 0. Accordingly, the IL-2 protein of formula (I) has the sequence of SEQ ID NO:34:

APTSSSTKKTQLQLEHLLLDLQMILNGINNYKNPKLTRMLTFKFYMPKKA
TELKHLQCLEEELKPLEEFGNSTQSKNFHLRPRDLISNINVIVLELKGSE
TTFMCEYADETATIVEFLNRWITFSQSIISTLT (SEQ ID NO:34)

In certain embodiments x of formula (I) is 0; SEQ A of formula (I) has the sequence of SEQ ID NO:36; SEQ B of formula (I) has the sequence of SEQ ID NO:35; SEQ C of formula (I) has the sequence of SEQ ID NO:3; y of formula (I) is 0 and z of formula (I) is 0. Accordingly, the IL-2 protein of formula (I) has the sequence of SEQ ID NO:79:

PASSSTKKTQLQLEHLLLDLQMILNGINNYKNPKLTRMLTFKFYMPKKAT
ELKHLQCLEEELKPLEEFGNSTQSKNFHLRPRDLISNINVIVLELKGSET
TFMCEYADETATIVEFLNRWITFSQSIISTLT (SEQ ID NO:79)

In certain embodiments x of formula (I) is 1; SEQ A of formula (I) has the sequence of SEQ ID NO:36; SEQ B of formula (I) has the sequence of SEQ ID NO:35; SEQ C of formula (I) has the sequence of SEQ ID NO:3; y of formula (I) is 0 and z of formula (I) is 0. Accordingly, the IL-2 protein of formula (I) has the sequence of SEQ ID NO:80:

APASSSTKKTQLQLEHLLLDLQMILNGINNYKNPKLTRMLTFKFYMPKKA
TELKHLQCLEEELKPLEEFGNSTQSKNFHLRPRDLISNINVIVLELKGSE
TTFMCEYADETATIVEFLNRWITFSQSIISTLT (SEQ ID NO:80)

In certain embodiments x of formula (I) is 0; SEQ A of formula (I) has the sequence of SEQ ID NO:1; SEQ B of formula (I) has the sequence of SEQ ID NO:35; SEQ C of formula (I) has the sequence of SEQ ID NO:4; y of formula (I) is 0 and z of formula (I) is 0. Accordingly, the IL-2 protein of formula (I) has the sequence of SEQ ID NO:81:

PTSSSTKKTQLQLEHLLLDLQMILNGINNYKNPKLTRMLTFKFYMPKKAT
ELKHLQCLEEELKPLEEFGNSTQSKNFHLRPRDLISNINVIVLELKGSET
TFMCEYADETATIVEFLNRWITFCQSIISTLT (SEQ ID NO:81)

In certain embodiments x of formula (I) is 1; SEQ A of formula (I) has the sequence of SEQ ID NO:1; SEQ B of formula (I) has the sequence of SEQ ID NO:35; SEQ C of formula (I) has the sequence of SEQ ID NO:4; y of formula (I) is 0 and z of formula (I) is 0. Accordingly, the IL-2 protein of formula (I) has the sequence of SEQ ID NO:82:

APTSSSTKKTQLQLEHLLLDLQMILNGINNYKNPKLTRMLTFKFYMPKKA
TELKHLQCLEEELKPLEEFGNSTQSKNFHLRPRDLISNINVIVLELKGSE
TTFMCEYADETATIVEFLNRWITFCQSIISTLT (SEQ ID NO:82)

In certain embodiments x of formula (I) is 0; SEQ A of formula (I) has the sequence of SEQ ID NO:36; SEQ B of formula (I) has the sequence of SEQ ID NO:35; SEQ C of formula (I) has the sequence of SEQ ID NO:4; y of formula (I) is 0 and z of formula (I) is 0. Accordingly, the IL-2 protein of formula (I) has the sequence of SEQ ID NO:83:

PASSSTKKTQLQLEHLLLDLQMILNGINNYKNPKLTRMLTFKFYMPKKAT
ELKHLQCLEEELKPLEEFGNSTQSKNFHLRPRDLISNINVIVLELKGSET
TFMCEYADETATIVEFLNRWITFCQSIISTLT (SEQ ID NO:83)

In certain embodiments x of formula (I) is 1; SEQ A of formula (I) has the sequence of SEQ ID NO:36; SEQ B of formula (I) has the sequence of SEQ ID NO:35; SEQ C of formula (I) has the sequence of SEQ ID NO:4; y of formula (I) is 0 and z of formula (I) is 0. Accordingly, the IL-2 protein of formula (I) has the sequence of SEQ ID NO:84:

APASSSTKKTQLQLEHLLLDLQMILNGINNYKNPKLTRMLTFKFYMPKKA
TELKHLQCLEEELKPLEEFGNSTQSKNFHLRPRDLISNINVIVLELKGSE
TTFMCEYADETATIVEFLNRWITFCQSIISTLT (SEQ ID NO:84)

In certain embodiments x of formula (I) is 0; SEQ A of formula (I) has the sequence of SEQ ID NO:1; SEQ B of formula (I) has the sequence of SEQ ID NO:11; SEQ C of formula (I) has the sequence of SEQ ID NO:3; y of formula (I) is 0; z of formula (I) is 1 and “Tag²” of formula (I) is a His tag comprising 6 histidines. Accordingly, the IL-2 protein of formula (I) has the sequence of SEQ ID NO:204:

PTSSSTKKTQLQLEHLLLDLQMILNGINNYKNPKLTFGNSTKFYMPKKAT
ELKHLQCLEEELKPLEEVLNLAQSKNFHLRPRDLISNINVIVLELKGSET
TFMCEYADETATIVEFLNRWITFSQSIISTLTHHHHHH (SEQ ID NO:
204)

In certain embodiments x of formula (I) is 1; SEQ A of formula (I) has the sequence of SEQ ID NO:1; SEQ B of formula (I) has the sequence of SEQ ID NO:11; SEQ C of formula (I) has the sequence of SEQ ID NO:3; y of formula (I) is 0; z of formula (I) is 1 and “Tag²” of formula (I) is a His tag comprising 6 histidines. Accordingly, the IL-2 protein of formula (I) has the sequence of SEQ ID NO:205:

APTSSSTKKTQLQLEHLLLDLQMILNGINNYKNPKLTFGNSTKFYMPKKA
TELKHLQCLEEELKPLEEVLNLAQSKNFHLRPRDLISNINVIVLELKGSE
TTFMCEYADETATIVEFLNRWITFSQSIISTLTHHHHHH (SEQ ID NO
:205)

In certain embodiments x of formula (I) is 0; SEQ A of formula (I) has the sequence of SEQ ID NO:36; SEQ B of formula (I) has the sequence of SEQ ID NO:11; SEQ C of formula (I) has the sequence of SEQ ID NO:3; y of formula (I) is 0; z of formula (I) is 1 and “Tag²” of formula (I) is a His tag comprising 6 histidines. Accordingly, the IL-2 protein of formula (I) has the sequence of SEQ ID NO:206:

PASSSTKKTQLQLEHLLLDLQMILNGINNYKNPKLTFGNSTKFYMPKKAT
ELKHLQCLEEELKPLEEVLNLAQSKNFHLRPRDLISNINVIVLELKGSET
TFMCEYADETATIVEFLNRWITFSQSIISTLTHHHHHH (SEQ ID NO:
206)

In certain embodiments x of formula (I) is 1; SEQ A of formula (I) has the sequence of SEQ ID NO:36; SEQ B of formula (I) has the sequence of SEQ ID NO:11; SEQ C of formula (I) has the sequence of SEQ ID NO:3; y of formula (I) is 0; z of formula (I) is 1 and “Tag²” of formula (I) is a His tag comprising 6 histidines. Accordingly, the IL-2 protein of formula (I) has the sequence of SEQ ID NO:207:

APASSSTKKTQLQLEHLLLDLQMILNGINNYKNPKLTFGNSTKFYMPKKA
TELKHLQCLEEELKPLEEVLNLAQSKNFHLRPRDLISNINVIVLELKGSE
TTFMCEYADETATIVEFLNRWITFSQSIISTLTHHHHHH (SEQ ID NO
:207)

In certain embodiments x of formula (I) is 0; SEQ A of formula (I) has the sequence of SEQ ID NO:1; SEQ B of formula (I) has the sequence of SEQ ID NO:11; SEQ C of formula (I) has the sequence of SEQ ID NO:4; y of formula (I) is 0; z of formula (I) is 1 and “Tag²” of formula (I) is a polyhistidine (His-tag). Accordingly, the IL-2 protein of formula (I) has the sequence of SEQ ID NO:208:

PTSSSTKKTQLQLEHLLLDLQMILNGINNYKNPKLTFGNSTKFYMPKKAT
ELKHLQCLEEELKPLEEVLNLAQSKNFHLRPRDLISNINVIVLELKGSET
TFMCEYADETATIVEFLNRWITFCQSIISTLTHHHHHH (SEQ ID NO:
208)

In certain embodiments x of formula (I) is 1; SEQ A of formula (I) has the sequence of SEQ ID NO:1; SEQ B of formula (I) has the sequence of SEQ ID NO:11; SEQ C of formula (I) has the sequence of SEQ ID NO:4; y of formula (I) is 0; z of formula (I) is 1 and “Tag²” of formula (I) is a His tag comprising 6 histidines. Accordingly, the IL-2 protein of formula (I) has the sequence of SEQ ID NO:209:

APTSSSTKKTQLQLEHLLLDLQMILNGINNYKNPKLTFGNSTKFYMPKKA
TELKHLQCLEEELKPLEEVLNLAQSKNFHLRPRDLISNINVIVLELKGSE
TTFMCEYADETATIVEFLNRWITFCQSIISTLTHHHHHH (SEQ ID NO
:209)

In certain embodiments x of formula (I) is 0; SEQ A of formula (I) has the sequence of SEQ ID NO:36; SEQ B of formula (I) has the sequence of SEQ ID NO:11; SEQ C of formula (I) has the sequence of SEQ ID NO:4; y of formula (I) is 0; z of formula (I) is 1 and “Tag²” of formula (I) is a His tag comprising 6 histidines. Accordingly, the IL-2 protein of formula (I) has the sequence of SEQ ID NO:210:

PASSSTKKTQLQLEHLLLDLQMILNGINNYKNPKLTFGNSTKFYMPKKAT
ELKHLQCLEEELKPLEEVLNLAQSKNFHLRPRDLISNINVIVLELKGSET
TFMCEYADETATIVEFLNRWITFCQSIISTLTHHHHHH (SEQ ID NO:
210)

In certain embodiments x of formula (I) is 1; SEQ A of formula (I) has the sequence of SEQ ID NO:36; SEQ B of formula (I) has the sequence of SEQ ID NO:11; SEQ C of formula (I) has the sequence of SEQ ID NO:4; y of formula (I) is 0; z of formula (I) is 1 and “Tag²” of formula (I) is a His tag comprising 6 histidines. Accordingly, the IL-2 protein of formula (I) has the sequence of SEQ ID NO:211:

APASSSTKKTQLQLEHLLLDLQMILNGINNYKNPKLTFGNSTKFYMPKKA
TELKHLQCLEEELKPLEEVLNLAQSKNFHLRPRDLISNINVIVLELKGSE
TTFMCEYADETATIVEFLNRWITFCQSIISTLTHHHHHH (SEQ ID NO
:211)

In certain embodiments x of formula (I) is 1; SEQ A of formula (I) has the sequence of SEQ ID NO:1; SEQ B of formula (I) has the sequence of SEQ ID NO:17; SEQ C of formula (I) has the sequence of SEQ ID NO:3; y of formula (I) is 0; z of formula (I) is 1 and “Tag²” of formula (I) is a His tag comprising 6 histidines. Accordingly, the IL-2 protein of formula (I) has the sequence of SEQ ID NO:229:

APTSSSTKKTQLQLEHLLLDLQMILNGINNYKNPKLTRMLFGNSTMPKKA
TELKHLQCLEEELKPLEEVLNLAQSKNFHLRPRDLISNINVIVLELKGSE
TTFMCEYADETATIVEFLNRWITFSQSIISTLTHHHHHH (SEQ ID NO
:229)

In certain embodiments x of formula (I) is 1; SEQ A of formula (I) has the sequence of SEQ ID NO:1; SEQ B of formula (I) has the sequence of SEQ ID NO:23; SEQ C of formula (I) has the sequence of SEQ ID NO:3; y of formula (I) is 0; z of formula (I) is 1 and “Tag²” of formula (I) is a His tag comprising 6 histidines. Accordingly, the IL-2 protein of formula (I) has the sequence of SEQ ID NO:230:

APTSSSTKKTQLQLEHLLLDLQMILNGINNYKNPKLTRMLTFKFYMPKKA
TELKHLQCLEFGNSTLEEVLNLAQSKNFHLRPRDLISNINVIVLELKGSE
TTFMCEYADETATIVEFLNRWITFSQSIISTLTHHHHHH (SEQ ID NO
:230)

In certain embodiments x of formula (I) is 1; SEQ A of formula (I) has the sequence of SEQ ID NO:1; SEQ B of formula (I) has the sequence of SEQ ID NO:11; SEQ C of formula (I) has the sequence of SEQ ID NO:3; y of formula (I) is 1; z of formula (I) is 0 and “Tag¹” of formula (I) is a His tag having the sequence SEQ ID NO:234. Accordingly, the IL-2 protein of formula (I) has the sequence of SEQ ID NO:231:

AHHHHHHGSDDDDKAPTSSSTKKTQLQLEHLLLDLQMILNGINNYKNPKL
TFGNSTKFYMPKKATELKHLQCLEEELKPLEEVLNLAQSKNFHLRPRDLI
SNINVIVLELKGSETTFMCEYADETATIVEFLNRWITFSQSIISTLT (S
EQ ID NO:231)

In certain embodiments the IL-2 protein of formula (I) has a sequence selected from the group consisting of SEQ ID NO:9; SEQ ID NO:10; SEQ ID NO:12; SEQ ID NO:13; SEQ ID NO:15; SEQ ID NO:16; SEQ ID NO:18; SEQ ID NO:19; SEQ ID NO:21; SEQ ID NO:22; SEQ ID NO:24; SEQ ID NO:25; SEQ ID NO:30; SEQ ID NO:31; SEQ ID NO:33; SEQ ID NO:34, SEQ ID NO:216; SEQ ID NO:217; SEQ ID NO:224; and SEQ ID NO:225.

In certain embodiments the IL-2 protein of formula (I) has a sequence selected from the group consisting of SEQ ID NO:9; SEQ ID NO:12; SEQ ID NO:15; SEQ ID NO:18; SEQ ID NO:21; SEQ ID NO:24; SEQ ID NO:30; SEQ ID NO:33; SEQ ID NO:216; and SEQ ID NO:224.

In certain embodiments the IL-2 protein of formula (I) has a sequence selected from the group consisting of SEQ ID NO:10; SEQ ID NO:13; SEQ ID NO:16; SEQ ID NO:19; SEQ ID NO:22; SEQ ID NO:25; SEQ ID NO:31; SEQ ID NO:34, SEQ ID NO:217; and SEQ ID NO:225.

In certain embodiments the IL-2 protein of formula (I) has the sequence of SEQ ID NO:9. In certain embodiments the IL-2 protein of formula (I) has the sequence of SEQ ID NO:10. In certain embodiments the IL-2 protein of formula (I) has the sequence of SEQ ID NO:12. In certain embodiments the IL-2 protein of formula (I) has the sequence of SEQ ID NO:13. In certain embodiments the IL-2 protein of formula (I) has the sequence of SEQ ID NO:15. In certain embodiments the IL-2 protein of formula (I) has the sequence of SEQ ID NO:16. In certain embodiments the IL-2 protein of formula (I) has the sequence of SEQ ID NO:18. In certain embodiments the IL-2 protein of formula (I) has the sequence of SEQ ID NO:19. In certain embodiments the IL-2 protein of formula (I) has the sequence of SEQ ID NO:21. In certain embodiments the IL-2 protein of formula (I) has the sequence of SEQ ID NO:22. In certain embodiments the IL-2 protein of formula (I) has the sequence of SEQ ID NO:24. In certain embodiments the IL-2 protein of formula (I) has the sequence of SEQ ID NO:25. In certain embodiments the IL-2 protein of formula (I) has the sequence of SEQ ID NO:30. In certain embodiments the IL-2 protein of formula (I) has the sequence of SEQ ID NO:31. In certain embodiments the IL-2 protein of formula (I) has the sequence of SEQ ID NO:33. In certain embodiments the IL-2 protein of formula (I) has the sequence of SEQ ID NO:34. In certain embodiments the IL-2 protein of formula (I) has the sequence of SEQ ID NO:216. In certain embodiments the IL-2 protein of formula (I) has the sequence of SEQ ID NO:217. In certain embodiments the IL-2 protein of formula (I) has the sequence of SEQ ID NO:224. In certain embodiments the IL-2 protein of formula (I) has the sequence of SEQ ID NO:225.

In certain embodiments, SEQ A of formula (I) may comprise at least one O-linked N1 glycan, at least one O-linked N2 glycan, a combination of at least one O-linked N1 and at least one O-linked N2 glycan or may be non-glycosylated. In certain embodiments the SEQ A of formula (I) comprises at least one, such as one, O-linked N1 glycan. In certain embodiments the SEQ A of formula (I) comprises at least one, such as one, O-linked N2 glycan. In certain embodiments the SEQ A of formula (I) comprises a combination of at least one, such as one, O-linked N1 glycan and at least one, such as one, O-linked N2 glycan. In certain embodiments the SEQ A of formula (I) is non-O-glycosylated. In certain embodiments, the ratio of O-linked N1 to O-linked N2 glycans is 1:1, 1:2, 1:3 or 1:4. In certain embodiments, the ratio of O-linked N1 to O-linked N2 glycans is 1:1. In certain embodiments, the ratio of O-linked N1 to O-linked N2 glycans is 1:2. In certain embodiments, the ratio of N1 to N2 glycans is 1:3. In certain embodiments, the ratio of N1 to N2 glycans is 1:4.

The IL-2 protein of formula (I) may in certain embodiments comprise at least one N-linked biantennary paucimannose glycan. In certain embodiments the IL-2 protein of formula (I) comprises at least one, such as one, N-linked G0 glycan. In certain embodiments the IL-2 protein of formula (I) comprises at least one, such as one, N-linked G1 glycan. In certain embodiments the IL-2 protein of formula (I) comprises at least one, such as one, N-linked G2 glycan. In certain embodiments the IL-2 protein of formula (I) comprises at least one, such as one, N-linked G0B glycan. In certain embodiments the IL-2 protein of formula (I) comprises at least one, such as one, N-linked G1B glycan. In certain embodiments the IL-2 protein of formula (I) comprises at least one, such as one, N-linked G2B glycan. In certain embodiments the IL-2 protein of formula (I) comprises at least one, such as one, N-linked G0F glycan. In certain embodiments the IL-2 protein of formula (I) comprises at least one, such as one, N-linked G1F glycan. In certain embodiments the IL-2 protein of formula (I) comprises at least one, such as one, N-linked G2F glycan. In certain embodiments the IL-2 protein of formula (I) comprises at least one, such as one, N-linked G0BF glycan. In certain embodiments the IL-2 protein of formula (I) comprises at least one, such as one, N-linked G1BF glycan. In certain embodiments the IL-2 protein of formula (I) comprises at least one, such as one, N-linked G2BF glycan. In certain embodiments the protein of formula (I) may comprise a mixture of biantennary paucimannose glycans. In certain embodiments the protein of formula (I) comprises N-linked tetra-antennary paucimannose glycans with more than two Gal residues, such as three, four, five or six Gal residues and/or more than two NeuAc residues, such as three or four NeuAc residues.

In certain embodiments the one or more N-linked or O-linked glycan extends the half-life of the protein of formula (I).

In certain embodiments the one or more N-linked or O-linked glycan shields the mutations introduced with the glycosylation motif from immune recognition. In certain embodiments, the one or more N-linked or O-linked glycan blocks recognition of the mutations introduced with the glycosylation motif by antibodies or T-cell receptors (TCR).

In certain embodiments the one or more N-linked or O-linked glycan can be used for affinity purification. For example, an N-linked glycan may be used for affinity purification via lectins.

It is understood that at least one glycan is present in the SEQ B moiety, but there may also be glycans elsewhere in the IL-2 protein of formula (I), such as in SEQ A or SEQ C.

In certain embodiments, the IL-2 protein of formula (I) is a biased IL-2.

In another aspect the present invention relates to an oligonucleotide sequence encoding the IL-2 protein of formula (I). Such oligonucleotide sequence may be selected from the group consisting of DNA, RNA and cDNA sequences. In certain embodiments the oligonucleotide sequence is a DNA sequence. In certain embodiments the oligonucleotide sequence is an RNA sequence. In certain embodiments the oligonucleotide sequence is a cDNA sequence. In certain embodiments the oligonucleotide encoding the IL-2 protein of formula (I) is for expression in a eukaryotic system, prokaryotic system or in a cell-free system. In certain embodiments the oligonucleotide sequence is for expression in a eukaryotic system. In certain embodiments the oligonucleotide sequence is for expression in a prokaryotic system. In certain embodiments the oligonucleotide sequence is for expression in a cell-free system. In certain embodiments the DNA sequence encoding the IL-2 protein of formula (I) has the sequence of SEQ ID NO:7. In certain embodiments the DNA sequence encoding the IL-2 protein of formula (I) has the sequence of SEQ ID NO:8. In certain embodiments the DNA sequence encoding the IL-2 protein of formula (I) has the sequence of SEQ ID NO:202. In certain embodiments the DNA sequence encoding the IL-2 protein of formula (I) has the sequence of SEQ ID NO:203. In certain embodiments the DNA sequence encoding the IL-2 protein of formula (I) has the sequence of SEQ ID NO:233. In certain embodiments the DNA sequence encoding the IL-2 protein of formula (I) has the sequence of SEQ ID NO:244. In certain embodiments the DNA sequence encoding the IL-2 protein of formula (I) has the sequence of SEQ ID NO:245. In certain embodiments the DNA sequence encoding the IL-2 protein of formula (I) has the sequence of SEQ ID NO:246. In certain embodiments the RNA sequence encoding the IL-2 protein of formula (I) is an mRNA sequence.

In certain embodiments the oligonucleotide sequence encoding the IL-2 protein of formula (I) is for expression in a prokaryotic system, such as a bacterial system selected from the group consisting of Escherichia coli; Bacillus sp., such as Bacillus subtilis; Corynebacterium sp., such as Corynebacterium glutamicum; and Pseudomonas fluorescens. In certain embodiments such oligonucleotide is a DNA sequence in the form of a plasmid.

In certain embodiments the oligonucleotide sequence encoding the IL-2 protein of formula (I) is for expression in a eukaryotic system, such as a eukaryotic system selected from the group consisting of mammalian systems, such as mammalian cells, such as Chinese hamster ovary cells (CHO), mouse myeloma lymphoblastoid (such as NS0 cells), Sp2/0 cells, mouse fibroblasts (such as NIH3T3 cells), and fully human cells, such as human embryotic kidney cells HEK 293, HeLa cells, human embryonic retinal cells (such as Crucell’s Per.C6) or human amniocyte cells (such as Glycotope and CEVEC); yeasts, such as Saccharomyces cerevisiae, Yarrowia lipolytica or Pichia pastoris; filamentous fungi, such as Aspergillus, Trichoderma or Myceliophthora thermophila; baculovirus-infected cells, such baculovirus-infected insect cells, such as Sf9, Sf21, Hi-5 strains; non-lytic insect cell expression systems, such as Sf9, Sf21, Hi-5, Schneider 2 cells or Schneider 3 cells; and plants or plant cells, such as Arabidopsis thaliana, Nicotiana benthamiana, Nicotiana tabacum, Medicago sativa, Lemna minor, or Physcomitrella patens and cells thereof. In certain embodiments the oligonucleotide sequence encoding the IL-2 protein of formula (I) is for expression in CHO cells. In certain embodiments the oligonucleotide sequence encoding IL-2 protein of formula (I) is for expression in a mammalian system. In certain embodiments such oligonucleotide is a DNA sequence in the form of a plasmid.

In certain embodiments the oligonucleotide encoding the IL-2 protein of formula (I) is for use in the treatment of a disease that can be treated with IL-2, in particular with a biased IL-2. In certain embodiments said disease that can be treated with IL-2, in particular with a biased IL-2, is cancer. In certain embodiments the oligonucleotide encoding the IL-2 protein of formula (I) for such use is a DNA, RNA or cDNA, in particular a modified DNA, modified RNA or modified cDNA. In certain embodiments such oligonucleotide for use in the treatment of a disease is administered to a patient, which in certain embodiments is a mammal, such as a cat, dog, horse, cow, sheep, non-human primate or a human.

In certain embodiments the oligonucleotide encoding the IL-2 protein of formula (I) for use in the treatment of a disease that can be treated with IL-2 is a DNA molecule, in particular a modified DNA molecule. In certain embodiments the DNA encoding the IL-2 protein of formula (I) is a modified DNA. In certain embodiments the modification of the DNA is selected from the group consisting of backbone modification, conformational constraint modification and chemical modification. In certain embodiments the conformational constraint modification of the DNA encoding the IL-2 protein of formula (I) is selected from the group consisting of locked nucleic acid (LNA), constrained 2′-O-ethyl- (cEt) and tricyclo-DNA (tcDNA). In certain embodiments the backbone modification of the DNA encoding the IL-2 protein of formula (I) is selected from the group consisting of 2′-O-methyl (2′-OMe), 2′-O-methoxy-ethyl (2′-MOE) and 2′-fluoro (2′-F) substitutions. In certain embodiments the modification of the DNA comprises a combination of at least two modifications selected from the group consisting of backbone modification, conformational constraint modification and chemical modification.

In certain embodiments the oligonucleotide encoding the IL-2 protein of formula (I) for use in the treatment of a disease that can be treated with IL-2 is a cDNA molecule, in particular a modified cDNA molecule.

In certain embodiments the oligonucleotide encoding the IL-2 protein of formula (I) for use in the treatment of a disease that can be treated with IL-2 is an RNA molecule, in particular a modified RNA molecule. In certain embodiments the RNA encoding the IL-2 protein of formula (I) is modified RNA. In certain embodiments the modification of the RNA is selected from the group consisting of backbone modification, ribose modification, conformational constraint modification and chemical modification. In certain embodiments the ribose modification of the RNA encoding the IL-2 protein of formula (I) is replacing phosphodiester (PO) with phophorothioate (PS) in the RNA backbone. In certain embodiments the backbone modification of the RNA encoding the IL-2 protein of formula (I) is selected from the group consisting of 2′-O-methyl (2′-OMe), 2′-O-methoxy-ethyl (2′-MOE) and 2′-fluoro (2′-F) substitutions. In certain embodiments the chemical modification of the RNA encoding the IL-2 protein of formula (I) is changes in the nucleobase. In certain embodiments the change in the nucleobase is phosphorodiamidate morpholino oligomers (PMO) or peptide nucleic acid (PNA). In certain embodiments the modification of the RNA comprises a combination of at least two modifications selected from the group consisting of backbone modification, ribose modification, conformational constraint modification and chemical modification.

In certain embodiments the RNA encoding the IL-2 protein of formula (I) is modified for improved delivery. In certain embodiments the modification for improved delivery is selected from the group consisting of chemical conjugation to certain moieties and incorporation into or attachment to nanoparticulate carriers. In certain embodiments the chemical conjugate is selected from the group consisting of polymers, cell-penetrating peptides (CPPs), lipids, antibodies, receptor ligands and aptamers. In certain embodiments the polymer is a dendrimer. In certain embodiments the polymer is selected from the group consisting of PEG, poly(lactide-co-glycolic acid) (PLGA) and polyphosphazenes. In certain embodiments the polymer is PEG. In certain embodiments the nanoparticulate carrier is a nanoparticulate carrier comprising lipids, polymers and/or peptides. In certain embodiments the nanoparticulate carrier comprising a lipid is selected from the group consisting of lipid nanoparticles (LNP) and exosomes. In certain embodiments the nanoparticulate carrier comprises a polymeric dendrimer. In certain embodiments the nanoparticulate carrier comprises a polymer selected from the group consisting of PEG, poly(lactide-co-glycolic acid) (PLGA) and polyphosphazenes.

In certain embodiments the lipid nanoparticle (LNP) used for delivery of the RNA encoding the IL-2 protein of formula (I) comprises an ionizable or cationic lipid or polymeric material, bearing tertiary or quaternary amines; a zwitterionic lipid (e.g., 1,2-dioleoyl-sn-glycero-3-phosphoethanolamine [DOPE]); cholesterol; a polyethylene glycol (PEG)-lipid or any combination thereof.

In certain embodiments the oligonucleotide encoding the IL-2 protein of formula (I) is delivered into a cell by a method selected from the group consisting of cationic lipids, biodegradable ionizable lipid, co-formulation into lipid nanoparticles (LNPs), nanoliposomes, emulsions, polymeric carrier, electroporation, lipofection, calcium phosphate precipitation, nanoparticle based transfection, virus based transfection, or transfection based on cationic polymers, such as DEAE-dextran or polyethylenimine. In certain embodiments the oligonucleotide encoding the IL-2 protein of formula (I) is delivered into a cell ex vivo. In certain embodiments the oligonucleotide encoding the IL-2 protein of formula (I) is delivered into a cell in vivo.

In certain embodiments the oligonucleotide encoding the IL-2 protein of formula (I) is for expression in a host cell of a human subject. In certain embodiments the oligonucleotide encoding the IL-2 protein of formula (I) is delivered into a host cell in vivo or ex vivo. In certain embodiments the oligonucleotide encoding the IL-2 protein of formula (I) is delivered into a host cell in vivo. In certain embodiments the oligonucleotide encoding the IL-2 protein of formula (I) is delivered into a host cell ex vivo. In certain embodiments the oligonucleotide encoding the IL-2 protein of formula (I) is delivered into a host cell of a human subject, which cell subsequently expresses the IL-2 protein of formula (I). In certain embodiments said host cell is an immune cell. In certain embodiments said immune cell is selected from the group consisting of T-cells, natural killer (NK) cells, myeloid cells, dendritic cells, red blood cells, macrophages, B-cells or any other genetically modified immune cell type

In certain embodiments the oligonucleotide encoding the IL-2 protein of formula (I) is delivered into a cell via virus-based transfection using a viral vector. In certain embodiments the viral vector comprises the oligonucleotide encoding the IL-2 protein of formula (I). In certain embodiments the viral vector is selected form the group consisting DNA-based viral vectors and RNA-based viral vectors.

In certain embodiments the DNA-based viral vector comprising the oligonucleotide encoding the IL-2 protein of formula (I) is selected from the group consisting of poxviruses, adenoviruses, adeno-associated viruses and herpes viruses. In certain embodiments the herpes virus is selected from the group consisting of herpes simplex virus 1 and herpes simplex virus 2. In certain embodiments the herpes simplex virus 1 has a deletion of at least one viral gene, such as a gene that is selected from the group consisting of ICP34.5, ICP6/UL39, and ICP47. In certain embodiments the herpes simplex virus 1 is selected from the group consisting of G207, HSV1716, NV1020 and Talimogene laherparepvec (Oncovex-GMCSF). In certain embodiments the DNA-based viral vector is an oncolytic virus. In certain embodiments the DNA-based oncolytic virus is selected from the group consisting of herpes simplex virus 1, adenoviruses, and poxviruses. In certain embodiments the oncolytic adenovirus is selected from the group consisting of H101, ColoAd1 (Ad11p/Ad3) and Onyx-015 (Ad⅖ dl1520).

In certain embodiments the RNA-based viral vector comprising the oligonucleotide encoding the IL-2 protein of formula (I) is selected from the group consisting of lentiviruses, retroviruses, alphaviruses, flaviviruses, rhabdoviruses, measles viruses, polioviruses, human foamy viruses and newcastle disease viruses. In certain embodiments the RNA-based viral vector is a retrovirus, such as a retrovirus selected from the group consisting of moloney murine leukemia virus, moloney murine sarcoma virus and murine stem cell virus. In certain embodiments the RNA-based viral vector is a lentivirus, such as a lentivirus selected from the group consisting of human immunodeficiency virus 1, human immunodeficiency virus 2 and equine infectious anemia virus. In certain embodiments the RNA-based viral vector is an alphavirus, such as an alphavirus selected from the group consisting of semliki forest virus, sindbis virus, venezuelan equine encephalitis virus and M1 virus. In certain embodiments the RNA-based viral vector is a flavivirus, such as a flavivirus selected from the group consisting of kunjin virus, west nile virus, yellow fever virus and dengue virus. In certain embodiments the RNA-based viral vector is a rhabdovirus, such as a rhabdovirus selected from the group consisting of Rabies and vesicular stomatitis virus. In certain embodiments the RNA-based viral vector is a measles virus, such as MV-Edm. In certain embodiments the RNA-based viral vector is a picornavirus, such as a picornavirus selected from the group consisting of a coxsackievirus and seneca valley virus.

In certain embodiments the RNA-based viral vector is an oncolytic virus. In certain embodiments the RNA-based oncolytic virus is selected from the group consisting of MV-Edm, newcastle disease virus, vesicular stomatitis virus, seneca valley virus and coxsackievirus.

In certain embodiments the viral vector is a virus-like vesicle (VLV). In certain embodiments the virus-like vesicle is a hybrid of components from two viruses. In certain embodiments the virus-like vesicle is a hybrid of components from alphavirus semliki forest virus (SFV) and rhabdovirus vesicular stomatitis virus (VSV).

It is understood that after successful delivery of the viral vector comprising the oligonucleotide encoding the IL-2 protein of formula (I) into a host cell, said host cell will express and secrete the IL-2 protein of formula (I).

In certain embodiments the oligonucleotide encoding the IL-2 protein of formula (I) is delivered as a vaccine formulation. In certain embodiments said vaccine formulation is selected from the group consisting of DNA based, RNA based, cell-based or viral based vaccines.

In certain embodiments the IL-2 protein of formula (I) is expressed in an engineered cell. In certain embodiments the engineered cell is an engineered immune cell. In certain embodiments the engineered immune cell is selected from the group consisting of T-cells, natural killer (NK) cells, myeloid cells, dendritic cells, red blood cells, macrophages, B-cells or any other genetically modified immune cell type.

In certain embodiments the IL-2 protein of formula (I) is expressed in an engineered T-cell. In certain embodiments the engineered T-cell is selected from the group consisting of TCR-engineered T (TCR-T) cells, chimeric antigen receptor-modified T (CAR-T) cells and T cells redirected for universal cytokine killing (TRUCK). In certain embodiments the engineered T-cell is a TCR-T cell. In certain embodiments the engineered T-cell is a CAR-T cell. In certain embodiments the engineered T-cell is a TRUCK.

In certain embodiments the IL-2 protein of formula (I) is expressed in an engineered α/β T-cell.

In certain embodiments the IL-2 protein of formula (I) is expressed in an engineered γ/δ T-cell.

In certain embodiments the IL-2 protein of formula (I) is expressed in an engineered natural killer T-cell.

In certain embodiments the IL-2 protein of formula (I) is expressed in an engineered NK-cell.

In certain embodiments the engineered NK-cell is a chimeric antigen receptor-modified NK (CAR-NK) cell. In certain embodiments the engineered NK-cell is a lymphokine-activated killers (LAK) cell. In certain embodiments the engineered NK-cell is a NK-92 cell.

In certain embodiments the IL-2 protein of formula (I) is expressed in an engineered myeloid cell. In certain embodiments the engineered macrophage is a CAR myeloid cell.

In certain embodiments the IL-2 protein of formula (I) is expressed in an engineered dendritic cell. In certain embodiments the engineered dendritic cell is a CAR dendritic cell.

In certain embodiments the IL-2 protein of formula (I) is expressed in an engineered macrophage. In certain embodiments the engineered macrophage is a CAR macrophage.

In certain embodiments the IL-2 protein of formula (I) is expressed in an engineered red blood cell. In certain embodiments the IL-2 protein of formula (I) is expressed in an engineered red blood cell and presented on the cell-surface.

In certain embodiments the IL-2 protein of formula (I) is expressed in an engineered B-cell. In certain embodiments the engineered macrophage is a CAR B-cell.

It is understood that the IL-2 protein of formula (I) expressed in an engineered immune cell may be secreted from said cell.

In certain embodiments the engineered immune cell expressing the IL-2 protein of formula (I) is used for a cell therapy. In certain embodiments the cell therapy is an adoptive immune cell therapy. In certain embodiments the adoptive immune cell therapy is autologous or heterologous. In certain embodiments the adoptive immune cell therapy is autologous. In certain embodiments the adoptive immune cell therapy is heterologous.

In certain embodiments the IL-2 protein of formula (I) may be used for the in vitro or ex vivo stimulation of cells, such as immune cells. In certain embodiments the IL-2 protein of formula (I) is for the stimulation of engineered immune cells. In certain embodiments the IL-2 protein of formula (I) is for the stimulation of cells selected from the group of T-cells, NK-cells, macrophages, and dendritic cells.

In another aspect the present invention relates to a method for the expression of a recombinant IL-2 protein of formula (I), said method comprising: a) culturing host cells expressing one or more genes encoding the IL-2 protein of formula (I); and b) separating said recombinant IL-2 protein of interest from the host cell culture.

In certain embodiments the host cells are prokaryotic cells, such as bacterial cells. In certain embodiments the host cells are selected from the group consisting of Escherichia coli; Bacillus sp., such as Bacillus subtilis; Corynebacterium sp., such as Corynebacterium glutamicum; and Pseudomonas fluorescens. In certain embodiments the host cells are Escherichia coli. In certain embodiments the host cells are a Bacillus sp. In certain embodiments the host cells are a Corynebacterium sp. In certain embodiments the host cells are Pseudomonas fluorescens.

In certain embodiments the host cells are eukaryotic cells. In certain embodiments the host cells are selected from the group consisting of yeasts, such as Saccharomyces cerevisiae or Pichia pastoris; filamentous fungi, such as Aspergillus, Trichoderma or Myceliophthora thermophila; baculovirus-infected cells, such as baculovirus-infected insect cells, such as Sf9, Sf21, Hi-5 strains, or baculovirus-infected mammalian cells, such as HeLa, human embryotic kidney cells HEK 293 or Chinese hamster ovary cells (CHO); mammalian systems, such as mouse myeloma lymphoblastoid (such as NS0 cells), mouse fibroblasts (such as NIH3T3 cells), CHO cells, and fully human cells, such as HEK 293 cells, human embryonic retinal cells (such as Crucell’s Per.C6) and human amniocyte cells (such as Glycotope and CEVEC); and non-lytic insect cell expression systems, such as Sf9, Sf21, Hi-5, Schneider 2 cells or Schneider 3 cells. In certain embodiments the host cells are yeast cells. In certain embodiments the host cells are Saccharomyces cerevisiae cells. In certain embodiments the host cells are Pichia pastoris cells. In certain embodiments the host cells are cells of a filamentous fungus. In certain embodiments the host cells are cells of an Aspergillus species. In certain embodiments the host cells are cells of a Trichoderma species. In certain embodiments the host cells are Myceliophthora thermophila cells. In certain embodiments the host cells are baculovirus-infected cells, such as a baculovirus-infected insect cells or baculovirus-infected mammalian cells. In certain embodiments the host cells are baculovirus-infected Sf9 cells. In certain embodiments the host cells are baculovirus-infected Sf21 cells. In certain embodiments the host cells are cells of a baculovirus-infected Hi-5 strain. In certain embodiments the host cells are baculovirus-infected HeLa cells. In certain embodiments the host cells are baculovirus-infected human kidney cells. In certain embodiments the host cells are baculovirus-infected Sf9 cells. In certain embodiments the host cells are baculovirus-infected CHO cells. In certain embodiments the host cells are mammalian cells. In certain embodiments the host cells are mouse myeloma lymphoblastoid cells. In certain embodiments the host cells are mouse fibroblast cells. In certain embodiments the host cells are CHO cells. In certain embodiments the host cells are HEK 293 cells. In certain embodiments the host cells are human embryotic retinal cells. In certain embodiments the host cells are human amniocyte cells. In certain embodiments the host cells are mouse fibroblast cells. In certain embodiments the host cells are non-lytic insect cell expression systems. In certain embodiments the host cells are Sf9 cells. In certain embodiments the host cells are Sf21 cells. In certain embodiments the host cells are Hi-5 cells. In certain embodiments the host cells are Schneider 2 cells. In certain embodiments the host cells are Schneider 3 cells. In certain embodiments the host cells are Arabidopsis thaliana cells. In certain embodiments the host cells are Nicotiana benthamiana cells. In certain embodiments the host cells are Nicotiana tabacum cells. In certain embodiments the host cells are Medicago sativa cells. In certain embodiments the host cells are Lemna minor cells. In certain embodiments the host cells are Physcomitrella patens cells.

In certain embodiments, the above mentioned host cells and organisms may be modified (genetically or in other ways) to change the native organism’s glycosylation, for example to obtain a glycosylation that better resembles or is identical to the glycosylation present in mammals, such as in humans.

In certain embodiments the expression host cells are modified to produce a specific glycosylation pattern or a more homogenous glycosylation pattern, for example by deleting or overexpressing certain glycosyltransferases, by introducing glycosyltransferases from other organisms or by introducing modified glycosyltransferases with altered substrate and/or acceptor specificities. The expression host cells may in certain embodiments be modified to synthesize specific carbohydrate residues not naturally present in the host cell.

One way to increase the yield of a secreted protein of interest is to improve the mechanism of cleaving off the signal or leader sequence directing the protein for secretion. Correct processing of the signal or leader sequence is a crucial step in the secretion pathway, as it liberates the N-terminus of the mature secreted protein and is usually required to achieve efficient secretion. Incomplete cleavage of the signal or leader sequence typically leads to intracellular accumulation of protein, although in some cases, incompletely processed product may be secreted as well.

In most expression systems, secretion is guided by a secretion signal peptide which is fused to the N-terminus of the protein to be secreted, and which is cleaved off by specific processing enzymes of the host cell, prior to or in conjunction with secretion. Accordingly, the IL-2 protein of formula (I) is in certain embodiments expressed with a secretion signal peptide, which is cleaved off by specific processing enzymes of the host cell, prior to or in conjunction with the secretion.

In mammalian expression systems, the signal peptide is in certain embodiments the signal peptide of any naturally secreted protein. In certain embodiments the signal peptide for mammalian expression systems is in certain embodiments thus the signal peptide of a naturally secreted protein. In certain embodiments the signal peptide for mammalian expression systems is a non-natural synthetic signal sequenced designed in silico or experimentally found to efficiently guide secretion. In certain embodiments the signal peptide has a sequence selected from the group consisting of SEQ ID NO:238, SEQ ID NO:239 and SEQ ID NO:240. In certain embodiments the signal peptide has the sequence of SEQ ID NO:238. In certain embodiments the signal peptide has the sequence of SEQ ID NO:239. In certain embodiments the signal peptide has the sequence of SEQ ID NO:240.

In certain embodiments the IL-2 protein of formula (I) with a signal peptide has a sequence selected form the group consisting of SEQ ID NO:248, SEQ ID NO:250 and SEQ ID NO:252. In certain embodiments the IL-2 protein of formula (I) with a signal peptide has the sequence of SEQ ID NO:248. In certain embodiments the IL-2 protein of formula (I) with a signal peptide has the sequence of SEQ ID NO:250. In certain embodiments the IL-2 protein of formula (I) with a signal peptide has the sequence of SEQ ID NO:252.

In E. coli, the signal sequence guiding the protein to periplasmic secretion can be the signal peptide of any bacterial naturally secreted to the periplasm. In certain embodiments the signal peptide for expression of the IL-2 protein of formula (I) in E. coli is selected from the group consisting of phoA, dsbA, gllI, mal, OmpA, OmpC, OmpT, pelB, torA, torT, EOX, STII, SfmC, lamB, MglB, MmAp, and tolB. In certain embodiments the signal peptide is a non-natural sequence designed in silico, or experimentally found to guide secretion efficiently.

In yeast expression systems, such as S. cerevisiae and Pichia pastoris, the leader sequence guiding expression may comprise a signal sequence and a propeptide, whereof the signal sequence guides the protein to be secreted to the ER and is cleaved off in conjunction with transport into the ER, and the propeptide is cleaved off in the Golgi apparatus by the Kex2 enzyme prior to secretion into the growth medium. The leader sequence may be the leader sequence of a naturally secreted enzyme or pheromone. In certain embodiments the leader sequence of the IL-2 protein of formula (I) for expression in a yeast expression system is thus selected from the group consisting of the S. cerevisiae mating factor Alpha leader sequence, the SUC2 leader sequence and the VOA1 leader sequence. In certain embodiments the leader sequence is from a secreted protein from another yeast or filamentous fungus, or it may be a non-natural leader sequence designed in silico, or it may be a leader sequence experimentally found to efficiently guide folding and secretion. The leader sequence may also have been experimentally identified form a large library of leader sequences, e.g. comprising many combinations of random amino acid substitutions.

Correct cleavage of the signal or leader sequence by the endogenous processing enzymes of the host cell is dependent on the sequence of amino acids immediately following the cleavage site, which constitute the N-terminus of the mature processed and secreted recombinant protein. In addition to the specific N-terminal amino acid sequence of the protein of interest, the accessibility of the N-terminus in the folded protein of interest may influence how efficiently the signal sequence or leader is processed. For example, a buried N-terminus may be inaccessible to the processing protease and will therefore be problematic for a secretion strategy.

Using prediction models built on available experimental data, the probability of cleavage of a certain amino acid sequence by the signal peptidase complex can be calculated. Such tools are available online, allowing a person skilled in the art to predict the likelihood of correct processing of the signal peptide in eukarya and bacteria. In yeast expression systems, the leader sequence typically comprises both a signal sequence, cleaved by the signal peptidase complex in the ER, and a propeptide, cleaved by a Kex2 furin protease in the Golgi. The recognition site for Kex2, KR, is well conserved among Kex2 substrates across yeast species. It is known that negatively charged amino acids are overrepresented in the P1′, P2′ and P4′ positions of Kex2 substrates. However, potential cleavage by Kex2 typically needs to be experimentally examined on a case-to-case basis.

It is well known to a person of ordinary skills in the art that correct processing of the signal or leader sequence is one of several features required for efficient secretion of correctly folded and soluble protein. Examples of important features are adequate rates of transcription and translation, co- or post translational translocation into the ER, folding and formation of correct disulfide bridges in the ER, and vesicular transport out of the cell. Experimental verification of any computer-aided prediction of secretion efficiency is therefore of essence.

It is known that intracellular accumulation of incorrectly folded or aggregated protein may negatively affect the physiology of the host cell, potentially inducing stress responses and causing decreased growth rate and cell fitness. Therefore, avoiding intracellular accumulation by improving processing of the signal or leader sequence, may result in increased growth rates, cell densities and cell mass productivity, positively contributing to the overall productivity of the protein of interest. In addition, a more fit cell line is more likely to be performing robustly across scales and cultivation conditions and better cope with process disturbances. Furthermore, it is generally recognized by persons skilled in the art that cell lines with normal growth rates and cell fitness have lower risk of instability than cell lines with reduced growth rates and cell fitness resulting from effects of transgene expression, such as intracellular accumulation of product. For a cell line with reduced growth rate conferred by transgene expression, the occurrence of an event that reduces transgene expression (e.g. a gene silencing event, mutation, or looping out of transgenes through direct-repeat recombination) results in a competitive growth advantage. Cells with reduced expression will rapidly outcompete other cells in the population still expressing the transgene at high levels, resulting in an unstable expression phenotype.

In certain embodiments the host cells expressing one or more genes encoding the IL-2 protein of formula (I) may comprise the one or more genes encoding for the IL-2 protein of formula (I) within their genome.

In another aspect the present invention relates to a conjugate comprising one or more of the IL-2 proteins of formula (I).

In certain embodiments said conjugate comprises at least one, such as one, two, three or four, moiety M_{mod} conjugated to the IL-2 protein of formula (I), which may be the same or different. Attachment of such moiety M_{mod} may be at the N-terminus, C-terminus, at an amino acid side chain or at an internal site of the IL-2 protein. In certain embodiments attachment of such moiety M_{mod} is at the N-terminus of the IL-2 protein of formula (I). In certain embodiments attachment of such moiety M_{mod} is at the C-terminus of the IL-2 protein of formula (I). In certain embodiments attachment of such moiety M_{mod} is at an internal site of the IL-2 moiety, such as at an amino acid side chain of the IL-2 protein of formula (I). If more than one moiety M_{mod} is attached to the IL-2 protein of formula (I), attachment may occur at any combination of attachment sites selected from the group consisting of the N-terminus, C-terminus and an internal site.

In certain embodiments M_{mod} is a substituent. Preferably, such substituent has a molecular weight ranging from 15 Da to 1 kDa.

In certain embodiments M_{mod} is a polymeric moiety. Such polymeric moiety may comprise a linear, branched or multi-arm polymer. In one embodiment the polymer is a linear polymer. In another embodiment the polymer is a branched polymer. Such branched polymer in certain embodiments has one, two, three, four or five branching points. From each branching point two, three or four polymer arms may extend. In another embodiment the polymer is a multi-arm polymer. Such multi-arm polymer may have 3, 4, 5, 6, 7 or 8 polymeric arms.

If M_{mod} is a polymeric moiety, such polymeric moiety in certain embodiments has a molecular weight ranging from 0.5 kDa to 1000 kDa, such as from 1 kDa to 1000 kDa, such as from 2 kDa to 500 kDa, from 3 kDa to 200 kDa, from 5 kDa to 120 kDa or from 7 to 40 kDa. In one embodiment such polymer has a molecular weight of about 0.5 kDa. In one embodiment such polymer has a molecular weight of about 1 kDa. In one embodiment such polymer has a molecular weight of about 2 kDa. In one embodiment such polymer has a molecular weight of about 3 kDa. In one embodiment such polymer has a molecular weight of about 4 kDa. In one embodiment such polymer has a molecular weight of about 5 kDa. In one embodiment such polymer has a molecular weight of about 7.5 kDa. In another embodiment such polymeric moiety has a molecular weight of about 10 kDa. In another embodiment such polymeric moiety has a molecular weight of about 15 kDa. In another embodiment such polymeric moiety has a molecular weight of about 20 kDa. In another embodiment such polymeric moiety has a molecular weight of about 30 kDa. In another embodiment such polymeric moiety has a molecular weight of about 40 kDa. In another embodiment such polymeric moiety has a molecular weight of about 50 kDa. In another embodiment such polymeric moiety has a molecular weight of about 70 kDa. In another embodiment such polymeric moiety has a molecular weight of about 80 kDa. In another embodiment such polymeric moiety has a molecular weight of about 90 kDa. In another embodiment such polymeric moiety has a molecular weight of about 100 kDa. In one embodiment such polymer has a molecular weight of 0.5 kDa. In one embodiment such polymer has a molecular weight of 1 kDa. In one embodiment such polymer has a molecular weight of 2 kDa. In one embodiment such polymer has a molecular weight of 3 kDa. In one embodiment such polymer has a molecular weight of 4 kDa. In one embodiment such polymer has a molecular weight of 5 kDa. In one embodiment such polymer has a molecular weight of 7.5 kDa. In another embodiment such polymeric moiety has a molecular weight of 10 kDa. In another embodiment such polymeric moiety has a molecular weight of 15 kDa. In another embodiment such polymeric moiety has a molecular weight of 20 kDa. In another embodiment such polymeric moiety has a molecular weight of 30 kDa. In another embodiment such polymeric moiety has a molecular weight of 40 kDa. In another embodiment such polymeric moiety has a molecular weight of 50 kDa. In another embodiment such polymeric moiety has a molecular weight of 70 kDa. In another embodiment such polymeric moiety has a molecular weight of 80 kDa. In another embodiment such polymeric moiety has a molecular weight of 90 kDa. In another embodiment such polymeric moiety has a molecular weight of 100 kDa.

If M_{mod} is a polymeric moiety, such polymeric moiety preferably comprises a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, alginate, mannans, pectins, rhamnogalacturonans, starches, hydroxyalkyl starches, hydroxyethyl starches and other carbohydrate-based polymers, xylans, and copolymers thereof.

In certain embodiments M_{mod} is a PEG-based polymer.

In certain embodiments M_{mod} is a hyaluronic acid-based polymer.

In certain embodiments M_{mod} comprises a peptide or protein moiety, which may be chemically conjugated to the IL-2 protein of formula (I). Preferably, this peptide or protein moiety M_{mod} is not a fragment of IL-2 or an IL-2 moiety.

M_{mod} in the form of a peptide or protein moiety may be a synthetic or natural protein moiety or a portion or variant thereof. Exemplary peptides and proteins include albumin; antibody domains, such as Fc domains or antigen binding domains of immunoglobulins; CTP, and CD25; each either in their naturally occurring form or as a variant or fragment thereof.

Attachment of M_{mod} to the IL-2 protein of formula (I) may be via a stable linkage.

In certain embodiments M_{mod} comprises a targeting moiety which is selected from the group consisting of antibodies, antibody fragments, affibodies, affilins, affimers, affitins, alphamabs, alphabodies, anticalins, avimers, DARPins, Fynomers®, Kunitz domain peptides, monobodies, nanoCLAMPs, cyclic peptides, peptides, heavy chain only antibodies, VHH antibodies or Nanobodies®, single chain variable Fragments (scFvs), natural or modified peptide or protein receptor ligands and small molecule inhibitors.

In certain embodiments this conjugate is an IL-2 conjugate or a pharmaceutically acceptable salt thereof of formula (Ia) or (Ib)

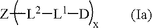

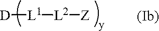

wherein
D comprises the IL-2 protein of formula (I);
L¹- is a linker moiety covalently and reversibly attached to -D;
L²- is a chemical bond or is a spacer moiety;
Z is a polymeric moiety or a substituted fatty acid moiety;
x is an integer selected from the group consisting of 1, 2, 3, 4, 5, 6, 7, 8, 9, 10, 11, 12, 13, 14, 15 and 16; and
y is an integer selected from the group consisting of 2, 3, 4 and 5.

In certain embodiments the conjugates of formula (Ia) and (Ib) release a biased IL-2 moiety or biased IL-2 protein of formula (I), wherein the ratio of Ratio_{biased} _{IL-2} to Ratio_{IL-2} _{of} _{SEQ} _{ID} _{NO:213} is larger than 1, preferably larger than 2, preferably larger then 3, preferably larger than 4 and even more preferably larger than 5. In certain embodiments the ratio of Ratio_{biased} _{IL-2} to Ratio_{IL-} ₂ of _{SEQ} _{ID} _{NO:213} is larger than 10, larger than 20, larger than 50, larger than 70, larger than 100, larger than 150 or larger than 200.

It is understood that -D of formula (Ia) and (Ib) may comprise at least one moiety M_{mod} as described elsewhere herein.

The IL-2 conjugate of formula (Ia) or (Ib) comprises at least one covalently and reversibly attached polymeric moiety and/or substituted fatty acid moiety -Z.

The addition of such at least one covalently and reversibly attached polymeric moiety and/or substituted fatty acid moiety is capable of extending the circulation half-life of the IL-2 moiety of formula (I), while its reversible attachment ensures sufficient pharmaceutical activity.

In certain embodiments the IL-2 conjugate is of formula (Ia) and comprises one moiety -Z, which is either a substituted fatty acid or a polymeric moiety. In certain embodiments -Z is a substituted fatty acid. In certain embodiments -Z is a polymeric moiety.

In certain embodiments the IL-2 conjugate is of formula (Ib) and comprises two moieties -Z, which may be the same or different. In certain embodiments both moieties -Z are a substituted fatty acid, which may be the same or different. In certain embodiments both moieties -Z are a polymeric moiety, which may be the same or different. In certain embodiments one moiety -Z is a substituted fatty acid and the other moiety -Z is a polymeric moiety.

In certain embodiments the IL-2 conjugate of is of formula (Ib) and comprises three moieties -Z, which may be the same or different. In certain embodiments all three moieties -Z are a substituted fatty acid, which may be the same or different. In certain embodiments all three moieties -Z are a polymeric moiety, which may be the same or different. In certain embodiments one or two moieties -Z are a substituted fatty acid and the remaining moiety/moieties -Z is/are a polymeric moiety.

In certain embodiments the IL-2 conjugate is of formula (Ib) and comprises four moieties -Z, which may be the same or different. In certain embodiments all four moieties -Z are a substituted fatty acid, which may be the same or different. In certain embodiments all four moieties -Z are a polymeric moiety, which may be the same or different. In certain embodiments one, two or three moieties -Z are a substituted fatty acid and the remaining moiety/moieties -Z is/are a polymeric moiety.

If -Z is a substituted fatty acid moiety it is preferably a substituted fatty acid moiety disclosed in WO 2005/027978 A2 and WO 2014/060512 A1, which are herewith incorporated by reference.

If -Z is a polymeric moiety, such polymeric moiety has in certain embodiments a molecular weight ranging from 1 kDa to 1000 kDa, such as from 2 kDa to 500 kDa, from 3 kDa to 200 kDa, from 5 kDa to 120 kDa, from 10 kDa to 100 kDa or from 15 kDa to 80 kDa. In certain embodiments -Z is a polymeric moiety having a molecular weight of about 2 kDa. In certain embodiments -Z is a polymeric moiety having a molecular weight of about 5 kDa. In certain embodiments -Z is a polymeric moiety having a molecular weight of about 10 kDa. In certain embodiments -Z is a polymeric moiety having a molecular weight of about 15 kDa. In certain embodiments -Z is a polymeric moiety having a molecular weight of about 20 kDa. In certain embodiments -Z is a polymeric moiety having a molecular weight of about 30 kDa. In certain embodiments -Z is a polymeric moiety having a molecular weight of about 40 kDa. In certain embodiments -Z is a polymeric moiety having a molecular weight of about 50 kDa. In certain embodiments -Z is a polymeric moiety having a molecular weight of about 60 kDa. In certain embodiments -Z is a polymeric moiety having a molecular weight of about 70 kDa. In certain embodiments -Z is a polymeric moiety having a molecular weight of about 80 kDa. In certain embodiments -Z is a polymeric moiety having a molecular weight of about 90 kDa. In certain embodiments -Z is a polymeric moiety having a molecular weight of about 100 kDa. In certain embodiments -Z is a polymeric moiety having a molecular weight of 2 kDa. In certain embodiments -Z is a polymeric moiety having a molecular weight of 5 kDa. In certain embodiments -Z is a polymeric moiety having a molecular weight of 10 kDa. In certain embodiments -Z is a polymeric moiety having a molecular weight of 15 kDa. In certain embodiments -Z is a polymeric moiety having a molecular weight of 20 kDa. In certain embodiments -Z is a polymeric moiety having a molecular weight of 30 kDa. In certain embodiments -Z is a polymeric moiety having a molecular weight of 40 kDa. In certain embodiments -Z is a polymeric moiety having a molecular weight of 50 kDa. In certain embodiments -Z is a polymeric moiety having a molecular weight of 60 kDa. In certain embodiments -Z is a polymeric moiety having a molecular weight of 70 kDa. In certain embodiments -Z is a polymeric moiety having a molecular weight of 80 kDa. In certain embodiments -Z is a polymeric moiety having a molecular weight of 90 kDa. In certain embodiments -Z is a polymeric moiety having a molecular weight of 100 kDa.

In certain embodiments -Z is a polymeric moiety comprising a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, alginate, mannans, pectins, rhamnogalacturonans, starches, hydroxyalkyl starches, hydroxyethyl starches and other carbohydrate-based polymers, xylans, and copolymers thereof.

In certain embodiments -Z is a peptide or protein moiety. Preferably, such peptide or protein moiety is not an IL-2 moiety or fragment thereof. Such peptide or protein moiety -Z may be chemically conjugated to -D via -L¹-L²- or may be translationally fused to -D via a reversible linker moiety -L¹-, in which case -L¹- is a peptide or protein moiety and -L²- is preferably a chemical bond. In certain embodiments such peptide or protein moiety -Z is chemically conjugated to -D via -L¹-L²-. In certain embodiments such peptide or protein moiety -Z is translationally fused to -D via a reversible linker moiety -L¹-, in which case -L¹- is a peptide or protein moiety and -L²- is preferably a chemical bond. It is understood that such peptide or protein reversible linker moiety -L¹- may be enzymatically or non-enzymatically degradable. To facilitate enzymatic degradation -L¹- may comprise a protease recognition site.

If -Z is a peptide or protein moiety it is in certain embodiments selected from the group consisting of moieties comprising the carboxyl-terminal peptide of the chorionic gonadotropin as described in US 2012/0035101 A1, which are herewith incorporated by reference; albumin moieties; random coil protein moieties and Fc fusion protein moieties.

In certain embodiments -Z comprises a random coil peptide or protein moiety.

In certain embodiments such random coil peptide or protein moiety comprises at least 25 amino acid residues and at most 2000 amino acids, such as 30 amino to 1500 amino acid residues or 50 to 500 amino acid residues.

In certain embodiments -Z comprises a random coil protein moiety of which at least 80%, such as at least 85%, at least 90%, at least 95%, at least 98% or at least 99%, of the total number of amino acids forming said random coil protein moiety are selected from alanine and proline. In certain embodiments at least 10%, but less than 75%, such as less than 65%, of the total number of amino acid residues of such random coil protein moiety are proline residues. In certain embodiments such random coil protein moiety is as described in WO 2011/144756 A1, which is hereby incorporated by reference in its entirety. In certain embodiments -Z comprises at least one moiety selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:51 and SEQ ID NO:61 as disclosed in WO2011/144756. A moiety comprising such random coil protein comprising alanine and proline is referred to herein as “PA” or “PA moiety”.

Accordingly, in certain embodiments -Z comprises a PA moiety.

In certain embodiments -Z comprises a random coil protein moiety of which at least 80%, such as at least 85%, at least 90%, at least 95%, at least 98% or at least 99% of the total number of amino acids forming said random coil protein moiety are selected from alanine, serine and proline. In certain embodiments at least 4%, but less than 40% of the total number of amino acid residues of such random coil protein moiety are proline residues. In certain embodiments such random coil protein moiety is as described in WO 2008/155134 A1, which is hereby incorporated by reference. In certain embodiments -Z comprises at least one moiety selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:40, SEQ ID NO:42, SEQ ID NO:44, SEQ ID NO:46, SEQ ID NO:50, SEQ ID NO:52, SEQ ID NO:54 and SEQ ID NO:56 as disclosed in WO 2008/155134 A1. A moiety comprising such random coil protein moiety comprising alanine, serine and proline is referred to herein as “PAS” or “PAS moiety”.

Accordingly, in certain embodiments -Z comprises a PAS moiety.

In certain embodiments -Z comprises a random coil protein moiety of which at least 80%, such as at least 85%, at least 90%, at least 95%, at least 98% or at least 99% of the total number of amino acids forming said random coil protein moiety are selected from alanine, glycine, serine, threonine, glutamate and proline. In certain embodiments such random coil protein moiety is as described in WO 2010/091122 A1, which is hereby incorporated by reference. In certain embodiments -Z comprises at least one moiety selected from the group consisting of SEQ ID NO:182, SEQ ID NO:183, SEQ ID NO:184; SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:759, SEQ ID NO:760, SEQ ID NO:761, SEQ ID NO:762, SEQ ID NO:763, SEQ ID NO:764, SEQ ID NO:765, SEQ ID NO:766, SEQ ID NO:767, SEQ ID NO:768, SEQ ID NO:769, SEQ ID NO:770, SEQ ID NO:771, SEQ ID NO:772, SEQ ID NO:773, SEQ ID NO:774, SEQ ID NO:775, SEQ ID NO:776, SEQ ID NO:777, SEQ ID NO:778, SEQ ID NO:779, SEQ ID NO:1715, SEQ ID NO:1716, SEQ ID NO:1718, SEQ ID NO:1719, SEQ ID NO:1720, SEQ ID NO:1721 and SEQ ID NO:1722 as disclosed in WO2010/091122A1. A moiety comprising such random coil protein moiety comprising alanine, glycine, serine, threonine, glutamate and proline is referred to herein as “XTEN” or “XTEN moiety”.

Accordingly, in certain embodiments -Z comprises an XTEN moiety.

In certain embodiments -Z is a hyaluronic acid-based polymer.

In certain embodiments -Z is a PEG-based moiety, such as a linear, branched or multi-arm PEG-based moiety. In certain embodiments -Z is a branched PEG-based moiety, such as a branched PEG-based moiety having one, two, three, four, five or six branching points. In certain embodiments -Z is a branched PEG-based moiety having one, two or three branching points. In certain embodiments -Z is a branched PEG-based moiety having one branching point. In certain embodiments -Z is a branched PEG-based moiety having two branching points. In certain embodiments -Z is a branched PEG-based moiety having three branching points.

Each branching point may be independently selected from the group consisting of —N<, —CH< and >C<.

In certain embodiments -Z comprises a moiety of formula (A)

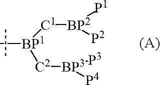

wherein
—BP¹<, —BP²<, —BP³< are independently of each other selected from the group consisting of —N< and —C(R⁸)<;
R⁸ is selected from the group consisting of H, C₁₋₆ alkyl, C₂₋₆ alkenyl and C₂₋₆ alkynyl;
—P¹, —P², —P³, —P⁴ are independently of each other a PEG-based chain comprising at least 40% PEG and having a molecular weight ranging from 3 to 40 kDa;
—C¹—, —C²— are independently of each other selected from the group consisting of C₁₋₅₀ alkyl, C₂₋₅₀ alkenyl, and C₂₋₅₀ alkynyl; wherein C₁₋₅₀ alkyl, C₂₋₅₀ alkenyl, and C₂₋ ₅₀ alkynyl are optionally substituted with one or more R⁹, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R¹⁰)—, —S(O)₂N(R¹⁰)—, —S(O)N(R¹⁰)—, —S(O)₂—, —S(O)—, —N(R¹⁰)S(O)₂N(R^{10a})—, —S—, —N(R¹⁰)—, —OC(OR¹⁰)(R^{10a})—, —N(R¹⁰)C(O)N(R^{10a})—, and —OC(O)N(R¹⁰)—;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8-to 30-membered carbopolycyclyl, and 8- to 30-membered heteropolycyclyl; wherein each T is independently optionally substituted with one or more R⁹, which are the same or different;
each R⁹ is independently selected from the group consisting of halogen, —CN, oxo (=O), -COOR¹¹, -OR¹¹, —C(O)R¹¹, —C(O)N(R¹¹R^{11a}), —S(O)₂N(R¹¹R^{11a}), —S(O)N(R¹¹R^{11a}), —S(O)₂R¹¹, —S(O)R¹¹, —N(R¹¹)S(O)₂N(R^{11a}R^{11b}), -SR¹¹, —N(R¹¹R^{11a}), —NO₂, —OC(O)R¹¹, —N(R¹¹)C(O)R^{11a}, —N(R¹¹)S(O)₂R^{11a}, —N(R¹¹)S(O)R^{11a}, —N(R¹¹)C(O)OR^{11a}, —N(R¹¹)C(O)N(R^{11a}R^{11b}), —OC(O)N(R¹¹R^{11a}), and C₁₋₆ alkyl; wherein C₁₋₆ alkyl is optionally substituted with one or more halogen, which are the same or different; and
each R¹⁰, R^{10a}, R¹¹, R^{11a} and R^{11b} is independently selected from the group consisting of —H, and C₁₋₆ alkyl, wherein C₁₋₆ alkyl is optionally substituted with one or more halogen, which are the same or different.

In certain embodiments —P¹, —P², —P³, —P⁴ are independently of each other a PEG-based chain comprising at least 50% PEG and having a molecular weight ranging from 3 to 40 kDa. In certain embodiments —P¹, —P², —P³, —P⁴ are independently of each other a PEG-based chain comprising at least 60% PEG and having a molecular weight ranging from 3 to 40 kDa. In certain embodiments —P¹, —P², —P³, —P⁴ are independently of each other a PEG-based chain comprising at least 70% PEG and having a molecular weight ranging from 3 to 40 kDa. In certain embodiments —P¹, —P², —P³, —P⁴ are independently of each other a PEG-based chain comprising at least 80% PEG and having a molecular weight ranging from 3 to 40 kDa.

In certain embodiments the molecular weight of a moiety —P¹, —P², —P³ and —P⁴ of formula (A) ranges independently of each other from 5 to 30 kDa, such as from 5 to 25 kDa or from 8 to 20 kDa. In certain embodiments the molecular weight of a moiety —P¹, —P², —P³ or —P⁴ may be about 5 kDa. In certain embodiments the molecular weight of a moiety —P¹, —P², —P³ or —P⁴ may be about 7 kDa. In certain embodiments the molecular weight of a moiety —P¹, —P², —P³ or —P⁴ may be about 10 kDa. In certain embodiments the molecular weight of a moiety —P¹, —P², —P³ or -P⁴ may be about 12 kDa. In certain embodiments the molecular weight of a moiety —P¹, —P², —P³ or -P⁴ may be about 15 kDa. In certain embodiments the molecular weight of a moiety —P¹, —P², —P³ or —P⁴ may be about 20 kDa. In certain embodiments the molecular weight of a moiety —P¹, —P², —P³ or —P⁴ may be about 25 kDa. In certain embodiments the molecular weight of a moiety -P¹, —P², —P³ or —P⁴ may bₑ _{about} 30 kDa. In certain embodiments the molecular weight of a moiety —P¹, —P², —P³ or —P⁴ may be 7 kDa. In certain embodiments the molecular weight of a moiety —P¹, —P², —P³ or —P⁴ may be 10 kDa. In certain embodiments the molecular weight of a moiety —P¹, —P², —P³ or —P⁴ may be 12 kDa. In certain embodiments the molecular weight of a moiety —P¹, —P², —P³ or —P⁴ may be 15 kDa. In certain embodiments the molecular weight of a moiety —P¹, —P², —P³ or —P⁴ may be 20 kDa. In certain embodiments the molecular weight of a moiety —P¹, —P², —P³ or —P⁴ may be 25 kDa. In certain embodiments the molecular weight of a moiety —P¹, —P², —P³ or —P⁴ may be 30 kDa.

In certain embodiments —P¹, —P², —P³ and —P⁴ of formula (A) have the same structure.

In certain embodiments —BP¹< of formula (A) is —N<.

In certain embodiments —BP²< and —BP³< of formula (A) have the same structure. In certain embodiments —BP²< and —BP³< of formula (A) are both —CH<.

In certain embodiments —C^{l}— and —C²— of formula (A) have the same structure. In certain embodiments —C^{l}— and —C²— of formula (A) are C₁₋₅₀ alkyl interrupted by one or more of the groups selected from the group consisting of —O—, —C(O)N(R¹⁰)— and 3- to 10-membered heterocyclyl; wherein the 3- to 10-membered heterocyclyl is substituted with at least one oxo (=O).

In certain embodiments —C¹— and —C²— of formula (A) are of formula (A-a)

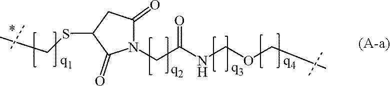

wherein
the dashed line marked with the asterisk indicates attachment to —BP′<;
the unmarked dashed line indicates attachment to —BP²< or —BP³<, respectively;
q1 is selected from the group consisting of 1, 2, 3, 4, 5, 6, 7 and 8;
q2 is selected from the group consisting of 1, 2, 3, 4, and 5;
q3 is selected from the group consisting of 1, 2, 3, 4, 5, 6, 7 and 8; and
q4 is selected from the group consisting of 1, 2 and 3.

In certain embodiments q1 of formula (A-a) is selected from the group consisting of 4, 5, 6, 7, and 8. In certain embodiments q1 of formula (A-a) is selected from the group consisting of 5, 6 and 7. In certain embodiments q1 of formula (A-a) is 1. In certain embodiments q1 of formula (A-a) is 2. In certain embodiments q1 of formula (A-a) is 3. In certain embodiments q1 of formula (A-a) is 4. In certain embodiments q1 of formula (A-a) is 5. In certain embodiments q1 of formula (A-a) is 6. In certain embodiments q1 of formula (A-a) is 7. In certain embodiments q1 of formula (A-a) is 8.

In certain embodiments q2 of formula (A-a) is selected from the group consisting of 1, 2 and 3. In certain embodiments q2 of formula (A-a) is 1. In certain embodiments q2 of formula (A-a) is 2. In certain embodiments q2 of formula (A-a) is 3. In certain embodiments q2 of formula (A-a) is 4. In certain embodiments q2 of formula (A-a) is 5.

In certain embodiments q3 of formula (A-a) is selected from the group consisting of 2, 3, 4, and 5. In certain embodiments q3 of formula (A-a) is selected from the group consisting of 2, 3 and 4. In certain embodiments q3 of formula (A-a) is 1. In certain embodiments q3 of formula (A-a) is 2. In certain embodiments q3 of formula (A-a) is 3. In certain embodiments q3 of formula (A-a) is 4. In certain embodiments q3 of formula (A-a) is 5. In certain embodiments q3 of formula (A-a) is 6. In certain embodiments q3 of formula (A-a) is 7. In certain embodiments q3 of formula (A-a) is 8.

In certain embodiments q4 of formula (A-a) is 1. In certain embodiments q4 of formula (A-a) is 2. In certain embodiments q4 of formula (A-a) is 3.

In certain embodiments —P¹, —P², —P³ and —P⁴ of formula (A) are independently of each other of formula (A-b)

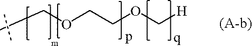

wherein
the dashed line indicates attachment to the remainder of -Z;
m is 0 or 1;
p is an integer ranging from 70 to 900; and
q is selected from the group consisting of 1, 2, 3, 4, 5, and 6.

In certain embodiments m of formula (A-b) is 0. In certain embodiments m of formula (A-b) is 1.

In certain embodiments p of formula (A-b) is an integer ranging from 115 to 680. In certain embodiments p of formula (A-b) is an integer ranging from 115 to 560. In certain embodiments p of formula (A-b) is an integer ranging from 185 to 450. In certain embodiments p of formula (A-b) is about 115. In certain embodiments p of formula (A-b) is about 160. In certain embodiments p of formula (A-b) is about 225. In certain embodiments p of formula (A-b) is about 270. In certain embodiments p of formula (A-b) is about 340. In certain embodiments p of formula (A-b) is about 450. In certain embodiments p of formula (A-b) is about 560.

In certain embodiments q of formula (A-b) is 1. In certain embodiments q of formula (A-b) is 2. In certain embodiments q of formula (A-b) is 3. In certain embodiments q of formula (A-b) is 4. In certain embodiments q of formula (A-b) is 5. In certain embodiments q of formula (A-b) is 6.

In certain embodiments -Z comprises a moiety of formula (A-c):

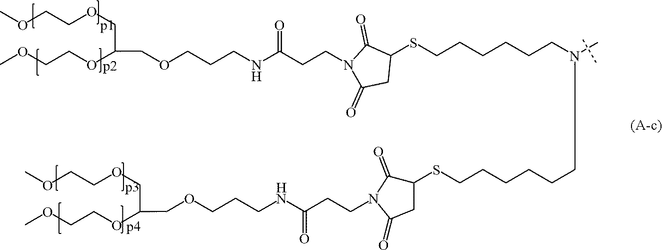

wherein p1, p2, p3, p4 are independently of each other an integer ranging from 70 to 900.

In certain embodiments p1 of formula (A-c) is an integer ranging from 115 to 680. In certain embodiments p1 of formula (A-c) is an integer ranging from 115 to 560. In certain embodiments p1 of formula (A-c) is an integer ranging from 185 to 450. In certain embodiments p1 of formula (A-c) is an integer ranging from 220 to 240. In certain embodiments p1 of formula (A-c) is about 115. In certain embodiments p1 of formula (A-c) is about 160. In certain embodiments p1 of formula (A-c) is about 225. In certain embodiments p1 of formula (A-c) is about 270. In certain embodiments p1 of formula (A-c) is about 340. In certain embodiments p1 of formula (A-c) is about 450. In certain embodiments p1 of formula (A-c) is about 560.

In certain embodiments p2 of formula (A-c) is an integer ranging from 115 to 680. In certain embodiments p2 of formula (A-c) is an integer ranging from 115 to 560. In certain embodiments p2 of formula (A-c) is an integer ranging from 185 to 450. In certain embodiments p2 of formula (A-c) is an integer ranging from 220 to 240. In certain embodiments p2 of formula (A-c) is about 115. In certain embodiments p2 of formula (A-c) is about 160. In certain embodiments p2 of formula (A-c) is about 225. In certain embodiments p2 of formula (A-c) is about 270. In certain embodiments p2 of formula (A-c) is about 340. In certain embodiments p2 of formula (A-c) is about 450. In certain embodiments p2 of formula (A-c) is about 560.

In certain embodiments p3 of formula (A-c) is an integer ranging from 115 to 680. In certain embodiments p3 of formula (A-c) is an integer ranging from 115 to 560. In certain embodiments p3 of formula (A-c) is an integer ranging from 185 to 450. In certain embodiments p3 of formula (A-c) is an integer ranging from 220 to 240. In certain embodiments p3 of formula (A-c) is about 115. In certain embodiments p3 of formula (A-c) is about 160. In certain embodiments p3 of formula (A-c) is about 225. In certain embodiments p3 of formula (A-c) is about 270. In certain embodiments p3 of formula (A-c) is about 340. In certain embodiments p3 of formula (A-c) is about 450. In certain embodiments p3 of formula (A-c) is about 560.

In certain embodiments p4 of formula (A-c) is an integer ranging from 115 to 680. In certain embodiments p4 of formula (A-c) is an integer ranging from 115 to 560. In certain embodiments p4 of formula (A-c) is an integer ranging from 185 to 450. In certain embodiments p4 of formula (A-c) is an integer ranging from 220 to 240. In certain embodiments p4 of formula (A-c) is about 115. In certain embodiments p4 of formula (A-c) is about 160. In certain embodiments p4 of formula (A-c) is about 225. In certain embodiments p4 of formula (A-c) is about 270. In certain embodiments p4 of formula (A-c) is about 340. In certain embodiments p4 of formula (A-c) is about 450. In certain embodiments p4 of formula (A-c) is about 560.

In certain embodiments p1, p2, p3 of formula (A-c) and p4 are identical. In certain embodiments p1, p2, p3 and p4 range from 220 to 240.

In certain embodiments -Z is a moiety as disclosed in WO 2012/02047 A1, which is herewith incorporated by reference.

In certain embodiments -Z is a moiety as disclosed in WO 2013/024048 A1, which is herewith incorporated by reference.

In certain embodiments the conjugate comprising one or more of the IL-2 proteins of formula (I) or a pharmaceutically acceptable salt thereof comprises a plurality of moieties -D, which are said IL-2 proteins of formula (I), conjugated via at least one moiety -L¹-L²- to at least one moiety Z′, wherein a moiety -L¹- is conjugated to -D via a reversible linkage and wherein a moiety -L²- is conjugated to Z′, wherein -L¹- and -L²- are used as defined for formula (Ia) and (Ib) and wherein Z′ is a water-insoluble hydrogel.

In certain embodiments such hydrogel Z′ comprises a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(alkylene glycols), such as poly(ethylene glycols) and poly(propylene glycol), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

In certain embodiments Z′ is a poly(alkylene glycol)-based or hyaluronic acid-based hydrogel.

In certain embodiments Z′ is a poly(propylene glycol)-based hydrogel.

In certain embodiments Z′ is a PEG-based hydrogel.

In certain embodiments Z′ is a PEG-based hydrogel as disclosed in WO2011/012715A1 or WO2014/056926A1, which are herewith incorporated by reference.

In certain embodiments Z′ is a hyaluronic acid-based hydrogel.

In certain embodiments Z′ is a hyaluronic acid-based hydrogel as disclosed in WO2018/175788A1, which is herewith incorporated by reference.

In certain embodiments Z′ is a hydrogel as disclosed in WO2013/036847 A1. In particular, in certain embodiments Z′ is a hydrogel produced by a method comprising the step of reacting at least a first reactive polymer with a cleavable crosslinker compound, wherein said cleavable crosslinker compound comprises a first functional group -Y¹ that reacts with the first reactive polymer and further comprises a moiety that is cleaved by elimination under physiological conditions wherein said moiety comprises a second functional group -Y² that reacts with a second reactive polymer. In certain embodiments the cleavable crosslinker compound is of formula (PL-1)

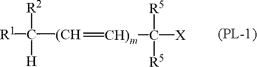

wherein
m is 0 or 1;
-X comprises a functional group capable of connecting to a reactive polymer that is amenable to elimination under physiological conditions and said second functional group -Y2;
at least one of -R¹, -R² and -R⁵ comprises said first functional group -Y¹ capable of connecting to a polymer;
one and only one of -R¹ and -R² is selected from the group consisting of —H, alkyl, arylalkyl, and heteroarylalkyl;
optionally, -R¹ and -R² may be joined to form a 3- to 8-membered ring;
at least one or both of -R¹ and -R² is independently selected from the group consisting of —CN, —NO₂, aryl, heteroaryl, alkenyl, alkynyl, -COR³, -SOR³, —SO₂R³ and —SR⁴;
-R³ is selected from the group consisting of —H, alkyl, aryl, arylalkyl, heteroaryl, heteroarylalkyl, —OR⁹ and -NR⁹₂;
-R⁴ is selected from the group consisting of alkyl, aryl, arylalkyl, heteroaryl and heteroarylalkyl;
each -R⁵ is independently selected from the group consisting of —H, alkyl, alkenylalkyl, alkynylalkyl, (OCH₂CH₂)ₚO-alkyl with p being an integer ranging from 1 to 1000, aryl, arylalkyl, heteroaryl and heteroarylalkyl;
each -R⁹ is independently selected from the group consisting of —H and alkyl or both -R⁹ together with the nitrogen to which they are attached form a heterocyclic ring;
and wherein the moiety of formula (PL-1) is optionally further substituted.

The following paragraphs describe such hydrogel in more detail.

In certain embodiments -X of formula (PL-1) is selected from the group consisting of succinimidyl carbonate, sulfosuccinimidyl carbonate halides, thioethers, esters, nitrophenyl carbonate, chloroformate, fluoroformate, optionally substituted phenols and formula (PL-2)

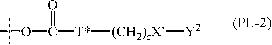

wherein
the dashed line indicates attachment to the remainder of formula (PL-1);
-T*- is selected from the group consisting of —O—, —S— and —NR⁶—;
z is an integer selected from the group consisting of 1, 2, 3, 4, 5 and 6;
-X′- is absent or is selected from the group consisting of —OR⁷— and —SR⁷—;
—Y² is a functional group capable of connecting with a reactive polymer;
-R⁶ is selected from the group consisting of —H, alkyl, aryl, heteroaryl, arylalkyl, and heteroarylalkyl; and
-R⁷ is selected from the group consisting of alkylene, phenylene and (OCH₂CH₂)ₚ, with p being an integer ranging from 1 to 1000.

In certain embodiments -X of formula (PL-1) comprises an activated carbonate such as succinimidyl carbonate, sulfosuccinimidyl carbonate, or nitrophenyl carbonate. In certain embodiments -X of formula (PL-1) comprises a carbonyl halide such as O(C═O)Cl or O(C═O)F. In certain embodiments -X of formula (PL-1) has the formula (PL-2). In certain embodiments -X of formula (PL-1) is OR⁷ or SR⁷, wherein R⁷ is optionally substituted alkylene, optionally substituted phenylene or (OCH₂CH₂)ₚ, wherein p is 1 to 1000.

In certain embodiments p of formula (PL-2) is an integer ranging from 1 to 100. In certain embodiments p of formula (PL-2) is an integer ranging from 1 to 10.

In certain embodiments —Y¹ of formula (PL-1) and —Y² of formula (PL-2) independently comprise N₃, NH₂, NH—CO₂^{t}Bu, SH, S^{t}Bu, maleimide, CO₂H, CO₂^{t}Bu, 1,3-diene, cyclopentadiene, furan, alkyne, cyclooctyne, acrylate or acrylamide, wherein ^{t}Bu is tert-butyl, and wherein when one of —Y¹ or —Y² comprises N₃ the other does not comprise alkyne or cyclooctyne; when one of —Y¹ or —Y² comprises SH the other does not comprise maleimide, acrylate or acrylamide; when one of —Y¹ or —Y² comprises NH₂ the other does not comprise CO₂H; when one of —Y¹ or —Y² comprises 1,3-diene or cyclopentadiene the other does not comprise furan.

In certain embodiments the cleavable crosslinker compound is of formula (PL-3)

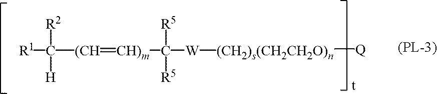

wherein
m is 0 or 1;
n is an integer selected from 1 to 1000;
s is 0, 1 or 2;
t is selected from the group consisting of 2, 4, 8, 16 and 32;
—W— is selected from the group consisting of —O(C═O)O—, —O(C═O)NH—, —O(C═O)S—, —O(C═O)NR⁶CH₂O— and —O(C═O)NR⁶S—;
-Q is a core group having a valency=t; which connects the multiple arms of the cleavable crosslinking compound,
   wherein t is an integer selected from 2, 4, 8, 16 and 32, and
   wherein -R¹, -R² and -R⁵ are defined as in formula (PL-1).

In certain embodiments t of formula (PL-3) is 2. In certain embodiments t of formula (PL-3) is 4. In certain embodiments t of formula (PL-3) is 8. In certain embodiments t of formula (PL-3) is 16. In certain embodiments t of formula (PL-3) is 32.

In certain embodiments -Q of formula (PL-3) has a structure selected from the group consisting of

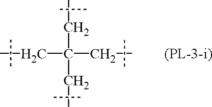

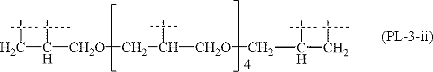

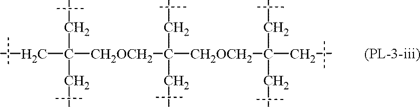

wherein the dashed lines indicate attachment to the remainder of the cleavable crosslinker compound.

In certain embodiments -Q of formula (PL-3) has the structure of (PL-3-i). In certain embodiments -Q of formula (PL-3) has the structure of (PL-3-ii). In certain embodiments -Q of formula (PL-3) has the structure of (PL-3-iii).

In certain embodiments the cleavable crosslinker compound is of formula (PL-3), wherein m is 0, n is approximately 100, s is 0, t is 4, —W— is —O(C═O)NH—, -Q has the structure of (PL-3i), -R² is H, one -R⁵ is —H and the other —R⁵ is (CH₂)₅N₃, and -R¹ is (4-chlorophenyl)SO₂, phenyl substituted with —SO₂, morpholino-SO₂, or —CN.

In certain embodiments —Y¹ of formula (PL-3) comprises N₃, NH₂, NH—CO₂^{t}Bu, SH, S^{t}Bu, maleimide, CO₂H, CO₂^{t}Bu, 1,3-diene, cyclopentadiene, furan, alkyne, cyclooctyne, acrylate or acrylamide, wherein ^{t}Bu is tert-butyl.

In certain embodiments each —Y¹ of formula (PL-1) or (PL-3) and —Y² of formula (PL-2) independently comprises N₃, NH₂, NH—CO₂^{t}Bu, SH, S^{t}Bu, maleimide, CO₂H, CO₂^{t}Bu, 1,3-diene, cyclopentadiene, furan, alkyne, cyclooctyne, acrylate or acrylamide.

In certain embodiments one of Y¹ and —Y² is azide and the other is a reactive functional group selected from the group consisting of acetylene, cyclooctyne, and maleimide. In certain embodiments one of Y^{l} and —Y² is thiol and the other is a reactive functional group selected from the group consisting of maleimide, acrylate, acrylamide, vinylsulfone, vinylsulfonamide, and halocarbonyl. In certain embodiments one of-Y¹ and —Y² is amine and the other is a selective reactive functional group selected from carboxylic acid and activated carboxylic acid. In certain embodiments one of-Y¹ and —Y² is maleimide and the other is a selective reactive functional group selected from the group consisting of 1,3-diene, cyclopentadiene, and furan.

In certain embodiments the first and any second polymer is selected from the group consisting of homopolymeric or copolymeric polyethylene glycols, polypropylene glycols, poly(N-vinylpyrrolidone), polymethacrylates, polyphosphazenes, polylactides, polyacrylamides, polyglycolates, polyethylene imines, agaroses, dextrans, gelatins, collagens, polylysines, chitosans, alginates, hyaluronans, pectins and carrageenans that either comprise suitable reactive functionalities or is of formula [Y³—(CH₂)ₛ(CH₂CH₂O)ₙ]ₜQ, wherein —Y³ is a reactive functional group, s is 0, 1 or 2, n is an integer selected from the group ranging from 10 to 1000, -Q is a core group having valency t, and t is an integer selected from the group consisting of 2, 4, 8, 16 and 32.

In certain embodiments the first polymer comprises a multi-arm polymer. In certain embodiments the first polymer comprises at least three arms. In certain embodiments the first polymer comprises at least four arms. In certain embodiments the first polymer comprises at least five arms. In certain embodiments the first polymer comprises at least six arms. In certain embodiments the first polymer comprises at least seven arms. In certain embodiments the first polymer comprises at least eight arms.

In certain embodiments the second polymer comprises a multi-arm polymer. In certain embodiments the second polymer comprises at least three arms. In certain embodiments the second polymer comprises at least four arms. In certain embodiments the second polymer comprises at least five arms. In certain embodiments the second polymer comprises at least six arms. In certain embodiments the second polymer comprises at least seven arms. In certain embodiments the second polymer comprises at least eight arms.

In certain embodiments the first polymer comprises a 2-arm polyethylene glycol polymer. In certain embodiments the first polymer comprises a 4-arm polyethylene glycol polymer. In certain embodiments the first polymer comprises an 8-arm polyethylene glycol polymer. In certain embodiments the first polymer comprises a 16-arm polyethylene glycol polymer. In certain embodiments the first polymer comprises a 32-arm polyethylene glycol polymer.

In certain embodiments the second polymer comprises a 2-arm polyethylene glycol polymer. In certain embodiments the second polymer comprises a 4-arm polyethylene glycol polymer. In certain embodiments the second polymer comprises an 8-arm polyethylene glycol polymer. In certain embodiments the second polymer comprises a 16-arm polyethylene glycol polymer. In certain embodiments the second polymer comprises a 32-arm polyethylene glycol polymer.

In certain embodiments the first and a second reactive polymer are reacted with said cleavable crosslinker compound, either sequentially or simultaneously.

In certain embodiments the first and second functional groups are the same.

Only in the context of formulas (PL-1), (PL-2) and (PL-3) the terms used have the following meaning:

The term “a moiety capable of being cleaved by elimination under physiological conditions” refers to a structure comprising a group H—C—(CH═CH)ₘ—C—X′ wherein m is 0 or 1 and X′ is a leaving group, wherein an elimination reaction as described above to remove the elements of HX′ can occur at a rate such that the half-life of the reaction is between 1 and 10,000 hours under physiological conditions of pH and temperature. Preferably, the half-life of the reaction is between 1 and 5,000 hours, and more preferably between 1 and 1,000 hours, under physiological conditions of pH and temperature. By physiological conditions of pH and temperature is meant a pH of between 7 and 8 and a temperature between 30 and 40° C.entigrade

The term “reactive polymer and reactive oligomer” refers to a polymer or oligomer comprising functional groups that are reactive towards other functional groups, most preferably under mild conditions compatible with the stability requirements of peptides, proteins, and other biomolecules. Suitable functional groups found in reactive polymers include maleimides, thiols or protected thiols, alcohols, acrylates, acrylamides, amines or protected amines, carboxylic acids or protected carboxylic acids, azides, alkynes including cycloalkynes, 1,3-dienes including cyclopentadienes and furans, alpha-halocarbonyls, and N-hydroxysuccinimidyl, N-hydroxysulfosuccinimidyl, or nitrophenyl esters or carbonates.

The term “functional group capable of connecting to a reactive polymer” refers to a functional group that reacts to a corresponding functional group of a reactive polymer to form a covalent bond to the polymer. Suitable functional groups capable of connecting to a reactive polymer include maleimides, thiols or protected thiols, acrylates, acrylamides, amines or protected amines, carboxylic acids or protected carboxylic acids, azides, alkynes including cycloalkynes, 1,3-dienes including cyclopentadienes and furans, alpha-halocarbonyls, and N-hydroxysuccinimidyl, N-hydroxysulfosuccinimidyl, or nitrophenyl esters or carbonates.

The term “substituted” refers to an alkyl, alkenyl, alkynyl, aryl, or heteroaryl group comprising one or more substituent groups in place of one or more hydrogen atoms. Substituent groups may generally be selected from halogen including F, CI, Br, and I; lower alkyl including linear, branched, and cyclic; lower haloalkyl including fluoroalkyl, chloroalkyl, bromoalkyl, and iodoalkyl; OH; lower alkoxy including linear, branched, and cyclic; SH; lower alkylthio including linear, branched, and cyclic; amino, alkylamino, dialkylamino, silyl including alkylsilyl, alkoxysilyl, and arylsilyl; nitro; cyano; carbonyl; carboxylic acid, carboxylic ester, carboxylic amide; aminocarbonyl; aminoacyl; carbamate; urea; thiocarbamate; thiourea; ketone; sulfone; sulfonamide; aryl including phenyl, naphthyl, and anthracenyl; heteroaryl including 5- member heteroaryls including as pyrrole, imidazole, furan, thiophene, oxazole, thiazole, isoxazole, isothiazole, thiadiazole, triazole, oxadiazole, and tetrazole, 6-member heteroaryls including pyridine, pyrimidine, pyrazine, and fused heteroaryls including benzofuran, benzothiophene, benzoxazole, benzimidazole, indole, benzothiazole, benzisoxazole, and benzisothiazole.

The properties of R¹ and R² may be modulated by the optional addition of electron-donating or electron-withdrawing substituents. By the term “electron-donating group” is meant a substituent resulting in a decrease in the acidity of the R¹R²CH; electron-donating groups are typically associated with negative Hammett σ or Taft σ* constants and are well- known in the art of physical organic chemistry. (Hammett constants refer to aryl/heteroaryl substituents, Taft constants refer to substituents on non-aromatic moieties.) Examples of suitable electron-donating substituents include lower alkyl, lower alkoxy, lower alkylthio, amino, alkylamino, dialkylamino, and silyl.

The term “electron-withdrawing group” refers to a substituent resulting in an increase in the acidity of the R¹R²CH group; electron-withdrawing groups are typically associated with positive Hammett σ or Taft σ* constants and are well-known in the art of physical organic chemistry. Examples of suitable electron-withdrawing substituents include halogen, difluoromethyl, trifluoromethyl, nitro, cyano, C(═O)—R^{X}, wherein -R^{X} is H, lower alkyl, lower alkoxy, or amino, or S(O)ₘR^{Y}, wherein m is 1 or 2 and -R^{Y} is lower alkyl, aryl, or heteroaryl. As is well-known in the art, the electronic influence of a substituent group may depend upon the position of the substituent. For example, an alkoxy substituent on the ortho- or para-position of an aryl ring is electron-donating, and is characterized by a negative Hammett σ constant, while an alkoxy substituent on the meta-position of an aryl ring is electron- withdrawing and is characterized by a positive Hammett σ constant.

The terms “alkyl”, “alkenyl”, and “alkynyl” include linear, branched or cyclic hydrocarbon groups of 1 to 8 carbons or 1 to 6 carbons or 1 to 4 carbons wherein alkyl is a saturated hydrocarbon, alkenyl includes one or more carbon-carbon double bonds and alkynyl includes one or more carbon-carbon triple bonds. Unless otherwise specified these contain 1 to 6 carbons.

The term “aryl” includes aromatic hydrocarbon groups of 6 to 18 carbons, preferably 6 to 10 carbons, including groups such as phenyl, naphthyl, and anthracenyl. “Heteroaryl” includes aromatic rings comprising 3 to 15 carbons containing at least one N, O or S atom, preferably 3 to 7 carbons containing at least one N, O or S atom, including groups such as pyrrolyl, pyridyl, pyrimidinyl, imidazolyl, oxazolyl, isoxazolyl, thiazolyl, isothiazolyl, quinolyl, indolyl, indenyl, and similar.

The term “halogen” includes fluoro, chloro, bromo and iodo.

The term “maleimido” is a group of the formula

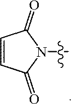

In certain embodiments Z′ is a hydrogel as disclosed in WO2020/206358 A1. In particular, in certain embodiments Z′ is a hydrogel produced by a method comprising the steps of
(a) providing a first prepolymer comprising a multi-arm polymer —P², wherein said first prepolymer is of formula (PL-4)

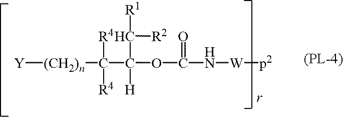

wherein
   n is an integer selected from 0, 1, 2, 3, 4, 5 and 6;
   r is an integer higher than 2;
   -Y is a reactive functional group for connecting said first prepolymer to a second prepolymer;
   -R¹ and -R² are independently an electron-withdrawing group, alkyl, or —H, and wherein at least one of -R¹ and -R² is an electron-withdrawing group;
   each -R⁴ is independently C₁-C₃ alkyl or the two -R⁴ form together with the carbon atom to which they are attached a 3- to 6-membered ring;
   —W— is absent or is 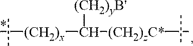
   wherein the dashed line marked with the asterisk indicates the attachment to —NH— and the unmarked dashed line indicates the attachment to —P²;
   each of x, y, and z is independently an integer selected from 0, 1, 2, 3, 4, 5 and 6;
   —B′ is —NH₂, -ONH₂, ketone, aldehyde, —SH, —OH, —CO₂H, carboxamide group, or a group comprising a cyclooctyne or bicyclononyne; and
   —C* is carboxamide, thioether, thiosuccinimidyl, triazole, or oxime;
(b) providing the second prepolymer comprising a multi-arm polymer —P¹ wherein each arm is terminated by a reactive functional group —Y″ that reacts with -Y of step (a);
(c) mixing the two prepolymers of steps (a) and (b) under conditions wherein -Y and -Y″ react to form a linkage -Y*-; and optionally
(d) isolating the resulting hydrogel.

Accordingly, -Z′ is a hydrogel obtainable from the method described above. In certain embodiments the hydrogel produced by the preceding method is degradable.

In certain embodiments -Y and —Y″ react under step (c) to form an insoluble hydrogel matrix comprising crosslinks of formula (PL-4′):

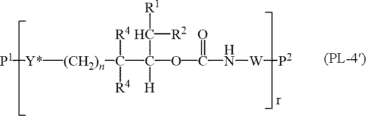

wherein n, r, —P¹, -Y*-, -R⁴, -R¹, -R², —W— and —P² are as defined above.

In certain embodiments n of formula (PL-4) or (PL-4′) is an integer selected from 1, 2, 3, 4, 5 and 6. In certain embodiments n of formula (PL-4) or (PL-4′) is an integer selected from 1, 2 and 3. In certain embodiments n of formula (PL-4) or (PL-4′) is an integer selected from 0, 1, 2 and 3. In certain embodiments n of formula (PL-4) or (PL-4′) is 1. In certain embodiments n of formula (PL-4) is 2. In certain embodiments n of formula (PL-4) or (PL-4′) is 3.

In certain embodiments the multi-arm —P² of formula (PL-4) or (PL-4′) is an r-armed polymer, wherein r is an integer selected from 2, 3, 4, 5, 6, 7, 8, 9, 10, 11 and 12. In certain embodiments r of formula (PL-4) or (PL-4′) is an integer selected from 2, 3, 4, 5, 6, 7 and 8. In certain embodiments r of formula (PL-4) or (PL-4′) is an integer selected from 2, 4, 6 and 8. In certain embodiments r of formula (PL-4) or (PL-4′) is 2. In certain embodiments r of formula (PL-4) or (PL-4′) is 4. In certain embodiments r of formula (PL-4) or (PL-4′) is 6. In certain embodiments r of formula (PL-4) or (PL-4′) is 8.

In certain embodiments —P² of formula (PL-4) or (PL-4′) has a molecular weight of at least 1 kDa. In certain embodiments —P² of formula (PL-4) or (PL-4′) has a molecular weight of 1 to 100 kDa. In certain embodiments —P² of formula (PL-4) or (PL-4′) has a molecular weight of 1 to 80 kDa. In certain embodiments —P² of formula (PL-4) or (PL-4′) has a molecular weight of 1 to 60 kDa. In certain embodiments —P² of formula (PL-4) or (PL-4′) has a molecular weight of 1 to 40 kDa. In certain embodiments —P² of formula (PL-4) or (PL-4′) has a molecular weight of 1 to 20 kDa. In certain embodiments —P² of formula (PL-4) or (PL-4′) has a molecular weight of 1 to 10 kDa. In certain embodiments —P² of formula (PL-4) or (PL-4′) has a molecular weight of 1 to 5 kDa. In certain embodiments —P² of formula (PL-4) or (PL-4′) has a molecular weight of about 20 kDa. In certain embodiments —P² of formula (PL-4) or (PL-4′) has a molecular weight of about 40 kDa. In certain embodiments —P² of formula (PL-4) or (PL-4′) has a molecular weight of about 60 kDa. In certain embodiments —P² of formula (PL-4) or (PL-4′) has a molecular weight of about 80 kDa.

In certain embodiments the multi-arm polymer —P¹ of step (b) is an r-armed polymer, wherein r is an integer selected from 2, 3, 4, 5, 6, 7, 8, 9, 10, 11 and 12. In certain embodiments the multi-arm —P¹ of step (b) is an r-armed polymer, wherein r is an integer selected from 2, 3, 4, 5, 6, 7 and 8. In certain embodiments the multi-arm —P¹ of step (b) is an r-armed polymer, wherein r is an integer selected from 2, 4, 6 and 8. In certain embodiments the multi-arm —P¹ of step (b) is an r-armed polymer, wherein r is 2. In certain embodiments the multi-arm —P¹ of step (b) is an r-armed polymer, wherein r is 4. In certain embodiments the multi-arm —P¹ of step (b) is an r-armed polymer, wherein r is 6. In certain embodiments the multi-arm —P¹ of step (b) is an r-armed polymer, wherein r is 8.

In certain embodiments —P¹ of step (b) has a molecular weight of at least 1 kDa. In certain embodiments the multi-arm polymer —P¹ of step (b) has a molecular weight of 1 to 100 kDa. In certain embodiments the multi-arm polymer —P¹ of step (b) has a molecular weight of 1 to 80 kDa. In certain embodiments the multi-arm polymer —P¹ of step (b) has a molecular weight of 1 to 60 kDa. In certain embodiments the multi-arm polymer —P¹ of step (b) has a molecular weight of 1 to 40 kDa. In certain embodiments the multi-arm polymer —P¹ of step (b) has a molecular weight of 1 to 20 kDa. In certain embodiments the multi-arm polymer —P¹ of step (b) has a molecular weight of 1 to 10 kDa. In certain embodiments the multi-arm polymer —P¹ of step (b) has a molecular weight of 1 to 5 kDa. In certain embodiments the multi-arm polymer -P¹ of step (b) has a molecular weight of about 20 kDa. In certain embodiments the multi-arm polymer —P¹ of step (b) has a molecular weight of about 40 kDa. In certain embodiments the multi-arm polymer —P¹ of step (b) has a molecular weight of about 60 kDa. In certain embodiments the multi-arm polymer —P¹ of step (b) has a molecular weight of about 80 kDa.

In certain embodiments —P¹ of step (b) and —P² of formula (PL-4) or (PL-4′) comprise poly(ethylene glycol) (PEG), poly(ethylene oxide) (PEO), poly(ethylene imine) (PEI), dextrans, hyaluronic acids, or co-polymers thereof. In certain embodiments —P¹ of step (b) and P² of formula (PL-4) or (PL-4′) are PEG-based polymers. In certain embodiments —P¹ of step (b) and —P² of formula (PL-4) or (PL-4′) are hyaluronic acid-based polymers.

In certain embodiments -R¹ and -R² of formula (PL-4) or (PL-4′) are independently electron-withdrawing groups, alkyl, or —H, and wherein at least one of -R¹ and -R² is an electron-withdrawing group.

In certain embodiments the electron-withdrawing group of -R¹ and -R² of formula (PL-4) or (PL-4′) is —CN, —NO₂, optionally substituted aryl, optionally substituted heteroaryl, optionally substituted alkenyl, optionally substituted alkynyl, -COR³, -SOR³, or —SO₂R³, wherein -R³ is —H, optionally substituted alkyl, optionally substituted aryl, optionally substituted arylalkyl, optionally substituted heteroaryl, optionally substituted heteroarylalkyl, —OR⁸ or -NR⁸₂, wherein each -R⁸ is independently —H or optionally substituted alkyl, or both -R⁸ groups are taken together with the nitrogen to which they are attached to form a heterocyclic ring; or —SR⁹, wherein -R⁹ is optionally substituted alkyl, optionally substituted aryl, optionally substituted arylalkyl, optionally substituted heteroaryl, or optionally substituted heteroarylalkyl.

In certain embodiments the electron-withdrawing group of -R¹ and -R² of formula (PL-4) or (PL-4′) is —CN. In certain embodiments the electron-withdrawing group of -R¹ and -R² of formula (PL-4) or (PL-4′) is —NO₂. In certain embodiments the electron-withdrawing group of -R¹ and -R² of formula (PL-4) or (PL-4′) is optionally substituted aryl containing 6 to 10 carbons. In certain embodiments the electron-withdrawing group of -R¹ and -R² of formula (PL-4) or (PL-4′) is optionally substituted phenyl, naphthyl, or anthracenyl. In certain embodiments the electron-withdrawing group of -R¹ and -R² of formula (PL-4) or (PL-4′) is optionally substituted heteroaryl comprising 3 to 7 carbons and containing at least one N, O, or S atom. In certain embodiments the electron-withdrawing group of -R¹ and -R² of formula (PL-4) or (PL-4′) is optionally substituted pyrrolyl, pyridyl, pyrimidinyl, imidazolyl, oxazolyl, isoxazolyl, thiazolyl, isothiazolyl, quinolyl, indolyl, or indenyl. In certain embodiments the electron-withdrawing group of -R¹ and -R² of formula (PL-4) or (PL-4′) is optionally substituted alkenyl containing 2 to 20 carbon atoms. In certain embodiments the electron- withdrawing group of -R¹ and -R² of formula (PL-4) or (PL-4′) is optionally substituted alkynyl containing 2 to 20 carbon atoms. In certain embodiments the electron-withdrawing group of -R¹ and -R² of formula (PL-4) or (PL-4′) is -COR³, -SOR³, or —SO₂R³, wherein R³ is —H, optionally substituted alkyl containing 1 to 20 carbon atoms, optionally substituted aryl, optionally substituted arylalkyl, optionally substituted heteroaryl, optionally substituted heteroarylalkyl, —OR⁸ or -NR⁸₂, wherein each -R⁸ is independently —H or optionally substituted alkyl containing 1 to 20 carbon atoms, or both -R⁸ groups are taken together with the nitrogen to which they are attached to form a heterocyclic ring. In certain embodiments the electron-withdrawing group of -R¹ and -R² of formula (PL-4) or (PL-4′) is —SR⁹, wherein -R⁹ is optionally substituted alkyl containing 1 to 20 carbon atoms, optionally substituted aryl, optionally substituted arylalkyl, optionally substituted heteroaryl, or optionally substituted heteroarylalkyl. In certain embodiments at least one of -R¹ and -R² is —CN or —SO₂R³.

In certain embodiments at least one of -R¹ and -R² of formula (PL-4) or (PL-4′) is —CN, -SOR³ or —SO₂R³. In certain embodiments at least one of -R¹ and -R² of formula (PL-4) or (PL-4′) is —CN or —SO₂R³. In certain embodiments at least one of -R¹ and -R² of formula (PL-4) or (PL-4′) is —CN or —SO₂R³, wherein -R³ is optionally substituted alkyl, optionally substituted aryl, or -NR⁸₂. In certain embodiments at least one of -R¹ and -R² of formula (PL-4) or (PL-4′) is —CN, —SO₂N(CH₃)₂, —SO₂CH₃, phenyl substituted with —SO₂, phenyl substituted with -SO₂ and —Cl, —SO₂N(CH₂CH₂)₂O, —SO₂CH(CH₃)₂, —SO₂N(CH₃)(CH₂CH₃), or -SO₂N(CH₂CH₂OCH₃)₂.

In certain embodiments each -R⁴ of formula (PL-4) or (PL-4′) is independently C₁-C₃ alkyl or taken together may form a 3- to 6-membered ring. In certain embodiments each -R⁴ of formula (PL-4) or (PL-4′) is independently C₁-C₃ alkyl. In certain embodiments both -R⁴ of formula (PL-4) or (PL-4′) are methyl.

In certain embodiments -Y and —Y″ are independently selected from the group consisting of amine, aminooxy, ketone, aldehyde, maleimidyl, thiol, alcohol, azide, 1,2,4,6-tetrazinyl, trans-cyclooctenyl, bicyclononynyl, cyclooctynyl, and protected variants thereof.

In certain embodiments Y and Y″ may react with each other such as in a selective way. For example, when -Y is amine, —Y″ is carboxylic acid, active ester, or active carbonate to yield a residual connecting functional group -Y*- that is amide or carbamate. As another example, when -Y is azide, —Y″ is alkynyl, bicyclononynyl, or cyclooctynyl to yield a residual connecting functional group -Y*- that is 1,2,3-triazole. As another example, when -Y is NH₂O, —Y″ is ketone or aldehyde to yield a residual connecting functional group -Y*- that is oxime. As another example, when -Y is SH, —Y″ is maleimide or halocarbonyl to yield a residual connecting functional group -Y*- that is thiosuccinimidyl or thioether. Similarly, these roles of -Y and —Y″ can be reversed to yield -Y*- of opposing orientation.

In certain embodiments -Y*- comprises an amide, oxime, 1,2,3-triazole, thioether, thiosuccinimide, or ether. In certain embodiments -Y*- is -L²-.

These conjugation reactions may be performed under conditions known in the art, for example when -Y is azide and —Y″ is cyclooctyne the conjugation occurs in any solvent wherein both components show adequate solubility, although it is known that aqueous solutions show more favorable reaction rates. When mixed in an appropriate solvent, typically an aqueous buffer at a pH of 2 to7 when -Y and —Y″ are azide/cyclooctyne, or at a pH of 6 to 9 when -Y and —Y″ are an activated ester and an amine, the -Y and —Y″ groups react to form an insoluble hydrogel matrix comprising crosslinks of formula (PL-4′). This process may be carried out in bulk phase, or under conditions of emulsification in a mixed organic/aqueous system so as to form microparticle suspensions such as microspheres that are suitable for injection.

In certain embodiments a conjugate comprising a hydrogel Z′ is produced by a method comprising the steps of
(a) providing a first prepolymer of formula (PL-4)
(b) reacting the prepolymer of formula (PL-4) with a linker-drug of formula (PL-5)

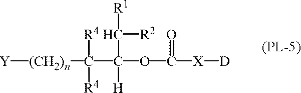

wherein
   n, -R¹, -R², -R⁴ and -Y are as defined in formula (PL-4);
   -D is a drug moiety;
   -X- is absent when -D is a drug moiety connected through an amine, or -X- is —N(R⁶)CH₂— when -D is a drug moiety connected through a phenol, alcohol, thiol, thiophenol, imidazole, or non-basic amine; wherein -R⁶ is optionally substituted C₁-C₆ alkyl, optionally substituted aryl, or optionally substituted heteroaryl;
   so that -Y of formula (PL-5) reacts with —B′ of formula (PL-4);
(c) providing the second prepolymer comprising a multi-arm polymer —P¹ wherein each arm is terminated by a reactive functional group —Y″ that reacts with -Y of step (a) and wherein embodiments for —P¹ are described above;
(d) mixing the two prepolymers of steps (a) and (b) under conditions wherein -Y and -Y″ react to form a residual connecting functional group -Y*-; and optionally
(e) isolating the resulting hydrogel.

In certain embodiments a conjugate is obtained by a method comprising the step of reacting a hydrogel Z′ with the linker-drug of formula (PL-5), wherein —B′ on the hydrogel Z′ reacts with -Y of formula (PL-5).

Only in the context of formulas (PL-4), (PL-4′) and (PL-5) the terms used have the following meaning:

The term “alkyl” refers to linear, branched, or cyclic saturated hydrocarbon groups of 1 to 20, 1 to 12, 1 to 8, 1 to 6, or 1 to 4 carbon atoms. In certain embodiments an alkyl is linear or branched. Examples of linear or branched alkyl groups include methyl, ethyl, n-propyl, isopropyl, n-butyl, t-butyl, isobutyl, sec-butyl, n-pentyl, n-hexyl, n-heptyl, n- octyl, n-nonyl, and n-decyl. In certain embodiments an alkyl is cyclic. Examples of cyclic alkyl groups include cyclopropyl, cyclobutyl, cyclopentyl, cyclopentadienyl, and cyclohexyl.

The term “alkoxy” refers to alkyl groups bonded to oxygen, including methoxy, ethoxy, isopropoxy, cyclopropoxy, and cyclobutoxy.

The term “alkenyl” refers to non-aromatic unsaturated hydrocarbons with carbon-carbon double bonds and 2 to 20, 2 to 12, 2 to 8, 2 to 6, or 2 to 4 carbon atoms.

The term “alkynyl” refers to non-aromatic unsaturated hydrocarbons with carbon-carbon triple bonds and 2 to 20, 2 to 12, 2 to 8, 2 to 6, or 2 to 4 carbon atoms.

The term “aryl” refers to aromatic hydrocarbon groups of 6 to 18 carbons, preferably 6 to 10 carbons, including groups such as phenyl, naphthyl, and anthracenyl. The term “heteroaryl” refers to aromatic rings comprising 3 to 15 carbons comprising at least one N, O or S atom, preferably 3 to 7 carbons comprising at least one N, O or S atom, including groups such as pyrrolyl, pyridyl, pyrimidinyl, imidazolyl, oxazolyl, isoxazolyl, thiazolyl, isothiazolyl, quinolyl, indolyl, and indenyl.

In certain embodiments alkenyl, alkynyl, aryl or heteroaryl moieties may be coupled to the remainder of the molecule through an alkyl linkage. Under those circumstances, the substituent will be referred to as alkenylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

The term “halogen” or “halo” refers to bromo, fluoro, chloro and iodo.

The term “heterocyclic ring” or “heterocyclyl” refers to a 3- to 15-membered aromatic or non-aromatic ring comprising at least one N, O, or S atom. Examples include piperidinyl, piperazinyl, tetrahydropyranyl, pyrrolidine, and tetrahydrofuranyl, as well as the exemplary groups provided for the term “heteroaryl” above. In certain embodiments a heterocyclic ring or heterocyclyl is non-aromatic. In certain embodiments a heterocyclic ring or heterocyclyl is aromatic.

The term “optionally substituted” refers to a group may be unsubstituted or substituted by one or more (e.g., 1, 2, 3, 4 or 5) of the substituents which may be the same or different. Examples of substituents include alkyl, alkenyl, alkynyl, halogen, —CN, —OR^{aa}, —SR^{aa}, —NR^{aa}R^{bb}, —NO₂, —C═NH(OR^{aa}), —C(O)R^{aa}, —OC(O)R^{aa}, —C(O)OR^{aa}, -C(O)NR^{aa}R^{bb,} —OC(O)NR^{aa}R^{bb}, —NR^{aa}C(O)R^{bb}, —NR^{aa}C(O)OR^{bb}, —S(O)R^{aa}, —S(O)₂R^{aa}, —NR^{aa}S(O)R^{bb}, —C(O)NR^{aa}S(O)R^{bb}, —NR^{aa}S(O)₂R^{bb}, —C(O)NR^{aa}S(O)₂R^{bb}, —S(O)NR^{aa}R^{bb}, —S(O)₂NR^{aa}R^{bb}, —P(O)(OR^{aa})(OR^{bb}), heterocyclyl, heteroaryl, or aryl, wherein the alkyl, alkenyl, alkynyl, cycloalkyl, heterocyclyl, heteroaryl, and aryl are each independently optionally substituted by -R^{cc}, wherein -R^{aa} and -R^{bb} are each independently —H, alkyl, alkenyl, alkynyl, heterocyclyl, heteroaryl, or aryl, or —R^{aa} and —R^{bb} are taken together with the nitrogen atom to which they attach to form a heterocyclyl, which is optionally substituted by alkyl, alkenyl, alkynyl, halogen, hydroxyl, alkoxy, or —CN, and wherein: each -R^{cc} is independently alkyl, alkenyl, alkynyl, halogen, heterocyclyl, heteroaryl, aryl, —CN, or —NO₂.

A moiety -L¹- may be attached to -D through the IL-2 moiety of formula (I), in particular through an amino acid residue of said IL-2 moiety present in -D. In certain embodiments -L¹- is attached to -D through the IL-2 moiety, in particular through an amino acid residue of the IL-2 moiety.

In certain embodiments all moieties -L¹- present in an IL-2 conjugate are attached to an amino acid residue of -D.

If -L¹- is attached to an amino acid residue of the IL-2 moiety, such amino acid residue may be a proteinogenic or non-proteinogenic amino acid residue of -D. In certain embodiments -L¹- is attached to a non-proteinogenic amino acid residue. In certain embodiments attachment of -L¹- is to a proteinogenic amino acid residue. If attachment occurs at a proteinogenic amino acid residue, said proteinogenic amino acid residue is in certain embodiments selected from the group consisting of cysteine, methionine, histidine, lysine, tryptophan, serine, threonine, tyrosine, aspartic acid, glutamic acid, glutamine and arginine. In certain embodiments such proteinogenic amino acid residue is selected from the group consisting of cysteine, histidine, lysine, tryptophan, serine, threonine, tyrosine, aspartic acid, glutamic acid and arginine.

In certain embodiments -L¹- is attached to a cysteine residue of -D. In certain embodiments -L¹- is attached to a histidine residue of -D. In certain embodiments -L¹- is attached to a lysine residue. In certain embodiments -L¹- is attached to a tryptophan residue. In certain embodiments -L¹-is attached to a serine residue. In certain embodiments -L¹- is attached to a threonine residue. In certain embodiments -L¹- is attached to a tyrosine residue. In certain embodiments -L¹- is attached to an aspartic acid residue. In certain embodiments -L¹- is attached to a glutamic acid residue. In certain embodiments -L¹- is attached to an arginine residue.

In certain embodiments at least one moiety -L¹- is attached to an amino acid residue of -D and one or more additional moieties -L¹- are attached to a modifying moiety present in -D.

The moiety -L¹- may be connected to -D through any type of linkage, provided that it is reversible. In certain embodiments -L¹- is connected to -D through a linkage selected from the group consisting of amide, ester, carbamate, acetal, aminal, imine, oxime, hydrazone, disulfide and acylguanidine. In certain embodiments -L¹- is connected to -D through a linkage selected from the group consisting of amide, ester, carbamate and acylguanidine. It is understood that these linkages may not be reversible per se, but that reversibility may be an effect of certain groups of atoms or moieties present in -L¹-.

In certain embodiments -L¹- is connected to -D through an ester linkage. In certain embodiments -L¹- is connected to -D through a carbamate linkage. In certain embodiments -L¹- is connected to -D through an acylguanidine. In certain embodiments -L¹- is connected to -D through an amide linkage.

In certain embodiments -L¹- is connected to -D via the nitrogen of an amine functional group of a side chain of a lysine residue of -D. In certain embodiments -L¹- is connected to -D via the nitrogen of an amine functional group of a side chain of a lysine residue of -D and the linkage formed between -D and -L¹- is a carbamate.

In certain embodiments -L¹- has a structure as disclosed in WO 2009/095479 A2. Accordingly, in certain embodiments the moiety -L¹- is of formula (II):

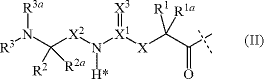

wherein the dashed line indicates attachment to a nitrogen of -D by forming an amide bond;
-X- is —C(R⁴R⁴a)—; —N(R⁴)—; —O—; —C(R⁴R^{4a})—C(R⁵R^{5a})—; —C(R⁵R^{5a})^{-C}(R⁴R^{4a})—; —C(R⁴R^{4a})—N(R⁶)—; —N(R⁶)—C(R⁴R^{4a})—; —C(R⁴R^{4a})—0—; —0—C(R⁴R^{4a})—; or —C(R⁷R^{7a})—;
X¹ is C; or S(O);
-X²- is —C(R⁸R^{8a})—; or —C(R⁸R^{8a})—C(R⁹R⁹ª)—;
=X³ is =O; =S; or =N-CN;
-R¹, -R^{1a}, -R², -R^{2a}, -R⁴, -R^{4a}, -R⁵, -R^{5a}, -R⁶, -R⁸, -R^{8a}, -R⁹, -R^{9a} are independently selected from the group consisting of -H; and C₁₋₆ alkyl;
-R³, -R^{3a} are independently selected from the group consisting of -H; and C₁₋₆ alkyl, provided that in case one of -R³, -R^{3a} or both are other than -H they are connected to the N to which they are attached through an sp³-hybridized carbon atom;
-R⁷ is —N(R¹⁰R^{10a}); or —NR¹⁰—(C═O)—R¹¹;
-R^{7a}, -R¹⁰, -R^{10a}, -R¹¹ are independently of each other -H; or C₁₋₆ alkyl;
optionally, one or more of the pairs -R^{1a}/-R^{4a}, -R¹ª/-R^{5a}, -R¹ª/-R⁷ª, -R^{4a}/-R^{5a}, -R^{8a}/-R^{9a} form a chemical bond;
optionally, one or more of the pairs -R¹/-R¹ª, -R²/-R^{2a},-R⁴/-R^{4a}, -R⁵/-R^{5a}, -R⁸/-R^{8a}, -R⁹/-R^{9a} are joined together with the atom to which they are attached to form a C₃₋₁₀ cycloalkyl; or 3- to 10-membered heterocyclyl;
optionally, one or more of the pairs -R¹/-R⁴, -R¹/-R⁵, -R¹/-R⁶, -R¹/-R⁷ª, -R⁴/-R⁵, -R⁴/-R⁶, -R⁸/-R⁹, -R²/-R³ are joined together with the atoms to which they are attached to form a ring A;
optionally, -R³/-R^{3a} are joined together with the nitrogen atom to which they are attached to form a 3- to 10-membered heterocycle;
A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl; and
wherein -L¹- is substituted with at least one -L²-Z or -L²-Z′ and wherein -L¹- is optionally further substituted, provided that the hydrogen marked with the asterisk in formula (II) is not replaced by -L²-Z, -L²-Z′ or a substituent.

In certain embodiments -L¹- is of formula (II), wherein the dashed line indicates attachment to a nitrogen of an amine of a lysine side chain of -D.

In certain embodiments -L¹- is of formula (II), wherein the dashed line indicates attachment to the nitrogen of the amine of the N-terminus of -D.

Preferably -L¹- of formula (II) is substituted with one moiety -L²-Z or -L²-Z′.

In certain embodiments -L¹- of formula (II) is not further substituted.

It is understood that if -R³/-R^{3a} of formula (II) are joined together with the nitrogen atom to which they are attached to form a 3- to 10-membered heterocycle, only such 3- to 10-membered heterocycles may be formed in which the atoms directly attached to the nitrogen are sp³-hybridized carbon atoms. In other words, such 3- to 10-membered heterocycle formed by -R³/-R^{3a} together with the nitrogen atom to which they are attached has the following structure:

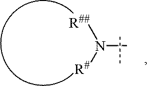

wherein
the dashed line indicates attachment to the rest of -L¹-;
the ring comprises 3 to 10 atoms comprising at least one nitrogen; and
R^{#} and R^{##} represent an sp³-hydridized carbon atom.

It is also understood that the 3- to 10-membered heterocycle may be further substituted.

Exemplary embodiments of suitable 3- to 10-membered heterocycles formed by -R³/-R^{3a} of formula (II) together with the nitrogen atom to which they are attached are the following:

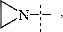

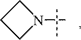

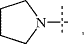

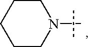

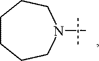

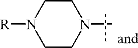

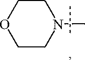

wherein
dashed lines indicate attachment to the rest of the molecule; and
-R is selected from the group consisting of -H and C₁₋₆ alkyl.

-L¹- of formula (II) may optionally be further substituted. In general, any substituent may be used as far as the cleavage principle is not affected, i.e. the hydrogen marked with the asterisk in formula (II) is not replaced and the nitrogen of the moiety

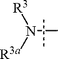

of formula (II) remains part of a primary, secondary or tertiary amine, i.e. -R³ and -R^{3a} are independently of each other -H or are connected to -N< through an sp³-hybridized carbon atom.

In certain embodiments -R¹ or -R¹ª of formula (II) is substituted with -L²-Z or -L²-Z′. In certain embodiments -R² or -R^{2a} of formula (II) is substituted with -L²-Z or -L²-Z′. In certain embodiments -R³ or -R^{3a} of formula (II) is substituted with -L²-Z or -L²-Z′. In certain embodiments -R⁴ of formula (II) is substituted with -L²-Z or -L²-Z′. In certain embodiments -R⁵ or -R^{5a} of formula (II) is substituted with -L²-Z or -L²-Z′ . In certain embodiments -R⁶ of formula (II) is substituted with -L²-Z or -L²-Z′. In certain embodiments -R⁷ or -R^{7a} of formula (II) is substituted with -L²-Z or -L²-Z′. In certain embodiments -R⁸ or -R^{8a} of formula (II) is substituted with -L²-Z or -L²-Z′. In certain embodiments -R⁹ or -R^{9a} of formula (II) is substituted with -L²-Z or -L²-Z′.

In certain embodiments -L¹- has a structure as disclosed in WO2016/020373A1. Accordingly, in certain embodiments the moiety -L¹- is of formula (III):

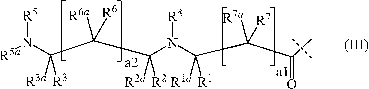

wherein
the dashed line indicates attachment to a primary or secondary amine or hydroxyl of -D by forming an amide or ester linkage, respectively;
-R¹, -R^{1a}, -R², -R^{2a}, -R³ and -R^{3a} are independently of each other selected from the group consisting of -H, —C(R⁸R^{8a}R^{8b}), —C(═O)R⁸, —C═N, —C(═NR⁸)R^{8a}, —CR⁸(═CR^{8a}R^{8b}), —C≡CR⁸ and -T;
-R⁴, -R⁵ and -R^{5a} are independently of each other selected from the group consisting of -H, —C(R⁹R^{9a}R^{9b}) and -T;
a1 and a2 are independently of each other 0 or 1;
each -R⁶, -R^{6a}, -R⁷, -R⁷ª, -R⁸, -R^{8a}, -R^{8b}, -R⁹, -R^{9a}, -R^{9b} are independently of each other selected from the group consisting of -H, halogen, -CN, -COOR¹⁰, -OR¹⁰, —C(O)R¹⁰, —C(O)N(R¹⁰R^{10a}), —S(O)₂N(R¹⁰R¹⁰ª), —S(O)N(R¹⁰R^{10a}), —S(O)₂R¹⁰, —S(O)R¹⁰, —N(R¹⁰)S(O)₂N(R^{10a}R^{10b}), -SR¹⁰, —N(R¹⁰R^{10a}), —NO₂, —OC(O)R¹⁰, —N(R¹⁰)C(O)R^{10a}, —N(R¹⁰)S(O)₂R^{10a}, —N(R¹⁰)S(O)R^{10a}, —N(R¹⁰)C(O)OR^{10a}, —N(R¹⁰)C(O)N(R^{10a}R^{10b}), —OC(O)N(R¹⁰R^{10a}), -T, C₁₋₂₀ alkyl, C₂₋₂₀ alkenyl, and C₂₋₂₀ alkynyl; wherein -T, C₁₋₂₀ alkyl, C₂₋₂₀ alkenyl, and C₂₋₂₀ alkynyl are optionally substituted with one or more -R¹¹, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)(R^{12a})—, —N(R¹²)C(O)N(R^{12a})—, and —OC(O)N(R¹²)—;
each -R¹⁰, —R^{10a}, —R^{10b} is independently selected from the group consisting of —H, -T, C_{1-}₂₀ alkyl, C₂₋₂₀ alkenyl, and C₂₋₂₀ alkynyl; wherein -T, C₁₋₂₀ alkyl, C₂₋₂₀ alkenyl, and C₂₋₂₀ alkynyl are optionally substituted with one or more -R¹¹, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)(R^{12a})—, —N(R¹²)C(O)N(R^{12a})—, and —OC(O)N(R¹²)—;
each T is independently of each other selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T is independently optionally substituted with one or more -R¹¹, which are the same or different;
each -R¹¹ is independently of each other selected from halogen, —CN, oxo (=O), -COOR¹³, -OR¹³, —C(O)R¹³, -C(O)N(R¹³R¹³ª), —S(O)₂N(R¹³R¹³ª), —S(O)N(R¹³R^{13a}), —S(O)₂R¹³, —S(O)R¹³, —N(R¹³)S(O)₂N(R^{13a}R^{13b}), -SR¹³, —N(R¹³R^{13a}), —NO₂, —OC(O)R¹³, —N(R¹³)C(O)R^{13a}, —N(R¹³)S(O)₂R^{13a}, —N(R¹³)S(O)R^{13a}, —N(R¹³)C(O)OR^{13a}, —N(R¹³)C(O)N(R^{13a}R^{13b}), —OC(O)N(R¹³R^{13a}), and C₁₋₆ alkyl; wherein C₁₋₆ alkyl is optionally substituted with one or more halogen, which are the same or different;
each -R¹², -R^{12a}, -R¹³, -R¹³ª, -R^{13b} is independently selected from the group consisting of —H, and C₁₋₆ alkyl; wherein C₁₋₆ alkyl is optionally substituted with one or more halogen, which are the same or different;
optionally, one or more of the pairs -R¹/-R^{1a}, -R²/-R^{2a}, -R³/-R^{3a}, -R⁶/-R^{6a}, -R⁷/-R^{7a} are joined together with the atom to which they are attached to form a C₃₋₁₀ cycloalkyl or a 3- to 10-membered heterocyclyl;
optionally, one or more of the pairs -R¹/-R², -R¹/-R³, -R¹/-R⁴, -R¹/-R⁵, -R¹/-R⁶, -R¹/-R⁷, -R²/-R³, -R²/-R⁴, -R²/-R⁵, -R²/-R⁶, -R²/-R⁷, -R³/-R⁴, -R³/-R⁵, -R³/-R⁶, -R³/-R⁷, -R⁴/-R⁵, -R⁴/-R⁶, -R⁴/-R⁷, -R⁵/-R ⁶, -R⁵/-R⁷, -R⁶/-R⁷ are joined together with the atoms to which they are attached to form a ring A;
A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl;
wherein -L¹-is substituted with at least one -L²-Z or -L²-Z′ and wherein -L¹- is optionally further substituted.

The optional further substituents of -L¹- of formula (III) are preferably as described above.

Preferably -L¹- of formula (III) is substituted with one moiety -L²-Z or -L²-Z′.

In certain embodiments -L¹- of formula (III) is not further substituted.

In certain embodiments -L¹- has a structure as disclosed in EP1536334B1, WO2009/009712A1, WO2008/034122A1, WO2009/143412A2, WO2011/082368A2, and US8618124B2, which are herewith incorporated by reference.

In certain embodiments -L¹- has a structure as disclosed in US8946405B2 and US8754190B2. Accordingly, in certain embodiments -L¹- is of formula (IV):

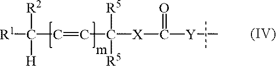

wherein
the dashed line indicates attachment to -D through a functional group of -D selected from the group consisting of —OH, —SH and —NH₂;
m is 0 or 1;
at least one or both of -R¹ and -R² is/are independently of each other selected from the group consisting of —CN, —NO₂, optionally substituted aryl, optionally substituted heteroaryl, optionally substituted alkenyl, optionally substituted alkynyl, —C(O)R³, —S(O)R³, —S(O)₂R³, and —SR⁴,
one and only one of -R¹ and -R² is selected from the group consisting of —H, optionally substituted alkyl, optionally substituted arylalkyl, and optionally substituted heteroarylalkyl;
-R³ is selected from the group consisting of —H, optionally substituted alkyl, optionally substituted aryl, optionally substituted arylalkyl, optionally substituted heteroaryl, optionally substituted heteroarylalkyl, —OR⁹ and —N(R⁹)₂;
-R⁴ is selected from the group consisting of optionally substituted alkyl, optionally substituted aryl, optionally substituted arylalkyl, optionally substituted heteroaryl, and optionally substituted heteroarylalkyl;
each -R⁵ is independently selected from the group consisting of —H, optionally substituted alkyl, optionally substituted alkenylalkyl, optionally substituted alkynylalkyl, optionally substituted aryl, optionally substituted arylalkyl, optionally substituted heteroaryl and optionally substituted heteroarylalkyl;
-R⁹ is selected from the group consisting of —H and optionally substituted alkyl;
—Y— is absent and —X- is —O— or —S—; or
—Y— is —N(Q)CH₂— and -X- is —O—;
Q is selected from the group consisting of optionally substituted alkyl, optionally substituted aryl, optionally substituted arylalkyl, optionally substituted heteroaryl and optionally substituted heteroarylalkyl;
optionally, -R¹ and -R² may be joined to form a 3 to 8-membered ring; and
optionally, both -R⁹ together with the nitrogen to which they are attached form a heterocyclic ring;
wherein -L¹- is substituted with -L²-Z or -L²-Z′ and wherein -L¹- is optionally further substituted.

Only in the context of formula (IV) the terms used have the following meaning:

The term “alkyl” as used herein includes linear, branched or cyclic saturated hydrocarbon groups of 1 to 8 carbons, or in some embodiments 1 to 6 or 1 to 4 carbon atoms.

The term “alkoxy” includes alkyl groups bonded to oxygen, including methoxy, ethoxy, isopropoxy, cyclopropoxy, cyclobutoxy, and similar.

The term “alkenyl” includes non-aromatic unsaturated hydrocarbons with carbon-carbon double bonds.

The term “alkynyl” includes non-aromatic unsaturated hydrocarbons with carbon-carbon triple bonds.

The term “aryl” includes aromatic hydrocarbon groups of 6 to 18 carbons, preferably 6 to 10 carbons, including groups such as phenyl, naphthyl, and anthracenyl. The term “heteroaryl” includes aromatic rings comprising 3 to 15 carbons containing at least one N, O or S atom, preferably 3 to 7 carbons containing at least one N, O or S atom, including groups such as pyrrolyl, pyridyl, pyrimidinyl, imidazolyl, oxazolyl, isoxazolyl, thiazolyl, isothiazolyl, quinolyl, indolyl, indenyl, and similar.

In some instance, alkenyl, alkynyl, aryl or heteroaryl moieties may be coupled to the remainder of the molecule through an alkylene linkage. Under those circumstances, the substituent will be referred to as alkenylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

The term “halogen” includes bromo, fluoro, chloro and iodo.

The term “heterocyclic ring” refers to a 4 to 8 membered aromatic or non-aromatic ring comprising 3 to 7 carbon atoms and at least one N, O, or S atom. Examples are piperidinyl, piperazinyl, tetrahydropyranyl, pyrrolidine, and tetrahydrofuranyl, as well as the exemplary groups provided for the term “heteroaryl” above.

When a ring system is optionally substituted, suitable substituents are selected from the group consisting of alkyl, alkenyl, alkynyl, or an additional ring, each optionally further substituted. Optional substituents on any group, including the above, include halo, nitro, cyano, —OR, —SR, —NR₂, —OCOR, —NRCOR, —COOR, —CONR₂, —SOR, —SO₂R, —SONR₂, —SO₂N R₂, wherein each R is independently alkyl, alkenyl, alkynyl, aryl or heteroaryl, or two R groups taken together with the atoms to which they are attached form a ring.

Preferably -L¹- of formula (IV) is substituted with one moiety -L²-Z or -L²-Z′.

In certain embodiments -L¹- has a structure as disclosed in WO2013/036857A1. Accordingly, in certain embodiments -L¹- is of formula (V):

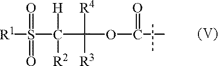

wherein
the dashed line indicates attachment to -D through an amine functional group of -D;
-R¹ is selected from the group consisting of optionally substituted C₁-C₆ linear, branched, or cyclic alkyl; optionally substituted aryl; optionally substituted heteroaryl; alkoxy; and -NR⁵₂;
-R² is selected from the group consisting of —H; optionally substituted C₁-C₆ alkyl; optionally substituted aryl; and optionally substituted heteroaryl;
-R³ is selected from the group consisting of —H; optionally substituted C₁-C₆ alkyl; optionally substituted aryl; and optionally substituted heteroaryl;
-R⁴ is selected from the group consisting of —H; optionally substituted C₁-C₆ alkyl; optionally substituted aryl; and optionally substituted heteroaryl;
each -R⁵ is independently of each other selected from the group consisting of —H; optionally substituted C₁-C₆ alkyl; optionally substituted aryl; and optionally substituted heteroaryl; or when taken together two —R⁵ can be cycloalkyl or cycloheteroalkyl;
wherein -L¹- is substituted with -L²-Z or -L²-Z′ and wherein -L¹- is optionally further substituted.

Only in the context of formula (V) the terms used have the following meaning:

“Alkyl”, “alkenyl”, and “alkynyl” include linear, branched or cyclic hydrocarbon groups of 1-8 carbons or 1-6 carbons or 1-4 carbons wherein alkyl is a saturated hydrocarbon, alkenyl includes one or more carbon-carbon double bonds and alkynyl includes one or more carbon-carbon triple bonds. Unless otherwise specified these contain 1-6 C.

“Aryl” includes aromatic hydrocarbon groups of 6-18 carbons, preferably 6-10 carbons, including groups such as phenyl, naphthyl, and anthracene “Heteroaryl” includes aromatic rings comprising 3-15 carbons containing at least one N, O or S atom, preferably 3-7 carbons containing at least one N, O or S atom, including groups such as pyrrolyl, pyridyl, pyrimidinyl, imidazolyl, oxazolyl, isoxazolyl, thiszolyl, isothiazolyl, quinolyl, indolyl, indenyl, and similar.

The term “substituted” means an alkyl, alkenyl, alkynyl, aryl, or heteroaryl group comprising one or more substituent groups in place of one or more hydrogen atoms. Substituents may generally be selected from halogen including F, Cl, Br, and I; lower alkyl including linear, branched, and cyclic; lower haloalkyl including fluoroalkyl, chloroalkyl, bromoalkyl, and iodoalkyl; OH; lower alkoxy including linear, branched, and cyclic; SH; lower alkylthio including linear, branched and cyclic; amino, alkylamino, dialkylamino, silyl including alkylsilyl, alkoxysilyl, and arylsilyl; nitro; cyano; carbonyl; carboxylic acid, carboxylic ester, carboxylic amide, aminocarbonyl; aminoacyl; carbamate; urea; thiocarbamate; thiourea; ketone; sulfone; sulfonamide; aryl including phenyl, naphthyl, and anthracenyl; heteroaryl including 5-member heteroaryls including as pyrrole, imidazole, furan, thiophene, oxazole, thiazole, isoxazole, isothiazole, thiadiazole, triazole, oxadiazole, and tetrazole, 6-member heteroaryls including pyridine, pyrimidine, pyrazine, and fused heteroaryls including benzofuran, benzothiophene, benzoxazole, benzimidazole, indole, benzothiazole, benzisoxazole, and benzisothiazole.

In certain embodiments -L¹- of formula (V) is substituted with one moiety -L²-Z or -L²-Z′.

In certain embodiments -L¹- has a structure as disclosed in US7585837B2. Accordingly, in certain embodiments -L¹- is of formula (VI):

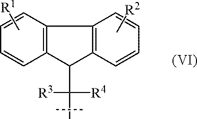

wherein
the dashed line indicates attachment to -D through an amine functional group of -D;
R¹ and R² are independently selected from the group consisting of hydrogen, alkyl, alkoxy, alkoxyalkyl, aryl, alkaryl, aralkyl, halogen, nitro, —SO₃H, —SO₂NHR⁵, amino, ammonium, carboxyl, PO₃H₂, and OPO₃H₂;
R³, R⁴, and R⁵ are independently selected from the group consisting of hydrogen, alkyl, and aryl;
wherein -L¹- is substituted with -L²-Z or -L²-Z′ and wherein -L¹- is optionally further substituted.

Suitable substituents for formulas (VI) are alkyl (such as C₁₋₆ alkyl), alkenyl (such as C₂₋₆ alkenyl), alkynyl (such as C₂₋₆ alkynyl), aryl (such as phenyl), heteroalkyl, heteroalkenyl, heteroalkynyl, heteroaryl (such as aromatic 4 to 7 membered heterocycle) or halogen moieties.

Only in the context of formula (VI) the terms used have the following meaning:

The terms “alkyl”, “alkoxy”, “alkoxyalkyl”, “aryl”, “alkaryl” and “aralkyl” mean alkyl radicals of 1-8, preferably 1-4 carbon atoms, e.g. methyl, ethyl, propyl, isopropyl and butyl, and aryl radicals of 6-10 carbon atoms, e.g. phenyl and naphthyl. The term “halogen” includes bromo, fluoro, chloro and iodo.

In certain embodiments -L¹- of formula (VI) is substituted with one moiety -L²-Z or L²-Z′.

In certain embodiments -L¹- has a structure as disclosed in WO2002/089789A1. Accordingly, in certain embodiments -L¹- is of formula (VII):

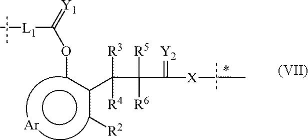

wherein
the dashed line indicates attachment to -D through an amine functional group of -D;
Y₁ and Y₂ are independently O, S or NR⁷;
R², R³, R⁴, R⁵, R⁶ and R⁷ are independently selected from the group consisting of hydrogen, C₁₋₆ alkyls, C₃₋₁₂ branched alkyls, C₃₋₈ cycloalkyls, C₁₋₆ substituted alkyls, C₃₋ ₈ substituted cycloalkyls, aryls, substituted aryls, aralkyls, C₁₋₆ heteroalkyls, substituted
C₁₋₆ heteroalkyls, C₁₋₆ alkoxy, phenoxy, and C₁₋₆ heteroalkoxy;
Ar is a moiety which when included in formula (VII) forms a multisubstituted aromatic hydrocarbon or a multi-substituted heterocyclic group;
X is a chemical bond or a moiety that is actively transported into a target cell, a hydrophobic moiety, or a combination thereof,
y is 0 or 1;
wherein -L¹- is substituted with -L²-Z or L²-Z′ and wherein -L¹- is optionally further substituted.

Only in the context of formula (VII) the terms used have the following meaning:

The term “alkyl” shall be understood to include, e.g. straight, branched, substituted C₁₋₁₂ alkyls, including alkoxy, C₃₋₈ cycloalkyls or substituted cycloalkyls, etc.

The term “substituted” shall be understood to include adding or replacing one or more atoms contained within a functional group or compounds with one or more different atoms.

Substituted alkyls include carboxyalkyls, aminoalkyls, dialkylaminos, hydroxyalkyls and mercaptoalkyls; substtued cycloalkyls include moieties such as 4-chlorocyclohexyl; aryls include moieties such as napthyl; substituted aryls include moieties such as 3-bromo-phenyl; aralkyls include moieties such as toluyl; heteroalkyls include moieties such as ethylthiophene; substituted heteroalkyls include moieties such as 3-methoxythiophone; alkoxy includes moieities such as methoxy; and phenoxy includes moieties such as 3-nitrophenoxy. Halo- shall be understood to include fluoro, chloro, iodo and bromo.

In certain embodiments -L¹- of formula (VII) is substituted with one moiety -L²-Z or -L²-Z′.

In certain embodiments -L¹- comprises a substructure of formula (VIII)

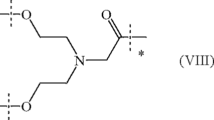

wherein
the dashed line marked with the asterisk indicates attachment to a nitrogen of -D by forming an amide bond;
the unmarked dashed lines indicate attachment to the remainder of -L¹-; and wherein -L¹- is substituted with -L²-Z or -L²-Z′ and wherein -L¹- is optionally further substituted.

In certain embodiments -L¹- is of formula (VIII), wherein the dashed line marked with the asterisk indicates attachment to a nitrogen of an amine of a lysine side chain of -D.

In certain embodiments -L¹- is of formula (VIII), wherein the dashed line marked with the asterisk indicates attachment to the nitrogen of the amine of the N-terminus of -D.

In certain embodiments -L¹- of formula (VIII) is substituted with one moiety -L²-Z or -L²-Z′.

In certain embodiments -L¹- of formula (VIII) is not further substituted.

In certain embodiments -L¹- comprises a substructure of formula (IX)

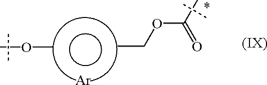

wherein
the dashed line marked with the asterisk indicates attachment to a nitrogen of -D by forming a carbamate bond;
the unmarked dashed lines indicate attachment to the remainder of -L¹-; and wherein -L¹- is substituted with -L²-Z or -L²-Z′ and wherein -L¹- is optionally further substituted.

In certain embodiments -L¹- is of formula (IX), wherein the dashed line marked with the asterisk indicates attachment to a nitrogen of an amine of a lysine side chain of -D.

In certain embodiments -L¹ - is of formula (IX), wherein the dashed line marked with the asterisk indicates attachment to the nitrogen of the amine of the N-terminus of -D.

In certain embodiments -L¹- of formula (IX) is substituted with one moiety -L²-Z or -L²-Z′.

In certain embodiments -L¹- of formula (IX) is not further substituted.

In certain embodiments -L¹- is of formula (IX-a):

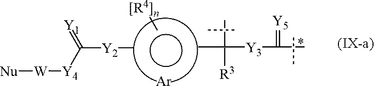

wherein
the dashed line marked with the asterisk indicates attachment to a nitrogen of -D and the unmarked dashed line indicates attachment to -L²-Z or -L²-Z′;
n is 0, 1, 2, 3, or 4;
═Y₁ is selected from the group consisting of =O and =S;
—Y₂— is selected from the group consisting of —O— and —S—;
—Y₃— is selected from the group consisting of —O— and —S—;
—Y₄— is selected from the group consisting of —O—, —NR⁵— and —C(R⁶R^{6a})^{_};
═Y₅ is selected from the group consisting of =O and =S;
—R³, —R⁵, —R⁶, —R^{6a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;
—R⁴ is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;
—W— is selected from the group consisting of C₁₋₂₀ alkyl optionally interrupted by one or more groups selected from the group consisting of C₃₋₁₀ cycloalkyl, 8- to 30-membered carbopolycyclyl, 3- to 10-membered heterocyclyl, —C(O)—, —C(O)N(R⁷)—, —O—, —S— and —N(R⁷)—;
—Nu is a nucleophile selected from the group consisting of —N(R⁷R^{7a}), —N(R⁷OH), —N(R⁷)—N(R^{7a}R^{7b}), —S(R⁷), —COOH,

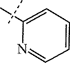

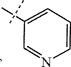

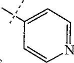

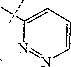

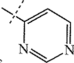

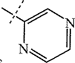

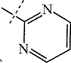

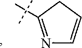

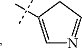

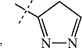

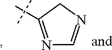

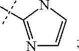

—Ar— is selected from the group consisting of

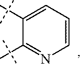

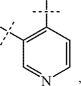

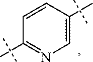

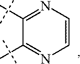

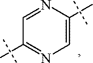

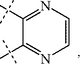

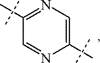

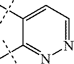

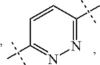

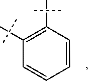

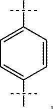

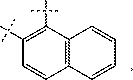

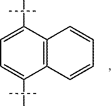

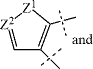

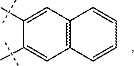

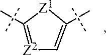

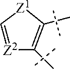

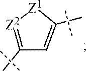

wherein
   dashed lines indicate attachment to the remainder of -L¹-,
   -Z¹- is selected from the group consisting of —O—, —S— and —N(R⁷)—, and
   -Z²- is —N(R⁷)—; and
   _{-R7}, —R^{7a}, —R^{7b} are independently of each other selected from the group consisting of —H, C₁₋₆ alkyl, C₂₋₆ alkenyl and C₂₋₆ alkynyl;
wherein -L¹- is optionally further substituted.

In certain embodiments -L¹- is of formula (IX-a), wherein the dashed line marked with the asterisk indicates attachment to a nitrogen of an amine of a lysine side chain of -D.

In certain embodiments -L¹- is of formula (IX-a), wherein the dashed line marked with the asterisk indicates attachment to the nitrogen of the amine of the N-terminus of -D.

In certain embodiments -L¹- of formula (IX-a) is not further substituted.

In certain embodiments -L¹- is of formula (IX-b):

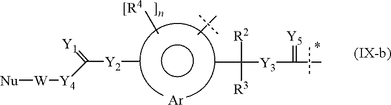

wherein
the dashed line marked with the asterisk indicates attachment to a nitrogen of -D and the unmarked dashed line indicates attachment to -L²-Z or -L²-Z′;
n is 0, 1, 2, 3, or 4;
═Y₁ is selected from the group consisting of ═O and ═S;
—Y₂— is selected from the group consisting of —O— and —S—;
—Y₃— is selected from the group consisting of —O— and —S—;
—Y₄— is selected from the group consisting of —O—, —NR⁵— and —C(R⁶R^{6a})^{_};
═Y₅ is selected from the group consisting of ═O and ═S;
-R², -R³, ^{_}R⁵, -R⁶, -R^{6a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;
-R⁴ is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;
—W— is selected from the group consisting of C₁₋₂₀ alkyl optionally interrupted by one or more groups selected from the group consisting of C₃₋₁₀ cycloalkyl, 8- to 30-membered carbopolycyclyl, 3- to 10-membered heterocyclyl, —C(O)—, —C(O)N(R⁷)—, —O—, —S— and —N(R⁷)—;
—Nu is a nucleophile selected from the group consisting of —N(R⁷R^{7a}), —N(R⁷OH), —N(R⁷)—N(R^{7a}R^{7b}), —S(R⁷),-COOH,

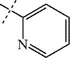

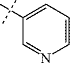

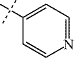

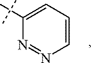

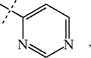

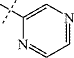

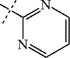

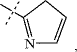

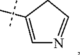

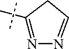

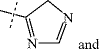

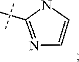

—Ar— is selected from the group consisting of

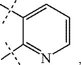

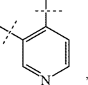

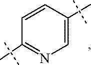

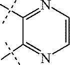

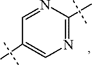

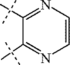

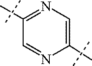

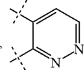

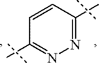

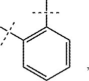

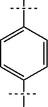

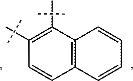

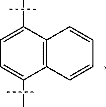

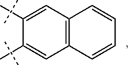

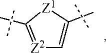

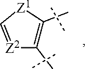

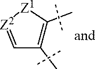

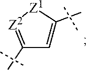

wherein
   dashed lines indicate attachment to the remainder of -L¹-,
   -Z¹- is selected from the group consisting of —O—, —S— and —N(R⁷)—, and
   -Z²- is —N(R⁷)—; and
   -R⁷, -R^{7a}, -R^{7b} are independently of each other selected from the group consisting of —H,
   C₁₋₆ alkyl, C₂₋₆ alkenyl and C₂₋₆ alkynyl;
wherein -L¹- is optionally further substituted.

In certain embodiments -L¹- is of formula (IX-b), wherein the dashed line marked with the asterisk indicates attachment to a nitrogen of an amine of a lysine side chain of -D.

In certain embodiments -L¹- is of formula (IX-b), wherein the dashed line marked with the asterisk indicates attachment to the nitrogen of the amine of the N-terminus of -D.

In certain embodiments -L¹- of formula (IX-b) is not further substituted.

In certain embodiments ═Y¹ of formula (IX-a) and (IX-b) is =O.

In certain embodiments —Y²— of formula (IX-a) and (IX-b) is —O—.

In certain embodiments —Y³— of formula (IX-a) and (IX-b) is —O—.

In certain embodiments —Y⁴— of formula (IX-a) and (IX-b) is —NR⁵—.

In certain embodiments ═Y⁵ of formula (IX-a) and (IX-b) is =O.

In certain embodiments n of formula (IX-a) and (IX-b) is 0 or 1. In certain embodiments n of formula (IX-a) and (IX-b) is 0. In certain embodiments n of formula (IX-a) and (IX-b) is 1.

In certain embodiments -R² of formula (IX-b) is selected from the group consisting of —H, methyl, ethyl, n-propyl, isopropyl, n-butyl, isobutyl, sec-butyl and tert-butyl. In certain embodiments -R² of formula (IX-b) is selected from the group consisting of —H, methyl, ethyl, n-propyl and isopropyl. In certain embodiments -R² of formula (IX-b) is selected from —H, methyl and ethyl. In certain embodiments -R² of formula (IX-b) is —H.

In certain embodiments -R³ of formula (IX-a) and (IX-b) is selected from the group consisting of —H, methyl, ethyl, n-propyl, isopropyl, n-butyl, isobutyl, sec-butyl and tert-butyl. In certain embodiments -R³ of formula (IX-a) and (IX-b) is selected from the group consisting of —H, methyl, ethyl, n-propyl and isopropyl. In certain embodiments -R³ of formula (IX-a) and (IX-b) is selected from —H, methyl and ethyl. In certain embodiments -R³ of formula (IX-a) and (IX-b) is —H.

In certain embodiments each -R⁴ of formula (IX-a) and (IX-b) is independently selected from methyl, ethyl, n-propyl, isopropyl, n-butyl, isobutyl, sec-butyl and tert-butyl. In certain embodiments -R⁴ of formula (IX-a) and (IX-b) is selected from the group consisting of methyl, ethyl, n-propyl and isopropyl. In certain embodiments -R⁴ of formula (IX-a) and (IX-b) is selected from methyl and ethyl.

In certain embodiments -R⁵ of formula (IX-a) and (IX-b) is selected from the group consisting of —H, methyl, ethyl, n-propyl, isopropyl, n-butyl, isobutyl, sec-butyl and tert-butyl. In certain embodiments -R⁵ of formula (IX-a) and (IX-b) is selected from the group consisting of —H, methyl, ethyl, n-propyl and isopropyl. In certain embodiments -R⁵ of formula (IX-a) and (IX-b) is selected from methyl and ethyl. In certain embodiments -R⁵ of formula (IX-a) and (IX-b) is methyl.

In certain embodiments -R⁶ and -R^{6a} of formula (IX-a) and (IX-b) are independently selected from the group consisting of —H, methyl, ethyl, n-propyl, isopropyl, n-butyl, isobutyl, sec-butyl and tert-butyl. In certain embodiments -R⁶ and -R^{6a} of formula (IX-a) and (IX-b) are independently selected from the group consisting of —H, methyl, ethyl, n-propyl and isopropyl. In certain embodiments -R⁶ and -R^{6a} of formula (IX-a) and (IX-b) are independently selected from —H, methyl and ethyl. In certain embodiments —R⁶ and —R^{6a} of formula (IX-a) and (IX-b) are both —H.

In certain embodiments Ar of formula (IX-a) and (IX-b) is phenyl. In certain embodiments Ar of formula (IX-a) and (IX-b) is

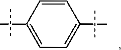

wherein the dashed lines indicate attachment to the remainder of the moiety of formula (IX-a) and (IX-b).

In certain embodiments W of formula (IX-a) and (IX-b) is C₁₋₂₀ alkyl, optionally interrupted with C₃₋₁₀ cycloalkyl, —C(O)—, —C(O)N(R⁷)—, —O—, —S— and —N(R⁷)—. In certain embodiments W of formula (IX-a) and (IX-b) is C₁₋₁₀ alkyl, optionally interrupted with C₃₋₁₀ cycloalkyl, —C(O)—, —C(O)N(R⁷)—, —O—, —S— and —N(R⁷)—. In certain embodiments W of formula (IX-a) and (IX-b) is C₁₋₆ alkyl, optionally interrupted with C₃₋₁₀ cycloalkyl, —C(O)—, —C(O)N(R⁷)—, —O—, —S— and —N(R⁷)—. In certain embodiments W of formula (IX-a) and (IX-b) is

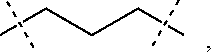

wherein the dashed lines indicate attachment to the remainder of the moiety of formula (IX-a) or (IX-b), respectively.

In certain embodiments —Nu of formula (IX-a) and (IX-b) is —N(R⁷R^{7a}).

In certain embodiments -R⁷, -R^{7a} and -R^{7b} of formula (IX-a) and (IX-b) are independently of each other selected from the group consisting of —H, methyl, ethyl, n-propyl, isopropyl, n-butyl, isobutyl, sec-butyl and tert-butyl. In certain embodiments -R⁷, -R^{7a} and -R^{7b} of formula (IX-a) and (IX-b) are independently of each other selected from —H, methyl, ethyl, n-propyl and isopropyl. In certain embodiments -R⁷, -R^{7a} and -R^{7b} of formula (IX-a) and (IX-b) are independently of each other selected from methyl or ethyl. In certain embodiments -R⁷, -R^{7a} and -R^{7b} of formula (IX-a) and (IX-b) are both methyl.

In certain embodiments -L¹- is of formula (IX-c)

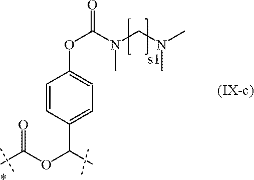

wherein
the dashed line marked with the asterisk indicates attachment to a nitrogen of -D;
the unmarked dashed line indicates attachment to -L²-Z or -L²-Z′; and
s1 is an integer selected from the group consisting of 1, 2, 3, 4, 5, 6, 7, 8, 9 and 10.

In certain embodiments -L¹- is of formula (IX-c), wherein the dashed line marked with the asterisk indicates attachment to a nitrogen of an amine of a lysine side chain of -D.

In certain embodiments -L¹- is of formula (IX-c), wherein the dashed line marked with the asterisk indicates attachment to the nitrogen of the amine of the N-terminus of -D.

In certain embodiments s1 of formula (IX-c) is an integer selected from the group consisting of 1, 2, 3, 4 and 5. In certain embodiments s1 of formula (IX-c) is 1. In certain embodiments s1 of formula (IX-c) is 2. In certain embodiments s1 of formula (IX-c) is 3. In certain embodiments s1 of formula (IX-c) is 4. In certain embodiments s1 of formula (IX-c) is 5.

In certain embodiments -L¹- is of formula (IX-d)

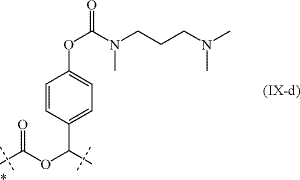

wherein
the dashed line marked with the asterisk indicates attachment to a nitrogen of -D; and
the unmarked dashed line indicates attachment to -L²-Z or -L²-Z′.

In certain embodiments -L¹- is of formula (IX-d), wherein the dashed line marked with the asterisk indicates attachment to a nitrogen of an amine of a lysine side chain of -D.

In certain embodiments -L¹- is of formula (IX-d), wherein the dashed line marked with the asterisk indicates attachment to the nitrogen of the amine of the N-terminus of -D.

In certain embodiments -L¹- has a structure as disclosed in WO2020/206358 A1. Accordingly, in certain embodiments the moiety -L¹- is of formula (X):

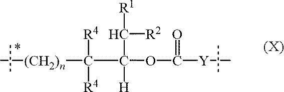

wherein
the unmarked dashed line indicates attachment to -D;
the dashed line marked with the asterisk indicates attachment to -L²-Z or -L²-Z′;
n is an integer selected from the group consisting of 0, 1, 2, 3, 4, 5 and 6;
-R¹ and -R² are independently an electron-withdrawing group, alkyl, or -H, and wherein at least one of -R¹ or -R² is an electron-withdrawing group;
each -R⁴ is independently C₁-C₃ alkyl or the two -R⁴ are taken together with the carbon atom to which they are attached to form a 3- to 6-membered ring; and
—Y— is absent when -D is a drug moiety connected through an amine, or —Y— is —N(R⁶)CH₂₋ when -D is a drug moiety connected through a phenol, alcohol, thiol, thiophenol, imidazole, or non-basic amine; wherein -R⁶ is optionally substituted
C₁-C₆ alkyl, optionally substituted aryl, or optionally substituted heteroaryl.

In certain embodiments n of formula (X) is an integer selected from 1, 2, 3, 4, 5 and 6. In certain embodiments n of formula (X) is an integer selected from 1, 2 and 3. In certain embodiments n of formula (X) is an integer from 0, 1, 2 and 3. In certain embodiments n of formula (X) is 1. In certain embodiments n of formula (X) is 2. In certain embodiments n of formula (X) is 3.

In certain embodiments the electron-withdrawing group of -R¹ and -R² of formula (X) is selected from the group consisting of —CN; —NO₂; optionally substituted aryl; optionally substituted heteroaryl; optionally substituted alkenyl; optionally substituted alkynyl; -COR³, -SOR³, or —SO₂R³, wherein -R³ is -H, optionally substituted alkyl, optionally substituted aryl, optionally substituted arylalkyl, optionally substituted heteroaryl, optionally substituted heteroarylalkyl, —OR⁸ or -NR⁸₂, wherein each -R⁸ is independently —H or optionally substituted alkyl, or both -R⁸ groups are taken together with the nitrogen to which they are attached to form a heterocyclic ring; or —SR⁹, wherein -R⁹ is optionally substituted alkyl, optionally substituted aryl, optionally substituted arylalkyl, optionally substituted heteroaryl, or optionally substituted heteroarylalkyl.

In certain embodiments the electron-withdrawing group of -R¹ and -R² of formula (X) is —CN. In certain embodiments the electron-withdrawing group of -R¹ and -R² of formula (X) is —NO₂. In certain embodiments the electron-withdrawing group of -R¹ and -R² of formula (X) is optionally substituted aryl comprising 6 to 10 carbons. In certain embodiments the electron-withdrawing group of -R¹ and -R² of formula (X) is optionally substituted phenyl, naphthyl, or anthracenyl. In certain embodiments the electron-withdrawing group of -R¹ and -R² of formula (X) is optionally substituted heteroaryl comprising 3 to 7 carbons and comprising at least one N, O, or S atom. In certain embodiments the electron-withdrawing group of -R¹ and -R² of formula (X) is optionally substituted pyrrolyl, pyridyl, pyrimidinyl, imidazolyl, oxazolyl, isoxazolyl, thiazolyl, isothiazolyl, quinolyl, indolyl, or indenyl. In certain embodiments the electron-withdrawing group of -R¹ and -R² of formula (X) is optionally substituted alkenyl containing 2 to 20 carbon atoms. In certain embodiments the electron-withdrawing group of -R¹ and -R² of formula (X) is optionally substituted alkynyl comprising 2 to 20 carbon atoms. In certain embodiments the electron-withdrawing group of -R¹ and -R² of formula (X) is -COR³, -SOR³, or —SO₂R³, wherein -R³ is —H, optionally substituted alkyl comprising 1 to 20 carbon atoms, optionally substituted aryl, optionally substituted arylalkyl, optionally substituted heteroaryl, optionally substituted heteroarylalkyl, —OR⁸ or -NR⁸₂, wherein each -R⁸ is independently —H or optionally substituted alkyl comprising 1 to 20 carbon atoms, or both -R⁸ groups are taken together with the nitrogen to which they are attached to form a heterocyclic ring. In certain embodiments the electron-withdrawing group of -R¹ and -R² of formula (X) is —SR⁹, wherein -R⁹ is optionally substituted alkyl comprising 1 to 20 carbon atoms, optionally substituted aryl, optionally substituted arylalkyl, optionally substituted heteroaryl, or optionally substituted heteroarylalkyl.

In certain embodiments at least one of -R¹ or -R² of formula (X) is —CN, -SOR³ or —SO₂R³. In certain embodiments at least one of -R¹ and -R² of formula (X) is —CN or —SO₂R³. In certain embodiments at least one of -R¹ and -R² of formula (X) is —CN or —SO₂R³, wherein -R³ is optionally substituted alkyl, optionally substituted aryl, or -NR⁸₂. In certain embodiments at least one of -R¹ and -R² of formula (X) is —CN, —SO₂N(CH₃)₂, —SO₂CH₃, phenyl substituted with —SO₂, phenyl substituted with —SO₂ and —Cl, —SO₂N(CH₂CH₂)₂O, —SO₂CH(CH₃)₂, —SO₂N(CH₃)(CH₂CH₃), or —SO₂N(CH₂CH₂OCH₃)₂.

In certain embodiments each -R⁴ of formula (X) is independently C₁-C₃ alkyl. In certain embodiments both -R⁴ are methyl.

In certain embodiments —Y— of formula (X) is absent. In certain embodiments —Y— of formula (X) is —N(R⁶)CH₂—.

In certain embodiments -L¹- is of formula (X), wherein n is 1, -R¹ is —CN, -R² is —H, and -R⁴ is —CH₃. In certain embodiments -L¹- is of formula (X), wherein n is 1, -R¹ is —SO₂N(CH₃)₂, -R² is —H, and -R⁴ is —CH₃. In certain embodiments -L¹- is of formula (X), wherein n is 1, -R¹ is SO₂CH₃, -R² is —H, and -R⁴ is —CH₃. In certain embodiments -L¹- is of formula (X), wherein n is 1, -R¹ is —SO₂N(CH₂CH₂)₂CHCH₃, -R² is —H, and -R⁴ is —CH₃. In certain embodiments -L¹- is of formula (X), wherein n is 1, -R¹ is phenyl substituted with —SO₂, -R² is —H, and -R⁴ is —CH₃. In certain embodiments -L¹- is of formula (X), wherein n is 1, -R¹ is phenyl substituted with —SO₂ and —Cl, -R² is —H, and -R⁴ is —CH₃. In certain embodiments -L¹- is of formula (X), wherein n is 1, -R¹ is —SO₂N(CH₂CH₂)₂O, -R² is —H, and -R⁴ is —CH₃. In certain embodiments -L¹- is of formula (X), wherein n is 1, -R¹ is —SO₂CH(CH₃)₂, -R² is —H, and -R⁴ is —CH₃. In certain embodiments -L¹- is of formula (X), wherein n is 1, -R¹ is —SO₂N(CH₃)(CH₂CH₃), -R² is —H, and -R⁴ is —CH₃. In certain embodiments -L¹- is of formula (X), wherein n is 1, -R¹ is —SO₂N(CH₂CH₂OCH₃)₂, -R² is —H, and -R⁴ is —CH₃. In certain embodiments -L¹- is of formula (X), wherein n is 1, -R¹ is phenyl substituted with-SO₂ and —CH₃, -R² is —H, and -R⁴ is —CH₃.

In certain embodiments -L¹- is of formula (X), wherein n is 2, -R¹ is —CN, -R² is —H, and -R⁴ is —CH₃. In certain embodiments -L¹- is of formula (X), wherein n is 2, -R¹ is —SO₂N(CH₃)₂, -R² is —H, and -R⁴ is —CH₃. In certain embodiments -L¹- is of formula (X), wherein n is 2, -R¹ is SO₂CH₃, -R² is —H, and -R⁴ is —CH₃. In certain embodiments -L¹- is of formula (X), wherein n is 2, -R¹ is —SO₂N(CH₂CH₂)₂CHCH₃, -R² is —H, and -R⁴ is —CH₃. In certain embodiments -L¹- is of formula (X), wherein n is 2, -R¹ is phenyl substituted with —SO₂, -R² is —H, and -R⁴ is —CH₃. In certain embodiments -L¹- is of formula (X), wherein n is 2, -R¹ is phenyl substituted with —SO₂ and —Cl, -R² is —H, and -R⁴ is —CH₃. In certain embodiments -L¹- is of formula (X), wherein n is 2, -R¹ is —SO₂N(CH₂CH₂)₂O, -R² is —H, and -R⁴ is —CH₃. In certain embodiments -L¹- is of formula (X), wherein n is 2, -R¹ is —SO₂CH(CH₃)₂, -R² is —H, and -R⁴ is —CH₃. In certain embodiments -L¹- is of formula (X), wherein n is 2, -R¹ is —SO₂N(CH₃)(CH₂CH₃), -R² is —H, and -R⁴ is —CH₃. In certain embodiments -L^{l}- is of formula (X), wherein n is 2, -R¹ is —SO₂N(CH₂CH₂OCH₃)₂, -R² is —H, and -R⁴ is —CH₃. In certain embodiments -L¹- is of formula (X), wherein n is 2, -R¹ is phenyl substituted with -SO₂ and —CH₃, -R² is —H, and -R⁴ is —CH₃.

In certain embodiments -L¹- is of formula (X), wherein n is 3, -R¹ is —CN, -R² is —H, and -R⁴ is —CH₃. In certain embodiments -L¹- is of formula (X), wherein n is 3, -R¹ is —SO₂N(CH₃)₂, -R² is —H, and -R⁴ is —CH₃. In certain embodiments -L¹- is of formula (X), wherein n is 3, -R¹ is SO₂CH₃, -R² is —H, and -R⁴ is —CH₃. In certain embodiments -L¹- is of formula (X), wherein n is 3, -R¹ is —SO₂N(CH₂CH₂)₂CHCH₃, -R² is —H, and —R⁴ is —CH₃. In certain embodiments -L¹- is of formula (X), wherein n is 3, -R¹ is phenyl substituted with —SO₂, -R² is —H, and -R⁴ is —CH₃. In certain embodiments -L¹- is of formula (X), wherein n is 3, -R¹ is phenyl substituted with —SO₂ and —Cl, -R² is —H, and -R⁴ is —CH₃. In certain embodiments -L¹- is of formula (X), wherein n is 3, -R¹ is —SO₂N(CH₂CH₂)₂O, -R² is —H, and -R⁴ is —CH₃. In certain embodiments -L¹- is of formula (X), wherein n is 3, -R¹ is —SO₂CH(CH₃)₂, -R² is —H, and -R⁴ is —CH₃. In certain embodiments -L¹- is of formula (X), wherein n is 3, -R¹ is —SO₂N(CH₃)(CH₂CH₃), -R² is —H, and -R⁴ is —CH₃. In certain embodiments -L¹- is of formula (X), wherein n is 3, -R¹ is —SO₂N(CH₂CH₂OCH₃)₂, -R² is —H, and -R⁴ is —CH₃. In certain embodiments -L¹- is of formula (X), wherein n is 3, -R¹ is phenyl substituted with -SO₂ and —CH₃, -R² is —H, and -R⁴ is —CH₃.

Only in the context of formula (X) the terms used have the following meaning:

The term “alkyl” refers to linear, branched, or cyclic saturated hydrocarbon groups of 1 to 20, 1 to 12, 1 to 8, 1 to 6, or 1 to 4 carbon atoms. In certain embodiments an alkyl is linear or branched. Examples of linear or branched alkyl groups include methyl, ethyl, n-propyl, isopropyl, n-butyl, t-butyl, isobutyl, sec-butyl, n-pentyl, n-hexyl, n-heptyl, n- octyl, n-nonyl, and n-decyl. In certain embodiments an alkyl is cyclic. Examples of cyclic alkyl groups include cyclopropyl, cyclobutyl, cyclopentyl, cyclopentadienyl, and cyclohexyl.

The term “alkoxy” refers to alkyl groups bonded to oxygen, including methoxy, ethoxy, isopropoxy, cyclopropoxy, and cyclobutoxy.

The term “alkenyl” refers to non-aromatic unsaturated hydrocarbons with carbon-carbon double bonds and 2 to 20, 2 to 12, 2 to 8, 2 to 6, or 2 to 4 carbon atoms.

The term “alkynyl” refers to non-aromatic unsaturated hydrocarbons with carbon-carbon triple bonds and 2 to 20, 2 to 12, 2 to 8, 2 to 6, or 2 to 4 carbon atoms.

The term “aryl” refers to aromatic hydrocarbon groups of 6 to 18 carbons, preferably 6 to 10 carbons, including groups such as phenyl, naphthyl, and anthracenyl. The term “heteroaryl” refers to aromatic rings comprising 3 to 15 carbons comprising at least one N, O or S atom, preferably 3 to 7 carbons comprising at least one N, O or S atom, including groups such as pyrrolyl, pyridyl, pyrimidinyl, imidazolyl, oxazolyl, isoxazolyl, thiazolyl, isothiazolyl, quinolyl, indolyl, and indenyl.

In certain embodiments alkenyl, alkynyl, aryl or heteroaryl moieties may be coupled to the remainder of the molecule through an alkyl linkage. Under those circumstances, the substituent will be referred to as alkenylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

The term “halogen” or “halo” refers to bromo, fluoro, chloro and iodo.

The term “heterocyclic ring” or “heterocyclyl” refers to a 3- to 15-membered aromatic or non-aromatic ring comprising at least one N, O, or S atom. Examples include piperidinyl, piperazinyl, tetrahydropyranyl, pyrrolidine, and tetrahydrofuranyl, as well as the exemplary groups provided for the term “heteroaryl” above. In certain embodiments a heterocyclic ring or heterocyclyl is non-aromatic. In certain embodiments a heterocyclic ring or heterocyclyl is aromatic.

The term “optionally substituted” refers to a group may be unsubstituted or substituted by one or more (e.g., 1, 2, 3, 4 or 5) of the substituents which may be the same or different. Examples of substituents include alkyl, alkenyl, alkynyl, halogen, —CN, —OR^{aa}, —SR^{aa}, —NRa^{a}R^{bb}, —NO₂, —C═NH(OR^{aa}), —C(O)R^{aa}, —OC(O)R^{aa}, —C(O)OR^{aa}, —C(O)NR^{aa}R^{bb,} —OC(O)NR^{aa}R^{bb}, —NR^{aa}C(O)R^{bb}, —NR^{aa}C(O)OR^{bb}, —S(O)R^{aa}, —S(O)₂R^{aa}, —NR^{aa}S(O)R^{bb,} —C(O)NR^{aa}S(O)R^{bb}, —NR^{aa}S(O)₂R^{bb}, —C(O)NR^{aa}S(O)₂R^{bb}, —S(O)NR^{aa}R^{bb}, —S(O)₂NR^{aa}R^{bb}, —P(O)(OR^{aa})(OR^{bb}), heterocyclyl, heteroaryl, or aryl, wherein the alkyl, alkenyl, alkynyl, cycloalkyl, heterocyclyl, heteroaryl, and aryl are each independently optionally substituted by -R^{cc}, wherein —R^{aa} and —R^{bb} are each independently —H, alkyl, alkenyl, alkynyl, heterocyclyl, heteroaryl, or aryl, or —R^{aa} and —R^{bb} are taken together with the nitrogen atom to which they attach to form a heterocyclyl, which is optionally substituted by alkyl, alkenyl, alkynyl, halogen, hydroxyl, alkoxy, or —CN, and wherein: each -R^{cc} is independently alkyl, alkenyl, alkynyl, halogen, heterocyclyl, heteroaryl, aryl, —CN, or —NO₂.

In certain embodiments -L²- is a chemical bond. In certain embodiments -L²- is a spacer moiety.

In certain embodiments -L²- is selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1})—, —S—, —N(R^{y1})—, —OC(OR^{y1})(R^{y1})—, —N(R^{y1})C(O)N(R^{y1})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, -N(R^{y3})S(O)₂N(R^{y3a})-, —S—, —N(R^{y3})^{_} -OC(OR^{y3})(R^{y3a})-, -N(R^{y3})C(O)N(R^{y3a})-, and —OC(O)N(R^{y3})—;
-R^{y1} and -R^{y1a} are independently of each other selected from the group consisting of —H, -T, C₁₋₅₀ alkyl, C₂₋₅₀ alkenyl, and C₂₋₅₀ alkynyl; wherein -T, C₁₋₅₀ alkyl, C₂₋₅₀ alkenyl, and C₂₋₅₀ alkynyl are optionally substituted with one or more -R^{y2}, which are the same or different, and wherein C_{I-50} alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4ª})-, —S—, -N(R^{y4} -OC(OR^{y4})(R^{y4a})-, -N(R^{y4})C(O)N(R^{y4a})-, and —OC(O)N(R^{y4})—;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8-to 30-membered carbopolycyclyl, and 8- to 30-membered heteropolycyclyl; wherein each T is independently optionally substituted with one or more -R^{y2}, which are the same or different;
each -R^{y2} is independently selected from the group consisting of halogen, —CN, oxo (=O), -COOR^{y5}, —OR^{y5}, —C(O)R^{y5}, -C(O)N(R^{y5} R^{y5a}), -S(O)₂N(R^{y5}R^{y5a}), -S(O)N(R^{y5}R^{y5a}), —S(O)₂R^{y5}, —S(O)R^{y5}, -N(R^{y5})S(O)₂N(R^{y5a}R^{y5b}), —SR^{y5}, -N(R^{y5}R^{y5a}), —NO₂, -OC(O)R^{y5} -N(R^{y5})C(O)R^{y5a}, -N(R^{y5})S(O)₂R^{y5a}, -N(R^{y5})S(O)R^{y5a}, -N(R^{y5})C(O)OR^{y5a}, -N(R^{Y5})C(O)N(R^{y5a}R^{y5b}), -OC(O)N(R^{y5}R^{y5a}), and C₁₋₆ alkyl; wherein C₁₋₆ alkyl is optionally substituted with one or more halogen, which are the same or different; and
each -R^{y3}, -R^{y3a}, -R^{y4}, -R^{y4a}, -R^{y5}, -R^{y5a} and -R^{y5b} is independently selected from the group consisting of —H, and C₁₋₆ alkyl, wherein C₁₋₆ alkyl is optionally substituted with one or more halogen, which are the same or different.

In certain embodiments -L²- is selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, -N(R^{y1})S(O)₂N(R^{y1a})-, —S—, —N(R^{y1})—, -OC(OR^{y1})(R^{y1a})-, -N(R^{y1})C(O)N(R^{y1a})-, —OC(O)N(R^{y1})—, C₁₋₅₀ alkyl, C₂₋₅₀ alkenyl, and C₂₋₅₀ alkynyl; wherein -T-, C₁₋₂₀ alkyl, C₂₋₂₀ alkenyl, and C₂₋₂₀ alkynyl are optionally substituted with one or more -R^{y2}, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, -N(R^{y3})S(O)₂N(R^{y3a})-, —S—, -N(R^{y3} -OC(OR^{y3})(R^{y3a})-, -N(R^{y3})C(O)N(R^{y3a})-, and —OC(O)N(R^{y3})—;
-R^{y1} and -R^{y1a} are independently of each other selected from the group consisting of —H, -T, C₁₋₁₀ alkyl, C₂₋₁₀ alkenyl, and C₂₋₁₀ alkynyl; wherein -T, C₁₋₁₀ alkyl, C₂₋₁₀ alkenyl, and C₂₋₁₀ alkynyl are optionally substituted with one or more -R^{y2}, which are the same or different, and wherein C₁-₁₀ alkyl, C₂₋₁₀ alkenyl, and C₂₋₁₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, -N(R^{y4})S(O)₂N(R^{y4a})-, —S—, -N(R^{y4} -OC(OR^{y4})(R^{y4a})-, -N(R^{y4})C(O)N(R^{y4a})-, and —OC(O)N(R^{y4})—;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8-to 30-membered carbopolycyclyl, and 8- to 30-membered heteropolycyclyl; wherein each T is independently optionally substituted with one or more -R^{y2}, which are the same or different;
-R^{y2} is selected from the group consisting of halogen, —CN, oxo (═O), -COOR^{y5,} —OR^{y5}, —C(O)R^{y5}, -C(O)N(R^{y5} R^{y5a}), -S(O)₂N(R^{y5}R^{y5a}), -S(O)N(R^{y5} R^{y5a}), —S(O)₂R^{y5}, —S(O)R^{y5}, -N(R^{y5})S(O)₂N(R^{y5a}R^{y5b}), —SR^{y5}, -N(R^{y5}R^{y5a}), -NO_{2,} —OC(O)R^{y5}, -N(R^{y5})C(O)R^{y5a}, -N(R^{y5})S(O)₂ R^{y5a}, -N(R^{y5})S(O)R^{y5a}, -N(R^{y5})C(O)OR^{y5a}, -N(R^{y5})C(O)N(R^{y5a}R^{y5b}), -OC(O)N(R^{y5}R^{ysa}), and C₁₋₆ alkyl; wherein C₁₋₆ alkyl is optionally substituted with one or more halogen, which are the same or different; and
each -R^{y3}, -R^{y3a}, -R^{y4}, -R^{y4a}, -R^{y5}, -R^{y1a} and -R^{y5b} is independently of each other selected from the group consisting of —H, and C₁₋₆ alkyl; wherein C₁₋₆ alkyl is optionally substituted with one or more halogen, which are the same or different.

In certain embodiments -L²- is selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, -N(R^{y1})S(O)₂N(R^{y1a})-, —S—, —N(R^{y1})—, -OC(OR^{y1})(R^{y1a})-, -N(R^{y1})C(O)N(R^{y1a})-, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more -R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, -N(R^{y3})S(O)₂N(R^{y3a})-, —S—, -N(R^{y3})--OC(OR^{y3})(R^{y3a})-, -N(R^{y3})C(O)N(R^{y3a})-, and —OC(O)N(R^{y3})—;
-R^{y1} and -R^{y1a} are independently selected from the group consisting of —H, -T, C₁₋₁₀ alkyl, C₂₋₁₀ alkenyl, and C₂₋₁₀ alkynyl;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8-to 30-membered carbopolycyclyl, and 8- to 30-membered heteropolycyclyl;
each -R^{y2} is independently selected from the group consisting of halogen, and C₁₋₆ alkyl; and
each -R^{y3}, -R^{y3a}, -R^{y4}, -R^{y4a}, -R^{y5}, -R^{y5a} and -R^{y5b} is independently of each other selected from the group consisting of —H, and C₁₋₆ alkyl; wherein C₁₋₆ alkyl is optionally substituted with one or more halogen, which are the same or different.

In certain embodiments -L²- is a C₁₋₂₀ alkyl chain, which is optionally interrupted by one or more groups independently selected from —O—, -T- and —C(O)N(R^{y1})—; and which C₁₋₂₀ alkyl chain is optionally substituted with one or more groups independently selected from —OH, -T and -C(O)N(R^{y6}R^{y6a}); wherein -R^{y1}, -R^{y6}, -R^{y6a} are independently selected from the group consisting of H and C₁₋₄ alkyl and wherein T is selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8-to 30-membered carbopolycyclyl, and 8- to 30-membered heteropolycyclyl.

In certain embodiments -L²- has a molecular weight in the range of from 14 g/mol to 750 g/mol.

In certain embodiments -L²- comprises a moiety selected from the group consisting of

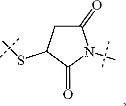

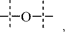

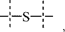

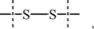

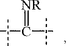

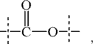

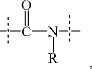

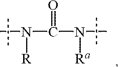

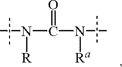

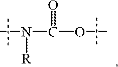

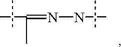

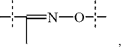

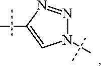

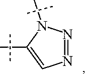

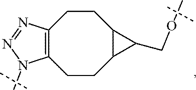

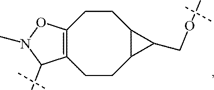

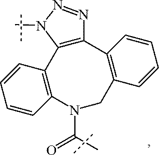

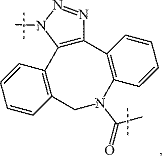

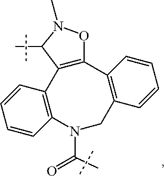

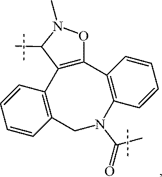

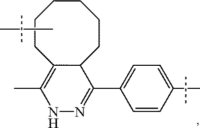

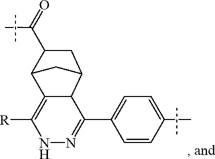

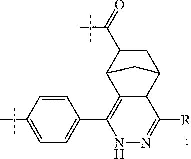

wherein
dashed lines indicate attachment to -L¹-, the remainder of -L²- or to -Z, respectively; and
-R and -R^{a} are independently of each other selected from the group consisting of —H, methyl, ethyl, propyl, butyl, pentyl and hexyl.

In certain embodiments -L²- is of formula (IX-e)

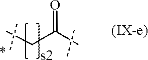

wherein
the dashed line marked with the asterisk indicates attachment to -L¹-;
the unmarked dashed line indicates attachment to -Z; and
s2 is an integer selected from the group consisting of 0, 1, 2, 3, 4, 5, 6, 7, 8, 9, 10, 11, 12, 13, 14, 15, 16, 17, 18, 19 and 20.

In certain embodiments s2 of formula (IX-e) is an integer selected from the group consisting of 1, 2, 3, 4, 5, 6, 7, 8, 9, 10, 11 and 12. In certain embodiments s2 of formula (IX-e) is an integer selected from the group consisting of 1, 2, 3, 4, 5, 6, 7 and 8. In certain embodiments s2 of formula (IX-e) is 1. In certain embodiments s2 of formula (IX-e) is 2. In certain embodiments s2 of formula (IX-e) is 3. In certain embodiments s2 of formula (IX-e) is 4. In certain embodiments s2 of formula (IX-e) is 5. In certain embodiments s2 of formula (IX-e) is 6. In certain embodiments s2 of formula (IX-e) is 7. In certain embodiments s2 of formula (IX-e) is 8.

In certain embodiments the moiety -L¹-L²- is of formula (IX-f)

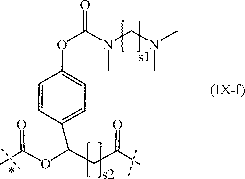

wherein
the dashed line marked with the asterisk indicates attachment to a nitrogen of -D;
the unmarked dashed line indicates attachment to -Z;
s1 is an integer selected from the group consisting of 1, 2, 3, 4, 5, 6, 7, 8, 9 and 10; and
s2 is an integer selected from the group consisting of 0, 1, 2, 3, 4, 5, 6, 7, 8, 9, 10, 11, 12, 13, 14, 15, 16, 17, 18, 19 and 20.

In certain embodiments -L¹- is of formula (IX-f), wherein the dashed line marked with the asterisk indicates attachment to a nitrogen of an amine of a lysine side chain of -D.

In certain embodiments -L¹- is of formula (IX-f), wherein the dashed line marked with the asterisk indicates attachment to the nitrogen of the amine of the N-terminus of -D.

Accordingly, the linkage between the moiety -L¹- and -D formed in the compound of formula (IX-f) is a carbamate.

In certain embodiments s1 of formula (IX-f) is an integer selected from the group consisting of 1, 2, 3, 4 and 5. In certain embodiments s1 of formula (IX-f) is 1. In certain embodiments s1 of formula (IX-f) is 2. In certain embodiments s1 of formula (IX-f) is 3. In certain embodiments s1 of formula (IX-f) is 4. In certain embodiments s1 of formula (IX-f) is 5.

In certain embodiments s2 of formula (IX-f) is an integer selected from the group consisting of 1, 2, 3, 4, 5, 6, 7, 8, 9, 10, 11 and 12. In certain embodiments s2 of formula (IX-f) is an integer selected from the group consisting of 1, 2, 3, 4, 5, 6, 7 and 8. In certain embodiments s2 of formula (IX-f) is 1. In certain embodiments s2 of formula (IX-f) is 2. In certain embodiments s2 of formula (IX-f) is 3. In certain embodiments s2 of formula (IX-e) is 4. In certain embodiments s2 of formula (IX-f) is 5. In certain embodiments s2 of formula (IX-e) is 6. In certain embodiments s2 of formula (IX-f) is 7. In certain embodiments s2 of formula (IX-f) is 8.

In certain embodiments s1 of formula (IX-f) is 3 and s2 of formula (IX-f) is 3.

In certain embodiments the IL-2 conjugate is of formula (Ia). In certain embodiments x is 1. In certain embodiments x is 2. In certain embodiments x is 3. In certain embodiments x is 4.

In certain embodiments the IL-2 conjugate is of formula (Ib). In certain embodiments y is 2. In certain embodiments y is 3. In certain embodiments y is 4.

In certain embodiments the moiety -L¹-L²-Z is of formula (XI)

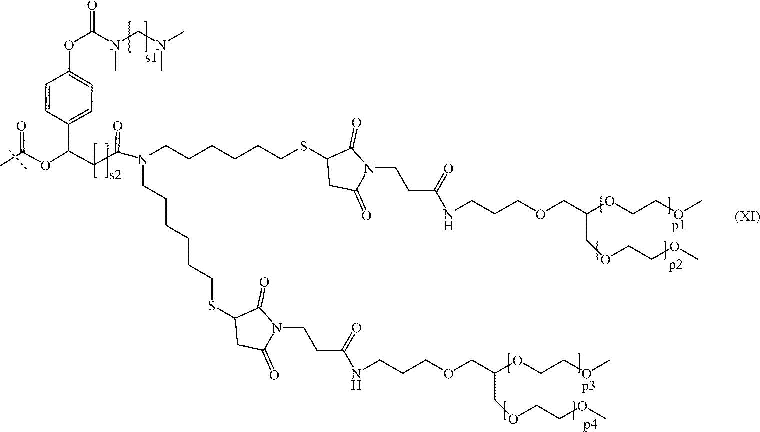

wherein the dashed line indicates attachment to a nitrogen of -D;
s1 is an integer selected from the group consisting of 1, 2, 3, 4, 5, 6, 7, 8, 9 and 10;
s2 is an integer selected from the group consisting of 0, 1, 2, 3, 4, 5, 6, 7, 8, 9, 10, 11, 12, 13, 14, 15, 16, 17, 18, 19 and 20; and
p1, p2, p3, p4 are independently of each other an integer ranging from 70 to 900.

In certain embodiments -L¹-L²-Z is of formula (XI), wherein the dashed line indicates attachment to a nitrogen of an amine of a lysine side chain of -D.

In certain embodiments -L¹-L²-Z is of formula (XI), wherein the dashed line indicates attachment to the nitrogen of the amine of the N-terminus of -D.

Accordingly, the linkage between the moiety -L¹- and -D formed in the compound of formula (XI) is a carbamate.

In certain embodiments s1 of formula (XI) is an integer selected from the group consisting of 1, 2, 3, 4 and 5. In certain embodiments s1 of formula (XI) is 1. In certain embodiments s1 of formula (XI) is 2. In certain embodiments s1 of formula (XI) is 3. In certain embodiments s1 of formula (XI) is 4. In certain embodiments s1 of formula (XI) is 5.

In certain embodiments s2 of formula (XI) is an integer selected from the group consisting of 1, 2, 3, 4, 5, 6, 7, 8, 9, 10, 11 and 12. In certain embodiments s2 of formula (XI) is an integer selected from the group consisting of 1, 2, 3, 4, 5, 6, 7 and 8. In certain embodiments s2 of formula (XI) is 1. In certain embodiments s2 of formula (XI) is 2. In certain embodiments s2 of formula (XI) is 3. In certain embodiments s2 of formula (XI) is 4. In certain embodiments s2 of formula (XI) is 5. In certain embodiments s2 of formula (XI) is 6. In certain embodiments s2 of formula (XI) is 7. In certain embodiments s2 of formula (XI) is 8.

In certain embodiments s1 of formula (XI) is 3 and s2 of formula (XI) is 3.

In certain embodiments p1 of formula (XI) is an integer ranging from 115 to 680. In certain embodiments p1 of formula (XI) is an integer ranging from 115 to 560. In certain embodiments p1 of formula (XI) is an integer ranging from 185 to 450. In certain embodiments p1 of formula (XI) is an integer ranging from 220 to 240. In certain embodiments p1 of formula (XI) is about 115. In certain embodiments p1 of formula (XI) is about 160. In certain embodiments p1 of formula (XI) is about 225. In certain embodiments p1 of formula (XI) is about 270. In certain embodiments p1 of formula (XI) is about 340. In certain embodiments p1 of formula (XI) is about 450. In certain embodiments p1 of formula (XI) is about 560.

In certain embodiments p2 of formula (XI) is an integer ranging from 115 to 680. In certain embodiments p2 of formula (XI) is an integer ranging from 115 to 560. In certain embodiments p2 of formula (XI) is an integer ranging from 185 to 450. In certain embodiments p2 of formula (XI) is an integer ranging from 220 to 240. In certain embodiments p2 of formula (XI) is about 115. In certain embodiments p2 of formula (XI) is about 160. In certain embodiments p2 of formula (XI) is about 225. In certain embodiments p2 of formula (XI) is about 270. In certain embodiments p2 of formula (XI) is about 340. In certain embodiments p2 of formula (XI) is about 450. In certain embodiments p2 of formula (XI) is about 560.

In certain embodiments p3 of formula (XI) is an integer ranging from 115 to 680. In certain embodiments p3 of formula (XI) is an integer ranging from 115 to 560. In certain embodiments p3 of formula (XI) is an integer ranging from 185 to 450. In certain embodiments p3 of formula (XI) is an integer ranging from 220 to 240. In certain embodiments p3 of formula (XI) is about 115. In certain embodiments p3 of formula (XI) is about 160. In certain embodiments p3 of formula (XI) is about 225. In certain embodiments p3 of formula (XI) is about 270. In certain embodiments p3 of formula (XI) is about 340. In certain embodiments p3 of formula (XI) is about 450. In certain embodiments p3 of formula (XI) is about 560.

In certain embodiments p4 of formula (XI) is an integer ranging from 115 to 680. In certain embodiments p4 of formula (XI) is an integer ranging from 115 to 560. In certain embodiments p4 of formula (XI) is an integer ranging from 185 to 450. In certain embodiments p4 of formula (XI) is an integer ranging from 220 to 240. In certain embodiments p4 of formula (XI) is about 115. In certain embodiments p4 of formula (XI) is about 160. In certain embodiments p4 of formula (XI) is about 225. In certain embodiments p4 of formula (XI) is about 270. In certain embodiments p4 of formula (XI) is about 340. In certain embodiments p4 of formula (XI) is about 450. In certain embodiments p4 of formula (XI) is about 560.

In certain embodiments p1, p2, p3 of formula (XI) and p4 are identical. In certain embodiments p1, p2, p3 and p4 range from 220 to 240.

In certain embodiments the moiety -L¹-L²-Z is of formula (XI-a)

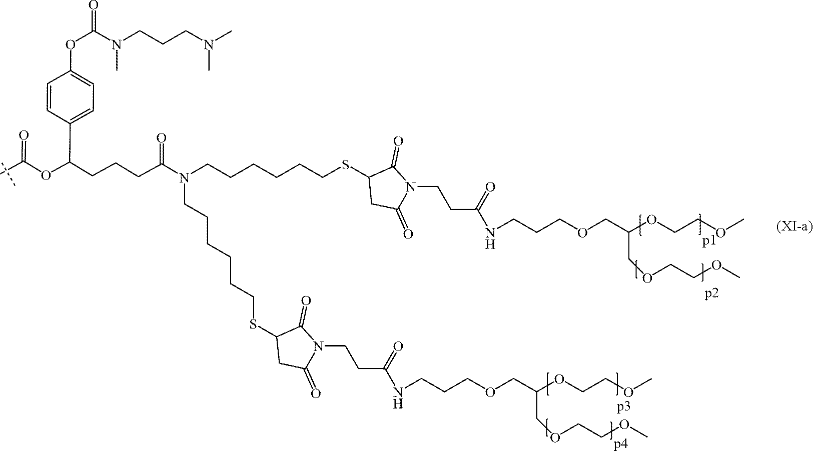

wherein the dashed line indicates attachment to a nitrogen of -D; and p1, p2, p3, p4 are independently of each other an integer ranging from 70 to 900.

In certain embodiments -L¹-L²-Z is of formula (XI-a), wherein the dashed line indicates attachment to a nitrogen of an amine of a lysine side chain of -D.

In certain embodiments -L¹-L²-Z is of formula (XI-a), wherein the dashed line indicates attachment to the nitrogen of the amine of the N-terminus of -D.

Accordingly, the linkage between the moiety -L¹- and -D formed in the compound of formula (XI-a) is a carbamate.

In certain embodiments p1 of formula (XI-a) is an integer ranging from 115 to 680. In certain embodiments p1 of formula (XI-a) is an integer ranging from 115 to 560. In certain embodiments p1 of formula (XI-a) is an integer ranging from 185 to 450. In certain embodiments p1 of formula (XI-a) is an integer ranging from 220 to 240. In certain embodiments p1 of formula (XI-a) is about 115. In certain embodiments p1 of formula (XI-a) is about 160. In certain embodiments p1 of formula (XI-a) is about 225. In certain embodiments p1 of formula (XI-a) is about 270. In certain embodiments p1 of formula (XI-a) is about 340. In certain embodiments p1 of formula (XI-a) is about 450. In certain embodiments p1 of formula (XI-a) is about 560.

In certain embodiments p2 of formula (XI-a) is an integer ranging from 115 to 680. In certain embodiments p2 of formula (XI-a) is an integer ranging from 115 to 560. In certain embodiments p2 of formula (XI-a) is an integer ranging from 185 to 450. In certain embodiments p2 of formula (XI-a) is an integer ranging from 220 to 240. In certain embodiments p2 of formula (XI-a) is about 115. In certain embodiments p2 of formula (XI-a) is about 160. In certain embodiments p2 of formula (XI-a) is about 225. In certain embodiments p2 of formula (XI-a) is about 270. In certain embodiments p2 of formula (XI-a) is about 340. In certain embodiments p2 of formula (XI-a) is about 450. In certain embodiments p2 of formula (XI-a) is about 560.

In certain embodiments p3 of formula (XI-a) is an integer ranging from 115 to 680. In certain embodiments p3 of formula (XI-a) is an integer ranging from 115 to 560. In certain embodiments p3 of formula (XI-a) is an integer ranging from 185 to 450. In certain embodiments p3 of formula (XI-a) is an integer ranging from 220 to 240. In certain embodiments p3 of formula (XI-a) is about 115. In certain embodiments p3 of formula (XI-a) is about 160. In certain embodiments p3 of formula (XI-a) is about 225. In certain embodiments p3 of formula (XI-a) is about 270. In certain embodiments p3 of formula (XI-a) is about 340. In certain embodiments p3 of formula (XI-a) is about 450. In certain embodiments p3 of formula (XI-a) is about 560.

In certain embodiments p4 of formula (XI-a) is an integer ranging from 115 to 680. In certain embodiments p4 of formula (XI-a) is an integer ranging from 115 to 560. In certain embodiments p4 of formula (XI-a) is an integer ranging from 185 to 450. In certain embodiments p4 of formula (XI-a) is an integer ranging from 220 to 240. In certain embodiments p4 of formula (XI-a) is about 115. In certain embodiments p4 of formula (XI-a) is about 160. In certain embodiments p4 of formula (XI-a) is about 225. In certain embodiments p4 of formula (XI-a) is about 270. In certain embodiments p4 of formula (XI-a) is about 340. In certain embodiments p4 of formula (XI-a) is about 450. In certain embodiments p4 of formula (XI-a) is about 560.

In certain embodiments p1, p2, p3 of formula (XI-a) and p4 are identical. In certain embodiments p1, p2, p3 and p4 range from 220 to 240.

In certain embodiments the conjugate of the present invention comprises an IL-2 moiety of SEQ ID NO:10, to which IL-2 moiety a moiety of formula (XI-a) is conjugated to the nitrogen of a primary amine of the N-terminus or a lysine side chain residue of the IL-2 moiety and p1, p2, p3 and p4 range from 220 to 240. In certain embodiments b3 is about 112. In certain embodiments the moiety of formula (XI-a) is conjugated to the nitrogen of a primary amine of a lysine side chain residue of the IL-2 moiety.

In certain embodiments the conjugate of the present invention comprises an IL-2 moiety of SEQ ID NO:13, to which IL-2 moiety a moiety of formula (XI-a) is conjugated to the nitrogen of a primary amine of the N-terminus or a lysine side chain residue of the IL-2 moiety and p1, p2, p3 and p4 range from 220 to 240. In certain embodiments b3 is about 112. In certain embodiments the moiety of formula (XI-a) is conjugated to the nitrogen of a primary amine of a lysine side chain residue of the IL-2 moiety.

In certain embodiments the conjugate of the present invention comprises an IL-2 moiety of SEQ ID NO:16, to which IL-2 moiety a moiety of formula (XI-a) is conjugated to the nitrogen of a primary amine of the N-terminus or a lysine side chain residue of the IL-2 moiety and p1, p2, p3 and p4 range from 220 to 240. In certain embodiments b3 is about 112. In certain embodiments the moiety of formula (XI-a) is conjugated to the nitrogen of a primary amine of a lysine side chain residue of the IL-2 moiety.

In certain embodiments the conjugate of the present invention comprises an IL-2 moiety of SEQ ID NO:19, to which IL-2 moiety a moiety of formula (XI-a) is conjugated to the nitrogen of a primary amine of the N-terminus or a lysine side chain residue of the IL-2 moiety and p1, p2, p3 and p4 range from 220 to 240. In certain embodiments b3 is about 112. In certain embodiments the moiety of formula (XI-a) is conjugated to the nitrogen of a primary amine of a lysine side chain residue of the IL-2 moiety.

In certain embodiments the conjugate of the present invention comprises an IL-2 moiety of SEQ ID NO:22, to which IL-2 moiety a moiety of formula (XI-a) is conjugated to the nitrogen of a primary amine of the N-terminus or a lysine side chain residue of the IL-2 moiety and p1, p2, p3 and p4 range from 220 to 240. In certain embodiments b3 is about 112. In certain embodiments the moiety of formula (XI-a) is conjugated to the nitrogen of a primary amine of a lysine side chain residue of the IL-2 moiety.

In certain embodiments the conjugate of the present invention comprises an IL-2 moiety of SEQ ID NO:25, to which IL-2 moiety a moiety of formula (XI-a) is conjugated to the nitrogen of a primary amine of the N-terminus or a lysine side chain residue of the IL-2 moiety and p1, p2, p3 and p4 range from 220 to 240. In certain embodiments b3 is about 112. In certain embodiments the moiety of formula (XI-a) is conjugated to the nitrogen of a primary amine of a lysine side chain residue of the IL-2 moiety.

In certain embodiments the conjugate of the present invention comprises an IL-2 moiety of SEQ ID NO:31, to which IL-2 moiety a moiety of formula (XI-a) is conjugated to the nitrogen of a primary amine of the N-terminus or a lysine side chain residue of the IL-2 moiety and p1, p2, p3 and p4 range from 220 to 240. In certain embodiments b3 is about 112. In certain embodiments the moiety of formula (XI-a) is conjugated to the nitrogen of a primary amine of a lysine side chain residue of the IL-2 moiety.

In certain embodiments the conjugate of the present invention comprises an IL-2 moiety of SEQ ID NO:34, to which IL-2 moiety a moiety of formula (XI-a) is conjugated to the nitrogen of a primary amine of the N-terminus or a lysine side chain residue of the IL-2 moiety and p1, p2, p3 and p4 range from 220 to 240. In certain embodiments b3 is about 112. In certain embodiments the moiety of formula (XI-a) is conjugated to the nitrogen of a primary amine of a lysine side chain residue of the IL-2 moiety.

In certain embodiments the conjugate of the present invention comprises an IL-2 moiety of SEQ ID NO:217, to which IL-2 moiety a moiety of formula (XI-a) is conjugated to the nitrogen of a primary amine of the N-terminus or a lysine side chain residue of the IL-2 moiety and p1, p2, p3 and p4 range from 220 to 240. In certain embodiments b3 is about 112. In certain embodiments the moiety of formula (XI-a) is conjugated to the nitrogen of a primary amine of a lysine side chain residue of the IL-2 moiety.

In certain embodiments the conjugate of the present invention comprises an IL-2 moiety of SEQ ID NO:225, to which IL-2 moiety a moiety of formula (XI-a) is conjugated to the nitrogen of a primary amine of the N-terminus or a lysine side chain residue of the IL-2 moiety and p1, p2, p3 and p4 range from 220 to 240. In certain embodiments b3 is about 112. In certain embodiments the moiety of formula (XI-a) is conjugated to the nitrogen of a primary amine of a lysine side chain residue of the IL-2 moiety.

Another aspect of the present invention is a pharmaceutical composition comprising at least one IL-2 protein of formula (I) or at least one IL-2 conjugate as described herein and at least one excipient. In certain embodiments such the pharmaceutical composition has a pH ranging from and including pH 3 to pH 8.

In certain embodiments such pharmaceutical composition is a liquid formulation. In certain embodiments the pharmaceutical composition is a suspension formulation. In certain embodiments the pharmaceutical composition is a dry formulation.

Such liquid, suspension or dry pharmaceutical composition comprises at least one excipient. Excipients used in parenteral formulations may be categorized as, for example, buffering agents, isotonicity modifiers, preservatives, stabilizers, anti-adsorption agents, oxidation protection agents, viscosifiers/viscosity enhancing agents, or other auxiliary agents. However, in some cases, one excipient may have dual or triple functions. In certain embodiments the at least one excipient comprised in the pharmaceutical composition of the present invention is selected from the group consisting of
(i) Buffering agents: physiologically tolerated buffers to maintain pH in a desired range, such as sodium phosphate, bicarbonate, succinate, histidine, citrate and acetate, sulphate, nitrate, chloride, pyruvate; antacids such as Mg(OH)₂ or ZnCO₃ may be also used;
(ii) Isotonicity modifiers: to minimize pain that can result from cell damage due to osmotic pressure differences at the injection depot; glycerin and sodium chloride are examples; effective concentrations can be determined by osmometry using an assumed osmolality of 285-315 mOsmol/kg for serum;
(iii) Preservatives and/or antimicrobials: multidose parenteral formulations require the addition of preservatives at a sufficient concentration to minimize risk of patients becoming infected upon injection and corresponding regulatory requirements have been established; typical preservatives include m-cresol, phenol, methylparaben, ethylparaben, propylparaben, butylparaben, chlorobutanol, benzyl alcohol, phenylmercuric nitrate, thimerosol, sorbic acid, potassium sorbate, benzoic acid, chlorocresol, and benzalkonium chloride;
(iv) Stabilizers: Stabilisation is achieved by strengthening of the protein-stabilising forces, by destabilisation of the denatured state, or by direct binding of excipients to the protein; stabilizers may be amino acids such as alanine, arginine, aspartic acid, glycine, histidine, lysine, proline, sugars such as glucose, sucrose, trehalose, polyols such as glycerol, mannitol, sorbitol, salts such as potassium phosphate, sodium sulphate, chelating agents such as EDTA, hexaphosphate, ligands such as divalent metal ions (zinc, calcium, etc.), other salts or organic molecules such as phenolic derivatives; in addition, oligomers or polymers such as cyclodextrins, dextran, dendrimers, PEG or PVP or protamine or HSA may be used;
(v) Anti-adsorption agents: Mainly ionic or non-ionic surfactants or other proteins or soluble polymers are used to coat or adsorb competitively to the inner surface of the formulation’s container; e.g., poloxamer (Pluronic F-68), PEG dodecyl ether (Brij 35), polysorbate 20 and 80, dextran, polyethylene glycol, PEG-polyhistidine, BSA and HSA and gelatins; chosen concentration and type of excipient depends on the effect to be avoided but typically a monolayer of surfactant is formed at the interface just above the CMC value;
(vi) Oxidation protection agents: antioxidants such as ascorbic acid, ectoine, methionine, glutathione, monothioglycerol, morin, polyethylenimine (PEI), propyl gallate, and vitamin E; chelating agents such as citric acid, EDTA, hexaphosphate, and thioglycolic acid may also be used;
(vii) Viscosifiers or viscosity enhancers: retard settling of the particles in the vial and syringe and are used in order to facilitate mixing and resuspension of the particles and to make the suspension easier to inject (i.e., low force on the syringe plunger); suitable viscosifiers or viscosity enhancers are, for example, carbomer viscosifiers like Carbopol 940, Carbopol Ultrez 10, cellulose derivatives like hydroxypropylmethylcellulose (hypromellose, HPMC) or diethylaminoethyl cellulose (DEAE or DEAE-C), colloidal magnesium silicate (Veegum) or sodium silicate, hydroxyapatite gel, tricalcium phosphate gel, xanthans, carrageenans like Satia gum UTC 30, aliphatic poly(hydroxy acids), such as poly(D,L- or L-lactic acid) (PLA) and poly(glycolic acid) (PGA) and their copolymers (PLGA), terpolymers of D,L-lactide, glycolide and caprolactone, poloxamers, hydrophilic poly(oxyethylene) blocks and hydrophobic poly(oxypropylene) blocks to make up a triblock of poly(oxyethylene)-poly(oxypropylene)-poly(oxyethylene) (e.g. Pluronic®), polyetherester copolymer, such as a polyethylene glycol terephthalate/polybutylene terephthalate copolymer, sucrose acetate isobutyrate (SAIB), dextran or derivatives thereof, combinations of dextrans and PEG, polydimethylsiloxane, collagen, chitosan, polyvinyl alcohol (PVA) and derivatives, polyalkylimides, poly (acrylamide-co-diallyldimethyl ammonium (DADMA)), polyvinylpyrrolidone (PVP), glycosaminoglycans (GAGs) such as dermatan sulfate, chondroitin sulfate, keratan sulfate, heparin, heparan sulfate, hyaluronan, ABA triblock or AB block copolymers composed of hydrophobic A-blocks, such as polylactide (PLA) or poly(lactide-co-glycolide) (PLGA), and hydrophilic B-blocks, such as polyethylene glycol (PEG) or polyvinyl pyrrolidone; such block copolymers as well as the abovementioned poloxamers may exhibit reverse thermal gelation behavior (fluid state at room temperature to facilitate administration and gel state above sol-gel transition temperature at body temperature after injection);
(viii) Spreading or diffusing agent: modifies the permeability of connective tissue through the hydrolysis of components of the extracellular matrix in the interstitial space such as but not limited to hyaluronic acid, a polysaccharide found in the intercellular space of connective tissue; a spreading agent such as but not limited to hyaluronidase temporarily decreases the viscosity of the extracellular matrix and promotes diffusion of injected drugs; and
(ix) Other auxiliary agents: such as wetting agents, viscosity modifiers, antibiotics, hyaluronidase; acids and bases such as hydrochloric acid and sodium hydroxide are auxiliary agents necessary for pH adjustment during manufacture.

Another aspect relates to the IL-2 protein of formula (I), the IL-2 conjugate or the pharmaceutical composition as described herein for use as a medicament.

Another aspect relates to the IL-2 protein of formula (I), the IL-2 conjugate or the pharmaceutical composition as described herein for use in the treatment of a disease which can be treated with IL-2.

Another aspect relates to the IL-2 protein of formula (I), the IL-2 conjugate or the pharmaceutical composition as described herein for the manufacture of a medicament for treating a disease which can be treated with IL-2.

Another aspect relates to a method of treating, controlling, delaying or preventing in a mammalian patient, preferably a human patient, in need of the treatment of one or more diseases which can be treated with IL-2, comprising the step of administering to said patient in need thereof a therapeutically effective amount of the IL-2 protein of formula (I), the IL-2 conjugate or the pharmaceutical composition as described herein.

In certain embodiments the disease which can be treated with IL-2 is cancer. Such cancer may be selected from the group consisting of liquid tumors, solid tumors and lymphomas.

A liquid lymphoma may be a leukemia or myeloid neoplasm, such as chronic lymphocytic leukemia (CLL), chronic myelogenous leukemia (CML), hairy cell leukemia, lymphoblastic leukemia, myeloid leukemia, plasma cell leukemia, acute lymphoblastic leukemia (ALL), acute myeloid leukemia (AML), myelodysplastic syndrome (MDS), myeloproliferative neoplasm (MPN), post-MPN AML, post-MDS AML, del(5q)-associated high risk MDS or AML, blast-phase chronic myelogenous leukemia, multiple myeloma, myelodysplastic syndromes, chronic myeloproliferative disorders, plasma cell neoplasm and Waldenstrom’s macroglobulinemia.

A solid tumor or lymphoma may be selected from the group consisting of lip and oral cavity cancer, oral cancer, liver cancer/hepatocellular cancer, primary liver cancer, lung cancer, lymphoma, malignant mesothelioma, malignant thymoma, skin cancer, intraocular melanoma, metastasic squamous neck cancer with occult primary, childhood multiple endocrine neoplasia syndrome, mycosis fungoides, nasal cavity and paranasal sinus cancer, nasopharyngeal cancer, neuroblastoma, oropharyngeal cancer, ovarian cancer, pancreatic cancer, parathyroid cancer, pheochromocytoma, pituitary tumor, adrenocortical carcinoma, AIDS-related malignancies, anal cancer, bile duct cancer, bladder cancer, brain and nervous system cancer, breast cancer, bronchial adenoma/carcinoid, gastrointestinal carcinoid tumor, carcinoma, colorectal cancer, endometrial cancer, esophageal cancer, extracranial germ cell tumor, extragonadal germ cell tumor, extrahepatic bile duct cancer, gallbladder cancer, gastric (stomach) cancer, gestational trophoblastic tumor, head and neck cancer, hypopharyngeal cancer, islet cell carcinoma (endocrine pancreas), kidney cancer/renal cell cancer, laryngeal cancer, pleuropulmonary blastoma, prostate cancer, transitional cell cancer of the renal pelvis and ureter, retinoblastoma, salivary gland cancer, sarcoma, Sezary syndrome, small intestine cancer, genitourinary cancer, malignant thymoma, thyroid cancer, Wilms’ tumor, cholangiocarcinoma, and also their related earlier stages of aberrant cell growth such as dysplasias, adenomas, and carcinoma in situs.

In certain embodiments the cancer is a liver cancer/hepatocellular cancer. In certain embodiments the cancer is a lung cancer. In certain embodiments the cancer is a lymphoma. In certain embodiments the cancer is a malignant thymoma. In certain embodiments the cancer is a skin cancer. In certain embodiments the cancer is a metastatic squamous neck cancer with occult primary. In certain embodiments the cancer is a neuroblastoma. In certain embodiments the cancer is an ovarian cancer. In certain embodiments the cancer is a pancreatic cancer. In certain embodiments the cancer is a bile duct cancer. In certain embodiments the cancer is a bladder cancer. In certain embodiments the cancer is a brain and nervous system cancer. In certain embodiments the cancer is a breast cancer. In certain embodiments the cancer is a gastrointestinal carcinoid tumor. In certain embodiments the cancer is a carcinoma. In certain embodiments the cancer is a colorectal cancer. In certain embodiments the cancer is an extrahepatic bile duct cancer. In certain embodiments the cancer is a gallbladder cancer. In certain embodiments the cancer is a gastric (stomach) cancer. In certain embodiments the cancer is a head and neck cancer. In certain embodiments the cancer is a kidney cancer/renal cell cancer. In certain embodiments the cancer is a prostate cancer. In certain embodiments the cancer is a sarcoma. In certain embodiments the cancer is a small intestine cancer. In certain embodiments the cancer is a genitourinary cancer.

Examples for lung cancer are non-small cell lung cancer and small cell lung cancer. In certain embodiments the cancer is a non-small cell lung cancer. In certain embodiment the cancer is a small cell lung cancer.

Example for lymphomas are AIDS-related lymphoma, primary central nervous system lymphoma, T-cell lymphoma, cutaneous T-cell lymphoma, Hodgkin’s lymphoma, Hodgkin’s lymphoma during pregnancy, non-Hodgkin’s lymphoma, non-Hodgkin’s lymphoma during pregnancy and angioimmunoblastic lymphoma.

Examples for skin cancer are melanoma and Merkel cell carcinoma. In certain embodiments the cancer is a skin cancer. In certain embodiments the cancer is a Merkel cell carcinoma.

An ovarian cancer may for example be an epithelial cancer, a germ cell tumor or a low malignant potential tumor. In certain embodiments the cancer is an epithelial cancer. In certain embodiments the cancer is a germ cell tumor. In certain embodiments the cancer is a low malignant potential tumor.

A pancreatic cancer may for example be an exocrine tumor/adenocarcinoma, pancreatic endocrine tumor (PET) or neuroendocrine tumor (NET). In certain embodiments the cancer is an exocrine tumor/adenocarcinoma. In certain embodiments the tumor is a pancreatic endocrine tumor. In certain embodiments the cancer is a neuroendocrine tumor.

Examples for brain and nervous system cancer are medulloblastoma, such as a childhood medulloblastoma, astrocytoma, ependymoma, neuroectodermal tumors, schwannoma, meningioma, pituitary adenoma and glioma. In certain embodiment the cancer is a medulloblastoma. In certain embodiments the cancer is a childhood medulloblastoma. In certain embodiments the cancer is an astrocytoma. In certain embodiments the cancer is an ependymoma. In certain embodiments the cancer is a neuroectodermal tumor. In certain embodiments the tumor is a schwannoma. In certain embodiments the cancer is a meningioma. In certain embodiments the cancer is a pituitary adenoma. In certain embodiments the cancer is a glioma.

An astrocytoma may be selected from the group consisting of giant cell glioblastoma, glioblastoma, secondary glioblastoma, primary adult glioblastoma, primary pediatric glioblastoma, oligodendroglial tumor, oligodendroglioma, anaplastic oligodendroglioma, oligoastrocytic tumor, oligoastrocytoma, anaplastic oligodendroglioma, oligoastrocytic tumor, oligoastrocytoma, anaplastic oligoastrocytoma, anaplastic astrocytoma, pilocytic astrocytoma, subependymal giant-cell astrocytoma, diffuse astrocytoma, pleomorphic xanthoastrocytoma and cerebellar astrocytoma.

Examples for a neuroectodermal tumor are a pineal primitive neuroectodermal tumor and a supratentorial primitive neuroectodermal tumor.

An ependymoma may be selected from the group consisting of subependymoma, ependymoma, myxopapillary ependymoma and anaplastic ependymoma.

A meningioma may be an atypical meningioma or an anaplastic meningioma.

A glioma may be selected from the group consisting of glioblastoma multiforme, paraganglioma, suprantentorial primordial neuroectodermal tumor (sPNET), brain stem glioma, childhood brain stem glioma, hypothalamic and visual pathway glioma, childhood hypothalamic and visual pathway glioma and malignant glioma.

Examples for breast cancer are breast cancer during pregnancy, triple negative breast cancer, ductal carcinoma in situ (DCIS), invasive ductal carcinoma (IDC), tubular carcinoma of the breast, medullary carcinoma of the breast, mucinous carcinoma of the breast, papillary carcinoma of the breast, cribriform carcinoma of the breast, invasive lobular carcinoma (ILC), inflammatory breast cancer, lobular carcinoma in situ (LCIS), male breast cancer, Paget’s disease of the nipple, phyllodes tumors of the breast and metastatic breast cancer. In certain embodiments the cancer is a breast cancer during pregnancy. In certain embodiments the cancer is a triple negative breast cancer. In certain embodiments the cancer is a ductal carcinoma in situ. In certain embodiments the cancer is an invasive ductal carcinoma. In certain embodiments the cancer is a tubular carcinoma of the breast. In certain embodiments the cancer is a medullary carcinoma of the breast. In certain embodiments the cancer is a mucinous carcinoma of the breast. In certain embodiments the cancer is a papillary carcinoma of the breast. In certain embodiments the cancer is a cribriform carcinoma of the breast. In certain embodiments the cancer is an invasive lobular carcinoma. In certain embodiments the cancer is an inflammatory breast cancer. In certain embodiments the cancer is a lobular carcinoma in situ. In certain embodiments the cancer is a male breast cancer. In certain embodiments the cancer is a Paget’s disease of the nipple. In certain embodiments the cancer is a phyllodes tumor of the breast. In certain embodiments the cancer is a metastatic breast cancer.

Examples for a carcinoma are neuroendocrine carcinoma, adrenocortical carcinoma and Islet cell carcinoma. In certain embodiments the cancer is a neuroendocrine carcinoma. In certain embodiments the cancer is an adrenocortical carcinoma. In certain embodiments the cancer is an Islet cell carcinoma.

Examples for a colorectal cancer are colon cancer and rectal cancer. In certain embodiments the cancer is a colon cancer. In certain embodiments the cancer is a rectal cancer.

A sarcoma may be selected from the group consisting of Kaposi’s sarcoma, osteosarcoma/malignant fibrous histiocytoma of bone, soft tissue sarcoma, Ewing’s family of tumors/sarcomas, rhabdomyosarcoma, clear cell sarcoma of tendon sheaths, central chondrosarcoma, central and periosteal chondroma, fibrosarcoma and uterine sarcoma. In certain embodiments the cancer may be a Kaposi’s sarcoma. In certain embodiments the cancer may be an osteosarcoma/malignant fibrous histiocytoma of bone. In certain embodiments the cancer may be a soft tissue sarcoma. In certain embodiments the cancer may be an Ewing’s family of tumors/sarcomas. In certain embodiments the cancer may be a rhabdomyosarcoma. In certain embodiments the cancer may be a clear cell sarcoma of tendon sheaths. In certain embodiments the cancer may be a central chondrosarcoma. In certain embodiments the cancer may be a central and periosteal chondroma. In certain embodiments the cancer may be a fibrosarcoma. In certain embodiments the cancer may be a uterine sarcoma.

Examples for a genitourinary cancer are testicular cancer, urethral cancer, vaginal cancer, cervical cancer, penile cancer and vulvar cancer. In certain embodiments the cancer may be a testicular cancer. In certain embodiments the cancer may be a urethral cancer. In certain embodiments the cancer may be a vaginal cancer. In certain embodiments the cancer may be a cervical cancer. In certain embodiments the cancer may be a penile cancer. In certain embodiments the cancer may be a vaginal cancer.

In certain embodiments the treatment with the IL-2 protein of formula (I), the IL-2 conjugate or the pharmaceutical composition of the present invention may be initiated prior to, concomitant with, or following surgical removal of a tumor or radiation therapy. In addition, such treatment may optionally be combined with at least one other cancer therapeutic, such as systemic immunotherapy or local intra-tumoral immunotherapy or intra-lymph node immunotherapy. Examples for the at least one cancer therapeutic, such as systemic immunotherapy, are as provided elsewhere herein for the one or more additional drug that may in certain embodiments be present in the pharmaceutical composition of the present invention.

In certain embodiments the IL-2 protein of formula (I), the IL-2 conjugate or the pharmaceutical composition of the present invention is administered systemically prior to, concomitant with, or following combination with at least one systemic immunotherapy or local intra-tumoral immunotherapy or intra-lymph node immunotherapy, prior to radiation therapy or surgical removal of the injected tumor. In certain embodiments the IL-2 protein of formula (I), the IL-2 conjugate or the pharmaceutical composition of the present invention is administered intratumorally prior to, concomitant with, or following combination with at least one systemic immunotherapy or local intra-tumoral immunotherapy or intra-lymph node immunotherapy, prior to radiation therapy or surgical removal of the injected tumor. In certain embodiments the conjugate, its pharmacologically acceptable salt or the pharmaceutical composition of the present invention is administered intratumorally prior to, concomitant with, or following combination with at least one systemic immunotherapy or local intra-tumoral immunotherapy or intra-lymph node immunotherapy, following radiation therapy or surgical removal of a tumor. In certain embodiments the IL-2 protein of formula (I), the IL-2 conjugate or the pharmaceutical composition of the present invention is administered into tumor draining lymph nodes prior to, concomitant with, or following surgical removal of a tumor or radiation therapy. In certain embodiments the IL-2 protein of formula (I), the IL-2 conjugate or the pharmaceutical composition of the present invention is administered into tumor draining lymph nodes prior to, concomitant with, or following combination with at least one systemic immunotherapy or local intra-tumoral immunotherapy or intra-lymph node immunotherapy, and prior to, concomitant with, or following surgical removal of a tumor or radiation therapy. In certain embodiments the IL-2 protein of formula (I), the IL-2 conjugate or the pharmaceutical composition of the present invention is administered intratumorally into metastatic tumors that may arise prior to or following surgical removal or radiation therapy of primary tumor. In certain embodiments the IL-2 protein of formula (I), the IL-2 conjugate or the pharmaceutical composition of the present invention is administered intratumorally into metastatic tumors that may arise prior to, concomitant with, or following combination with at least one systemic immunotherapy or local intra-tumoral immunotherapy or intra-lymph node immunotherapy, and prior to, concomitant with, or following surgical removal or radiation therapy of primary tumor. In certain embodiments at least one systemic therapy is administered prior to surgical removal of a tumor or radiation therapy, followed by systemic administration or intra-tumoral administration or intra-lymph node administration of the IL-2 protein of formula (I), the IL-2 conjugate or the pharmaceutical composition of the present invention. In certain embodiments intra-tumoral administration of the IL-2 protein of formula (I), the IL-2 conjugate or the pharmaceutical composition of the present invention is administered first, followed by subsequent treatment in combination with at least one systemic therapy or local intra-tumoral immunotherapy or intra-lymph node immunotherapy. In certain embodiments at least one systemic therapy is administered prior to surgical removal of a tumor, followed by administration the IL-2 protein of formula (I), the IL-2 conjugate or the pharmaceutical composition of the present invention systemically or to draining lymph nodes or to the tumor bed following surgery or by intra-tumoral administration in tumor not removed by surgery.

In certain embodiments the IL-2 protein of formula (I), the IL-2 conjugate or the pharmaceutical composition of the present invention is administered to the patient prior to, simultaneously with, or after administration of one or more additional drug, which one or more additional drug is in certain embodiments selected from the group consisting of pattern recognition receptor agonists (PRRA), cytotoxic/chemotherapeutic agents, immune checkpoint inhibitors or antagonists, immune checkpoint agonists, immune activating receptor agonists, multi-specific drugs, antibody-drug conjugates (ADC), antibody-adjuvant conjugates (AAC), radionuclides or targeted radionuclide therapeutics, DNA damage repair inhibitors, tumor metabolism inhibitors, protein kinase inhibitors, chemokine and chemoattractant receptor agonists, chemokine or chemokine receptor antagonists, cytokine receptor agonists, death receptor agonists, CD47 or SIRPα antagonists, oncolytic drugs, signal converter proteins, epigenetic modifiers, tumor peptides or tumor vaccines, heat shock protein (HSP) inhibitors, proteolytic enzymes, ubiquitin and proteasome inhibitors, adhesion molecule antagonists, hormones including hormone peptides and synthetic hormones, and adoptive cellular therapies such as Tumor Infiltrating Lymphocyte (TIL) therapy, Chimeric Antigen Receptor (CAR) therapy, T cell therapy, Natural Killer (NK) cell therapy, CAR-T therapy, CAR-NK therapy, CAR-γδ therapy, CAR-Macrophage therapy, or any other cellular therapy with a genetically modified or genetically unmodified immune cell type.

The PRRA may be selected from the group consisting of Toll-like receptor (TLR) agonists, NOD-like receptor agonists (NLRs), RIG-I-like receptor agonists, cytosolic DNA sensor agonists, STING agonists, and aryl hydrocarbon receptor agonists (AhR).

In certain embodiments the PRRA is a Toll-like receptor agonist, such as a Toll-like receptor agonists selected from the group consisting of agonists of TLR½, such as peptidoglycans, lipoproteins, Pam3CSK4, Amplivant, SLP-AMPLIVANT, HESPECTA, ISA101 and ISA201; agonists of TLR2, such as LAM-MS, LPS-PG, LTA-BS, LTA-SA, PGN-BS, PGN-EB, PGN-EK, PGN-SA, CL429, FSL-1, Pam2CSK4, Pam3CSK4, zymosan, CBLB612, SV-283, ISA204, SMP105, heat killed Listeria monocytogenes; agonists of TLR3, such as poly(A:U), poly(I:C) (poly-ICLC), rintatolimod, apoxxim, IPH3102, poly-ICR, PRV300, RGCL2, RGIC.1, Riboxxim (RGC100, RGIC100), Riboxxol (RGIC50)), synthetic natural or modified double stranded RNA, synthetic natural or modified nucleic acid oligomers and Riboxxon; agonists of TLR4, such as lipopolysaccharides (LPS), neoceptin-3, glucopyranosyl lipid adjuvant (GLA), GLA-SE, G100, GLA-AF, clinical center reference endotoxin (CCRE), monophosphoryl lipid A, grass MATA MPL, PEPA10, ONT-10 (PET-Lipid A, oncothyreon), G-305, ALD046, CRX527, CRX675 (RC527, RC590), GSK1795091, OM197MPAC, OM294DP, tumor targeted TLR4 agonists, and SAR439794; agonists of TLR2/4, such as lipid A, OM174 and PGN007; agonists of TLR5, such as flagellin, entolimod, mobilan, protectan CBLB501; agonists of TLR6/2, such as diacylated lipoproteins, diacylated lipopeptides, FSL-1, MALP-2 and CBLB613; agonists of TLR7, such as CL264, CL307, imiquimod (R837), TMX-101, TMX-201, TMX-202, TMX302, gardiquimod, S-27609, 851, UC-IV150, 852A (3M-001, PF-04878691), loxoribine, polyuridylic acid, GSK2245035, GS-9620, RO6864018 (ANA773, RG7795), RO7020531, isatoribine, AN0331, ANA245, ANA971, ANA975, DSP0509, DSP3025 (AZD8848), GS986, MBS2, MBS5, RG7863 (RO6870868), sotirimod, SZU101, synthetic natural or modified single stranded RNA, synthetic nucleic acids, synthetic natural or modified nucleic acid oligomers, tumor targeted TLR7 agonists, and TQA3334; agonists of TLR8, such as ssPolyUridine, ssRNA40, TL8-506, XG-1-236, VTX-2337 (motolimod), VTX-1463, VTX378, VTX763, DN1508052, SBT6050, synthetic natural or modified single stranded RNA, synthetic nucleic acids, synthetic natural or modified nucleic acid oligomers, tumor targeted TLR8 agonists, and GS9688; agonists of TLR⅞, such as TransCon™ TLR⅞ agonist, CL075, CL097, poly(dT), resiquimod (R-848, VML600, S28463), MEDI9197 (3M-052), NKTR262, DV1001, IMO4200, IPH3201, synthetic natural or modified single stranded RNA, synthetic nucleic acids, synthetic nucleic acid oligomers, BDC-1001, other tumor targeted TLR⅞ agonists and VTX1463; agonists of TLR9, such as CpG DNA, CpG ODN, lefitolimod (MGN1703), SD-101, QbG10, CYT003, CYT003-QbG10, DUK-CpG-001, CpG-7909 (PF-3512676), GNKG168, EMD 1201081, IMO-2125, IMO-2055, CpG10104, AZD1419, AST008, IMO2134, MGN1706, IRS 954, 1018 ISS, actilon (CPG10101), ATP00001, AVE0675, AVE7279, CMP001, DIMS0001, DIMS9022, DIMS9054, DIMS9059, DV230, DV281, EnanDIM, heplisav (V270), kappaproct (DIMS0150), NJP834, NPI503, SAR21609, synthetic natural or modified nucleic acid oligomers and tolamba; and agonists of TLR7/9, such as DV1179.

In certain embodiments the one or more additional drug is an agonist of TLR½. In certain embodiments the one or more additional drug is an agonist of TLR2. In certain embodiments the one or more additional drug is an agonist of TLR3. In certain embodiments the one or more additional drug is an agonist of TLR4. In certain embodiments the one or more additional drug is an agonist of TLR2/4. In certain embodiments the one or more additional drug is an agonist of TLR5. In certain embodiment the one or more additional drug is an agonist of TLR6/2. In certain embodiments the one or more additional drug is an agonist of TLR7. In certain embodiments the one or more additional drug is an agonist of TLR8. In certain embodiments the one or more additional drug is an agonist of TLR⅞. In certain embodiments the one or more additional drug is an agonist of TLR9.

Examples for CpG ODN are ODN 1585, ODN 2216, ODN 2336, ODN 1668, ODN 1826, ODN 2006, ODN 2007, ODN BW006, ODN D-SL01, ODN 2395, ODN M362 and ODN D-SL03.

In certain embodiments the one or more additional drug is resiquimod. In certain embodiments the one or more additional drug is imiquimod.

In certain embodiments the one or more additional drug is resiquimod in its free form. In certain embodiments the one or more additional drug is a conjugate comprising a polymer, to which one or more moieties of formula (A-i) are conjugated (A-i), wherein
the dashed line indicates attachment to the polymer; and
n is an integer selected from the group consisting of 1, 2, 3, 4, 5, 6, 7, 8, 9, 10, 11, 12, 13, 14, 15, 16, 17, 18, 19 or 20.

In certain embodiments the polymer to which the one or more moieties of formula (A-i) are conjugated is a water-soluble polymer. In certain embodiments such water-soluble polymer is a PEG-based or hyaluronic acid-based polymer. In certain embodiments the polymer is a PEG-based polymer. In certain embodiments the polymer is a hydrogel, such as a PEG-based or hyaluronic acid-based hydrogel. In certain embodiments the hydrogel is a PEG-based hydrogel. In certain embodiments n of formula (A-i) is 1. In certain embodiments n of formula (A-i) is 2. In certain embodiments n of formula (A-i) is 3. In certain embodiments n of formula (A-i) is 4.

In certain embodiments the one or more additional drugs is a conjugate comprising a PEG-based hydrogel to which a multitude of the moieties of formula (A-i) is conjugated, wherein n in formula (A-i) is 2. In certain embodiments the one or more additional drugs is compound 12 or 14 from WO2020/141221A1 as shown on page 217 and 219, respectively, which are herewith incorporated by reference. In certain embodiments the one or more additional drugs is compound 12 from WO2020/141221A1 as shown on page 217. In certain embodiments the one or more additional drugs is compound 14 from WO2020/141221A1 as shown on page 219. Compounds 12 and 14 can be synthesized as disclosed in WO2020/141221A1.

In certain embodiments the PRRA is a NOD-like receptor agonist. If the one or more additional drug is a NOD-like receptor agonist, such NOD-like receptor agonist may be selected from the group consisting of agonists of NODI1, such as C12-iE-DAP, C14-Tri-LAN-Gly, iE-DAP, iE-Lys, and Tri-DAP; and agonists of NOD2, such as L18-MDP, MDP, M-TriLYS, murabutide and N-glycolyl-MDP. In certain embodiments the one or more additional drug is an agonist of NOD1. In certain embodiments the one or more additional drug is an agonist of NOD2.

In certain embodiments the PRRA is a RIG-I-like receptor agonist. If the one or more additional drug is a RIG-I-like receptor agonist, such RIG-I-like receptor agonist may be selected from the group consisting of 3p-hpRNA, 5′ppp-dsRNA, 5′ppp RNA (M8), 5′OH RNA with kink (CBS-13-BPS), 5′PPP SLR, KIN100, KIN 101, KIN1000, KIN1400, KIN1408, KIN1409, KIN1148, KIN131A, poly(dA:dT), SB9200, RGT100 and hiltonol.

In certain embodiments the PRRA is a cytosolic DNA sensor agonist. If the one or more additional drug is a cytosolic DNA sensor agonist, such cytosolic DNA sensor agonist may be selected from the group consisting of cGAS agonists, dsDNA-EC, G3-YSD, HSV-60, ISD, ODN TTAGGG (A151), poly(dG:dC) and VACV-70.

In certain embodiments the PRRA is a STING agonist. If the one or more additional drug is a STING agonist, such STING agonist may be selected from the group consisting of MK-1454, ADU-S100 (MIW815), 2′3′-cGAMP, 3′3′-cGAMP, c-di-AMP, c-di-GMP, cAIMP (CL592), cAIMP difluor (CL614), cAIM(PS)2 difluor (Rp/Sp) (CL656), 2′2′-cGAMP, 2′3′-cGAM(PS)2 (Rp/Sp), 3′3′-cGAM fluorinated, c-di-AMP fluorinated, 2′3′-c-di-AMP, 2′3′-c-di-AM(PS)2 (Rp,Rp), c-di-GMP fluorinated, 2′3′-c-di-GMP, c-di-IMP, c-di-UMP and DMXAA (vadimezan, ASA404). In certain embodiments the one or more additional drug is MK-1454. In certain embodiments the one or more additional drug is ADU-S100 (MIW815). In certain embodiments the one or more additional drug is 2′3′-cGAMP.

In certain embodiments the PRRA is an aryl hydrocarbon receptor agonist. If the one or more additional drug is an aryl hydrocarbon receptor (AhR) agonist, such AhR agonist may be selected from the group consisting of FICZ, ITE and L-kynurenine.

In certain embodiments the one or more additional drug is a cytotoxic/chemotherapeutic agent. In certain embodiments the one or more additional drug is an immune checkpoint inhibitor or antagonist. In certain embodiments the one or more additional drug is an immune activating receptor agonist. In certain embodiments the one or more additional drug is a multi-specific drug. In certain embodiments the one or more additional drug is an antibody-drug conjugate (ADC). In certain embodiments the one or more additional drug is an antibody-adjuvant conjugate (AAC). In certain embodiments the one or more additional drug is a radionuclide or a targeted radionuclide therapeutic. In certain embodiments the one or more additional drug is DNA damage repair inhibitor. In certain embodiments the one or more additional drug is a tumor metabolism inhibitor. In certain embodiments the one or more additional drug is a pattern recognition receptor agonist. In certain embodiments the one or more additional drug is a protein kinase inhibitor. In certain embodiments the one or more additional drug is a chemokine and chemoattractant receptor agonist. In certain embodiments the one or more additional drug is a chemokine or chemokine receptor antagonist. In certain embodiments the one or more additional drug is a cytokine receptor agonist. In certain embodiments the one or more additional drug is a death receptor agonist. In certain embodiments the one or more additional drug is a CD47 antagonist. In certain embodiments the one or more additional drug is a SIRPα antagonist. In certain embodiments the one or more additional drug is an oncolytic drug. In certain embodiments the one or more additional drug is a signal converter protein. In certain embodiments the one or more additional drug is an epigenetic modifier. In certain embodiments the one or more additional drug is a tumor peptide or tumor vaccine. In certain embodiments the one or more additional drug is a heat shock protein (HSP) inhibitor. In certain embodiments the one or more additional drug is a proteolytic enzyme. In certain embodiments the one or more additional drug is a ubiquitin and proteasome inhibitor. In certain embodiments the one or more additional drug is an adhesion molecule antagonist. In certain embodiments the one or more additional drug is a hormone including hormone peptides and synthetic hormones.

The cytotoxic or chemotherapeutic agent may be selected from the group consisting of alkylating agents, anthracyclines, pyrrolobenzodiazepines, nitrogen mustards, platinum agents, anti-metabolites, anti-microtubule agents, topoisomerase inhibitors, cytotoxic antibiotics, auristatins, enediynes, lexitropsins, duocarmycins, cyclopropylpyrroloindoles, puromycin, dolastatins, maytansine derivatives, alkylsufonates, triazenes and piperazine.

The alkylating agent may be selected from the group consisting of nitrogen mustards, such as mechlorethamine, cyclophosphamide, melphalan, chlorambucil, ifosfamide and busulfan; nitrosoureas, such as N-nitroso-N-methylurea, carmustine, lomustine, semustine, fotemustine and streptozotocin; tetrazines, such as dacarbazine, mitozolomide and temozolomide; ethylenimines, such as altretamine; aziridines, such as thiotepa, mitomycin and diaziquone; cisplatin and derivatives, such as cisplatin, carboplatin, oxaliplatin; and non-classical alkylating agents, such as procarbazine and hexamethylmelamine.

The anti-metabolite may be selected from the group consisting of anti-folates, such as methotrexate and pemetrexed; fluoropyrimidines, such as fluorouracil and capecitabine; deoxynucleoside analogues, such as cytarabine, gemcitabine, decitabine, azacytidine, fludarabine, nelarabine, cladribine, clofarabine and pentostatin; and thiopurines, such as thioguanine and mercaptopurine.

The anti-microtubule agent may be selected from the group consisting of Vinca alkaloids, such as vincristine, vinblastine, vinorelbine, vindesine and vinflunine; taxanes, such as paclitaxel and docetaxel; podophyllotoxins and derivatives, such as podophyllotoxin, etoposide and teniposide; stilbenoid phenol and derivatives, such as zybrestat (CA4P); and BNC105.

The topoisomerase inhibitor may be selected from the group consisting of topoisomerase I inhibitors, such as irinotecan, topotecan and camptothecin; and topoisomerase II inhibitors, such as etoposide, doxorubicin, mitoxantrone, teniposide, novobiocin, merbarone and aclarubicin.

The cytotoxic antibiotic may be selected from the group consisting of anthracyclines, such as doxorubicin, daunorubicin, epirubicin and idarubicin; pirarubicin, aclarubicin, bleomycin, mitomycin C, mitoxantrone, actinomycin, dactinomycin, adriamycin, mithramycin and tirapazamine.

The auristatin may be selected from the group consisting of monomethyl auristatin E (MMAE) and monomethyl auristatin F (MMAF).

The enediyne may be selected from the group consisting of neocarzinostatin, lidamycin (C-1027), calicheamicins, esperamicins, dynemicins and golfomycin A.

The maytansine derivative may be selected from the group consisting of ansamitocin, mertansine (emtansine, DM1) and ravtansine (soravtansine, DM4).

The immune checkpoint inhibitor or antagonist may be selected from the group consisting of inhibitors of CTLA-4 (cytotoxic T-lymphocyte-associated protein 4), such as ipilimumab, tremelimumab, MK-1308, FPT155, PRS010, BMS-986249, BPI-002, CBT509, JS007, ONC392, TE1254, IBI310, BR02001, CG0161, KN044, PBI5D3H5, BCD145, ADU1604, AGEN1884, AGEN1181, CS1002 and CP675206; inhibitors of PD-1 (programmed death 1), such as pembrolizumab, nivolumab, pidilizumab, AMP-224, BMS-936559, cemiplimab and PDR001; inhibitors of PD-L1 (programmed cell death protein 1), such as MDX-1105, MEDI4736, atezolizumab, avelumab, BMS-936559 and durvalumab; inhibitors of PD-L2 (programmed death-ligand 2); inhibitors of KIR (killer-cell immunoglobulin-like receptor), such as lirlumab (IPH2102) and IPH2101; inhibitors of B7-H3, such as MGA271; inhibitors of B7-H4, such as FPA150; inhibitors of BTLA (B- and T-lymphocyte attenuator); inhibitors of LAG3 (lymphocyte-activation gene 3), such as IMP321 (eftilagimod alpha), relatlimab, MK-4280, AVA017, BI754111, ENUM006, GSK2831781, INCAGN2385, LAG3Ig, LAG525, REGN3767, Sym016, Sym022, TSR033, TSR075 and XmAb22841; inhibitors of TIM-3 (T-cell immunoglobulin and mucin-domain containing-3), such as LY3321367, MBG453, and TSR-022; inhibitors of VISTA (V-domain Ig suppressor of T cell activation), such as JNJ-61610588; inhibitors of ILT2/LILRB1 (Ig-like transcript 2/leukocyte Ig-like receptor 1); inhibitor of ILT3/LILRB4 (Ig-like transcript 3/leukocyte Ig-like receptor 4); inhibitors of ILT4/LILRB2 (Ig-like transcript 4/leukocyte Ig-like receptor 2), such as MK-4830; inhibitors of TIGIT (T cell immunoreceptor with Ig and ITIM domains), such as MK-7684, PTZ-201, RG6058 and COM902; inhibitors of NKG2A, such as IPH-2201; inhibitors of PVRIG, such as COM701; inhibitors of TREM1 such as PY314; and inhibitors of TREM2 such as PY159.

One example of a an inhibitor of CTLA-4 is an anti-CTLA4 conjugate or a pharmaceutically acceptable salt thereof, wherein said conjugate comprises a plurality of anti-CTLA4 moieties -D_{CTLA4} covalently conjugated via at least one moiety -L¹-L²- to a polymeric moiety Z, wherein -L¹- is covalently and reversibly conjugated to -D_{CTLA4} and -L²- is covalently conjugated to Z and wherein -L¹- is a linker moiety and -L²- is a chemical bond or a spacer moiety, wherein the moieties -L¹-, -L²- and Z are as described elsewhere herein for the conjugate of the present invention. In certain embodiments -D_{CTLA4} is selected from the group consisting of wild-type F_{c} anti-CTLA4 antibodies, F_{c} enhanced for effector function/FcyR binding anti-CTLA4 antibodies, anti-CTLA4 antibodies conditionally active in tumor microenvironment, anti-CTLA4 small molecules, CTLA4 antagonist fusion proteins, anti-CTLA4 anticalins, anti-CTLA4 nanobodies and anti-CTLA4 multispecific biologics based on antibodies, scFVs or other formats. In certain embodiments -D_{CTLA4} is ipilimumab. In certain embodiments -D_{CTLA4} is tremelimumab. In certain embodiments the anti-CTLA4 conjugate has the following structure:

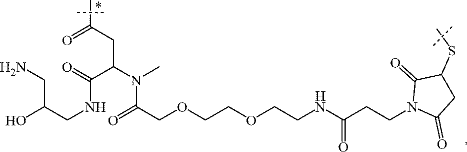

wherein
the dashed line marked with the asterisk indicates attachment to the nitrogen of an amine functional group of -D_{CTLA4}, in particular to the nitrogen of an amine functional group of ipilimumab; and
the unmarked dashed line indicates attachment to Z, such as a hydrogel, in particular to a crosslinked hyaluronic acid hydrogel.

It is understood that a multitude of moieties -D_{CTLA4}-L¹-L²- are connected to Z, if Z is a hydrogel, such as a crosslinked hyaluronic acid hydrogel.

In certain embodiments the nitrogen of an amine functional group of -D_{CTLA4} and in particular of ipilimumab is an amine of a lysine residue. In certain embodiments the nitrogen of an amine functional group of -D_{CTLA4} and in particular of ipilimumab is the N-terminal amine.

In certain embodiments the one or more additional drug is an inhibitor of CTLA4 as described above.

The immune activating receptor agonist may be selected from the group consisting of agonists of CD27, such as recombinant CD70, such as HERA-CD27L, and varlilumab (CDX-1127); agonists of CD28, such as recombinant CD80, recombinant CD86, TGN1412 and FPT155; agonists of CD40, such as recombinant CD40L, CP-870,893, dacetuzumab (SGN-40), Chi Lob 7/4, ADC-1013 and CDX1140; agonists of 4-1BB (CD137), such as recombinant 4-1BBL, urelumab, utomilumab and ATOR-1017; agonists of OX40, such as recombinant OX40L, MEDI0562, GSK3174998, MOXR0916 and PF-04548600; agonists of GITR, such as recombinant GITRL, TRX518, MEDI1873, INCAGN01876, MK-1248, MK-4166, GWN323 and BMS-986156; and agonists of ICOS, such as recombinant ICOSL, JTX-2011 and GSK3359609.

The multi-specific drug may be selected from the group consisting of biologics and small molecule immune checkpoint inhibitors. Examples for biologics are multi-specific immune checkpoint inhibitors, such as CD137/HER2 multispecifics, PD-(L)⅟LAG3 antagonists (for example FS118, MGD013), CTLA4/LAG3 antagonists (for example XmAb22841) and CTLA4/PD-(L)1 antagonists (for example XmAb20717, MGD019); multispecific immune activating receptor agonists, immunocytokines and multi-specific immune checkpoint agonists. Such multi-specific immune checkpoint agonists may be selected from the group consisting of Ig superfamily agonists, such as ALPN-202, FPT155, TGN1412, GSK3359609, JTX-2011; TNF superfamily agonists, such as FAP-4-1BBL (RG7826), OX40-41BB (FS120) ATOR-1015, ATOR-1144, ALG.APV-527, lipocalin/PRS-343, PRS344/ONC0055, FAP-CD40 DARPin, MP0310 DARPin, FAP-0X40 DARPin, EGFR-CD40 DARPin, EGFR41BB/CD137 DARPin, EGFR-0X40/DARFPin, HER2-CD40 DARPin, HER2-41BB/CD137 DARPin, HER2-0X40 DARPin, FIBRONECTIN ED-B-CD40 DARPin, FIBRONECTIN ED-B-41BB/CD137 and FIBRONECTIN ED-B-0X40 DARPin; CD3 multispecific agonists, such as blinatumomab, solitomab, MEDI-565, ertumaxomab, anti-HER2/CD3, 1Fab-immunoblobulin G TDB, GBR 1302, MGD009, MGD007, EGFRBi, EGFR-CD Probody, RG7802, PF-06863135, PF-06671008, AMG212/BAY2010112, CD3-5T4, XmAb14045, XmAb13676, XmAb18087, S80880, REGN1979, REGN5458, REGN4018, RG6026, Mosunetuzumab, EM801, ERY974, RG6194, AMG420, AMG330, AMG 212, AMG 596, AMG 160, AMG 427, AMG 562, AMG 673, AMG 701, AMG 757, AFM13, AMF24, AFM26, AFM11. TNB-486, TNB-383B, GEN3013, JNJ-63709178, JNJ-63898081, JNJ-64007957, JNJ-64407564, JNJ-67571244, AMV564, APVO414 (MOR209, ES414), APVO436, HPN424, HPN536, HPN217, HPN 328 and other multispecific CD3 agonists or T cell receptor (TCR) agonists including γδ TCR agonists targeting T cell activity towards a tumor cell antigen or viral antigen or expressing cell; Natural Killer (NK) cell receptor multispecific agonists targeting an activating NK receptor and a target tumor cell antigen, such as NKG2D multispecific agonists, NKp30 multispecific agonists, NKp44 multispecific agonists, NKp46 multispecific agonists, NKp80 multispecific agonists, NKG2C multispecific agonists, 2B4 (CD244) multispecific agonists, CD32a multispecific agonists, CD64 multispecific agonists, multispecific agonists that bind to a tumor antigen as well as activating receptors such as NKG2D or NKp30 or other NK receptors listed above as well as binding to F_{c} receptors such as TriNKeTs, and CD16 multispecific agonists, such as 1633 BiKE, 161533 TriKE, OXS-3550, OXS-C3550, AFM13 and AFM24; and other therapeutic antibodies capable of binding a target antigen as well as F_{c} receptors such as CD16, CD32a, CD64.

Other examples of immune activating receptor agonists include Dectin agonists (Imprime PGG), recombinant NKG2D ligands, ligand or modifiers of yδ TCR signaling such as anti-BTN3A1 mAbs or anti- BTN2A1 mAbs or Vγ9/Vδ2 TCR activating ligand such as phospho antigens and pyrophosphate antigens, or agents which increase endogenous Vγ9/Vδ2 ligands such as bisphosphonates like pamidronate and zoledronate.

An example for a small molecule immune checkpoint inhibitor is CA-327 (TIM3/PD-L1 antagonist).

The antibody-drug conjugate may be selected from the group consisting of ADCs targeting hematopoietic cancers, such as gemtuzumab ozogamicin, brentuximab vedotin, inotuzumab ozogamicin, SAR3419, BT062, SGN-CD19A, IMGN529, MDX-1203, polatuzumab vedotin (RG7596), pinatuzumab vedotin (RG7593), RG7598, milatuzumab-doxorubicin and OXS-1550; and ADCs targeting solid tumor antigens, such as trastuzumab emtansine, glembatumomab vedotin, SAR56658, AMG-172, AMG-595, BAY-94-9343, BIIB015, vorsetuzumab mafodotin (SGN-75), ABT-414, ASG-5ME, enfortumab vedotin (ASG-22ME), ASG-16M8F, IMGN853, indusatumab vedotin (MLN-0264), vadortuzumab vedotin (RG7450), sofituzumab vedotin (RG7458), lifastuzumab vedotin (RG7599), RG7600, DEDN6526A (RG7636), PSMA TTC, 1095 from Progenics Pharmaceuticals, lorvotuzumab mertansine, lorvotuzumab emtansine, IMMU-130, sacituzumab govitecan (IMMU-132), PF-06263507 and MEDI0641.

The antibody-adjuvant conjugate may be a boltbody, such as the boltbodies described in WO2018112108A1 and WO2018009916A1. In certain embodiments the boltbody is selected from the group consisting of BDC-1001 and BDC-2034. In certain embodiments the boltbody is BDC-1001. In certain embodiments the boltbody is BDC-2034.

In certain embodiments the boltbody has the structure of formula (BT-I)

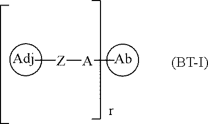

wherein Ab is an antibody moiety;
A is an unmodified amino acid sidechain in the antibody moiety or a modified amino acid sidechain in the antibody moiety;
Z is a linking moiety;
Adj is an adjuvant moiety; and
r is an integer selected from 1 to 10.

It is understood that r amino acid side chains of a moiety Ab of formula (BT-I) are connected to a moiety Adj-Z.

In certain embodiments A of formula (BT-I) comprises an amino acid sidechain in the antibody moiety comprising an amine functional group.

In certain embodiments the boltbody has the structure of formula (BT-II)

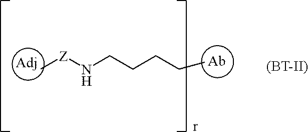

wherein
Ab is an antibody moiety;

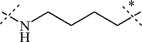

represents a sidechain of a lysine residue of Ab, wherein
   the unmarked dashed line indicates attachment to Z and the dashed line marked with the asterisk indicates attachment to the alpha carbon of the lysine residue;
   Adj is an adjuvant moiety;
   r is an integer selected from 1 to 10;
   and Z is a divalent linking moiety having an ethylene glycol group or a glycine residue.

It is understood that the moiety

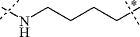

of formula (BT-II) corresponds to A of formula (BT-I). Likewise, it is understood that r lysine side chain moieties of moiety Ab of formula (BT-II) are connected to a moiety Adj-Z.

In certain embodiments Z of formulas (BT-I) and (BT-II) is bonded to Adj via an amide bond, a C—N single bond, a C—O single bond, or a C—C single bond, and to Ab via an amide bond or a C—N single bond.

In certain embodiments Z of formulas (BT-I) and (BT-II) is bonded to a nitrogen group of Adj and a nitrogen group of Ab. In such embodiments Z of formulas (BT-I) and (BT-II) is bonded to adjacent nitrogen groups via amide bonds, C—N single bonds, or a combination thereof.

In some embodiments Z of formulas (BT-I) and (BT-II) comprises a PEG moiety.

In certain embodiments Z of formulas (BT-I) and (BT-II) comprises at least 2 ethylene glycol groups, such as at least 3 ethylene glycol groups, at least 4 ethylene glycol groups, at least 5 ethylene glycol groups, at least 6 ethylene glycol groups, at least 7 ethylene glycol groups, at least 8 ethylene glycol groups, at least 9 ethylene glycol groups, at least 10 ethylene glycol groups, at least 11 ethylene glycol groups, at least 12 ethylene glycol groups, at least 13 ethylene glycol groups, at least 14 ethylene glycol groups, at least 15 ethylene glycol groups, at least 16 ethylene glycol groups, at least 17 ethylene glycol groups, at least 18 ethylene glycol groups, at least 19 ethylene glycol groups, at least 20 ethylene glycol groups, at least 21 ethylene glycol groups, at least 22 ethylene glycol groups, at least 23 ethylene glycol groups, at least 24 ethylene glycol groups, or at least 25 ethylene glycol groups.

In certain embodiments Z of formulas (BT-I) and (BT-II) comprises 2 ethylene glycol groups, 3 ethylene glycol groups, 4 ethylene glycol groups, 5 ethylene glycol groups, 6 ethylene glycol groups, 8 ethylene glycol groups, 12 ethylene glycol groups, 24 ethylene glycol groups, or 25 ethylene glycol groups.

In certain embodiments Z of formulas (BT-I) and (BT-II) comprises a glycine residue.

In certain embodiments Z of formulas (BT-I) and (BT-II) comprises at least 2 glycine residues, such as at least 3 glycine residues, at least 4 glycine residues, at least 5 glycine residues, at least 6 glycine residues, at least 7 glycine residues, at least 8 glycine residues, at least 9 glycine residues, at least 10 glycine residues, at least 11 glycine residues, at least 12 glycine residues, at least 13 glycine residues, at least 14 glycine residues, at least 15 glycine residues, at least 16 glycine residues, at least 17 glycine residues, at least 18 glycine residues, at least 19 glycine residues, at least 20 glycine residues, at least 21 glycine residues, at least 22 glycine residues, at least 23 glycine residues, at least 24 glycine residues, or at least 25 glycine residues.

In certain embodiments Z of formulas (BT-I) and (BT-II) comprises 2 glycine residues, 3 glycine residues, 4 glycine residues, 5 glycine residues, 6 glycine residues, 8 glycine residues, 12 glycine residues, 24 glycine residues, or 25 glycine residues.

In certain embodiments Z of formulas (BT-I) and (BT-II) further comprises a divalent cyclohexylene group

In certain embodiments Ab of formulas (BT-I) and (BT-II) comprises an antibody binding domain that binds to an antigen selected from the group consisting of CDH1, CD19, CD20, CD29, CD30, CD38, CD40, CD47, CEA, EpCAM, MUC1, MUC16, EGFR, VEGF, HER2, SLAMF7, PDGFRa, gp75, CTLA4, PD-1, PD-L1, PD-L2, LAG-3, B7-H4, KIR, TNFRSF4, OX40L, IDO-1, IDO-2, CEACAM1, BTLA, TIM3, A2Ar, VISTA, CLEC4C (BDCA-2, DLEC, CD303, CLECSF7), CLEC4D (MCL, CLECSF8), CLEC4E (Mincle), CLEC6A (Dectin-2), CLEC5A (MDL-1, CLECSF5), CLEC1B (CLEC-2), CLEC9A (DNGR-1), and CLEC7A (Dectin-1).

In certain embodiments Ab of formulas (BT-I) and (BT-II) comprises an antibody binding domain that binds to HER2. In certain embodiments Ab of formulas (BT-I) and (BT-II) comprises an antibody binding domain that binds to EGFR. In certain embodiments Ab of formulas (BT-I) and (BT-II) comprises an antibody binding domain that binds to CCR8. In certain embodiments Ab of formulas (BT-I) and (BT-II) comprises an antibody binding domain that binds to PD-L1. In certain embodiments Ab of formulas (BT-I) and (BT-II) comprises an antibody binding domain that binds to CEA.

In certain embodiments Ab of formulas (BT-I) and (BT-II) comprises an antibody selected from the group consisting of pembrolizumab, nivolumab, atezolizumab, avelumab, ipilimumab, obinutuzumab, trastuzumab, cetuximab, rituximab, pertuzumab, bevacizumab, daratumumab, etanercept, olaratumab, elotuzumab, margetuximab, and a biosimilar thereof.

In certain embodiments Ab of formulas (BT-I) and (BT-II) comprises trastuzumab. In certain embodiments Ab of formulas (BT-I) and (BT-II) comprises pembrolizumab. In certain embodiments Ab of formulas (BT-I) and (BT-II) comprises nivolumab.

In certain embodiments Adj of formulas (BT-I) and (BT-II) comprises a PRRA.

In certain embodiments Adj of formulas (BT-I) and (BT-II) is a PRRA selected from the group consisting of toll-like receptors (TLR) agonists, c-type lectin receptors (CLR) agonists, NOD-like receptors (NLR) agonists, Rig-I-like receptors (RLR) agonists, stimulator of interferon genes (STING) agonists and combination thereof.

In certain embodiments Adj of formulas (BT-I) and (BT-II) is a TLR agonist, such as a TLR agonist selected from the group consisting of TLR1 agonists, TLR2 agonists, TLR3 agonists, TLR4 agonists, TLR5 agonists, TLR6 agonists, TLR7 agonists, TLR⅞ agonists, TLR8 agonists, TLR9 agonists, TLR10 agonists, TLR11 agonists and combination thereof.

In certain embodiments Adj of formulas (BT-I) and (BT-II) is a TLR agonist selected from the group consisting of CL264, CL401, CL413, CL419, CL553, CL572, Pam₃CSK₄, and Pam₂CSK₄.

In certain embodiments, Adj of formulas (BT-I) and (BT-II) is selected from the group consisting of

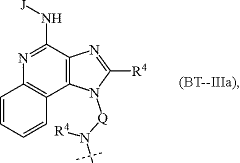

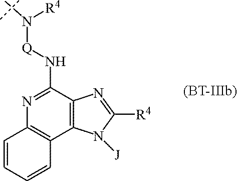

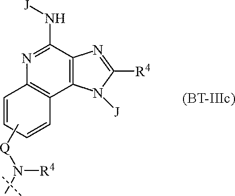

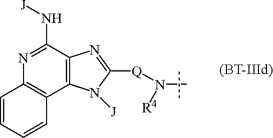

wherein
each -J is independently selected from the group consisting of —H, —OR⁴, and -R⁴;
each -R⁴ is independently selected from the group consisting of —H, an alkyl, heteroalkyl, cycloalkyl, heterocycloalkyl, aryl, heteroaryl, arylalkyl, and heteroarylalkyl group comprising from 1, 2, 3, 4, 5, 6, 7, or 8 carbon units;
-Q- is absent or is selected from the group consisting of alkyl, heteroalkyl, cycloalkyl, heterocycloalkyl, aryl, heteroaryl, arylalkyl, and heteroarylalkyl comprising from 1, 2, 3, 4, 5, 6, 7, or 8 carbon units; and
the dashed line indicates attachment to Z.

In certain embodiments Adj of formulas (BT-I) and (BT-II) is of formula

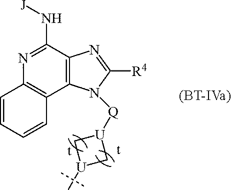

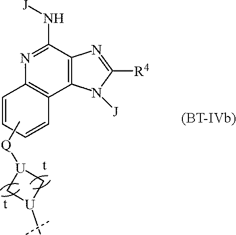

wherein
each -J is independently selected from the group consisting of —H, —OR⁴, or -R⁴;
each -R⁴ is independently selected from the group consisting of —H, alkyl, heteroalkyl, cycloalkyl, heterocycloalkyl, aryl, heteroaryl, arylalkyl, and heteroarylalkyl comprising from 1, 2, 3, 4, 5, 6, 7, or 8 carbon units;
each —U— is independently —CH— or —N—, wherein at least one —U— is —N—;
each t is independently an integer selected from 1, 2 and 3;
-Q- is absent or is selected from the group consisting of alkyl, heteroalkyl, cycloalkyl, heterocycloalkyl, aryl, heteroaryl, arylalkyl, and heteroarylalkyl comprising from 1, 2, 3, 4, 5, 6, 7, or 8 carbon units, and
the dashed line indicates attachment to Z.

In certain embodiments, Adj of formulas (BT-I) and (BT-II) is selected from the group consisting of formulas

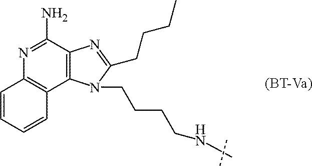

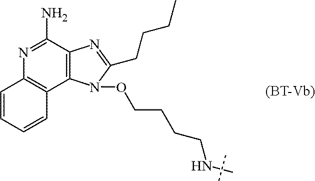

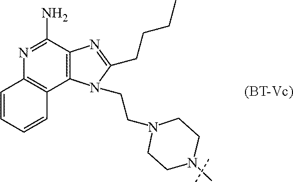

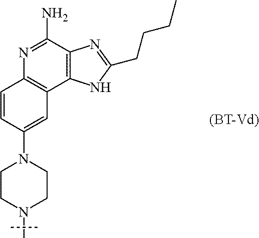

wherein the dashed line indicates attachment to Z.

In certain embodiments Adj of formulas (BT-I) and (BT-II) is selected from the adjuvant moieties disclosed in paragraphs [118] to [136] of WO2018112108A1.

In certain embodiments the boltbody comprises more than one distinct adjuvant moiety.

In certain embodiments the boltbody has the structure of formula (BT-VI)

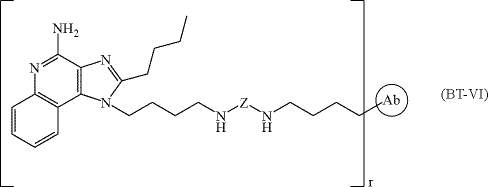

wherein
Ab is an antibody moiety;

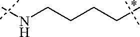

represents a sidechain of a lysine residue of Ab, wherein
   the unmarked dashed line indicates attachment to Z and the dashed line marked with the asterisk indicates attachment to the alpha carbon of the lysine residue;
   r is an integer selected from 1 to 10; and
   Z is a divalent linking moiety comprising at least one ethylene glycol group or at least one glycine residue.

In certain embodiments Z of formula (BT-VI) is used as defined for formulas (BT-I) and (BT-II).

In certain embodiments the boltbody has the structure of formula (BT-VII)

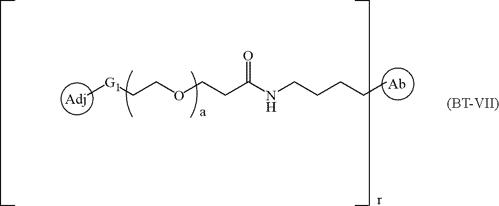

wherein
Ab is an antibody moiety comprising (i) an antigen binding domain and (ii) an F_{c} domain;
Adj is an adjuvant moiety of formula (BT-IVb)

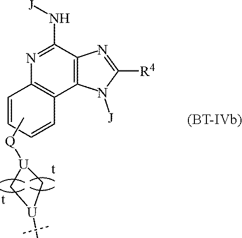

wherein
   -R⁴ is selected from the group consisting of alkyl, heteroalkyl, cycloalkyl, heterocycloalkyl, aryl, heteroaryl, arylalkyl, and heteroarylalkyl comprising from 1 to 8 carbons;
   each -J is —H;
   each —U— is —N—;
   each t is 2;
   -Q- is absent;
   the dashed line indicates attachment to G₁;
   -G₁- is a bond;
   a is an integer selected from 1 to 40; and
   r is an integer selected from 1 to 10.

In certain embodiments the boltbody has the structure of formula (BT-VII)

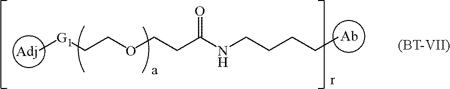

wherein
Ab is trastuzumab;
Adj is an adjuvant moiety of formula (BT-IVb)

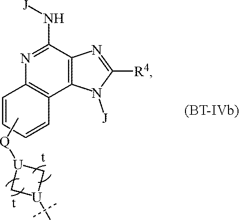

wherein
   -R4 is butyl;
   each -J is —H;
   each —U— is —N—;
   each t is 2;
   -Q- is absent;
   the dashed line indicates attachment to -G₁-;
   -G₁- is a bond;
   a is an integer selected from 1 to 40; and
   r is an integer selected from 1 to 4.

In certain embodiments the boltbody has the structure of formula (BT-VIII)

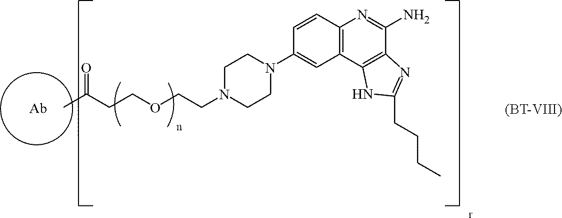

wherein
r is an integer selected from 1 to 10;
n is an integer selected from about 2 to about 25; and
Ab is an antibody moiety.

In certain embodiments r of formula (BT-VIII) is 1. In certain embodiments r of formula (BT-VIII) is 2. In certain embodiments r of formula (BT-VIII) is 3. In certain embodiments r of formula (BT-VIII) is 4.

In certain embodiments n of formula (BT-VIII) is an integer selected from 6, 7, 8, 9, 10, 11 and 12. In certain embodiments n of formula (BT-VIII) is an integer selected from 8, 9, 10, 11 and 12. In certain embodiments n of formula (BT-VIII) is 10.

In certain embodiments Ab of formula (BT-VIII) comprises an antigen binding domain that binds HER2, EGFR, PD-L1 or CEA. In certain embodiments the antibody moiety of formula (BT-VIII) comprises an antigen binding domain that binds HER2. In certain embodiments Ab of formula (BT-VIII) comprises an antigen binding domain that binds EGFR. In certain embodiments Ab of formula (BT-VIII) comprises an antigen binding domain that binds PD-L1. In certain embodiments Ab of formula (BT-VIII) comprises an antigen binding domain that binds CEA.

Only in the context of formulas (BT-IIIa), (BT-IIIb), (BT-IIIc), (BT-IIId), (BT-IVa), and (BT-IVb) the terms used have the following meaning:

The term “alkyl” refers to a straight or branched, saturated, aliphatic radical having the number of carbon atoms indicated. Alkyl may include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl comprises methyl, ethyl, propyl, isopropyl, butyl, isobutyl, sec-butyl, tert-butyl, pentyl, isopentyl, hexyl. Alkyl may also comprise alkyl groups having up to 30 carbons atoms, such as heptyl, octyl, nonyl, decyl. Alkyl groups may be substituted or unsubstituted. “Substituted alkyl” groups may be substituted with one or more groups selected from halo, hydroxy, amino, oxo (═O), alkylamino, amido, acyl, nitro, cyano, and alkoxy.

The term “aryl” refers to an aromatic ring system having any suitable number of ring atoms and any suitable number of rings. Aryl groups may comprise any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups may be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl.

The term “carbocycle” refers to a saturated or partially unsaturated, monocyclic, fused bicyclic, or bridged poly cyclic ring assembly containing from 3 to 12 ring atoms, or the number of atoms indicated. Carbocycles may include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic carbocyclic rings include, for example, cyclopropyl, cyclobutyl, cyclopentyl, cyclohexyl, and cyclooctyl. Saturated bicyclic and polycyclic carbocyclic rings include, for example, norbornane, [2.2.2] bicyclooctane, decahydronaphthalene and adamantane. Carbocyclic groups may also be partially unsaturated, having one or more double or triple bonds in the ring. Representative carbocyclic groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene.

The term “heteroalkyl” refers to an alkyl group, wherein one or more carbon atoms are optionally and independently replaced with heteroatom selected from N, O, and S.

The term “heterocycle” refers to heterocycloalkyl groups and heteroaryl groups. “Heteroaryl,” by itself or as part of another substituent, refers to a monocyclic or fused bicyclic or tricyclic aromatic ring assembly containing 5 to 16 ring atoms, where from 1 to 5 of the ring atoms are a heteroatom such as N, O or S. Additional heteroatoms may also be useful, such as B, Al, Si and P. The heteroatoms may be oxidized to form moieties such as, such as —S(O)— and —S(O)₂—. Heteroaryl groups may include any number of ring atoms, such as 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms may be include in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. The heteroaryl group may include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups may also be fused to aromatic ring systems, such as a phenyl ring, to form members, such as benzopyrroles, such as indole and isoindole, benzopyri dines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups may be substituted or unsubstituted. “Substituted heteroaryl” groups may be substituted with one or more groups selected from halo, hydroxy, amino, oxo (=O), alkylamino, amido, acyl, nitro, cyano, and alkoxy. Heteroaryl groups may be linked via any position on the ring. For example, pyrrole includes 1-, 2- and 3 -pyrrole, pyridine includes 2-, 3- and 4-pyridine, imidazole includes 1-, 2-, 4- and 5-imidazole, pyrazole includes 1-, 3-, 4-and 5-pyrazole, triazole includes 1-, 4- and 5-triazole, tetrazole includes 1- and 5-tetrazole, pyrimidine includes 2-, 4-, 5- and 6- pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3 -thiophene, furan includes 2- and 3 -furan, thiazole includes 2-, 4- and 5-thiazole, isothiazole includes 3-, 4- and 5-isothiazole, oxazole includes 2-, 4- and 5- oxazole, isoxazole includes 3-, 4- and 5-isoxazole, indole, such as 1-, 2-and 3-indole, isoindole, such as 1- and 2-isoindole, quinoline, such as 2-, 3- and 4-quinoline, isoquinoline, such as 1-, 3- and 4-isoquinoline, quinazoline, such as 2- and 4-quinoazoline, cinnoline, such as 3- and 4-cinnoline, benzothiophene, such as 2- and 3 -benzothiophene, and benzofuran, such as 2- and 3 -benzofuran.

The term “heterocycloalkyl,” by itself or as part of another substituent, refers to a saturated ring system having from 3 to 12 ring members and from 1 to 4 heteroatoms of N, O and S. Additional heteroatoms may also be useful, such as B, Al, Si and P. The heteroatoms may be oxidized to form moieties, such as —S(O)— and —S(O)₂—. Heterocycloalkyl groups may include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms may be included in the heterocycloalkyl groups, such as 1, 2, 3, or 4, or 1 to 2, 1 to 3, 1 to 4, 2 to 3, 2 to 4, or 3 to 4. The heterocycloalkyl group may include groups such as aziridine, azetidine, pyrrolidine, piperidine, azepane, azocane, quinuclidine, pyrazolidine, imidazolidine, piperazine (1,2-, 1,3-and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocycloalkyl groups may also be fused to aromatic or non-aromatic ring systems to form members, such as indoline. Heterocycloalkyl groups may be unsubstituted or substituted. “Substituted heterocycloalkyl” groups may be substituted with one or more groups selected from halo, hydroxy, amino, oxo (═O), alkylamino, amido, acyl, nitro, cyano, and alkoxy. Heterocycloalkyl groups may be linked via any position on the ring. For example, aziridine may be 1- or 2-aziridine, azetidine may be 1- or 2- azetidine, pyrrolidine may be 1-, 2- or 3 -pyrrolidine, piperidine may be 1-, 2-, 3- or 4-piperidine, pyrazolidine may be 1-, 2-, 3-, or 4-pyrazolidine, imidazolidine may be 1-, 2-, 3- or 4-imidazolidine, piperazine may be 1-, 2-, 3- or 4-piperazine, tetrahydrofuran may be 1- or 2-tetrahydrofuran, oxazolidine may be 2-, 3-, 4- or 5-oxazolidine, isoxazolidine may be 2-, 3-, 4- or 5-isoxazolidine, thiazolidine may be 2-, 3-, 4- or 5-thiazolidine, isothiazolidine may be 2-, 3-, 4- or 5-isothiazolidine, and morpholine may be 2-, 3- or 4-morpholine.

The term “arylalkyl” refers to any aryl derivative of an alkyl group. In certain embodiments one or more aryl moieties may be coupled to the remainder of the molecule through an alkyl linkage. Under those circumstances, the substituent will be referred to as an arylalkyl, indicating that an alkylene moiety is between the aryl moiety and the molecule to which the aryl is coupled. Representative arylalkyl groups include phenylmethyl, phenylethyl, phenylpropyl, phenylisopropyl, phenylbutyl, phenyl-isobutyl, phenyl-sec-butyl, phenyl-tert-butyl, phenylpentyl, phenyl-isopentyl, phenylhexyl, naphthylmethyl, naphthylethyl, naphthylpropyl, naphthylisopropyl, naphthylbutyl, naphthyl-isobutyl, naphthyl-sec-butyl, naphthyl-tert-butyl, naphthylpentyl, naphthyl-isopentyl, naphthylhexyl, biphenylmethyl, biphenylethyl, biphenylpropyl, biphenylisopropyl, biphenylbutyl, biphenyl-isobutyl, biphenyl-sec-butyl, biphenyl-tert-butyl, biphenylpentyl, biphenyl-isopentyl, and biphenylhexyl,

The term “heteroarylalkyl” refers to an arylalkyl group wherein one or more carbon atoms are optionally and independently replaced with heteroatom selected from N, O, and S.

In certain embodiments the one or more additional drug is a radionuclide which may be selected from the group consisting of β-emitters, such as ¹⁷⁷Lutetium, ¹⁶⁶Holmium, ¹⁸⁶Rhenium, ¹⁸⁸Rhenium, ⁶⁷Copper, ¹⁴⁹Promethium, ¹⁹⁹Gold, ⁷⁷Bromine, ¹⁵³Samarium, ¹⁰⁵Rhodium, ⁸⁹Strontium, ⁹⁰Yttrium, ¹³¹Iodine; α-emitters, such as ²¹³Bismuth, ²²³Radium, ²²⁵Actinium, ²¹¹Astatine; and Auger electron-emitters, such as ⁷⁷Bromine, ¹¹¹Indium, ¹²³Iodine and ¹²⁵Iodine.

The targeted radionuclide therapeutics may be selected from the group consisting of zevalin (⁹⁰Y-ibritumomab tiuxetan), bexxar (¹³¹I-tositumomab), oncolym (¹³¹I-Lym 1), lymphocide (⁹⁰Y-epratuzumab), cotara (¹³¹I-chTNT-⅟B), labetuzumab (⁹⁰Y or ¹³¹I-CEA), theragyn (⁹⁰Y-pemtumomab), licartin (¹³¹I-metuximab), radretumab (¹³¹I-L19) PAM4 (⁹⁰Y-clivatuzumab tetraxetan), xofigo (²²³Ra dichloride), lutathera (¹⁷⁷Lu-DOTA-Tyr³-Octreotate) and ¹³¹I-MIBG.

The DNA damage repair inhibitor may be selected from the group consisting of poly (ADP-ribose) polymerase (PARP) inhibitors, such as olaparib, rucaparib, niraparib, veliparib, CEP 9722 and E7016; CHK1/CHK2 dual inhibitors, such as AZD7762, V158411, CBP501 and XL844; CHK1 selective inhibitors, such as PF477736, MK8776/SCH900776, CCT244747, CCT245737, LY2603618, LY2606368/prexasertib, AB-IsoG, ARRY575, AZD7762, CBP93872, ESP01, GDC0425, SAR020106, SRA737, V158411 and VER250840; CHK2 inhibitors, such as CCT241533 and PV1019; ATM inhibitors, such as AZD0156, AZD1390, KU55933, M3541 and SX-RDS1; ATR inhibitors, such as AZD6738, BAY1895344, M4344 and M6620 (VX-970); and DNA-PK inhibitors, such as M3814.

The tumor metabolism inhibitor may be selected from the group consisting of inhibitors of the adenosine pathway, inhibitors of the tryptophan metabolism and inhibitors of the arginine pathway.

Examples for an inhibitor of the adenosine pathway are inhibitors of A2AR (adenosine A2A receptor), such as ATL-444, istradefylline (KW-6002), MSX-3, preladenant (SCH-420,814), SCH-58261, SCH412,348, SCH-442,416, ST-1535, caffeine, VER-6623, VER-6947, VER-7835, vipadenant (BIIB-014), ZM-241,385, PBF-509 and V81444; inhibitors of CD73, such as IPH53 and SRF373; and inhibitors of CD39, such as IPH52.

Examples for an inhibitor of the tryptophan metabolism are inhibitors of IDO, such as indoximod (NLG8189), epacadostat, navoximod, BMS-986205 and MK-7162; inhibitors of TDO, such as 680C91; and IDO/TDO dual inhibitors.

Examples for inhibitors of the arginine pathway are inhibitors of arginase, such as INCB001158.

The protein kinase inhibitor may be selected from the group consisting of receptor tyrosine kinase inhibitors, intracellular kinase inhibitors, cyclin dependent kinase inhibitors, phosphoinositide-3-kinase inhibitors, mitogen-activated protein kinase inhibitors, inhibitors of nuclear factor kappa-β kinase (IKK), and Wee-1 inhibitors.

Examples for receptor tyrosine kinase inhibitors are EGF receptor inhibitors, such as afatinib, cetuximab, erlotinib, gefitinib, pertuzumab and margetuximab; VEGF receptor inhibitors, such as axitinib, lenvatinib, pegaptanib and linifanib (ABT-869); C-KIT Receptor inhibitors, such as CDX0158 (KTN0158); ERBB2 (HER2) inhibiors, such as herceptin (trastuzumab); ERBB3 receptor inhibitors, such as CDX3379 (MEDI3379, KTN3379) and AZD8931 (sapitinib); FGF receptor inhibitors, such as erdafitinib; AXL receptor inhibitors, such as BGB324 (BGB 324, R 428, R428, bemcentinib) and SLC391; and MET receptor inhibitors, such as CGEN241.

Examples for intracellular kinase inhibitors are Bruton’s tyrosine kinase (BTK) inhibitors, such as ibrutinib, acalabrutinib, GS-4059, spebrutinib, BGB-3111, HM71224, zanubrutinib, ARQ531, BI-BTK1 and vecabrutinib; spleen tyrosine kinase inhibitors, such as fostamatinib; Bcr-Abl tyrosine kinase inhibitors, such as imatinib and nilotinib; Janus kinase inhibitors, such as ruxolitinib, tofacitinib and fedratinib; and multi-specific tyrosine kinase inhibitors, such as bosutinib, crizotinib, cabozantinib, dasatinib, entrectinib, lapatinib, mubritinib, pazopanib, sorafenib, sunitinib, SU6656 and vandetanib.

One example of a tyrosine kinase inhibitor is a tyrosine kinase inhibitor (“TKI”) conjugate or a pharmaceutically acceptable salt thereof, wherein said conjugate comprises a plurality of TKI moieties -D_{TKI} covalently conjugated via at least one moiety -L¹-L²- to a polymeric moiety Z, wherein -L¹- is covalently and reversibly conjugated to -D_{TKI} and -L²- is covalently conjugated to Z and wherein -L¹- is a linker moiety and -L²- is a chemical bond or a spacer moiety, wherein the moieties -L¹-, -L²- and Z are as described elsewhere herein for the conjugate of the present invention. In certain embodiments -D_{TKI} is selected from the group consisting of receptor tyrosine kinase inhibitors, intracellular kinase inhibitors, cyclin dependent kinase inhibitors, phosphoinositide-3-kinase (PI3K) inhibitors, mitogen-activated protein kinase inhibitors, inhibitors of nuclear factor kappa-β kinase (IKK), and Wee-1 inhibitors. In certain embodiments -D_{TKI} is axitinib. In certain embodiments -D_{TKI} is lenvatinib. In certain embodiments -D_{TKI} is pegaptanib. In certain embodiments -D_{TKI} is linifanib.

Examples for cyclin dependent kinase inhibitors are ribociclib, palbociclib, abemaciclib, trilaciclib, purvalanol A, olomucine II and MK-7965.

Examples for phophoinositide-3-kinase inhibitors are IPI549, GDc-0326, pictilisib, serabelisib, IC-87114, AMG319, seletalisib, idealisib and CUDC907.

Examples for mitogen-activated protein kinase inhibitors are Ras/farnesyl transferase inhibitors, such as tipirafinib and LB42708; Raf inhibitors, such as regorafenib, encorafenib, vemurafenib, dabrafenib, sorafenib, PLX-4720, GDC-0879, AZ628, lifirafenib, PLX7904 and RO5126766; MEK inhibitors, such as cobimetinib, trametinib, binimetinib, selumetinib, pimasertib, refametinib and PD0325901; ERK inhibitors, such as MK-8353, GDC-0994, ulixertinib and SCH772984.

Examples for inhibitors of nuclear factor kappa-β kinase (IKK) are BPI-003 and AS602868.

An example of a Wee-1 inhibitor is adavosertib.

The chemokine receptor and chemoattractant receptor agonist may be selected from the group consisting of CXC chemokine receptors, CC chemokine receptors, C chemokine receptors, CX3C chemokine receptors and chemoattractant receptors.

The CXC chemokine receptor may be selected from the group consisting of CXCR1 agonists, such as recombinant CXCL8 and recombinant CXCL6; CXCR2 agonists, such as recombinant CXCL8, recombinant CXCL1, recombinant CXCL2, recombinant CXCL3, recombinant CXCL5, recombinant CXCL6, MGTA 145 and SB251353; CXCR3 agonists, such as recombinant CXCL9, recombinant CXCL10, recombinant CXCL11 and recombinant CXCL4; CXCR4 agonists, such as recombinant CXCL12, ATI2341, CTCE0214, CTCE0324 and NNZ4921; CXCR5 agonists, such as recombinant CXCL13; CXCR6 agonists, such as recombinant CXCL16; and CXCL7 agonists, such as recombinant CXCL11.

The CC chemokine receptor may be selected from the group consisting of CCR1 agonists, such as recombinant CCL3, ECI301, recombinant CCL4, recombinant CCL5, recombinant CCL6, recombinant CCL8, recombinant CCL9/10, recombinant CCL14, recombinant CCL15, recombinant CCL16, recombinant CCL23, PB103, PB105 and MPIF1; CCR2 agonists, such as recombinant CCL2, recombinant CCL8, recombinant CCL16, PB103 and PB105; CCR3 agonists, such as recombinant CCL11, recombinant CCL26, recombinant CCL7, recombinant CCL13, recombinant CCL15, recombinant CCL24, recombinant CCL5, recombinant CCL28 and recombinant CCL18; CCR4 agonists, such as recombinant CCL3, ECI301, recombinant CCL5, recombinant CCL17 and recombinant CCL22; CCR5 agonists, such as recombinant CCL3, ECI301, recombinant CCL5, recombinant CCL8, recombinant CCL11, recombinant CCL13, recombinant CCL14, recombinant CCL16, PB103 and PB105; CCR6 agonists, such as recombinant CCL20; CCR7 agonists, such as recombinant CCL19 and recombinant CCL21; CCR8 agonists, such as recombinant CCL1, recombinant CCL16, PB103 and PB105; CCR9 agonists, such as recombinant CCL25; CCR10 agonists, such as recombinant CCL27 and recombinant CCL28; and CCR11 agonists, such as recombinant CCL19, recombinant CCL21 and recombinant CCL25.

The C chemokine receptors may be a XCR1 agonist, such as recombinant XCL1 or recombinant XCL2.

The CX3C chemokine receptors may be a CX3CR1 agonist, such as recombinant CX3CL1.

The chemoattractant receptors may be selected from the group consisting of formyl peptide receptor agonists, such as N-formyl peptides, N-formylmethionine-leucyl-phenylalanine, enfuvirtide, T21/DP107, annexin A1, Ac2-26 and Ac9-25; C5a receptor agonists; and chemokine-like receptor 1 agonists, such as chemerin.

The chemokine antagonists may be selected from the group consisting of inhibitors of CXCL chemokines, such as UNBS5162; inhibitors of CXCL8, such as BMS986253 and PA620; inhibitors of CXCL10, such as TM110, eldelumab and NI0801; inhibitors of CXCL12, such as NOX-A12 and JVS100; inhibitors of CXCL13, such as VX5; inhibitors of CCL2, such as PA508, ABN912, AF2838, BN83250, BN83470, C243, CGEN54, CNTO888, NOXE36, VT224 and SSR150106; inhibitors of CCL5, such as HGS1025 and NI0701; inhibitors of CCL2/CCL5, such as BKTP46; inhibitors of CCL5/FMLP receptor, such as RAP160; inhibitors of CCL11, such as bertilimumab and RAP701; inhibitors of CCL5/CXCL4, such as CT2008 and CT2009; inhibitors of CCL20, such as GSK3050002; and inhibitors of CX3CL1, such as quetmolimab.

The chemokine receptor antagonists may be selected from the group consisting of inhibitors of CXCR1, such as repertaxin, CCX832, FX68 and KB03; inhibitors of CXCR2, such as AZD5069, AZD5122, AZD8309, GSK1325756, GSK1325756H, PS291822, SB332235 and SB656933; inhibitors of CXCR1/CXCR2, such as DF1970, DF2156A, DF2162, DF2755A, reparixin, SX576, SX682, PACG31P, AZD4721 and PA401; inhibitors of CXCR3; inhibitors of CXCR4, such as BL8040; inhibitors of CXCR4/E-selectin, such as GMI1359; inhibitors of CXCR6, such as CCX5224; inhibitors of CCR1, such as AZD4818, BAY865047, BMS817399, CCX354, CCX634, CCX9588, CP481715, MLN3701, MLN3897, PS031291, PS375179 and PS386113; inhibitors of CCR2, such as AZD2423, BL2030, BMS741672, CCX140, CCX598, CCX872, CCX915, CNTX6970, INCB3284, INCB3344, INCB8696, JNJ17166864, JNJ27141491, MK0812, OPLCCL2LPM, PF4136309, serocion, STIB0201, STIB0211, STIB0221, STIB0232, STIB0234, TAK202, TPI526; inhibitors of CCR2/CCR5, such as PF04634817, RAP103 and TBR652; inhibitors of CCR2/CCR5/CCR8, such as RAP310; inhibitors of CCR3, such as ASM8, AXP1275, BMS639623, CM101, DPC168, GW766994, GW824575, MT0814, OPLCCL11LPM and QAP642; inhibitors of CCR4, such as AT008, AZD2098, CCX6239, FLX193, FLX475, GBV3019, GSK2239633, IC487892 and poteligeo; inhibitors of CCR5, such as 5P12-RANTES, AZD5672, AZD8566, CMPD167, ESN196, GSK706769, GW873140, HGS004, INCB15050, INCB9471, L872, microbicide, PF232798, PRO140, RAP101, SAR113244, SCH350634, SCH351125, SCH417690, selzentry, TAK779, TBR220, TD0232 and VX286; inhibitors of CCR5/CXCR4, such as AMD887, ND401 and SP01A; inhibitors of CCR6, such as CCX507, CCX9664 and STIB100X; inhibitors of CCR6, such as CCX025, CCX507, CCX807, eut22, MLN3126, POL7085, traficet-EN; inhibitors of CXCR3, such as AMG487, AT010, STIA120X; inhibitors of CXCR4, such as AD114, AD214, ALX0651, ALX40-4C, AMD070, AT007, AT009, BKT170, BMS936564, celixafor, CTCE9908, GBV4086, GSK812397, KRH2731, KRH3140, LY2510924, LY2624587, mozobil, OPLCXCL12LPM, PF06747143, POL6326, Q122, revixil, TG0054, USL311, X4P001 and X4P002; and inhibitors of CXCR7, such as CCX650 and CCX662.

The cytokine receptor agonist may be selected from the group consisting of mRNAs, DNAs or plasmids encoding the genes for IL-2, IL-15, IL-7, IL-10, IL-12, IL-21, IFNα IL-17, IFNβ, IFNγ, IL-18, IL-27, TNFα, GM-CSF, FLT3L, LTα, LTβ and TRAIL and recombinant proteins, such as agonists of IL-2/IL-15 β/γ receptors, agonists of IL-10 receptor, agonists of IL-12 receptor, agonists of IL-18 receptor, agonists of IL-21 receptor, agonists of IL-7 receptor, agonists of IFNα/β receptor, agonists of IFN γ receptor, agonists of FLT3 receptor, agonists of GM-CSF receptor, agonists of LTα receptor, agonists of LTβ receptor, and agonists of TNFα receptor.

Examples for agonists of IL-10 receptor are AG011, dekavil, EG10, IL10Nanocap, Ilodecakin, AM0010, tenovil and VT310 VIRON.

Examples for agonists of IL-12 receptor are recombinant IL-12 p70, recombinant IL-12 p35, AM0012, AS1409, dodekin, HemaMax, LipoVIL12, MSB0010360N, Ad-RTS-hIL-12, tavokinogene telseplasmid, exoIL-12 and NHS-IL12.

An example for an agonist of IL-18 receptor is SB485232.

An example for an agonist of IL-21 receptor is BMS982470 (denenicokin).

Examples for agonists of IL-7 receptor are CYT107, CYT99007 and GX-17.

An example for an agonist of FLT3R is CDX-301.

Examples for agonist of TNFα receptor are L19-TNFα, aurimune, beromun, BreMel/TNFα, fibromun, refnot and TNFPEG20.

The death receptor agonists may be selected from the group consisting of TRAILR1/DR4 agonists, such as AMG951 (dulanermin), APG350, APG880, HGSETR1 (mapatumumab) and SL231; and TRAILR2/DR5 agonists, such as AMG655, DS8273, HGSETR2 (lexatumumab), HGSTR2J, IDD004/GEN1029, INBRX109, LBY135, MEDI3039, PRO95780, RG7386 and TAS266.

The CD47 antagonists may be selected from the group consisting of ALX148, CC-90002, Hu5F9G4, SRF231, TI061, TTI-621, TTI-622, AO176, IBI188, IMC002, recombinant SIRPα and LYN00301.

An example for a SIRPα antagonist is FSI89 or recombinant CD47.

Examples for oncolytic drugs are CAVATAK, BCG, mobilan, TG4010, Pexa-Vec (JX-594), JX-900, JX-929 and JX-970.

Examples for signal converter proteins are Fn14-TRAIL (KAHR101), CD80-Fc (FTP155), CTLA4-FasL (KAHR102), PD1-41BBL (DSP 105), PD-L1-41BB (PRS-344, NM21-1480, FS222), PD1-CD70 (DSP 106) and SIRPα-41BBL (DSP 107).

The epigenetic modifiers may be selected from the group consisting of DNA methyltransferase inhibitors, lysine-specific demethylase 1 inhibitors, Zeste homolog 2 inhibitors, bromodomain and extra-terminal motif (BET) protein inhibitors such as GSK525762, and histone deacetylase (HDAC) inhibitors such as beleodaq, SNDX275 and CKD-M808.

Examples for tumor peptides/vaccines are NY-ESO, WT1, MART-1, IO102 and PF-06753512 and personalized cancer vaccines using patient derived tumor sequences or neoantigens.

Examples for heat shock protein (HSP) inhibitors are inhibitors of HSP90, such as PF-04929113 (SNX-5422).

Examples of proteolytic enzymes are recombinant hyaluronidase, such as rHuPH20 and PEGPH20.

The ubiquitin and proteasome inhibitors may be selected from the group consisting of ubiquitin-specific protease (USP) inhibitors, such as P005091; 20S proteasome inhibitors, such as bortezimib, carfilzomib, ixazomib, oprozomib, delanzomib and celastrol; and immunoproteasome inhibitors, such as ONX-0914.

The adhesion molecule antagonists may be selected from the group consisting of β2-integrin antagonists and selectin antagonists.

The hormones may be selected from the group consisting of hormone receptor agonists and hormone receptor antagonists.

An example for a hormone receptor agonist are somatostatin receptor agonists, such as somatostatin, lanreotide, octreotide, FX125L, FX141L and FX87L.

Example for hormone receptor antagonists are anti-androgens, anti-estrogens and anti-progestogens. Examples for anti-androgens are steroidal antiandrogens, such as cyproterone acetate, megestrol acetate, chlormadinone acetate, spironolactone, oxendolone and osaterone acetate; nonsteroidal anti-androgens, such as flutamide, bicalutamide, nilutamide, topilutamide, enzalutamide and apalutamide; androgen synthesis inhibitors, such as ketoconazole, abiraterone acetate, seviteronel, aminoglutethimide, finasteride, dutasteride, epristeride and alfatradiol. Examples for anti-estrogens are selective estrogen receptor modulators (SERMs), such as tamoxifen, clomifene, Fareston and raloxifene; ER silent antagonists and selective estrogen receptor degrader (SERD), such as fulvestrant; aromatase inhibitors, such as anastrozole, letrozole, exemestane, vorozole, formestane and fadrozole; and anti-gonadotropins, such as testosterone, progestogens and GnRH analogues. Examples for anti-progestogens are mifepristone, lilopristone and onapristone.

Examples of cellular therapy include CAR therapies such as CAR-T therapies such as tisagenlecleucel, axicabtagene ciloleucel, bb21217, LCAR-B38M, JCARH125, MCARH171, JNJ-4528, idecabtagene vicleucel (bb2121), SCRI-CAR19x22; CAR therapies targeting tumor antigens such as CAR therapies targeting CD19 expressing cells, CAR therapies targeting CD22 expressing cells, CAR therapies targeting BCMA expressing cells, CAR therapies targeting HER2 expressing cells, CAR therapies targeting CD138 expressing cells, CAR therapies targeting CD133 expressing cells, CAR therapies targeting BCMA expressing cells, CAR therapies targeting CEA expressing cells, CAR therapies targeting Claudin 18.2 expressing cells, CAR therapies targeting EGFR expressing cells, CAR therapies targeting EGFRvIII expressing cells, CAR therapies targeting Eph2A expressing cells, CAR therapies targeting EpCAM expressing cells, CAR therapies targeting GD2 expressing cells, CAR therapies targeting GPC3 expressing cells, CAR therapies targeting MSLN expressing cells, CAR therapies targeting 5T4 expressing cells, CAR therapies targeting LMP1 expressing cells, CAR therapies targeting PD-L1 expressing cells, CAR therapies targeting PSMA expressing cells, CAR therapies targeting FRα expressing cells, and CAR therapies targeting MUC1 expressing cells. Examples of cellular therapy include TIL therapy, NK therapy, Cytokine induced memory NK cell therapy, NK cell therapy with ex vivo expanded cells. Examples of cellular therapy include therapy with αβ or γδ T cells which may be engineered to express a tumor antigen or tumor neoantigen specific T Cell Receptor or which may have been expanded in the context of tumor antigen or tumor neoantigens.

In certain embodiments the patient is a mammalian patient, such as a human patient.

Administration of the IL-2 protein of formula (I), the IL-2 conjugate or the pharmaceutical composition as described herein may be done by external application, injection or infusion, including intraarticular, periarticular, intradermal, subcutaneous, intramuscular, intravenous, intraosseous, intraperitoneal, intrathecal, intracapsular, intraorbital, intratumoral, intravitreal, intratympanic, intravesical, intracardiac, transtracheal, subcuticular, subcapsular, subarachnoid, intraspinal, intraventricular, intrasternal injection and infusion, direct delivery to the brain via implanted device allowing delivery of the invention or the like to brain tissue or brain fluids (e.g., Ommaya Reservoir), direct intracerebroventricular injection or infusion, injection or infusion into brain or brain associated regions, injection into the subchoroidal space, retro-orbital injection and ocular instillation, preferably via subcutaneous injection. In certain embodiments administration is via subcutaneous injection.

In another aspect the present invention relates to an IL-2 protein sequence of formula (I-i)

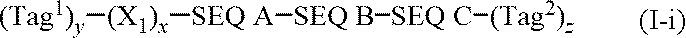

wherein
SEQ A has at least 89% sequence identity with SEQ ID NO:1;
SEQ B has at least 76% sequence identity to SEQ ID NO:2 and comprises at least one glycosylation motif;
SEQ C has at least 91% sequence identity to SEQ ID NO:4;
Tag¹ and Tag² are independently a tag moiety;
X₁ is an amino acid residue selected from the group consisting of arginine, asparagine, aspartic acid, cysteine, glutamine, glutamic acid, glycine, histidine, isoleucine, leucine, lysine, methionine, phenylalanine, serine, threonine, tryptophan, tyrosine and valine; and
x is 0 or 1;
y is 0 or 1; and
z is 0 or 1.

Specific embodiments for SEQ A, SEQ B, SEQ C, Tag¹, Tag², x, y and z are as disclosed for the IL-2 protein of formula (I).

In certain embodiments X₁ of formula (I-i) is an arginine residue. In certain embodiments X₁ of formula (I-i) is an asparagine residue. In certain embodiments X₁ of formula (I-i) is an aspartic acid residue. In certain embodiments X₁ of formula (I-i) is a cysteine residue. In certain embodiments X₁ of formula (I-i) is a glutamine residue. In certain embodiments X₁ of formula (I-i) is a glutamic acid residue. In certain embodiments X₁ of formula (I-i) is a glycine residue. In certain embodiments X₁ of formula (I-i) is a histidine residue. In certain embodiments X₁ of formula (I-i) is an isoleucine residue. In certain embodiments X₁ of formula (I-i) is a leucine residue. In certain embodiments X₁ of formula (I-i) is a lysine residue. In certain embodiments X₁ of formula (I-i) is a methionine residue. In certain embodiments X₁ of formula (I-i) is a phenylalanine residue. In certain embodiments X₁ of formula (I-i) is a serine residue. In certain embodiments X₁ of formula (I-i) is a threonine residue. In certain embodiments X₁ of formula (I-i) is a tryptophan residue. In certain embodiments X₁ of formula (I-i) is a tyrosine residue. In certain embodiments X₁ of formula (I-i) is a valine residue.

In another aspect the present invention relates to an oligonucleotide sequence encoding the IL-2 protein of formula (I-i). Specific embodiments for such oligonucleotides are as described elsewhere herein for the oligonucleotides encoding the IL-2 protein of formula (I), with the exception that the IL-2 protein of formula (I) is replaced with the IL-2 protein of formula (I-i).

In another aspect the present invention relates to a conjugate comprising one or more of the IL-2 proteins of formula (I-i). Specific embodiments for this conjugate comprising one or more of the IL-2 proteins of formula (I-i) are as described elsewhere herein for the conjugates comprising one or more of the IL-2 proteins of formula (I), with the exception that the IL-2 protein of formula (I) is replaced with the IL-2 protein of formula (I-i).

In another aspect the present invention relates to a pharmaceutical composition comprising at least one IL-2 protein of formula (I-i) or at least one IL-2 conjugate comprising one or more such IL-2 proteins of formula (I-i) and at least one excipient. Specific embodiments for such pharmaceutical composition are as described elsewhere herein for the pharmaceutical compositions comprising at least one IL-2 proteins of formula (I) or at least one IL-2 conjugate comprising one or more of the IL-2 proteins of formula (I), with the exception that the IL-2 protein of formula (I) is replaced with the IL-2 protein of formula (I-i).

Another aspect relates to the IL-2 protein of formula (I-i), the IL-2 conjugate comprising at least one IL-2 protein of formula (I-i) or the pharmaceutical composition comprising such IL-2 protein or IL-2 conjugate for use as a medicament.

Another aspect relates to the IL-2 protein of formula (I-i), the IL-2 conjugate comprising at least one IL-2 protein of formula (I-i) or the pharmaceutical composition comprising such IL-2 protein or IL-2 conjugate for use in the treatment of a disease which can be treated with IL-2. Specific embodiments for the disease which can be treated with IL-2 are as described elsewhere herein.

Another aspect relates to the IL-2 protein of formula (I-i), the IL-2 conjugate comprising at least one IL-2 protein of formula (I-i) or the pharmaceutical composition comprising such IL-2 protein or IL-2 conjugate for the manufacture of a medicament for treating a disease which can be treated with IL-2. Specific embodiments for the disease which can be treated with IL-2 are as described elsewhere herein.

Another aspect relates to a method of treating, controlling, delaying or preventing in a mammalian patient, preferably a human patient, in need of the treatment of one or more diseases which can be treated with IL-2, comprising the step of administering to said patient in need thereof a therapeutically effective amount of the IL-2 protein of formula (I-i), the IL-2 conjugate comprising at least one IL-2 protein of formula (I-i) or the pharmaceutical composition comprising such IL-2 protein or IL-2 conjugate. Specific embodiments for the disease which can be treated with IL-2 are as described elsewhere herein.

In certain embodiments the IL-2 protein of formula (I-i), the IL-2 conjugate comprising at least one IL-2 protein of formula (I-i) or the pharmaceutical composition comprising such IL-2 protein or IL-2 conjugate is administered to the patient prior to, simultaneously with, or after administration of one or more additional drug. Specific embodiments for such one or more additional drug are as described elsewhere herein.

In another aspect the present invention relates to an IL-2 protein sequence of formula (I-ii)

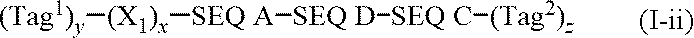

wherein
SEQ A has at least 89% sequence identity with SEQ ID NO:1;
SEQ D has at least 76% sequence identity to SEQ ID NO:2;
SEQ C has at least 91% sequence identity to SEQ ID NO:4;
Tag¹ and Tag² are independently a tag moiety;
X₁ is an amino acid residue selected from the group consisting of arginine, asparagine, aspartic acid, cysteine, glutamine, glutamic acid, glycine, histidine, isoleucine, leucine, lysine, methionine, phenylalanine, serine, threonine, tryptophan, tyrosine and valine; and
x is 0 or 1;
y is 0 or 1; and
z is 0 or 1.

Specific embodiments for SEQ A, SEQ C, Tag¹, Tag², x, y and z are as disclosed elsewhere herein.

In certain embodiments SEQ D of formula (I-ii) has at least 78% sequence identity to SEQ ID NO:2. In certain embodiments SEQ D of formula (I-ii) has at least 80% sequence identity to SEQ ID NO:2. In certain embodiments SEQ D of formula (I-ii) has at least 82% sequence identity to SEQ ID NO:2. In certain embodiments SEQ D of formula (I-ii) has at least 84% sequence identity to SEQ ID NO:2. In certain embodiments SEQ D of formula (I-ii) has at least 87% sequence identity to SEQ ID NO:2. In certain embodiments SEQ D of formula (I-ii) has at least 89% sequence identity to SEQ ID NO:2. In certain embodiments SEQ D of formula (I-ii) has at least 91% sequence identity to SEQ ID NO:2. In certain embodiments SEQ D of formula (I-ii) has at least 93% sequence identity to SEQ ID NO:2. In certain embodiments SEQ D of formula (I-ii) has at least 95% sequence identity to SEQ ID NO:2.

In certain embodiments SEQ D of formula (I-ii) comprises eleven amino acid changes compared to SEQ ID NO:2. In certain embodiments SEQ D of formula (I-ii) comprises ten amino acid changes compared to SEQ ID NO:2. In certain embodiments SEQ D of formula (I-ii) comprises nine amino acid changes compared to SEQ ID NO:2. In certain embodiments SEQ D of formula (I-ii) comprises eight amino acid changes compared to SEQ ID NO:2. In certain embodiments SEQ D of formula (I-ii) comprises seven amino acid changes compared to SEQ ID NO:2. In certain embodiments SEQ D of formula (I-ii) comprises six amino acid changes compared to SEQ ID NO:2. In certain embodiments SEQ D of formula (I-ii) comprises five amino acid changes compared to SEQ ID NO:2. In certain embodiments SEQ D of formula (I-ii) comprises four amino acid changes compared to SEQ ID NO:2. In certain embodiments SEQ D of formula (I-ii) comprises three amino acid changes compared to SEQ ID NO:2. In certain embodiments SEQ D of formula (I-ii) comprises two amino acid changes compared to SEQ ID NO:2.

In certain embodiments SEQ D of formula (I-ii) has a sequence selected from the group consisting of

YKNPFADSTLTFKFYMPKKATELKHLQCLEEELKPLEEVLNLAQSK (SE
Q ID NO:346);

YKNPKLTFADSTKFYMPKKATELKHLQCLEEELKPLEEVLNLAQSK (SE
Q IDNO:347);

YKNPKLTRFADSTFYMPKKATELKHLQCLEEELKPLEEVLNLAQSK (SE
Q IDNO:348);

YKNPKLTRMLFADSTMPKKATELKHLQCLEEELKPLEEVLNLAQSK (SE
Q IDNO:349);

YKNPKLTRMLTFADSTPKKATELKHLQCLEEELKPLEEVLNLAQSK (SE
Q IDNO:350);

YKNPKLTRMLTFFADSTKKATELKHLQCLEEELKPLEEVLNLAQSK (SE
Q IDNO:351);

YKNPKLTRMLTFKFADSTKATELKHLQCLEEELKPLEEVLNLAQSK (SE
Q IDNO:352);

YKNPKLTRMLTFKFFADSTATELKHLQCLEEELKPLEEVLNLAQSK (SE
Q ID NO:353);

YKNPKLTRMLTFKFYMPKKATELKHLQCLEFADSTLEEVLNLAQSK (SE
Q IDNO:354);

YKNPKLTRMLTFKFYMPKKATELKHLQCLEEFADSTEEVLNLAQSK (SE
Q IDNO:355);

and YKNPKLTRMLTFKFYMPKKATELKHLQCLEEELKPLEEVLNFADST
(SEQ IDNO:356).

In certain embodiments SEQ D of formula (I-ii) has a sequence selected from the group consisting of YKNPFGDSTLTFKFYMPKKATELKHLQCLEEELKPLEEVLNLAQSK (SEQ ID NO:357);

YKNPKLTFGDSTKGDSTKFMPKKATELKHLQCLEEELKPLEEVLNLAQSK
(SEQ IDNO:358);

YKNPKLTRFGDSTFYMPKKATELKHLQCLEEELKPLEEVLNLAQSK (SE
Q IDNO:359);

YKNPKLTRMLFGDSTMPKKATELKHLQCLEEELKPLEEVLNLAQSK (SE
Q IDNO:360);

YKNPKLTRMLTFGDSTPKKATELKHLQCLEEELKPLEEVLNLAQSK (SE
Q IDNO:361);

YKNPKLTRMLTFFGDSTKKATELKHLQCLEEELKPLEEVLNLAQSK (SE
Q IDNO:362);

YKNPKLTRMLTFKFGDSTKATELKHLQCLEEELKPLEEVLNLAQSK (SE
Q IDNO:363);

YKNPKLTRMLTFKFFGDSTATELKHLQCLEEELKPLEEVLNLAQSK (SE
Q ID NO:364);

YKNPKLTRMLTFKFYMPKKATELKHLQCLEFGDSTLEEVLNLAQSK (SE
Q IDNO:365);

YKNPKLTRMLTFKFYMPKKATELKHLQCLEEFGDSTEEVLNLAQSK (SE
Q IDNO:366); and

YKNPKLTRMLTFKFYMPKKATELKHLQCLEEELKPLEEVLNFGDST (SE
Q IDNO:367).

In certain embodiments SEQ D of formula (I-ii) further comprises at least one amino acid mutation occurring at an amino acid position selected from the group consisting of K5, R8, M9, T11, F12, K13, F14, Y15, E31, E32 and L42, based on SEQ ID NO:2 or at the corresponding positions of homologs or variants thereof. In certain embodiments the at least one amino acid mutation comprises a mutation at an amino acid position selected from the group consisting of F12, Y15, E31, E32 and L42 based on SEQ ID NO:2 or at the corresponding positions of homologs or variants thereof. In certain embodiments the at least one amino acid mutation comprises a mutation at amino acid position K5 based on SEQ ID NO:2 or at the corresponding positions of homologs or variants thereof. In certain embodiments the at least one amino acid mutation comprises a mutation at amino acid position R8 based on SEQ ID NO:2 or at the corresponding positions of homologs or variants thereof. In certain embodiments the at least one amino acid mutation comprises a mutation at amino acid position M9 based on SEQ ID NO:2 or at the corresponding positions of homologs or variants thereof. In certain embodiments the at least one amino acid mutation comprises a mutation at amino acid position T11 based on SEQ ID NO:2 or at the corresponding positions of homologs or variants thereof. In certain embodiments the at least one amino acid mutation comprises a mutation at amino acid position F12 based on SEQ ID NO:2 or at the corresponding positions of homologs or variants thereof. In certain embodiments the at least one amino acid mutation comprises a mutation at amino acid position K13 based on SEQ ID NO:2 or at the corresponding positions of homologs or variants thereof. In certain embodiments the at least one amino acid mutation comprises a mutation at amino acid position F14 based on SEQ ID NO:2 or at the corresponding positions of homologs or variants thereof. In certain embodiments the at least one amino acid mutation comprises a mutation at amino acid position Y15 based on SEQ ID NO:2 or at the corresponding positions of homologs or variants thereof. In certain embodiments the at least one amino acid mutation comprises a mutation at amino acid position E31 based on SEQ ID NO:2 or at the corresponding positions of homologs or variants thereof. In certain embodiments the at least one amino acid mutation comprises a mutation at amino acid position E32 based on SEQ ID NO:2 or at the corresponding positions of homologs or variants thereof. In certain embodiments the at least one amino acid mutation comprises a mutation at amino acid position L42 based on SEQ ID NO:2 or at the corresponding positions of homologs or variants thereof.

In certain embodiments X₁ of formula (I-ii) is an arginine residue. In certain embodiments X₁ of formula (I-ii) is an asparagine residue. In certain embodiments X₁ of formula (I-ii) is an aspartic acid residue. In certain embodiments X₁ of formula (I-ii) is a cysteine residue. In certain embodiments X₁ of formula (I-ii) is a glutamine residue. In certain embodiments X₁ of formula (I-ii) is a glutamic acid residue. In certain embodiments X₁ of formula (I-ii) is a glycine residue. In certain embodiments X₁ of formula (I-ii) is a histidine residue. In certain embodiments X₁ of formula (I-ii) is an isoleucine residue. In certain embodiments X₁ of formula (I-ii) is a leucine residue. In certain embodiments X₁ of formula (I-ii) is a lysine residue. In certain embodiments X₁ of formula (I-ii) is a methionine residue. In certain embodiments X₁ of formula (I-ii) is a phenylalanine residue. In certain embodiments X₁ of formula (I-ii) is a serine residue. In certain embodiments X₁ of formula (I-ii) is a threonine residue. In certain embodiments X₁ of formula (I-ii) is a tryptophan residue. In certain embodiments X₁ of formula (I-ii) is a tyrosine residue. In certain embodiments X₁ of formula (I-ii) is a valine residue.

In certain embodiments the IL-2 protein of formula (I-ii) has the sequence

PTSSSTKKTQLQLEHLLLDLQMILNGINNYKNPKLTFGDSTKFYMPKKAT
ELKHLQCLEEELKPLEEVLNLAQSKNFHLRPRDLISNINVIVLELKGSET
TFMCEYADETATIVEFLNRWITFSQSIISTLT (SEQ ID NO:334).

In certain embodiments the IL-2 protein of formula (I-ii) has the sequence

PTSSSTKKTQLQLEHLLLDLQMILNGINNYKNPKLTFADSTKFYMPKKAT
ELKHLQCLEEELKPLEEVLNLAQSKNFHLRPRDLISNINVIVLELKGSET
TFMCEYADETATIVEFLNRWITFSQSIISTLT (SEQ ID NO:335).

In certain embodiments the IL-2 protein of formula (I-ii) has the sequence

PTSSSTKKTQLQLEHLLLDLQMILNGINNYKNPKLTRMLFGDSTMPKKAT
ELKHLQCLEEELKPLEEVLNLAQSKNFHLRPRDLISNINVIVLELKGSET
TFMCEYADETATIVEFLNRWITFSQSIISTLT (SEQ ID NO:336).

In certain embodiments the IL-2 protein of formula (I-ii) has the sequence

PTSSSTKKTQLQLEHLLLDLQMILNGINNYKNPKLTRMLFADSTMPKKAT
ELKHLQCLEEELKPLEEVLNLAQSKNFHLRPRDLISNINVIVLELKGSET
TFMCEYADETATIVEFLNRWITFSQSIISTLT (SEQ ID NO:337).

In certain embodiments the IL-2 protein of formula (I-ii) has the sequence

PTSSSTKKTQLQLEHLLLDLQMILNGINNYKNPKLTRMLTFKFYMPKKAT
ELKHLQCLEFGDSTLEEVLNLAQSKNFHLRPRDLISNINVIVLELKGSET
TFMCEYADETATIVEFLNRWITFSQSIISTLT (SEQ ID NO:338).

In certain embodiments the IL-2 protein of formula (I-ii) has the sequence

PTSSSTKKTQLQLEHLLLDLQMILNGINNYKNPKLTRMLTFKFYMPKKAT
ELKHLQCLEFADSTLEEVLNLAQSKNFHLRPRDLISNINVIVLELKGSET
TFMCEYADETATIVEFLNRWITFSQSIISTLT (SEQ ID NO:339).

In certain embodiments the IL-2 protein of formula (I-ii) has the sequence

APTSSSTKKTQLQLEHLLLDLQMILNGINNYKNPKLTFGDSTKFYMPKKA
TELKHLQCLEEELKPLEEVLNLAQSKNFHLRPRDLISNINVIVLELKGSE
TTFMCEYADETATIVEFLNRWITFSQSIISTLT (SEQ ID NO:340).

In certain embodiments the IL-2 protein of formula (I-ii) has the sequence

APTSSSTKKTQLQLEHLLLDLQMILNGINNYKNPKLTFADSTKFYMPKKA
TELKHLQCLEEELKPLEEVLNLAQSKNFHLRPRDLISNINVIVLELKGSE
TTFMCEYADETATIVEFLNRWITFSQSIISTLT (SEQ ID NO:341).

In certain embodiments the IL-2 protein of formula (I-ii) has the sequence

APTSSSTKKTQLQLEHLLLDLQMILNGINNYKNPKLTRMLFGDSTMPKKA
TELKHLQCLEEELKPLEEVLNLAQSKNFHLRPRDLISNINVIVLELKGSE
TTFMCEYADETATIVEFLNRWITFSQSIISTLT (SEQ ID NO:342).

In certain embodiments the IL-2 protein of formula (I-ii) has the sequence

APTSSSTKKTQLQLEHLLLDLQMILNGINNYKNPKLTRMLFADSTMPKKA
TELKHLQCLEEELKPLEEVLNLAQSKNFHLRPRDLISNINVIVLELKGSE
TTFMCEYADETATIVEFLNRWITFSQSIISTLT (SEQ ID NO:343).

In certain embodiments the IL-2 protein of formula (I-ii) has the sequence

APTSSSTKKTQLQLEHLLLDLQMILNGINNYKNPKLTRMLTFKFYMPKKA
TELKHLQCLEFGDSTLEEVLNLAQSKNFHLRPRDLISNINVIVLELKGSE
TTFMCEYADETATIVEFLNRWITFSQSIISTLT (SEQ ID NO:344).

In certain embodiments the IL-2 protein of formula (I-ii) has the sequence

APTSSSTKKTQLQLEHLLLDLQMILNGINNYKNPKLTRMLTFKFYMPKKA
TELKHLQCLEFADSTLEEVLNLAQSKNFHLRPRDLISNINVIVLELKGSE
TTFMCEYADETATIVEFLNRWITFSQSIISTLT (SEQ ID NO:345).

In certain embodiments the IL-2 protein of formula (I-ii) is obtainable by enzymatic or chemical removal of N-linked or O-linked glycans from an IL-2 protein of formula (I) or (I-i).

In certain embodiments the IL-2 protein of formula (I-ii) is obtainable by enzymatic removal of the one or more N-linked glycan from an IL-2 protein of formula (I) or (I-i) mediated by peptide-N-glycosidase F (PNgaseF).

In certain embodiments the IL-2 protein of SEQ ID NO:334 is obtainable by PNgaseF treatment of the IL-2 protein of SEQ ID NO:9.

In certain embodiments the IL-2 protein of SEQ ID NO:340 is obtainable by PNgaseF treatment of the IL-2 protein of SEQ ID NO:10.

In certain embodiments the IL-2 protein of SEQ ID NO:335 is obtainable by PNgaseF treatment of the IL-2 protein of SEQ ID NO:12.

In certain embodiments the IL-2 protein of SEQ ID NO:341 is obtainable by PNgaseF treatment of the IL-2 protein of SEQ ID NO:13.

In certain embodiments the IL-2 protein of SEQ ID NO:336 is obtainable by PNgaseF treatment of the IL-2 protein of SEQ ID NO:15.

In certain embodiments the IL-2 protein of SEQ ID NO:342 is obtainable by PNgaseF treatment of the IL-2 protein of SEQ ID NO:16.

In certain embodiments the IL-2 protein of SEQ ID NO:337 is obtainable by PNgaseF treatment of the IL-2 protein of SEQ ID NO:18.

In certain embodiments the IL-2 protein of SEQ ID NO:343 is obtainable by PNgaseF treatment of the IL-2 protein of SEQ ID NO:19.

In certain embodiments the IL-2 protein of SEQ ID NO:338 is obtainable by PNgaseF treatment of the IL-2 protein of SEQ ID NO:21.

In certain embodiments the IL-2 protein of SEQ ID NO:344 is obtainable by PNgaseF treatment of the IL-2 protein of SEQ ID NO:22.

In certain embodiments the IL-2 protein of SEQ ID NO:339 is obtainable by PNgaseF treatment of the IL-2 protein of SEQ ID NO:24.

In certain embodiments the IL-2 protein of SEQ ID NO:345 is obtainable by PNgaseF treatment of the IL-2 protein of SEQ ID NO:25.

In another aspect the present invention relates to an oligonucleotide sequence encoding the IL-2 protein of formula (I-ii). Specific embodiments for such oligonucleotides are as described elsewhere herein for the oligonucleotides encoding the IL-2 protein of formula (I), with the exception that the IL-2 protein of formula (I) is replaced with the IL-2 protein of formula (I-ii).

In another aspect the present invention relates to a conjugate comprising one or more of the IL-2 proteins of formula (I-ii). Specific embodiments for this conjugate comprising one or more of the IL-2 proteins of formula (I-ii) are as described elsewhere herein for the conjugates comprising one or more of the IL-2 proteins of formula (I), with the exception that the IL-2 protein of formula (I) is replaced with the IL-2 protein of formula (I-ii).

In another aspect the present invention relates to a pharmaceutical composition comprising at least one IL-2 protein of formula (I-ii) or at least one IL-2 conjugate comprising one or more such IL-2 proteins of formula (I-ii) and at least one excipient. Specific embodiments for such pharmaceutical composition are as described elsewhere herein for the pharmaceutical compositions comprising at least one IL-2 proteins of formula (I) or at least one IL-2 conjugate comprising one or more of the IL-2 proteins of formula (I), with the exception that the IL-2 protein of formula (I) is replaced with the IL-2 protein of formula (I-ii).

Another aspect relates to the IL-2 protein of formula (I-ii), the IL-2 conjugate comprising at least one IL-2 protein of formula (I-ii) or the pharmaceutical composition comprising such IL-2 protein or IL-2 conjugate for use as a medicament.

Another aspect relates to the IL-2 protein of formula (I-ii), the IL-2 conjugate comprising at least one IL-2 protein of formula (I-ii) or the pharmaceutical composition comprising such IL-2 protein or IL-2 conjugate for use in the treatment of a disease which can be treated with IL-2. Specific embodiments for the disease which can be treated with IL-2 are as described elsewhere herein.

Another aspect relates to the IL-2 protein of formula (I-ii), the IL-2 conjugate comprising at least one IL-2 protein of formula (I-ii) or the pharmaceutical composition comprising such IL-2 protein or IL-2 conjugate for the manufacture of a medicament for treating a disease which can be treated with IL-2. Specific embodiments for the disease which can be treated with IL-2 are as described elsewhere herein.

Another aspect relates to a method of treating, controlling, delaying or preventing in a mammalian patient, such as a human patient, in need of the treatment of one or more diseases which can be treated with IL-2, comprising the step of administering to said patient in need thereof a therapeutically effective amount of the IL-2 protein of formula (I-ii), the IL-2 conjugate comprising at least one IL-2 protein of formula (I-ii) or the pharmaceutical composition comprising such IL-2 protein or IL-2 conjugate. Specific embodiments for the disease which can be treated with IL-2 are as described elsewhere herein.

In certain embodiments the IL-2 protein of formula (I-ii), the IL-2 conjugate comprising at least one IL-2 protein of formula (I-ii) or the pharmaceutical composition comprising such IL-2 protein or IL-2 conjugate is administered to the patient prior to, simultaneously with, or after administration of one or more additional drug. Specific embodiments for such one or more additional drug are as described elsewhere herein.

### Materials and Methods

### Materials

All materials were commercially available except where stated otherwise.

### Methods

### Determination of Protein Concentration

Protein concentration was determined using calculated molecular extinction coefficient and molecular weight on an Eppendorf BioSpectrometer^{®} basic using the settings pre-defined by the manufacturer.

### PNGase F Assay

Purified IL-2 variants were mixed with or without PNGase F (Promega, #V4831, LOT# 0000386354) and treated according to the manufacturer’s instructions. Subsequently, samples were reduced and heat-treated prior to loading on an SDS-PAGE gel. Protein bands were stained with Bio-Safe Coomassie G-250 stain (Bio-Rad, #161078) following the manufacturer’s instructions. Glycosylated IL-2 variants expressed in HEK293-6E and CHO cells migrate in the SDS-PAGE as a double band at approximately 18-20 kDa. After treatment with PNGase F, a prominent downshift to approximately 15-17 kDa is observed, indicating efficient N-glycosylation of the IL-2 variants expressed in CHO cells.

### Peptide Map Analysis

10 µg IL-2 variants were mixed with 1 µg Pierce™ Trypsin/Lys-C Protease (from Thermo Fisher) in 100 mM Hepes buffer pH 8.0. The reaction mixture was incubated for 18±1 hours at 37±1° C. Undigested sample controls were prepared in the same buffer without any protease. After the incubation, the samples/controls were briefly centrifuged and 20 µL UPLC-MS water were added. Diluted samples were analyzed using Agilent 1290 UHPLC system connected to a QTof mass spectrometer. The analysis was performed utilizing 0.075 % (V/V) trifluoroacetic acid in water (mobile phase A), 0.06 % (V/V) trifluoroacetic acid in MeCN (mobile phase B). The Halo C18, 100×2.1, 2.7 µm, 90 Å column was employed running the gradient shown in the Table 1 below:

TABLE 1

Gradient

| Time [min] | %B |
|---|---|
| 0 | 0.5 |
| 1.0 | 0.5 |
| 9.2 | 25.7 |
| 10.5 | 33.0 |
| 26.0 | 61.0 |
| 26.2 | 99.9 |
| 33.0 | 99.9 |
| 34.0 | 0.5 |
| 40.0 | 0.5 |

Note: flow rate = 0.5 mL/min, CT = 21° C.

The flow rate is set to 0.5 mL/min. The column oven was set to 21° C.

### Example 1: Expression of Glycosylated IL2 in HEK293-6E

### Vector Construction

IL-2 glycosylation variants were custom made and sourced from an external supplier where expression of the proteins was performed from 293-6E cells followed by standard purification strategies known to the person skilled in the art. The following proteins were prepared: protein 1a (SEQ ID NO:205) corresponding to DNA sequence SEQ ID NO:7; protein 1b (SEQ ID NO:229) corresponding to DNA sequence SEQ ID NO:8; protein 1c (SEQ ID NO:230) corresponding to DNA sequence SEQ ID NO:202; and protein 1d (SEQ ID NO:232) corresponding to DNA sequence SEQ ID NO:203:

The coding DNA sequence encoding SEQ ID NO:205, SEQ ID NO:229, SEQ ID NO:230, and SEQ ID NO:232 were codon optimized for human expression including an N-terminal human kappa light chain leader sequence and a C terminal hexahistidine tag and inserted into the EcoRI and BamHI sites of pTT5 mammalian expression vectors. The constructed plasmids were transformed into E. coli strains (DH5a/TOP10) for propagation respectively in appropriate scale. NucleoBond Xtra Maxi Plus EF kit (MN 740426) was used for large scale plasmid generation. Purified plasmids were checked by agarose gels and confirmed by sequencing.

### Protein Expression

For mammalian cell expression, plasmids for each protein were transfected into HEK293-6E cells obtained from the CNRC-NRC (“Conseil National de Recherches Canada”) with PEI (Polyscience, Cat# 23966). The cells were cultured in incubator shakers at standard cell culture conditions. Conditioned medium was harvested 6-7 days post transfection.

### Protein Purification and Quality Control

Conditioned medium (CM) was recovered by centrifugation to discard cell debris and then filtered. The clarified CM was loaded onto a Ni-excel (GE) column and protein purification was performed according to the manufacturer’s instructions. The concentrated material was then further purified with a superdex 75 column (GE).

Purified proteins were analyzed by SDS-PAGE (with and without PNGase F (NEB) pretreatment), SEC-HPLC and endotoxin measurement with the Endosafe-PTS kit (Charles River).

### Results

Of the constructs described, protein 1a expressed well with similar yields (0.97 mg) as seen for a non-N-glycosylated IL-2 control (protein 1d, 1.0 mg). Surprisingly, at least 90% of protein 1a migrated at a higher molecular weight (approximately 18 kDa) as compared to control non-N-glycosylated IL-2 protein 1d (15 kDa) suggesting the presence of N-glycans in 1a which decrease mobility in SDS-PAGE. When protein 1a was treated with Protein N-glycosidase F (PNGase F) to remove N-glycans, the treated protein migrated uniformly at approximately 16 kDa, similar to wild type IL-2 (protein 1d), indicating that nearly all of protein 1a was N-glycosylated. As expected, in PNGase F treated wells an additional ~36 kDa band was also present corresponding to the molecular weight of PGNase F itself.

### Example 2: Expression of Glycosylated IL2 With a Cleavable HIS Tag in CHO Cells

### Vector Construction

IL-2 glycosylation variants were custom made and sourced from an external supplier where expression of the proteins was performed from CHO cells followed by standard purification strategies known to the person skilled in the art. The following protein was prepared: protein 1e (SEQ ID NO:231) corresponding to DNA SEQ ID NO:233.

Protein 1e was subsequently treated with EnteroKinase to create final product protein 1f (SEQ ID NO:10).

The coding DNA sequence SEQ ID NO:233 was codon optimized for CHO expression including an N-terminal human kappa light chain leader sequence an N terminal hexahistidine tag, and an N terminal Enterokinase cleavage site to remove the His tag and inserted into a pTT5 mammalian expression vectors. The constructed plasmids were transformed into E. coli strains (DH5a/TOP10) for propagation respectively in appropriate scale. NucleoBond Xtra Maxi Plus EF kit (MN 740426) was used for large scale plasmid generation. Purified plasmids were checked by agarose gels and confirmed by sequencing.

### Protein Expression

Plasmids for each protein were transfected into EXPI-CHO cells obtained from Thermo Fisher with PEI Max (Polyscience, Cat# 24765). The cells were cultured in incubator shakers at standard cell culture conditions. Conditioned medium was harvested 9 days post transfection.

### Protein Purification and Quality Control

Conditioned medium (CM) was recovered by centrifugation to discard cell debris and then filtered using Sartopore 2 filters (Sartorius). The clarified CM was loaded onto a Ni-excel (GE) column and protein purification was performed according to the manufacturer’s instructions. Eluted proteins were incubated with EK protease (GenScript, Z03004-100) to cleave the His tag. The material was further purified with another nickel column followed by a superdex 75 column (GE). Purified proteins were analyzed by SDS-PAGE, SEC-HPLC and endotoxin measurement with the Endosafe-PTS kit (Charles River).

### Results

Similar to expression of N-glycosylated IL2 seen in 293 cells (protein 1a), at least 90% of N-glycosylated IL2 expressed in CHO cells (protein 1f derived from protein 1e) uniformly demonstrated a higher band with decreased migration of approximately ~17 KDa as compared with separate experiments with control IL2 (~15 kDa), indicating surprisingly efficient N-glycosylation of protein 1f. The migration pattern of the CHO derived His tag removed N-glycosylated IL2 (protein 1f) was as expected slightly smaller (~17 kDa) than the 293 expressed construct (protein 1a) containing a His tag (~18 kDa) is in agreement with the 6xHis tag’s MW of 840 Da.

### Example 3: Bioactivity of 293 Produced Glycosylated IL2 in Human Primary Cell pSTAT5 Assays

Bioactivity of control wild type IL2 protein 1d as well as glycoengineered IL2 protein 1a from 293 expression from Example 1 was tested by an external vendor using human blood. Phosphorylation of STAT5 (pSTAT5), a proximal IL2 receptor signaling biomarker, was examined after treatment of samples with protein 1d or protein 1a. Heparinized human whole blood was drawn and equilibrated to room temperature, aliquoted to plates and warmed to 37° C. prior to addition of protein 1d or protein 1a. A dose titration of protein 1d or protein 1a was prepared and prewarmed to 37° C. before addition to cells. After addition of protein 1d or protein 1a, cells were incubated at 37° C. for 30 minutes. At the end of the incubation period, the red blood cells were lysed and the cells fixed. Following washing the cells were then stained for surface antigens, washed and permeabilized with methanol followed by washes and intracellular staining for pSTAT5 and additional lineage markers. The following markers were used to define cell populations of interest: CD8+ T cells were defined as CD3+ CD4- CD8+; CD4+ T cells were defined as CD3+ CD4+ CD8-; Regulatory T cells (Tregs) were defined as CD3+ CD4+ CD8- CD25+ FOXP3+; Natural Killer (NK) cells were defined as CD3- CD56 or CD16+.

### Results

Half maximal effective concentrations (EC50s) for CD8+ T cells, NK cells and Tregs were determined after treatment with either wild type IL-2 control (1d) or N-glycosylated IL2 (1a). Importantly, CD8+ T cells and NK cells express low resting levels of the high affinity alpha subunit of the IL-2 Receptor (IL2Rα/CD25) while Tregs express high levels of IL2Rα (CD25). For this reason analysis in changes in relative potency of IL-2 variants between CD8+ T cells or NK cells vs Tregs is a good measure of the activity to IL-2R βγ (as reflected by CD8+ and NK cells potency) vs activity to IL-2R αβγ as reflected by Treg potency. We examined the potency across these three cell subsets by quantifying both the median fluorescence intensity (MFI) of pSTAT5 as well as the percentage of pSTAT5 positive cells. A summary of the pSTAT5 MFI EC50 values is shown in Table 2:

TABLE 2

Ave EC50 Values (ng/ml) based on pSTAT5 MFI values

| Population | WT-IL2 (1d) | N-glycosylated IL2 (1a) | Fold reduction in potency |
|---|---|---|---|
| CD8+ T Cells | 8.756 | 12.630 | 1.44 |
| NK Cells | 2.296 | 3.070 | 1.34 |
| T Regulatory Cells | 0.018 | 7.926 | 436.56 |

Using the pSTAT5 MFI metric, N-glycosylated IL-2 protein 1a demonstrated similar potency as compared to wild type IL-2 control for CD8+ T cells and NK cells (1.44 and 1.34 fold reduction in potency vs wild type IL-2, respectively, Table 2). However, N-glycosylated IL-2 protein 1a demonstrated strikingly reduced potency as compared to wild type IL-2 control for Tregs (436.56 fold reduction in potency, Table 2).

A summary of the percent pSTAT5 positive EC50 values is shown in Table 3:

TABLE 3

Ave EC50 Values (ng/ml) based on pSTAT5 percent positive values

| Population | WT-IL2 (1d) | N-glycosylated IL2 (1a) | Fold reduction in potency |
|---|---|---|---|
| CD8+ T Cells | 1.075 | 1.694 | 1.58 |
| NK Cells | 0.184 | 0.371 | 2.01 |
| T Regulatory Cells | 0.002 | 1.065 | 598.46 |

Similarly, using the Percent pSTAT5 positive metric, N-glycosylated IL-2 protein 1a demonstrated similar potency as compared to wild type IL-2 control for CD8+ T cells and NK cells (1.58 and 2.01 fold reduction in potency vs wild type IL-2, respectively). However, N-glycosylated IL-2 protein 1a demonstrated strikingly reduced potency as compared to wild type IL-2 (1d) control for Tregs (598.46 fold reduction in potency, Table 3).

These data indicate that the N-glycosylated IL-2 variant protein 1a displays similar IL-2R βγ activity (as reflected by CD8+ and NK cells potency) but reduced IL-2R αβγ activity (as reflected by Treg potency) as compared to wild type IL-2 (1d). These data demonstrate the “non-IL-2Rα” bias of protein 1a.

### Example 4: Bioactivity of (His Tag Removed) CHO Produced Glycosylated IL2 in Human Primary Cell pSTAT5 Assays

Bioactivity of control wild type IL2 protein 1d as well as glycoengineered IL2 protein 1f from CHO cell expression from Example 2 were tested by an external vendor using human blood. Phosphorylation of STAT5 (pSTAT5), a proximal IL2 receptor signaling biomarker, was examined after treatment of samples with protein 1d or protein 1f. Heparinized human whole blood was drawn and equilibrated to room temperature, aliquoted to plates and warmed to 37° C. prior to addition of protein 1d or protein 1f. A dose titration of protein 1d or protein 1f was prepared and prewarmed to 37° C. before addition to cells. After addition of protein 1d or protein 1f, cells were incubated at 37° C. for 30 minutes. At the end of the incubation period, the red blood cells are lysed and the cells fixed. Following washing the cells are then stained for surface antigens, washed and permeabilized with methanol followed by washes and intracellular staining for pSTAT5 and additional lineage markers. The following markers were used to define cell populations of interest: CD8+ T cells were defined as CD3+ CD4- CD8+; CD4+ T cells were defined as CD3+ CD4+ CD8-; Regulatory T cells (Tregs) were defined as CD3+ CD4+ CD8- CD25+ FOXP3+; Natural Killer (NK) cells were defined as CD3- CD56 or CD16+.

### Results

Half maximal effective concentrations (EC50s) for CD8+ T cells, NK cells and Tregs were determined after treatment with either wild type IL-2 control (1d) or N-glycosylated IL2 (1f). Importantly, CD8+ T cells and NK cells express low resting levels of the high affinity alpha subunit of the IL-2 Receptor (IL2Rα/CD25) while Tregs express high levels of IL2Rα (CD25). For this reason analysis in changes in relative potency of IL-2 variants between CD8+ T cells or NK cells vs Tregs is a good measure of the activity to IL-2R βγ (as reflected by CD8+ and NK cells potency) vs activity to IL-2R αβγ as reflected by Treg potency. We examined the potency across these three cell subsets by quantifying both the median fluorescence intensity (MFI) of pSTAT5 as well as the percentage of pSTAT5 positive cells. A summary of the pSTAT5 MFI EC50 values is shown in Table 4:

TABLE 4

Ave EC50 Values (ng/ml) based on pSTAT5 MFI values

| Population | WT-IL2 (1d) | N-glycosylated IL2 (1a) (1f) | Fold reduction in potency |
|---|---|---|---|
| CD8+ T Cells | 25.70 | 54.79 | 2.13 |
| NK Cells | 6.02 | 7.35 | 1.22 |
| T Regulatory Cells | 0.09 | 33.47 | 371.83 |

Using the pSTAT5 MFI metric, N-glycosylated IL-2 protein 1f demonstrated similar potency as compared to wild type IL-2 control for CD8+ T cells and NK cells (2.13 and 1.22 fold reduction in potency vs wild type IL-2, respectively, Table 4). However, N-glycosylated IL-2 protein 1f demonstrated strikingly reduced potency as compared to wild type IL-2 control for Tregs (371.83 fold reduction in potency, Table 4).

These data indicate that the N-glycosylated IL-2 variant protein 1f displays similar IL-2R βγ activity (as reflected by CD8+ and NK cells potency) but reduced IL-2R αβγ activity (as reflected by Treg potency) as compared to wild type IL-2 (1d). These data demonstrate the “non-IL-2Rα” bias of protein 1f expressed in CHO cells.

### Example 5: Expression of Glycosylated IL2 in CHO Cells

### Vector Construction

The amino acid sequences of protein 2a (SEQ ID NO:237) and protein 1f (SEQ ID NO:10) were backtranslated and codon optimized for expression in CHO cells, each in combination with three different N-terminal secretion signal peptides (signal peptide 3a (SEQ ID NO:238), signal peptide 3b (SEQ ID NO:239) and signal peptide 3c (SEQ ID NO:240)), resulting in protein 2b (SEQ ID NO:247; signal peptide 3a + protein 2a), protein 2c (SEQ ID NO:251; signal peptide 3c + protein 2a), protein 2d (SEQ ID NO:249; signal peptide 3b + protein 2a), protein 2e (SEQ ID NO:248; signal peptide 3a + protein 1f), protein 2f (SEQ ID NO:252; signal peptide 3c + protein 1f) and protein 2 g (SEQ ID NO:250; signal peptide 3b + protein 1f).

The resulting DNA sequences encoding protein 2b (SEQ ID NO:241), protein 2c (SEQ ID NO:243), protein 2d (SEQ ID NO:242), protein 2e (SEQ ID NO:244), protein 2f (SEQ ID NO:246) and protein 2 g (SEQ ID NO:245) were synthesized and cloned into the expression vector pPur. The resulting plasmids were maxi-prepared from E.coli DH5 alpha cultures (Invitrogen), using PureLink HiPure Plasmid Maxiprep Kit (Invitrogen).

### Protein Expression

CHO cells were transfected with the plasmids described above and stable cell pools (pool 4a: expressing protein 2b; pool 4b: expressing protein 2c; pool 4c: expressing protein 2d; pool 4d: expressing protein 2e; pool 4e: encoding protein 2f; and pool 4f: expressing protein 2 g) were developed. For protein expression, stable pools were cultivated in fed-batch mode in shake flasks. Fed-batch cultivations were terminated on Day 10, except for the pool 4f, for which the cultivation was terminated on Day 7. Cell number and viability were assessed using the Guava^{®} Easycyte HT (Merck Millipore). The stable cell pools (pools 4a, 4b, 4c, 4d, 4e, and 4f) were also cultivated in Ambr15 Bioreactors (Sartorius) in fed-batch mode using commercial growth and feed media, for a total of 14 days. Cell number and viability were analyzed by BioProfile® FLEX2 (Nova).

### Analytical Methods

IL-2 protein in the supernatant was quantified with ELISA (IL-2 Human kit, Abcam #ab100566, Lot #GR3344842) and qualitatively analyzed by Western Blot, using Rabbit anti-human IL-2 (Abcam, Cat. Ab9618) as the primary antibody and Donkey anti-rabbit IgG-HRP (Jackson Cat. 711-036-152) as the secondary antibody. Western Blot detection was by absorbance using TMB (3,3′,5,5′-tetramethylbenzidine) substrate. As size references in Western Blot analysis, a molecular weight ladder (Novex® Sharp Pre-Stained Protein Standard, Invitrogen, Cat no LC5801) and recombinant E.coli-derived hIL-2 (Abcam, Cat no ab9619) were used.

### Protein Purification

Process 1: IL-2 variants were purified by loading of clarified CM on a Superdex 200 16/600 size-exclusion chromatography column (GE Healthcare). Fractions were collected and analyzed by SDS-PAGE and Coomassie Blue staining. Bands corresponding to monomeric IL2 were combined and buffer exchanged for loading on a HiScreen Capto Q anion-exchange chromatography column (GE Healthcare). Proteins were eluted by applying a linear NaCl gradient, followed by a second linear NaCl gradient. Fractions were collected and analyzed by SDS-PAGE and Coomassie Blue staining to identify bands corresponding to monomeric IL-2. Purified IL-2 was concentrated and buffered exchange into storage and/or assay buffers for subsequent experiments.

Process 2: IL-2 variants were alternatively purified using a process consisting of 4 column steps. Here, clarified CM was pH -adjusted to pH 6.0 and concentrated. The cell supernatant was then loaded on an HiScreen Capto MMC column (GE Healthcare). After washing the column, IL-2 proteins were eluted and a subsequent linear NaCl gradient was performed. Fractions were collected and analyzed by SDS-PAGE and Coomassie Blue staining. Bands corresponding to monomeric IL2 were combined and pH-adjusted to pH 7.4 for loading on a HiScreen Capto Blue chromatography column (GE Healthcare). Proteins were eluted by applying a step NaCl gradient, followed by a linear NaCl Fractions were collected and analyzed by SDS-PAGE and Coomassie Blue staining to identify fractions containing to monomeric IL-2. The combined IL-2 fractions from the Capto Blue run were loaded on a HiScreen Capto Adhere column (GE Healthcare) and purified in a flow through chromatography step. Monomeric IL-2 fractions were identified by SDS-PAGE and Coomassie staining, buffer exchanged and concentrated. The IL-2 protein sample was then loaded on a HiScreen Capto MMC column (GE Healthcare) and purified as described above. Purified IL-2 was concentrated to ~0.5 mg/mL and buffer exchanged into storage and/or assay buffers for subsequent experiments.

### Results

For expression in shake flasks, the concentration of recombinant IL-2 protein in the culture supernatants of cell lines expressing protein 2a (4a, 4b and 4c) and protein 1f (4d, 4e and 4f) were measured by ELISA on Day 8 and Day 10 of the fed-batch cultivations (Table 5). Both on Day 8 and Day 10, the concentrations of protein 1f were several-fold higher than concentrations of protein 2a. For the constructs with signal peptide 3a, the expression level of protein 1f was 11.0-fold higher than protein 2a on Day 8 and 10.0-fold higher on Day 10; for the constructs with signal peptide 3c the fold-change was 3.7-fold on Day 8 and 6.0-fold on Day 10 and for the constructs with signal peptide 3b expression level of protein 1f on Day7 was 12.9-fold higher than protein 2a expression level on Day 8.

TABLE 5

Cell concentrations, Viability and Titer of IL-2 mutein on Day 8 and Day 10 of fed-batch cultivations. *4f was terminated on Day 7, therefore values for Day 7 are presented for this construct

| | Day7/Day 8 | | | Day 10 | | |
|---|---|---|---|---|---|---|
| | Cell concentration (cells/mL) | Viability (%) | Titer (mg/L) | Cell concentration (cells/mL) | Viability (%) | Titer (mg/L) |
| 4a | 2.46E+09 | 96.7 | 47 | 2.49E+07 | 97.4 | 84 |
| 4b | 3.98E+07 | 94.3 | 82 | 5.44E+07 | 95.0 | 108 |
| 4c | 8.33E+06 | 91.4 | 15 | 8.45E+06 | 88.6 | 20 |
| 4d | 3.00E+07 | 95.2 | 516 | 3.31E+07 | 97.3 | 836 |
| 4e | 1.78E+07 | 94.0 | 302 | 1.91E+07 | 93.5 | 651 |
| 4f* | 8.01E+06 | 82.6 | 194 | NA | NA | NA |

Western blots of non-reduced samples from 4a, 4b and 4c culture supernatants from Day 10 of the fed-batch cultivation showed a double band at a size of 14-15 kDa, corresponding well with the expectation of non-glycosylated and O-glycosylated forms of IL-2 being produced by CHO cells. The lower product band, corresponding to non-glycosylated protein 2a product, had the same size as non-glycosylated, E.coli-derived wt hIL-2 reference standard loaded on the same gel. In contrast, Western blot of non-reduced samples from 4d (Day 10), 4e (Day 10) and 4f (Day 7) showed a clear upward shift in product size compared to the 4a, 4b and 4c samples and hIL-2 reference standard, indicating efficient N-glycosylation of protein 1f. The shift was nearly complete, as only a weak, barely visible band was present at the size corresponding to non-glycosylated hIL-2. The product band of 4d, 4e and 4f also had the form of a double band on the Western Blot, indicating that the N-glycosylated product contained a fraction of O-glycosylated product. In addition to the described bands, a weaker product band of approximately twice the size of the main product band was also detected on the Western blots for both protein 2a (4a, 4b and 4c) and protein 1f (4d, 4e and 4f), indicating some dimer formation.

For expression in Ambr15 bioreactors, the concentration of recombinant IL-2 mutein in the culture supernatants of cell lines expressing protein 2a (4a, 4b and 4c) and protein 1f (4d, 4e and 4f) were measured by ELISA on Day 10 and Day 14 of the fed-batch cultivations (Table 6). Both on Day 10 and Day 14, the concentrations of protein 1f were several-fold higher than concentrations of protein 2a. For the constructs with signal peptide 3a, the expression level of protein 1f was 10.1-fold higher than protein 2a on Day 10 and 10.9-fold higher on Day 14 and for the constructs with signal peptide 3c the fold-change was 4.5-fold on Day 10 and 2.8-fold on Day 14. For the constructs with signal peptide 3b the protein 2a titer was very low (0.03 g/L) at Day 10 and undetectable Day 14, while it was substantial for protein 1f at Day 10 and Day 14 (0.56 g/L and 1.1 g/L, respectively).

TABLE 6

Cell concentrations, Viability and Titer of IL-2 mutein on Day 8 and Day 10 of fed-batch cultivations in Ambr15 Bioreactors

| | Day10 | | | Day 14 | | |
|---|---|---|---|---|---|---|
| | Cell concentration (×10⁶ cells/mL) | Viability (%) | Titer (g/L) | Cell concentration (×10⁶ cells cells/mL) | Viability (%) | Titer (g/L) |
| 4a | 32.73 | 95.8 | 0.09 | 23.90 | 83.0 | 0.23 |
| 4b | 37.65 | 93.3 | 0.19 | 24.59 | 76.4 | 0.70 |
| 4c | 2.75 | 24.6 | 0.03 | 0.07 | 0.9 | 0.00 |
| 4d | 34.39 | 94.5 | 0.91 | 23.63 | 77.1 | 2.50 |
| 4e | 29.57 | 95.1 | 0.86 | 23.14 | 84.1 | 1.93 |
| 4f | 28.03 | 93.0 | 0.56 | 18.48 | 70.6 | 1.10 |

### Example 6: Identity Analysis of Glycosylated IL-2 Expressed in CHO Cells

The identity of protein 1f (SEQ ID NO:10) was confirmed performing a peptide map. The wildtype occurring O-glycosylation site was identified and the heterogeneous glycosylation pattern characterized. The introduced N-glycosylation site was identified and the heterogeneous glycosylation pattern characterized. No “free” LTFGNSTK could be detected (within the detection limit), indicating that all IL-2 variants carry an N-glycosylation at the designated introduced N-glycosylation site.

### Example 7: SPR Analysis of IL-2 Compounds

Binding kinetics of protein 5a (IL-2 of SEQ ID NO:213; Peprotech, cat # AF-200-02) and protein 1f (SEQ ID NO:1 0) were assessed using surface plasmon resonance (SPR) spectroscopy with a Biacore instrument (T200, GE Healthcare) with immobilized extracellular domains of the IL-2 receptor subunits. In short, carboxymethylated dextran biosensor chips (CM5, GE Healthcare) were activated with N-(3-Dimethylaminopropyl)-N′-ethylcarbodiimide (EDC) and N-hydroxysuccinimide (NHS) according to the supplier’s instructions. Immobilization of a monoclonal mouse anti-human IgG (Fc) antibody was also performed according to the supplier’s instructions (GE Healthcare, order number BR-1008-39).

For the determination of binding kinetics to IL-2Rα the following chip preparation was used: Human IL-2 Receptor alpha, Fc-Tag (Symansis, New Zealand) was diluted in HBS-EP running buffer (GE Healthcare, 10 mM HEPES, 150 mM NaCl, 3 mM EDTA, 0.05% Surfactant P20, pH 7.4) to approx. 0.67 µg/mL and immobilized at a flow rate of 50 µL/min for 60 s to achieve 80 - 100 response units (RU).

For the determination of binding kinetics to IL-2Rβ the following chip preparation was used: Human IL-2 Receptor beta, Fc-Tag (Symansis, New Zealand) was diluted in HBS-EP running buffer (GE Healthcare, 10 mM HEPES, 150 mM NaCl, 3 mM EDTA, 0.05% Surfactant P20, pH 7.4) to around 0.80 µg/mL and immobilized at a flow rate of 50 µL/min for 60 s to achieve 140 - 180 response units (RU).

For kinetic measurements on both receptor subunit setups, protein 5a or protein 1f were analysed in multi-cycle kinetics and therefore injected in at least five different concentrations in HBS-EP running buffer at 25° C. (50 µL/min flow rate, 60 s contact time, 300 s dissociation time). Double referenced data (subtracted data from the reference flow cell and the buffer only samples) was analysed via a kinetic fit (1:1 binding model) to determine K_{D}, kₐ and k_{d}. Regeneration after each cycle was performed with 3 M MgCl₂.

The obtained data is summarized in Table 7.

TABLE 7

Summary of SPR binding data to IL-2 receptor subunits

| Compound | K_{D} to IL-2Rα [nM] | K_{D} to IL-2Rβ [nM] | K_{D}-Ratio (α/β) |
|---|---|---|---|
| protein 5a | 8.32 | 427 | 0.02 |
| protein 1f | > 4000 | 993 | > 4.02 |

These results show that protein 1f has a strongly decreased affinity for IL-2Rα compared to protein 5a, while the affinity towards IL-2Rβ is in a similar range.

### Example 8: Calculation of Bias Ratio

To test whether protein 1f is a biased IL-2, it was calculated whether the following requirement is met

$$\frac{{\text{Ratio}}_{\text{protein} \text{1f}}}{{\text{Ratio}}_{\text{protein} \text{5a}}} > 1$$

wherein

$${\text{Ratio}}_{\text{protein} \text{1f}} \text{=} \frac{{\text{K}}_{\text{D}} \text{protein} \text{1f} \text{to} \text{IL-2R} \text{α}}{{\text{K}}_{\text{D}} \text{protein} \text{1f} \text{to} \text{IL-2R} \text{β}}$$

$${\text{Ratio}}_{\text{protein} \text{5a}} \text{=} \frac{{\text{K}}_{\text{D}} \text{protein} \text{5a} \text{to} \text{IL-2R} \text{α}}{{\text{K}}_{\text{D}} \text{protein} \text{5a} \text{to} \text{IL-2R} \text{β}}$$

with
“K_{D} protein 1f to IL-2Rα” being the K_{D} of protein 1f to IL-2Rα,
“K_{D} protein 1f to IL-2Rβ” being the K_{D} of protein 1f to IL-2Rβ,
“K_{D} protein 5a to IL-2Rα” being the K_{D} of protein 5a to IL-2Rα, and
“K_{D} protein 5a to IL-2Rβ” being the K_{D} of protein 5a to IL-2Rβ.

Using the above-mentioned formula, protein 1f is clearly a biased IL-2, as the calculated ratio of Ratioₚᵣₒₜₑᵢₙ 1f to Ratioₚᵣₒₜₑᵢₙ 5a is larger than 200.

### Example 9: Expression of Glycosylated IL-2 Protein 6 in a Clonal Stable CHO Cell Line

To establish a clonal stable CHO cell line expressing protein 6 (SEQ ID NO:10), cells from the stable cell pool 4d (Example 5) were supertransfected with a second expression vector, containing the same IL-2 encoding DNA sequence as the expression vector used to construct cell pool 4d (SEQ ID NO:244, see Example 5), but carrying a different selection marker. Following supertransfection, single clone candidates were isolated using a ClonePix-2 instrument (Molecular Devices) and single clones were screened for growth (Celigo S, Nexcelom) and protein 6 titer in the supernatants (ELISA as described in Example 5). Selected single clones were evaluated by fed-batch cultivation in Ambr15 Bioreactors (Sartorius) using commercial growth and feed media, for a total of 14 days. Cell number, viability and titer of protein 6 were measured over the time-course of the fed-batch cultivations as described in Example 5. Based on cell culture performance and titer of protein 6, the stable clonal cell line 9 was selected as the top clone to be used for further material generation. To verify the stability of clonal cell line 9, cells were passaged in 5 mL spin tubes for a total of 60 generations, and the performance of cells banked at the beginning, mid and end of the study were compared in fed-batch cultivations. The results showed that the cell line was stable, with no decrease in final protein 6 titer over the 60 generations.

The stable clonal cell line 9 expressing protein 6 was cultivated in fed-batch mode in shaking flasks using commercial growth and feed media and harvested on Day10. Cell number and viability were analyzed by a ViCell BLU cell counter (Beckman Coulter).

### Example 10: Purification of Protein 6 From Clonal Stable CHO Cell Line

Protein 6 was purified as described in process 2 of Example 5. The purified protein was buffer exchanged into storage and/or assay buffers and concentrated to 1 mg/mL for subsequent experiments.

### Example 11: Preparation of Conjugate 7

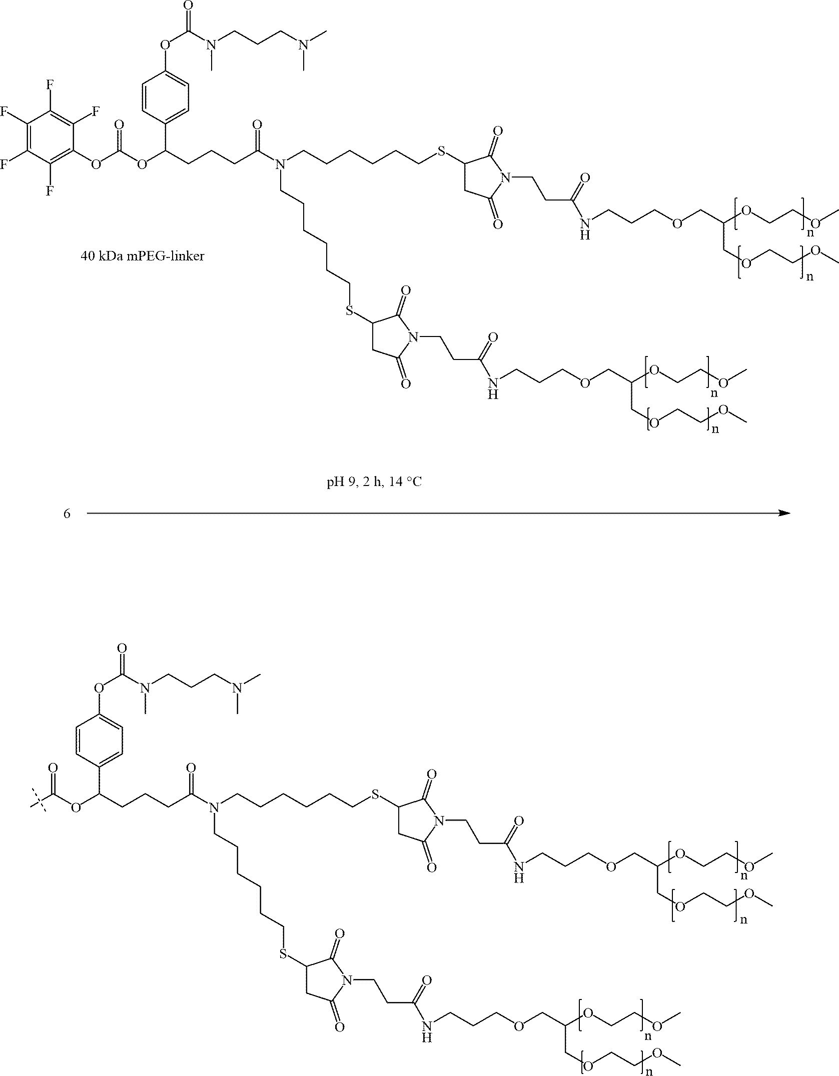

The dashed line indicates attachment to a nitrogen of a primary amine of the N-terminus or a lysine side chain of protein 6 and each n is an integer from 200 to 250.

2.5 mL of protein 6 formulated at 3.47 mg/mL in 20 mM sodium phosphate, 140 mM NaCl, pH 7.4 were buffer exchanged to 100 mM borate, pH 9.0 using a HiPrep 26/10 Desalting column connected to an ÄKTA system. Collected protein solution was concentrated using centrifugal filters (MWCO 3 kDa) to give a final volume of 1.55 mL solution with a concentration of 4.80 mg/mL as determined via photometric concentration determination at 280 nm using an extinction coefficient of 0.662 mL·mg⁻¹·cm⁻¹. 0.0932 g of 40 kDa mPEG-linker reagent (synthesis can be performed as described for the compound 17ca in the patent WO2009/133137 example 7 using compound 16c and 1A from the same patent) were dissolved in 1.07 mL cold water to give a stock solution of 2.1∗10⁻³ mol/L. The solution was stored on ice. 1.55 mL of the protein solution were diluted to 4 mg/mL by addition of 100 mM borate, pH 9.0. Then, 933 µL of the cooled 40 kDa mPEG-linker reagent stock solution were added (corresponding to 4 mol eq. with respect to the protein). The conjugation mixture was placed in a water bath at 14° C. for 2 h. The pH was shifted to pH 4 by addition of 933 µL of water and 3.732 mL of 200 mM sodium acetate, pH 3.6. After incubation at 25° C. overnight, the conjugate with one single 40 kDa mPEG-linker attached (monoconjugate) was isolated from the reaction mixture using a HiScreen Capto MMC ImpRes column connected to an AKTA system. A linear salt gradient from 10 mM succinic acid, pH 5.0 to 10 mM succinic acid, 800 mM NaCl, pH 5.0 in 12 column volumes was applied at 1.2 mL/min. The peak containing mainly monoconjugate eluting during the gradient was collected. NaCl content of this fraction was adjusted to 150 mM by addition of 10 mM succinic acid, pH 5.0. Upon concentration to 2.18 mL at 1.49 mg/mL using centrifugal filters (MWCO 10 kDa), the sample was diluted with 115 µL of 10 mM succinic acid, 150 mM NaCl, 1 % Tween20, pH 5.0 and 963 µL of 10 mM succinic acid, 150 mM NaCl, 0.05 % Tween20, pH 5.0 to a final concentration of 1 mg/mL protein 6 equivalents (based on molecular weight of the protein without taking the glycosylation into account) to create conjugate 7.

### Example 12: Preparation of Conjugate Release Mixture 8

150 µL of conjugate 7 were pH shifted to pH 9.0 by dilution with 62 µL 50 mM boric acid pH 10.0. The sample was incubated at 37° C. in an incubator for 24 hours resulting in conjugate release mixture 8. After incubation the percentage of released protein 6 was determined by RP-HPLC using an Acquity UPLC Peptide BEH C18 column (Waters, 300 Å, 2.1 × 50 mm, 1.7 µm) on a HPLC system. The column temperature was maintained at 30° C. and the flow was set to 0.25 mL/min. UV detection was performed at 215 and 280 nm. The analysis was performed utilizing 0.05 % (V/V) trifluoroacetic acid in water (eluent A) and 0.04 % (V/V) trifluoroacetic acid in acetonitrile (eluent B). HPLC gradient is described in Table 8.

TABLE 8

HPLC Gradient

| Time [min] | Eluent B [%] |
|---|---|
| 0.0 | 33 |
| 0.2 | 33 |
| 2.5 | 46 |
| 10.5 | 59 |
| 14 | 90 |
| 17 | 90 |
| 17.5 | 33 |
| 24 | 33 |

The content of released protein 6 was determined using a calibration curve of purified reference material in six different concentrations applying the same RP-HPLC conditions. UV detection was performed at 280 nm. Conjugate release mixture 8 was used without purification and therefore mainly contains protein 6 and cleaved 40 kDa mPEG-linker as well as >10% residual conjugate 7.

### Example 13: In Vitro Release Kinetics of Protein 6 From Conjugate 7

In vitro release kinetics of protein 6 from conjugate 7 was determined at pH 7.4 and 37° C. to mimic physiological pH and temperature conditions. For this purpose, conjugate 7 was buffer exchanged into 25 mM HEPES, 135 mM NaCl, 1 mM EDTA, 10 mM L-Methionine, 2 mg/mL Pluronic F-68, pH 7.4 using an Äkta system and two connected HiTrap desalting columns (Cytiva) using a flow rate of 2 mL/min and UV detection at 215 and 280 nm. The buffer exchanged samples were incubated at 37° C. under temperature-controlled conditions in a water bath for up to one week (168 h). Determination of linker cleavage and release of protein 6 was performed after acidification of the samples by reversed-phase high pressure liquid chromatography on a 1260 Infinity II system (Agilent Technologies) equipped with an Acquity UPLC Peptide BEH C18 column (300 Å, 1.7 µm, 2.1 mm × 50 mm). The analysis was performed utilizing 0.05 % (V/V) trifluoroacetic acid in water (eluent A) and 0.04 % (V/V) trifluoroacetic acid in MeCN (eluent B). HPLC gradient is described in Table 8 of example 12.

The release was quantified at 5 different time points by integration of the peak of conjugate 7 and release-related species (protein 6 and cleaved 40 kDa mPEG-linker species) in the respective RP-HPLC chromatogram at 215 nm. The percentage of liberated species was plotted against the incubation time and curve fitting software was used to apply a nonlinear one-phase association fit (set plateau at 95%) to determine the half-life time of the linker cleavage kinetics. The in vitro linker half-life time has been determined with 65 hours (95% confidence interval = 52 - 83 hours) at pH 7.4 and 37° C.

### Example 14: Bioactivity of Control IL-2 or Clonal, Stable CHO Produced Protein 6 in Human Peripheral Blood Mononuclear Cell Lymphocyte Subsets

To evaluate the cell type specific immunostimulatory effects of control IL-2 (1d), or clonal, stable CHO produced protein 6 in primary human cells, human peripheral blood mononuclear cells (PBMCs) from two donors were incubated with various concentrations of 1d or 6 for 30 minutes and analyzed by flow cytometry for intracellular levels of phosphorylated STAT5 (pSTAT5) in unique cell subsets such as Tregs which predominately express IL-2Rα/β/γ as well as CD8+ T cells (CD8) and NK cells which predominately express IL-2Rβ/γ. All subsets were analyzed for median fluorescence intensities of pSTAT5 and EC₅₀ values were calculated for each compound across all cell types.

Compared to 1d, 6 demonstrated substantially reduced potency in Tregs while maintaining nearly identical potency to CD8+ T cells and NK cells. For example, in human blood 1d demonstrated an EC₅₀ value for pSTAT5 in Tregs of 0.02 ng/ml while 6 showed a considerably higher EC₅₀ value of 38.01 ng/ml. In contrast, 1d demonstrated an EC₅₀ values for pSTAT5 in human NK cells of 15.91 ng/ml while 6 showed an almost identical EC₅₀ value of 11.66 ng/ml. Similar to results seen for NK cells, 1d demonstrated an EC₅₀ values for pSTAT5 in human CD8+ T cells of 82.98 ng/ml while 6 showed an almost identical EC₅₀ value of 78.82 ng/ml. (Table 9). These results suggest that 6 demonstrates substantially reduced (~1900 fold lower) potency on IL-2Rα/β/γ⁺ cells such as Tregs but maintains potency for IL-2Rβ/γ⁺ cells such as NK cells and CD8⁺ T cells.

TABLE 9

Summary of pSTAT5 Potency Values in Human PBMCs

| EC₅₀ (ng/mL) | Treg | NK | CD8 |
|---|---|---|---|
| 1d | 0.02 | 15.91 | 82.98 |
| 6 | 38.01 | 11.66 | 78.82 |

### Example 15: Receptor Binding of Protein 6 in IL-2Rα and IL2-Rβ Binding Experiments and Calculation of Bias

Binding kinetics of protein 5a (IL-2 of SEQ ID NO:213; Peprotech, cat # AF-200-02) and protein 6 (SEQ ID NO:10) were assessed using surface plasmon resonance (SPR) spectroscopy with a Biacore instrument (T200, GE Healthcare) with immobilized extracellular domains of the IL-2 receptor subunits. In short, carboxymethylated dextran biosensor chips (CM5, GE Healthcare) were activated with N-(3-Dimethylaminopropyl)-N′-ethylcarbodiimide (EDC) and N-hydroxysuccinimide (NHS) according to the supplier’s instructions. Immobilization of a monoclonal mouse anti-human IgG (Fc) antibody was also performed according to the supplier’s instructions (GE Healthcare, order number BR-1008-39).

For the determination of binding kinetics to IL-2Rα the following chip preparation was used: Human IL-2 Receptor alpha, Fc-Tag (Symansis, New Zealand) was diluted in HBS-EP running buffer (GE Healthcare, 10 mM HEPES, 150 mM NaCl, 3 mM EDTA, 0.05% Surfactant P20, pH 7.4) to approx. 0.25 µg/mL and immobilized at a flow rate of 50 µL/min for 60 s to achieve 90 - 110 response units (RU).

For the determination of binding kinetics to IL-2Rβ the following chip preparation was used: Human IL-2 Receptor beta, Fc-Tag (Symansis, New Zealand) was diluted in HBS-EP running buffer (GE Healthcare, 10 mM HEPES, 150 mM NaCl, 3 mM EDTA, 0.05% Surfactant P20, pH 7.4) to around 0.25 µg/mL and immobilized at a flow rate of 50 µL/min for 60 s to achieve 300 - 330 response units (RU).

For kinetic measurements on both receptor subunit setups, protein 5a or protein 1f were analysed in multi-cycle kinetics and therefore injected in at least five different concentrations in HBS-EP running buffer at 25° C. (50 µL/min flow rate, 60 s contact time, 300 s dissociation time). Double referenced data (subtracted data from the reference flow cell and the buffer only samples) was analysed via a kinetic fit (1:1 binding model) to determine K_{D}, kₐ and k_{d}. Regeneration after each cycle was performed with 3 M MgCl₂.

TABLE 10

Summary of SPR binding data to IL-2 receptor subunits

| Compound | K_{D} to IL-2Rα [nM] | K_{D} to IL-2Rβ [nM] | K_{D}-Ratio (α/β) |
|---|---|---|---|
| protein 6 | >2000 | 740 | >2.7 |
| protein 5a | 16.2 | 654 | 0.0248 |

The results show that protein 6 has a strongly decreased affinity for IL-2Rα compared to protein 5a, while the affinity towards IL-2Rβ is in a similar range (Table 10).

To test whether protein 6 is a biased IL-2, it was calculated whether the following requirement is met

$$\frac{{\text{Ratio}}_{\text{protein} \text{6}}}{{\text{Ratio}}_{\text{protein} \text{5a}}} > 1$$

wherein

$${\text{Ratio}}_{\text{protein} \text{6}} \text{=} \frac{{\text{K}}_{\text{D}} \text{protein} \text{6} \text{to} \text{IL-2R} \text{α}}{{\text{K}}_{\text{D}} \text{protein} \text{6} \text{to} \text{IL-2R} \text{β}}$$

$${\text{Ratio}}_{\text{protein} \text{5a}} \text{=} \frac{{\text{K}}_{\text{D}} \text{protein} \text{5a} \text{to} \text{IL-2R} \text{α}}{{\text{K}}_{\text{D}} \text{protein} \text{5a} \text{to} \text{IL-2R} \text{β}}$$

with
“K_{D} protein 6 to IL-2Rα” being the K_{D} of protein 6 to IL-2Ra,
“K_{D} protein 6 to IL-2Rβ” being the K_{D} of protein 6 to IL-2Rβ,
“K_{D} protein 5a to IL-2Rα” being the K_{D} of protein 5a to IL-2Rα, and
“K_{D} protein 5a to IL-2Rβ” being the K_{D} of protein 5a to IL-2Rβ.

Using the above-mentioned formula, protein 6 is clearly a biased IL-2, as the calculated ratio of Ratioₚᵣₒₜₑᵢₙ 6 to Ratioₚᵣₒₜₑᵢₙ 5a is larger than 100.

### Example 16: Bioactivity of a Clonal Stable CHO Produced Protein 6 in HH pSTAT5 and CTLL-2 pSTAT5 Assays

Bioactivity of control wild type IL2 protein 5a as well as glycoengineered IL2 protein 6 was tested by an external vendor using a STAT5 phosphorylation assay (pSTAT5) with the HH and CTLL-2 cell lines. The HH cell line is an IL-2-Rβ/γ expressing human T-lymphoblast cell line derived from the blood of a patient with cutaneous T-cell lymphoma. The CTLL-2 cell line is an IL-2-Rα/β/γ expressing murine T lymphocyte cell line. Half maximal effective concentrations (EC50s) for STAT5 phosphorylation for HH and CTLL-2 cells were measured in duplicates and analyzed with a 4-parameter logistic fit.

TABLE 11

Summary of pSTAT5 EC50 data with HH and CTLL-2 cells

| Compound | EC50 HH cells (µg/mL) | EC50 CTLL-2 (µg/mL) | Ratio (E50 CTLL-2 cells/EC50 HH cell) |
|---|---|---|---|
| Protein 6 | 0.0966 | 3.3945 | 35.1398 |
| Release mixture 8 | 0.0398 | 1.5704 | 39.4573 |
| Protein 5a | 0.0561 | 0.0076 | 0.1355 |

The results show that protein 6 has a strongly decreased potency for IL-2Rα/β/γ⁺ (CTLL-2 cells) compared to protein 5a, while the potency towards IL-2Rβ/γ⁺ (HH cells) is in a similar range (Table 11). Similar results are obtained for conjugate release mixture 8 that also shows strongly decreased potency for IL-2Rα/β/γ⁺ (CTLL-2 cells) compared to protein 5a, while the potency towards IL-2Rβ/γ⁺ (HH cells) is in a similar range (Table 11). Hence, conjugate release mixture 8 shows no significant loss in potency and IL-2R preference compared to protein 6.

To test whether protein 6 is a biased IL-2, it was calculated whether the following requirement is met

$$\frac{{\text{Ratio}}_{\text{protein} \text{6}}}{{\text{Ratio}}_{\text{protein} \text{5a}}} > 1$$

wherein

$${\text{Ratio}}_{\text{protein} \text{6}} \text{=} \frac{\text{EC50} \text{protein} \text{6} \text{to} \text{CTLL-2 cells}}{\text{EC50} \text{protein} \text{6} \text{to} \text{HH cells}}$$

$${\text{Ratio}}_{\text{protein} \text{5a}} \text{=} \frac{\text{EC50} \text{protein} \text{5a} \text{to} \text{CTLL-2 cells}}{\text{EC50} \text{protein} \text{5a} \text{to} \text{HH cells}}$$

with
“EC50 protein 6 to CTLL-2 cell” being the EC50 of protein 6 to CTLL-2 cells,
“EC50 protein 6 to HH cells” being the EC50 of protein 6 to HH cells,
“EC50 protein 5a to CTLL-2 cells” being the EC50 of protein 5a to CTLL-2 cells, and
“EC50 protein 5a to HH cells” being the EC50 of protein 5a to HH cells

Using the above-mentioned formula, protein 6 is clearly a biased IL-2, as the calculated ratio of Ratioₚᵣₒₜₑᵢₙ 6 to Ratioₚᵣₒₜₑᵢₙ 5a is larger than 250. Conjugate release mixture 8 also shows a bias towards IL-2RØ/y.

Abbreviation

| | |
|---|---|
| °C | degree Celsius |
| bp | base pair |
| CAR | Chimeric Antigen Receptor |
| cDNA | complementary DNA |
| CHO | Chinese hamster ovary |
| DNA | Deoxyribonucleic acid |
| DO | Dissolved oxygen |
| h | Hour |
| HEK | Human Embryonic Kidney |
| IL-2 | interleukin-2 |
| IL-2R | interleukin-2 receptor |
| kDa | Kilodalton |
| MES | 4-Morpholineethanesulfonic acid |
| mol eq. | molar equivalents |
| mPEG | methoxypolyethylene glycol |
| MWCO | molecular weight cut-off |
| NaCl | Sodium chloride |
| NK cell | Natural killer cell |
| P. pastoris | Pichia pastoris |
| PBS | Phosphate buffered saline |
| RNA | Ribonucleic acid |
| RP | reversed phase |
| RP-HPLC | Reversed-phase high-performance liquid chromatography |
| SDS-PAGE | Sodium dodecyl sulfate polyacrylamide gel electrophoresis |
| SEC | Size exclusion chromatography |
| TBS | Tris-buffered saline |
| TCEP | (Tris(2-carboxethyl)phosphine hydrochloride) |
| UPLC | Ultra performance liquid chromatography |
| VCD | Viable cell density |

1. An IL-2 protein sequence of formula (I)
(Tag¹)_{y} - (Ala)ₓ - SEQ A - SEQ B - SEQ C - (Tag²)_{z} (I), wherein
SEQ A has at least 89% sequence identity with SEQ ID NO: 1;
SEQ B has at least 76% sequence identity to SEQ ID NO:2 and comprises at least one glycosylation motif;
SEQ C has at least 91% sequence identity to SEQ ID NO:4;
Tag¹ and Tag² are independently a tag moiety;
Ala is an alanine residue;
x is 0 or 1;
y is 0 or 1; and
z is 0 or 1.

2. The IL-2 protein of claim 1, wherein SEQ A is selected from the group consisting of SEQ ID NO: 1 and SEQ ID NO:36.

3. The IL-2 protein of claim 1, wherein the at least one glycosylation motif in SEQ B is an N-glycosylation motif or an O-glycosylation motif.

4. The IL-2 protein of claim 1, wherein the at least one N-glycosylation motif in SEQ B has the amino acid sequence X₁X₂NX₃X₄ (SEQ ID NO:254), wherein
X₁ is any proteinogenic or non-proteinogenic amino acid or is absent;
X₂ is any proteinogenic or non-proteinogenic amino acid or is absent;
N is asparagine;
X₃ is any proteinogenic or non-proteinogenic amino acid except proline; and
X₄ is selected from the group consisting of threonine, serine and cysteine.

5. The IL-2 protein of claim 1, wherein the at least one N-glycosylation motif in SEQ B is FGNST (SEQ ID NO:307) or FANST (SEQ ID NO:331).

6. The IL-2 protein of claim 1, wherein SEQ B is selected from the group consisting of SEQ ID NO:11, SEQ ID NO:14, SEQ ID NO:17, SEQ ID NO:20, SEQ ID NO:23, SEQ ID NO:26, SEQ ID NO:32, SEQ ID NO:35, SEQ ID NO:214 and SEQ ID NO:215.

7. The IL-2 protein of claim 1, wherein SEQ B has the sequence of SEQ ID NO: 11.

8. The IL-2 protein of claim 1, wherein SEQ C is selected from the group consisting of SEQ ID NO:3, SEQ ID NO:4, and SEQ ID NO:212.

9. The IL-2 protein of claim 1, wherein x of formula (I) is 1.

10. The IL-2 protein of claim 1, wherein y and z of formula (I) are 0.

11. The IL-2 protein of claim 1, wherein the IL-2 protein has a sequence selected from the group consisting of SEQ ID NO: 10; SEQ ID NO: 13; SEQ ID NO:16; SEQ ID NO:19; SEQ ID NO:22; SEQ ID NO:25; SEQ ID NO:31; SEQ ID NO:34; SEQ ID NO:217; and SEQ ID NO:225.

12. The IL-2 protein of claim 1, wherein the IL-2 protein has the sequence SEQ ID NO: 10.

13. The IL-2 protein of claim 1, wherein the IL-2 protein is a biased IL-2.

14. A conjugate comprising one or more of the IL-2 proteins of claim 1.

15. The conjugate of claim 14, wherein the conjugate is an IL-2 conjugate or a pharmaceutically acceptable salt thereof of formula (Ia) or (Ib) 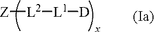 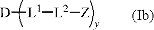
wherein
D comprises the IL-2 protein of formula (I);
L¹- is a linker moiety covalently and reversibly attached to -D;
L²- is a chemical bond or is a spacer moiety;
Z is a polymeric moiety or a substituted fatty acid moiety;
x is an integer selected from the group consisting of 1, 2, 3, 4, 5, 6, 7, 8, 9, 10, 11, 12, 13, 14, 15 and 16; and
y is an integer selected from the group consisting of 2, 3, 4 and 5.

16. (canceled)

17. (canceled)

18. The conjugate or the pharmaceutically acceptable salt thereof of claim 1, wherein -Z is a PEG-based polymeric moiety.

19. (canceled)

20. (canceled)

21. (canceled)

22. (canceled)

23. (canceled)

24. (canceled)

25. The conjugate or the pharmaceutically acceptable salt thereof of claim 14, wherein -L¹- is of formula (IX-a): 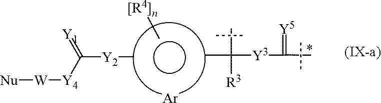
wherein
the dashed line marked with the asterisk indicates attachment to a nitrogen of -D and the unmarked dashed line indicates attachment to -L²-Z;
n is 0, 1, 2, 3, or 4;
═Y₁, is selected from the group consisting of =O and =S;
Y₂— is selected from the group consisting of —O— and —S—;
Y₃— is selected from the group consisting of —O— and —S—;
Y₄— is selected from the group consisting of —O—, —NR⁵— and —C(R⁶R^{6a})—;
═Ys is selected from the group consisting of =O and =S;
R³, -R⁵, -R⁶, -R^{6a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;
R⁴ is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;
W- is selected from the group consisting of C₁₋₂₀ alkyl optionally interrupted by one or more groups selected from the group consisting of C₃₋₁₀ cycloalkyl, 8- to 30-membered carbopolycyclyl, 3- to 10-membered heterocyclyl, —C(O)—, —C(O)N(R⁷)—, —O—, —S— and —N(R⁷)—;
Nu is a nucleophile selected from the group consisting of —N(R⁷R^{7a}), —N(R⁷OH), —N(R⁷)—N(R^{7a}R^{7b}), —S(R⁷), —COOH, 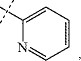 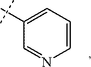 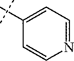 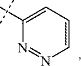 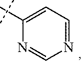 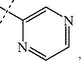 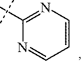 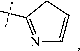 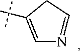 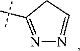 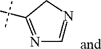 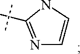
Ar— is selected from the group consisting of 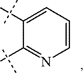 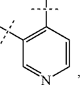 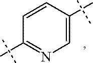 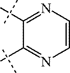 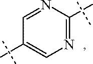 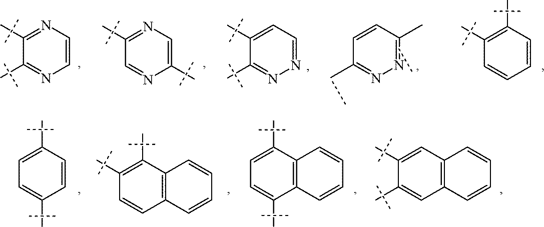 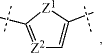 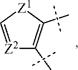 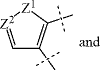 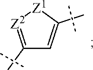
wherein
dashed lines indicate attachment to the remainder of -L¹-,
Z¹- is selected from the group consisting of —O—, —S— and —N(R⁷)—, and
Z²- is —N(R⁷)—; and
R⁷, -R^{7a}, -R^{7b} are independently of each other selected from the group consisting of —H, C₁₋₆ alkyl, C₂₋₆ alkenyl and C₂₋₆ alkynyl;
wherein -L¹- is optionally further substituted.

26. (canceled)

27. (canceled)

28. (canceled)

29. The conjugate or the pharmaceutically acceptable salt thereof of claim 14, wherein -L²- is selected from the group consisting of -T-, —C(O)O—, —O—, —C(O)—, C(O)N(R^{y1})—, —S(O)₂N(R^{y1})—, —S(O)N(R^{y1})—, —S(O)₂—, —S(O)—, -N(R^{y1})S(O)₂N(R^{y1a})-, S—,N(R^{y1})—, -OC(OR^{y1})(R^{y1a})-, -N(R^{y1})C(O)N(R^{y1a})-, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, C(O)N(R^{y3})—, —S(O)₂N(R^{y3})—, —S(O)N(R^{y3})—, —S(O)₂—, —S(O)—, -N(R^{y3})S(O)₂N(R^{y3a})-, S—,N(R^{y3})—, -OC(OR^{y3})(R^{y3a})-, —N(R^{y3})C(O)N(R^{Y3a})—, and —OC(O)N(R^{y3})—;
R^{y1} and -R^{y1a} are independently of each other selected from the group consisting of —H, -T, C₁₋₅₀alkyl, C₂₋₅₀ alkenyl, and C₂₋₅₀ alkynyl; wherein -T, C₁₋₅₀ alkyl, C₂₋₅₀ alkenyl, and C₂₋₅₀ alkynyl are optionally substituted with one or more -R^{y2}, which are the same or different, and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, N(R^{y4})S(O)₂N(R^{y4a})-, —S—, —N(R^{y4})—, -OC(OR^{y4})(R^{y4a})-, -N(R^{y4})C(O)N(R^{y4a})-, and —OC(O)N(R^{y4})—;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8-to 30-membered carbopolycyclyl, and 8- to 30-membered heteropolycyclyl; wherein each T is independently optionally substituted with one or more -R^{y2}, which are the same or different;
each -R^{y2} is independently selected from the group consisting of halogen, —CN, oxo (=O), -COOR^{y5}, —OR^{y5}, —C(O)R^{y5}, -C(O)N(R^{y5}R^{y5a}), -S(O)₂N(R^{y5}R^{y5a}), S(O)N(R^{y5}R^{y5a}), —S(O)₂R^{y5}, —S(O)R^{y5}, -N(R^{y5})S(O)₂N(R^{y5a}R^{y5b}), —SR^{y5}, -N(P^{y5}R^{y5a}), NO₂, —OC(O)R^{y5}, -N(R^{y5})C(O)R^{y5a}, -N(R^{y5})S(O)₂R^{y5a}, -N(R^{y5})S(O)R^{y5a}, N(R^{y5})C(O)OR^{y5a}, -N(R^{y5})C(O)N(R^{y5a}R^{y5b}), -OC(O)N(R^{y5}R^{y5a}), and C₁₋₆ alkyl; wherein C₁₋₆ alkyl is optionally substituted with one or more halogen, which are the same or different; and
each -R^{y3}, -R^{y3a}, -R^{y4}, -R^{y4a}, -R^{y5}, -R^{y5a} and -R^{y5b} is independently selected from the group consisting of —H, and C₁₋₆ alkyl, wherein C₁₋₆ alkyl is optionally substituted with one or more halogen, which are the same or different.

30. (canceled)

31. (canceled)

32. (canceled)

33. A pharmaceutical composition comprising at least one IL-2 protein of claim 1 and at least one excipient.

34. A method of treating, controlling, delaying or preventing in a mammalian patient in need of the treatment of one or more diseases which can be treated with IL-2, comprising the step of administering to the patient in need thereof a therapeutically effective amount of the IL-2 protein of claim 1 .

35. The method of claim 20, wherein the disease which can be treated with IL-2 is cancer .

36-43. (canceled)
